(19) United States
(12) Patent Application Publication
(10) Pub. No.: US 2025/0340556 A1
(43) Pub. Date: Nov. 6, 2025

(54) PYRIMIDOPYRIMIDONE COMPOUNDS AND METHODS OF USE THEREOF

(71) Applicant: Soltego, Inc., Newport Beach, CA (US)

(72) Inventors: Braham Shroot, Antibes (FR); Beatrice Le Varlet, Montelier (FR); Yanke Liang, Belmont, MA (US)

(21) Appl. No.: 18/866,441

(22) PCT Filed: May 17, 2023
(86) PCT No.: PCT/US2023/022555

§ 371 Date: Nov. 15, 2024

Related U.S. Application Data
(60) Provisional application No. 63/342,799, filed on May 17, 2022.

(51) Int. Cl. C07D 487/04; A61K 31/519; C07D 498/18

(52) U.S. Cl. CPC C07D 487/04; A61K 31/519; C07D 498/18

(57) ABSTRACT

Provided are, inter alia, compounds having a structure of Formula (I)-(VI), pharmaceutical compositions including the same, and methods of use. In one aspect, compounds are provided that can inhibit salt-inducible kinases (SIK) and methods of treating a disease or disorder using the compounds. Further provided is a method of producing compounds of Formula (I) or (II).

### CROSS-REFERENCE TO RELATED APPLICATIONS

This application relates to and claims priority from U.S. Patent Application No. 63/342,779 filed on May 17, 2022, the entire disclosure of which is incorporated herein by reference.

### BACKGROUND

Treatments for many dermatological disorders have recognized shortcomings including various side effects and limited effectiveness. Agents that increase skin pigmentation could have many beneficial dermatological effects ranging from improving inflammatory skin disorders and providing sun protection to purely cosmetic applications. It thus would be desirable to have new treatments for dermatological disorders and imperfections.

### SUMMARY

In one aspect, we provide compounds that inhibit salt-inducible kinases for treatment or prevention of dermatological disorders, skin-related disorders in a subject.

In a preferred aspect, provided are compounds having a structure of Formula (I) or (II),

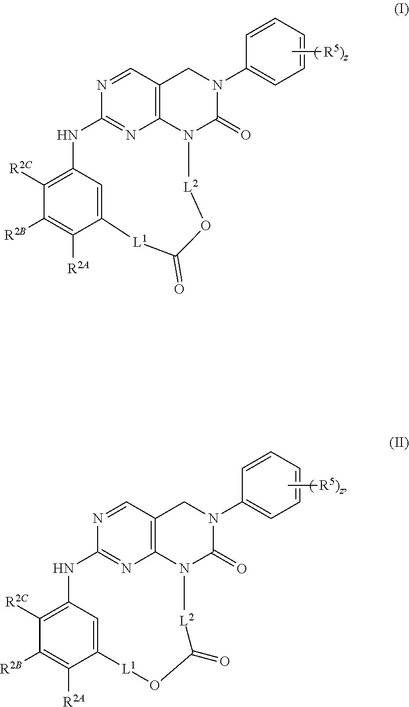

wherein:
each L¹ and L² is independently a bond, substituted or unsubstituted alkylene, or substituted or unsubstituted alkenylene;
each R^{2A}, R^{2B} and R^{2C} is independently hydrogen, halogen, —CX²₃, —CHX²₂, —CH₂X², —OCX², —OCH₂X², —OCHX²₂, —OR^{2F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is independently halogen, —CX⁵₃, —CHX⁵₂, —CH₂X⁵, —OCX⁵₃, —OCH₂X⁵, —OCHX⁵₂, —OR^{5F}, or substituted or unsubstituted C₁-C₄ alkyl;
z is an integer of 0 to 5;
each X² and X⁵ is independently —F, —Br, —Cl, or —I; and
each R^{2F} and R^{5F} is hydrogen, or substituted or unsubstituted alkyl.

In a preferred aspect, provided are compounds having a structure of Formula (III) or (IV), A compound having a structure of Formula (III) or (IV).

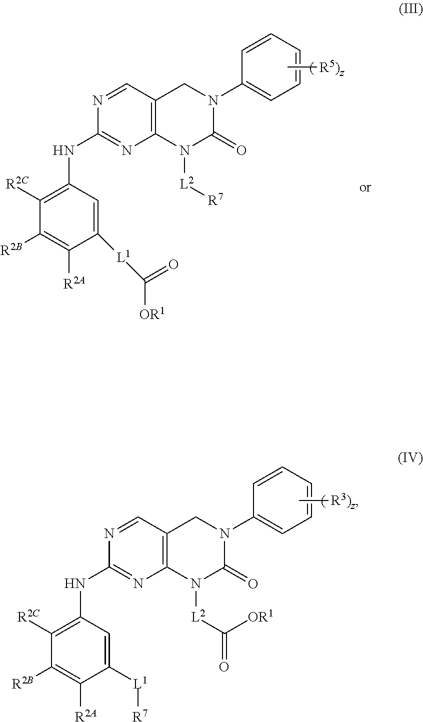

wherein:
R¹ is independently a hydrogen, substituted or unsubstituted alkyl, or substituted or unsubstituted alkenyl;
each L¹ and L² is independently a bond, substituted or unsubstituted alkylene, or substituted or unsubstituted alkenylene;
each R^{2A}, R^{2B} and R^{2C} is independently hydrogen, halogen, —CX²₃, —CHX²₂, —CH₂X², —OCX²₃, —OCH₂X², —OCHX²₂, —OR^{2F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is independently halogen, —CX⁵₃, —CHX⁵₂, —CH₂X⁵, —OCX⁵₃, —OCH₂X⁵, —OCHX⁵₂, —OR^{5F}, or substituted or unsubstituted C₁-C₄ alkyl;
z is an integer of 0 to 5;
R⁷ is —OR^{7F}, or substituted or unsubstituted alkenyl;
each X² and X⁵ is independently —F, —Br, —Cl, or —I; and
each R^{2F}, R^{5F} and OR^{7F} is independently hydrogen, or substituted or unsubstituted alkyl.

In a preferred aspect, provided is a method of producing a compound having the structure of formula (I) or (II),

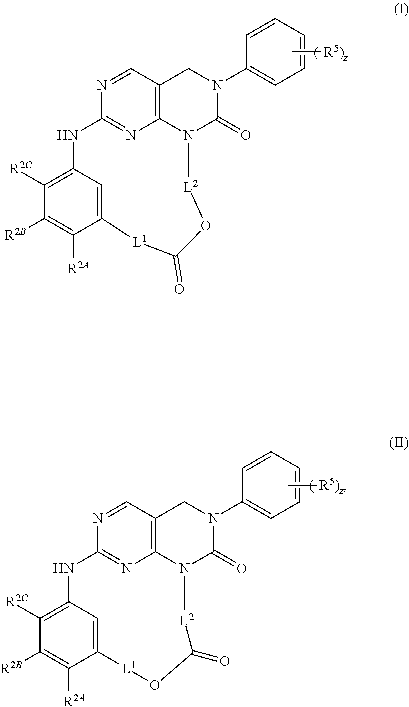

the method comprising:
providing a compound having the structure of formula (III) or (IV),

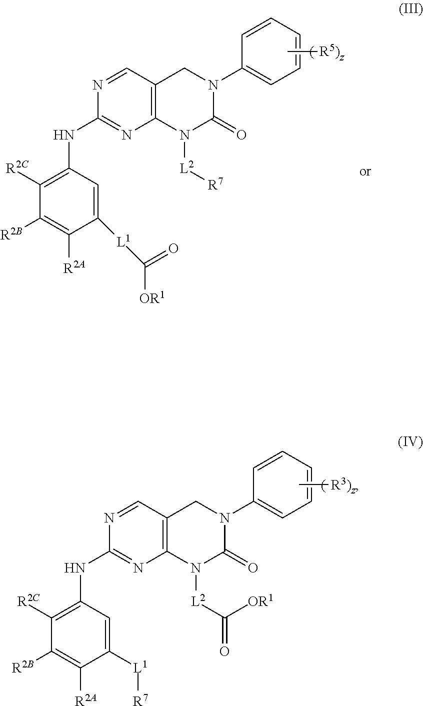

wherein, in Formulae (I) to (IV):
R¹ is independently a hydrogen, substituted or unsubstituted alkyl, or substituted or unsubstituted alkenyl:
each L¹ and L² is independently a bond, substituted or unsubstituted alkylene, or substituted or unsubstituted alkenylene;
each R^{2A}, R^{2B} and R^{2C} is independently hydrogen, halogen, —CX²₃, —CHX²₂, —CH₂X², —OCX²₃, —OCH₂X², —OCHX²₂, —OR^{2F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is independently halogen, —CX⁵₃, —CHX⁵₂, —CH₂X⁵, —OCX⁵₃, —OCH₂X⁵, —OCHX⁵₂, —OR^{5F}, or substituted or unsubstituted C₁-C₄ alkyl;
z is an integer of 0 to 5;
R⁷ is —OR^{7F}, or substituted or unsubstituted alkenyl;
each X² and X⁵ is independently —F, —Br, —Cl, or —I; and
each R^{2F}, R^{5F} and R^{7F} is independently hydrogen, or substituted or unsubstituted alkyl.

In a preferred aspect, provided are compositions, or pharmaceutical compositions including one or more compounds as described herein.

In a preferred aspect, provided are methods for treating a subject suffering from or susceptible to a skin-related disorder or disease. The methods include administering to the subject an effective amount of the compound or the composition as described herein.

In a preferred aspect, provided are methods for treating a subject suffering from or susceptible to rosacea, comprising, administering to the subject an effective amount of the compound or the composition as described herein.

In a preferred aspect, provided are methods of increasing pigmentation in a tissue of a subject, said method comprising administering to the subject the compound or the composition as described herein., in an amount sufficient to increase melanin production, thereby increasing pigmentation in the tissue of the subject.

In a preferred aspect, provided are methods of increasing cellular DNA stability or repair in the skin tissue of a subject in need thereof, comprising administering to the subject the compound or the composition as described herein, in an amount sufficient to decrease apoptosis and/or thymine dimer formation in the cellular DNA of the skin tissue, thereby increasing cellular DNA stability in the skin tissue of the subject.

Other aspects of the inventions are disclosed infra.

### BRIEF DESCRIPTION OF THE DRAWINGS

FIGS. 1A-1B show exemplary macrocyclic lactones.

FIG. 2 depicts effects of the compounds on B16 4A5 melanin content. *=vs untreated control; $=vs DMSO 0.06%.

FIG. 3 depicts the results of melanin staining in the basal layer of the epidermis on all batches.

FIG. 4 depicts the surface percentage of melanin in the basal cell layer and in the suprabasal layers of the epidermis quantified by image analysis.

FIG. 5 depicts the results of melanin staining in the basal layer of the epidermis on all batches.

FIG. 6 depicts the surface percentage occupied by melanin in the basal cell layer of the epidermis.

FIG. 7 depicts Fontana Masson staining images of human ex vivo explants cultured for 9 Days without treatment (A), following UV irradiation (B), vehicle (C), 0.2% SLT-048 (D), 2% SLT-048 (E), magnification of basal and suprabasal melanin capping (F).

FIG. 8 depicts the results of melanin staining in the basal layer of the epidermis following treatment with SLT-045.

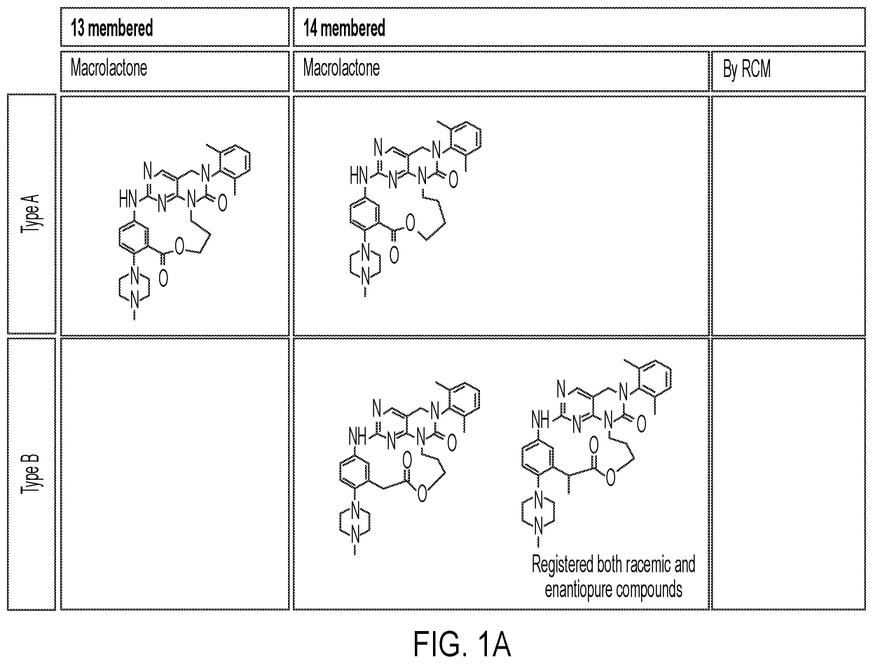

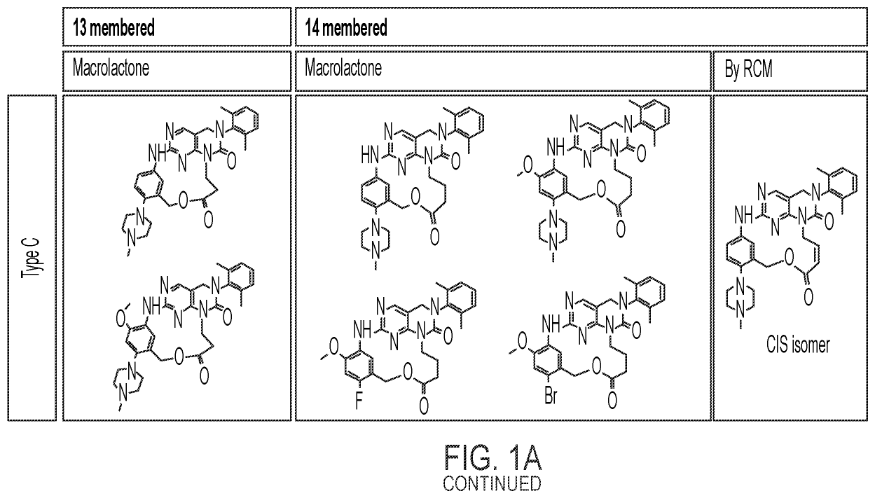

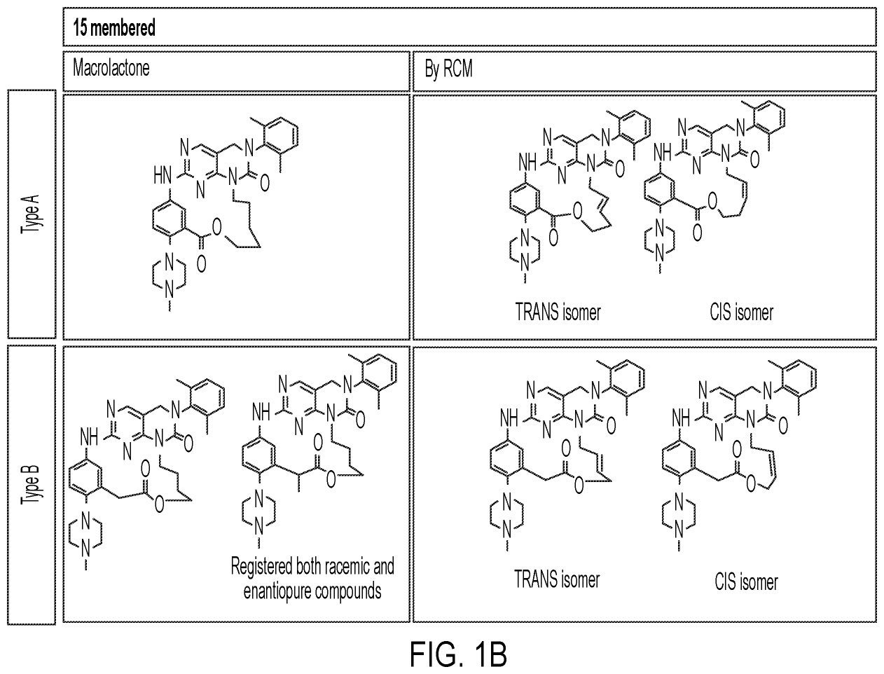

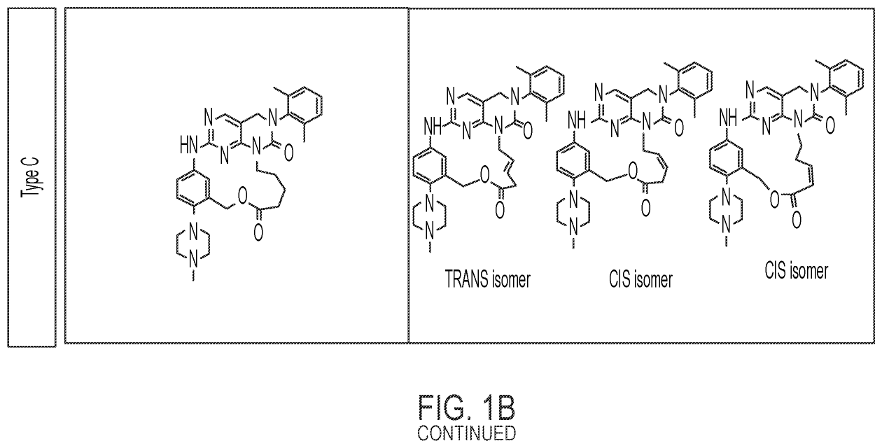

### DETAILED DESCRIPTION

### Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl (“Me”), ethyl (“Et”), n-propyl (“Pr”), isopropyl (“iPr”), n-butyl (“Bu”), t-butyl (“t-Bu”), isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl, or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Similar to the substituents described for the alkyl radical, substituents for the aryl and heteroaryl groups are varied and are selected from, for example: —OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R″′)═NR″″, —NR—C(NR′R″)═NR″′, —S(O)R′, —S(O)₂R′, —S(O)₂NR′R″, —NRSO₂R′, —NR′NR″R′, —ONR′R″, —NR′C(O)NR″NR″′R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R″′, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R″′, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R″′)_{d}—, where s and d are independently integers of from 0 to 3, and X′ is —O—, —NR′—, —S—, —S(O)—, —S(O)₂—, or —S(O)₂NR′—. The substituents R, R′, R″, and R″′ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
(i) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(ii) alkyl (e.g., C₁-C₆ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
(a) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHL, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —S H, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as emoliants, humectants, lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure including, but not limited to, DMSO and Transcutol.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

The term “EC₅₀” or “half maximal effective concentration” as used herein refers to the concentration of a molecule (e.g., small molecule, drug, antibody, chimeric antigen receptor or bispecific antibody) capable of inducing a response which is halfway between the baseline response and the maximum response after a specified exposure time. In embodiments, the EC₅₀ is the concentration of a molecule (e.g., small molecule, drug, antibody, chimeric antigen receptor or bispecific antibody) that produces 50% of the maximal possible effect of that molecule.

The terms “treating”, or “treatment” refers to any indicia of success in the therapy or amelioration of an injury, disease, pathology or condition, including any objective or subjective parameter such as abatement: remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

### Compounds

Provided herein are, inter alia, macrocyclic lactones.

In an aspect, provided is a compound having a structure of Formula (I) or (II),

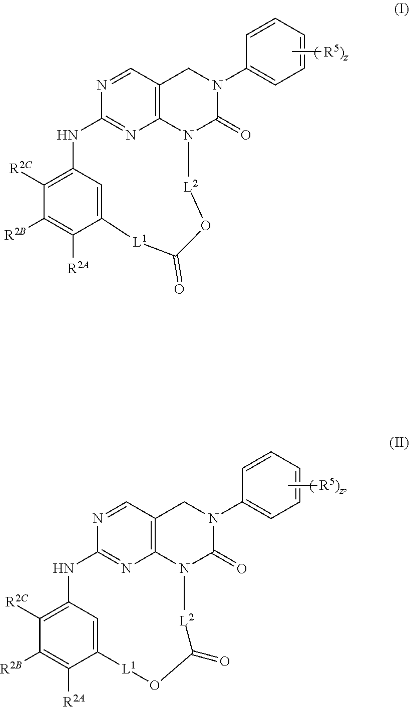

wherein:
   each L¹ and L² is independently a bond, substituted or unsubstituted alkylene, or substituted or unsubstituted alkenylene;
   each R^{2A}, R^{2B} and R^{2C} is independently hydrogen, halogen, —CX²₃, —CHX²₂, —CH₂X², —OCX²₃, —OCH₂X², —OCHX²₂, —OR^{2F} substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R⁵ is independently halogen, —CX⁵₃, —CHX⁵₂, —CH₂X⁵, —OCX⁵₃, —OCH₂X⁵, —OCHX⁵₂, —OR^{5F}, or substituted or unsubstituted C₁-C₄ alkyl,
   z is an integer of 0 to 5;
   each X² and X⁵ is independently —F, —Br, —Cl, or —I; and
   each R^{2F} and R^{5F} is hydrogen, or substituted or unsubstituted alkyl.

In embodiments, z is 1. In embodiments, z is 2. In embodiments, z is 3.

In embodiments, R⁵ is independently unsubstituted C₁-C₄ alkyl. In embodiments, R⁵ is independently unsubstituted methyl. In embodiments, R⁵ is independently unsubstituted ethyl. In embodiments, R⁵ is independently unsubstituted isopropyl. In embodiments, R⁵ is independently unsubstituted propyl. In embodiments, R⁵ is independently unsubstituted butyl. In embodiments, R⁵ is independently unsubstituted t-butyl.

In embodiments, z is 2 and R⁵ is unsubstituted methyl.

In embodiments, the compound has the structure of Formula (I-a) or (II-a),

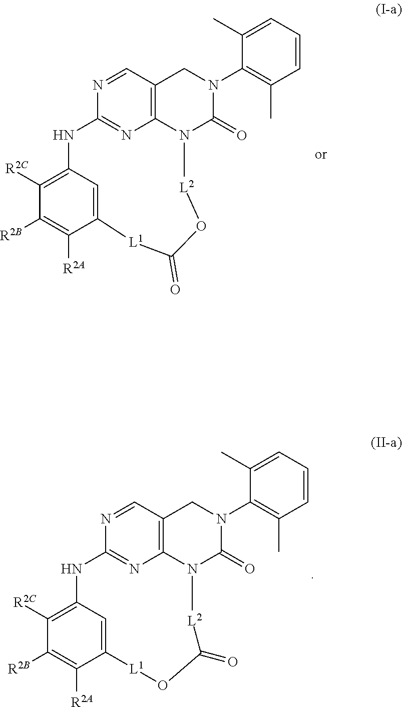

L¹, L², R^{2A}, R^{2B}, and R^{2C} are as described herein.

In embodiments, R^{2A} is substituted or unsubstituted heterocycloalkyl, or substituted or unsubstituted aryl. In embodiments, R^{2A} is substituted or unsubstituted heterocycloalkyl. In embodiments, R^{2A} is substituted or unsubstituted aryl. Ine embodiments, R^{2A} is substituted or unsubstituted piperazinyl. In embodiments, R^{2A} is substituted or unsubstituted phenyl.

In embodiments, R^{2A} is

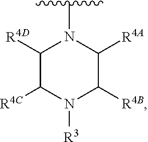

wherein:
R³ is hydrogen or substituted or unsubstituted alkyl;
Each R^{4A}, R^{4B}, R^{4C}, and R^{4D} is independently hydrogen, halogen, —CX⁴₃, —CHX⁴₂, —CH₂X⁴, —OCX⁴₃, —OCH₂X⁴, —OCHX⁴₂, —OR^{4F}, or substituted or unsubstituted alkyl;
X⁴ is independently —F, —Br, —Cl, or —I; and R^{4F} is hydrogen, or substituted or unsubstituted alkyl.

In embodiments, the compound has the structure of Formula (I-b) or (II-b),

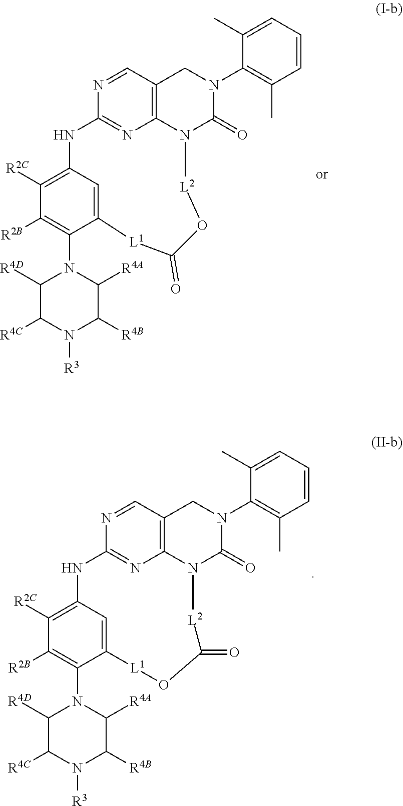

L¹, L², R^{2B}, R^{2C}, R³, R^{4A}, R^{4B}, R^{4C}, and R^{4D} are as described herein.

In embodiments, L¹ is a bond, or R⁶-substituted or unsubstituted C₁-C₄ alkylene. In embodiments, L¹ is a bond. In embodiments, L¹ is substituted or unsubstituted C₁-C₄ alkylene. In embodiments, L¹ is unsubstituted C₁-C₄ alkylene. In embodiments, L¹ is substituted C₁-C₄ alkylene. In embodiments, L¹ is unsubstituted methylene. In embodiments, L¹ is unsubstituted ethylene. In embodiments, L¹ is unsubstituted propylene. In embodiments, L¹ is unsubstituted isopropylene. In embodiments, L¹ is unsubstituted butylene.

In embodiments, L¹ is R⁶-substituted or unsubstituted C₁-C₄ alkylene. In embodiments, R⁶ is halogen, or unsubstituted C₁-C₄ alkylene. In embodiments, L¹ is methyl substituted C₁-C₄ alkylene. In embodiments, L¹ is methyl-substituted methylene. In embodiments, L¹ is methyl-substituted ethylene. In embodiments, L¹ is methyl-substituted propylene. In embodiments, L¹ is methyl-substituted butylene.

In embodiments, the compound has the structure of formula (I-c),

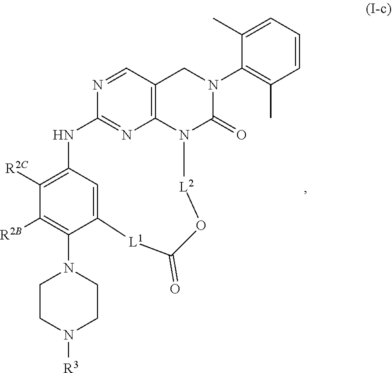

wherein:
   L¹ is a bond, or R⁶-substituted or unsubstituted C₁-C₄ alkylene;
   L² is a substituted or unsubstituted C₂-C₅ alkylene, or substituted or unsubstituted C₂-C₅ alkenylene; and
   R⁶ is halogen, or unsubstituted C₁-C₄ alkylene.

In embodiments, the compound has the structure of formula (II-c),

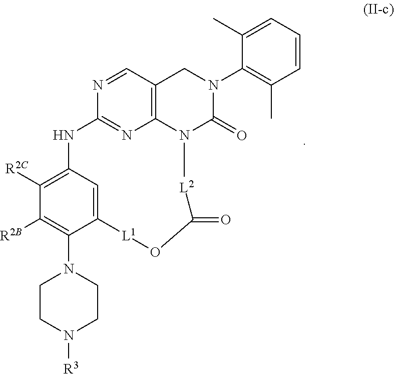

wherein:
   L¹ is a R⁶-substituted or unsubstituted C₁-C₄ alkylene;
   L² is a substituted or unsubstituted C₂-C₅ alkylene, or substituted or unsubstituted C₂-C₅ alkenylene; and
   R⁶ is halogen, or unsubstituted C₁-C₄ alkylene.

Exemplary compounds having a structure of Formula (I-c) or (II-c) are presented in Table 1.

TABLE 1

| Compound | Structure | Compound | Structure |
|---|---|---|---|
| SLT-023 | 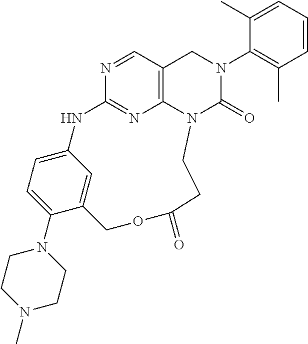 [SMILES: Cc1cccc(C)c1N1Cc2cnc3nc2N(CCC(=O)OCc2cc(ccc2N2CCN(C)CC2)N3)C1=O] | SLT-026 | 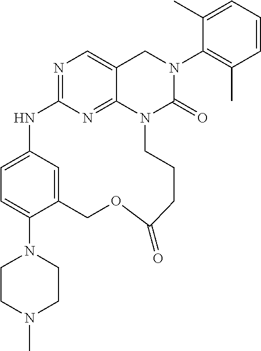 [SMILES: Cc1cccc(C)c1N1Cc2cnc3nc2N(CCCC(=O)OCc2cc(ccc2N2CCN(C)CC2)N3)C1=O] |
| | SLT-023 | | SLT-026 |
| SLT-073 | 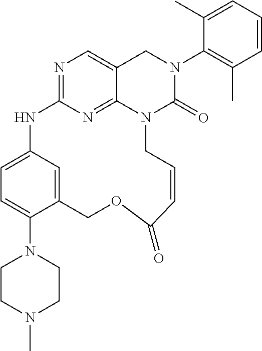 [SMILES: Cc1cccc(C)c1N1Cc2cnc3nc2N(C/C=C\C(=O)OCc2cc(ccc2N2CCN(C)CC2)N3)C1=O] | SLT-033 | 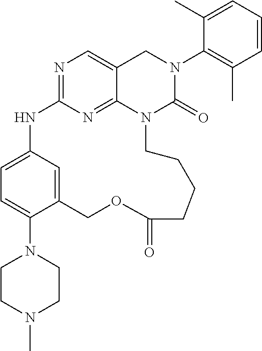 [SMILES: Cc1cccc(C)c1N1Cc2cnc3nc2N(CCCCC(=O)OCc2cc(ccc2N2CCN(C)CC2)N3)C1=O] |
| | SLT-073 | | SLT-033 |
| SLT-062 | 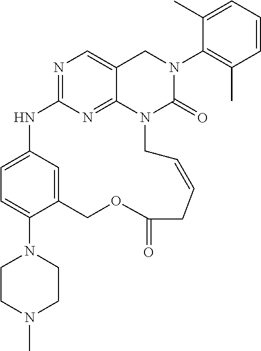 [SMILES: Cc1cccc(C)c1N1Cc2cnc3nc2N(C/C=C\CC(=O)OCc2cc(ccc2N2CCN(C)CC2)N3)C1=O] | SLT-061 | 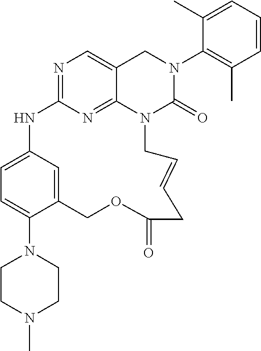 [SMILES: Cc1cccc(C)c1N1Cc2cnc3nc2N(C/C=C/CC(=O)OCc2cc(ccc2N2CCN(C)CC2)N3)C1=O] |
| | SLT-062 | | SLT-061 |
| SLT-088 | 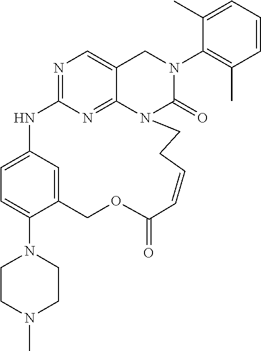 [SMILES: Cc1cccc(C)c1N1Cc2cnc3nc2N(CC/C=C\C(=O)OCc2cc(ccc2N2CCN(C)CC2)N3)C1=O] | SLT-041 | 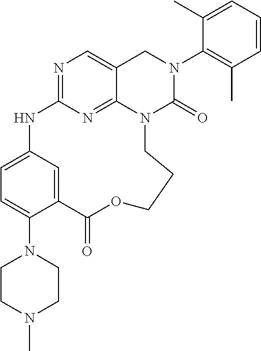 [SMILES: Cc1cccc(C)c1N1Cc2cnc3nc2N(CCCOC(=O)c2cc(ccc2N2CCN(C)CC2)N3)C1=O] |
| | | | SLT-041 |
| SLT-042 | 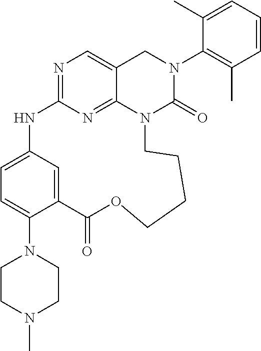 [SMILES: Cc1cccc(C)c1N1Cc2cnc3nc2N(CCCCOC(=O)c2cc(ccc2N2CCN(C)CC2)N3)C1=O] | SLT-038 | 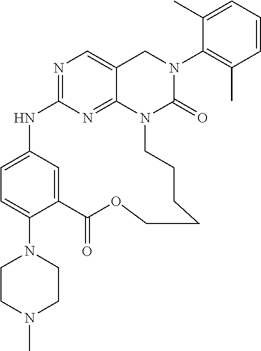 [SMILES: Cc1cccc(C)c1N1Cc2cnc3nc2N(CCCCCOC(=O)c2cc(ccc2N2CCN(C)CC2)N3)C1=O] |
| | SLT-042 | | SLT-038 |
| SLT-089 | 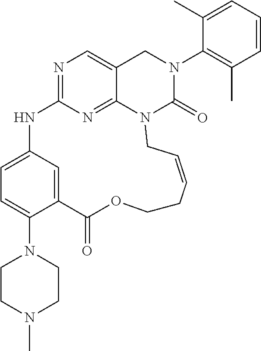 [SMILES: Cc1cccc(C)c1N1Cc2cnc3nc2N(C/C=C\CCOC(=O)c2cc(ccc2N2CCN(C)CC2)N3)C1=O] | SLT-079 | 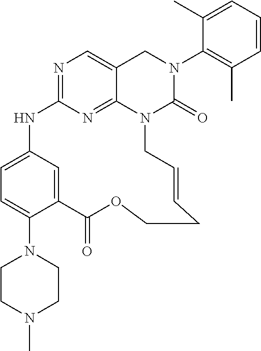 [SMILES: Cc1cccc(C)c1N1Cc2cnc3nc2N(C/C=C/CCOC(=O)c2cc(ccc2N2CCN(C)CC2)N3)C1=O] |
| | | | SLT-079 |
| SLT-048 | 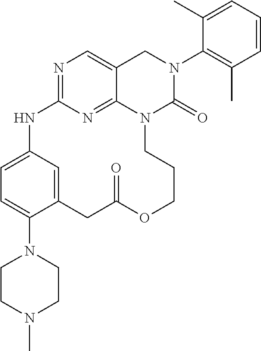 [SMILES: Cc1cccc(C)c1N1Cc2cnc3nc2N(CCCOC(=O)Cc2cc(ccc2N2CCN(C)CC2)N3)C1=O] | SLT-059 | 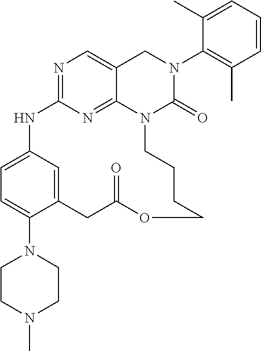 [SMILES: Cc1cccc(C)c1N1Cc2cnc3nc2N(CCCCOC(=O)Cc2cc(ccc2N2CCN(C)CC2)N3)C1=O] |
| | SLT-048 | | SLT-059 |
| SLT-069 | 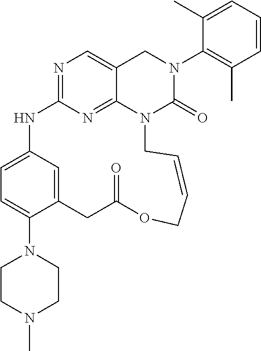 [SMILES: Cc1cccc(C)c1N1Cc2cnc3nc2N(C/C=C\COC(=O)Cc2cc(ccc2N2CCN(C)CC2)N3)C1=O] | SLT-068 | 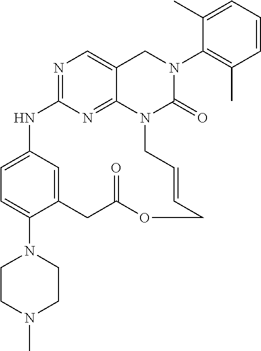 [SMILES: Cc1cccc(C)c1N1Cc2cnc3nc2N(C/C=C/COC(=O)Cc2cc(ccc2N2CCN(C)CC2)N3)C1=O] |
| | SLT-069 | | SLT-068 |
| SLT-071 | 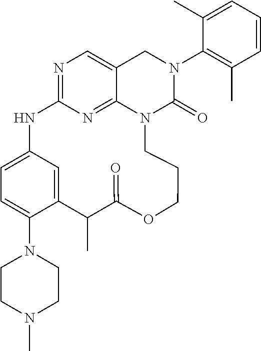 [SMILES: Cc1cccc(C)c1N1Cc2cnc3nc2N(CCCOC(=O)C(C)c2cc(ccc2N2CCN(C)CC2)N3)C1=O] | SLT-070 | 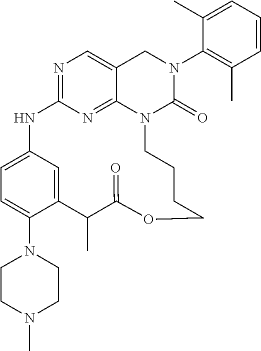 [SMILES: Cc1cccc(C)c1N1Cc2cnc3nc2N(CCCCOC(=O)C(C)c2cc(ccc2N2CCN(C)CC2)N3)C1=O] |
| | SLT-071 | | SLT-070 |

In embodiments, R^{2A} is halogen. In embodiments, R^{ZA} is —F. In embodiments, R^{2A} is —Cl. In embodiments, R^{2A} is —Br. In embodiments, R^{2A} is —I.

In embodiments, exemplary compounds of Formula (I-a) or (II-a) having R^{ZA} of halogen are presented in Table 2.

TABLE 2

| Compound | Structure | Compound | Structure |
|---|---|---|---|
| SLT-032 | 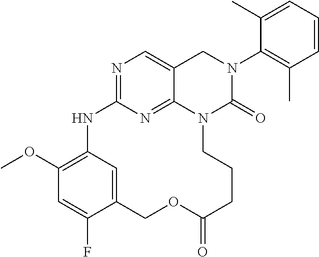 [SMILES: COc1cc(F)c2cc1Nc1ncc3c(n1)N(CCCC(=O)OC2)C(=O)N(c1c(C)cccc1C)C3] | SLT-058 | 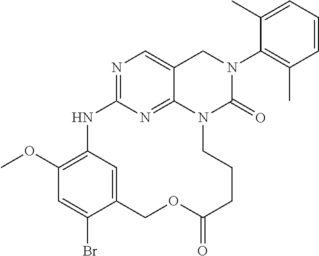 [SMILES: COc1cc(Br)c2cc1Nc1ncc3c(n1)N(CCCC(=O)OC2)C(=O)N(c1c(C)cccc1C)C3] |
| | SLT-032 | | SLT-058 |

In embodiments, each R^{2B} and R^{2C} is independently hydrogen, or —OR^{2F}. In embodiments, R^{2F} is hydrogen or unsubstituted C₁-C₄ alkyl. In embodiments, R^{2B} is hydrogen, —OCH₃, or —OCH₂CH₃. In embodiments, R^{2C} is hydrogen, —OCH₃, or —OCH₂CH₃.

In embodiments, R^{2B} is hydrogen and R^{2C} is —OCH₃. In embodiments, R^{2B} is —OCH₃ and R^{2C} is hydrogen. In embodiments, R^{2B} and R^{2C} are hydrogen. In embodiments, R^{2B} and R^{2C} are —OCH₃.

In embodiments, R³ is hydrogen. In embodiments, R³ is —CH₃.

In an aspect, provided is a compound having a structure of Formula (III) or (IV),

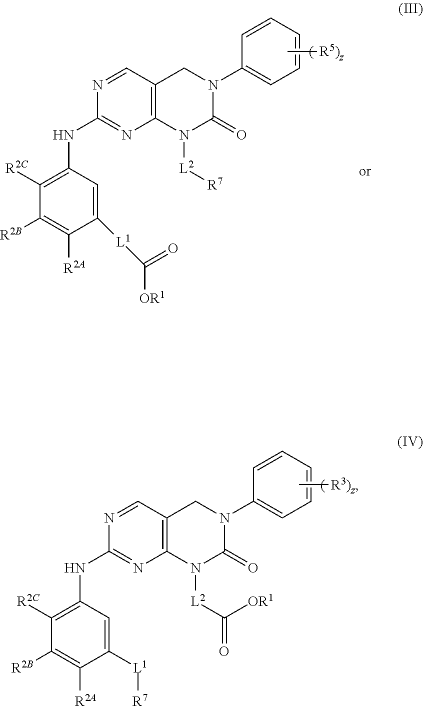

wherein:
   R¹ is a hydrogen, substituted or unsubstituted alkyl, or substituted or unsubstituted alkenyl;
   Each L¹ and L² is independently a bond, substituted or unsubstituted alkylene, or substituted or unsubstituted alkenylene;
   Each R^{2A}, R^{2B} and R^{2C} is independently hydrogen, halogen, —CX²₃, —CHX²₂, —CH₂X², —OCX², —OCH₂X², —OCHX²₂, —OR^{2F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R⁵ is independently halogen, —CX⁵₃, —CHX⁵₂, —CH₂X⁵, —OCX⁵₃, —OCH₂X⁵, —OCHX⁵₂, —OR^{5F}, or substituted or unsubstituted C₁-C₄ alkyl;
   z is an integer of 0 to 5;
   R⁷ is —OR^{7F} or substituted or unsubstituted alkenyl;
   each X² and X⁵ is independently —F, —Br, —Cl, or —I; and
   each R^{2F}, R^{5F} and OR^{7F} is independently hydrogen, or substituted or unsubstituted alkyl.

In embodiments, R¹ is hydrogen, unsubstituted C₁-C₄ alkyl, or R¹ is hydrogen, unsubstituted C₁-C₄ alkyl, or unsubstituted C₁-C₄ alkenyl. In embodiments, R¹ is hydrogen. In embodiments, R¹ is substituted or unsubstituted C₁-C₄ alkyl. In embodiments, R¹ is unsubstituted C₁-C₄ alkyl. In embodiments, R¹ is unsubstituted methyl. In embodiments, R¹ is unsubstituted ethyl. In embodiments, R¹ is unsubstituted propyl. In embodiments, R¹ is unsubstituted isopropyl. In embodiments, R¹ is unsubstituted butyl. In embodiments, R¹ is unsubstituted t-butyl. In embodiments, R¹ is substituted or unsubstituted C₁-C₄ alkenyl. In embodiments, R¹ is unsubstituted C₁-C₄ alkenyl. In embodiments, R¹ is unsubstituted methenyl. In embodiments, R¹ is unsubstituted ethenyl. In embodiments, R¹ is unsubstituted propenyl. In embodiments, R¹ is unsubstituted isopropenyl. In embodiments, R¹ is unsubstituted butenyl. In embodiments, R¹ is unsubstituted t-butenyl.

In embodiments, z is 1. In embodiments, z is 2. In embodiments, z is 3.

In embodiments, R¹ is independently unsubstituted C₁-C₄ alkyl. In embodiments, R⁵ is independently unsubstituted methyl. In embodiments, R⁵ is independently unsubstituted ethyl. In embodiments, R¹ is independently unsubstituted isopropyl. In embodiments, R⁵ is independently unsubstituted propyl. In embodiments, R⁵ is independently unsubstituted butyl. In embodiments, R⁵ is independently unsubstituted t-butyl.

In embodiments, the compound has the structure of Formula (I-a) or (I-a),

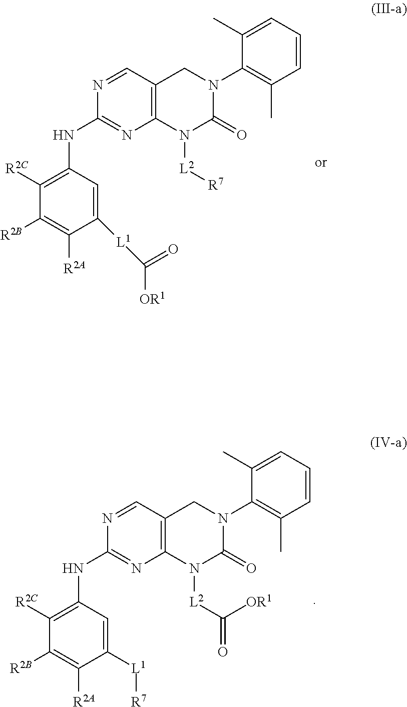

L¹, L², R¹, R^{2A}, R^{2B}, R^{2C}, and R⁷ are as described herein.

In embodiments, R^{2A} is substituted or unsubstituted heterocycloalkyl, or substituted or unsubstituted aryl. In embodiments, R^{2A} is substituted or unsubstituted heterocycloalkyl. In embodiments, R^{2A} is substituted or unsubstituted aryl. Ine embodiments, R^{2A} is substituted or unsubstituted piperazinyl. In embodiments, R^{2A} is substituted or unsubstituted phenyl.

In embodiments, R^{2A} is

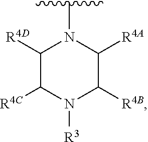

wherein:
R³ is hydrogen or substituted or unsubstituted alkyl;
Each R^{4A}, R^{4B}, R^{4C}, and R^{4D} is independently hydrogen, halogen, —CX⁴₃, —CHX⁴₂, —CH₂X⁴, —OCX⁴₃, —OCH₂X⁴, —OCHX⁴₂, —OR^{4F}, or substituted or unsubstituted alkyl;
X⁴ is independently —F, —Br, —Cl, or —I; and R^{4F} is hydrogen, or substituted or unsubstituted alkyl.

In embodiments, the compound has the structure of Formula (III-b) or (IV-b),

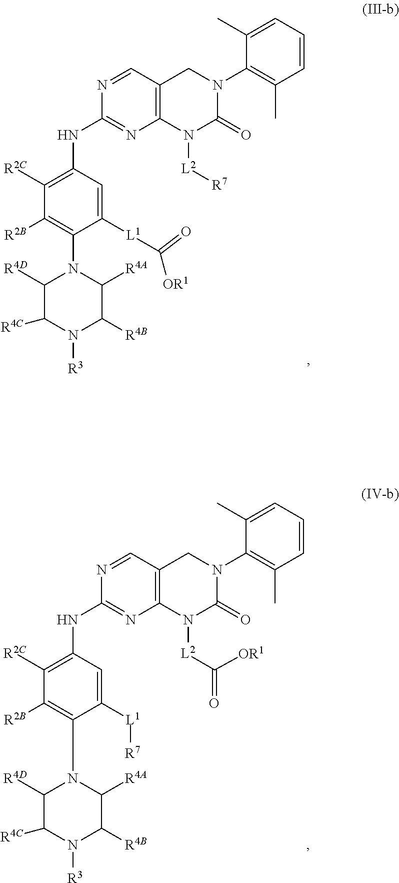

L¹, L², R¹, R^{2B}, R^{2C}, R³, R^{4A}, R^{4B}, R^{4C}, R^{4D}, and R⁷ are as described herein.

In embodiments, L¹ is a bond, or R⁶-substituted or unsubstituted C₁-C₄ alkylene. In embodiments, L¹ is a bond. In embodiments, L¹ is substituted or unsubstituted C₁-C₄ alkylene. In embodiments, L¹ is unsubstituted C₁-C₄ alkylene. In embodiments, L¹ is substituted C₁-C₄ alkylene. In embodiments, L¹ is unsubstituted methylene. In embodiments, L¹ is unsubstituted ethylene. In embodiments, L¹ is unsubstituted propylene. In embodiments, L¹ is unsubstituted isopropylene. In embodiments, L¹ is unsubstituted butylene.

In embodiments, L¹ is R⁶-substituted or unsubstituted C₁-C₄ alkylene. In embodiments, R⁶ is halogen, or unsubstituted C₁-C₄ alkylene. In embodiments, L¹ is methyl substituted C₁-C₄ alkylene. In embodiments, L¹ is methyl-substituted methylene. In embodiments, L¹ is methyl-substituted ethylene. In embodiments, L¹ is methyl-substituted propylene. In embodiments, L¹ is methyl-substituted butylene.

In embodiments, the compound has the structure of formula (III-c),

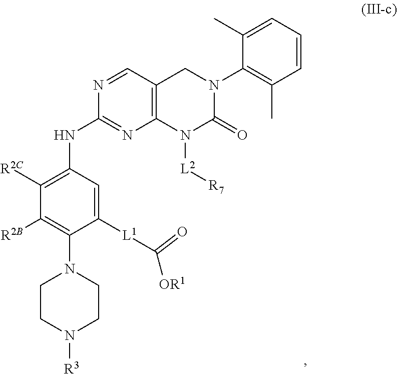

wherein;
L¹ is a bond, or R⁶-substituted or unsubstituted C₁-C₄ alkylene;
L² is a substituted or unsubstituted C₂-C₅ alkylene, or substituted or unsubstituted C₂-C₅ alkenylene; and
R⁶ is halogen, or unsubstituted C₁-C₄ alkylene.
   R¹, R^{1B}, R^{2C}, R³, and R⁷ are as described herein.

In embodiments, the compound has the structure of formula (IV-c),

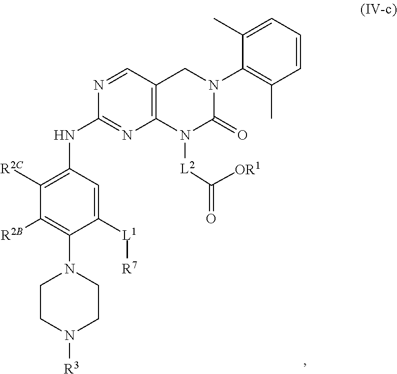

wherein:
   L¹ is a R⁶-substituted or unsubstituted C₁-C₄ alkylene;
   L² is a substituted or unsubstituted C₂-C₅ alkylene, or substituted or unsubstituted C₂-C₅ alkenylene; and
   R⁶ is halogen, or unsubstituted C₁-C₄ alkylene.

R¹, R^{2B}, R^{2C}, R³, and R⁷ are as described herein.

In embodiments, R⁷ is —OH or unsubstituted C₁-C₄ alkenyl. In embodiments, R⁷ is —OH. In embodiments, R⁷ is unsubstituted C₁-C₄ alkenyl. In embodiments, R⁷ is unsubstituted methenyl. In embodiments, R⁷ is unsubstituted ethenyl. In embodiments, R⁷ is unsubstituted propenyl. In embodiments, R⁷ is unsubstituted isopropenyl. In embodiments, R⁷ is unsubstituted butenyl. In embodiments, R⁷ is unsubstituted t-butenyl.

Exemplary compounds having a structure of Formula (III-c) or (IV-c) are presented in Table 3.

TABLE 3

| Compound | Structure | Compound | Structure |
|---|---|---|---|
| | 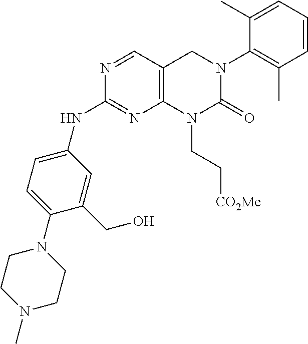 [SMILES: COC(=O)CCN1C(=O)N(c2c(C)cccc2C)Cc2cnc(Nc3ccc(N4CCN(C)CC4)c(CO)c3)nc21] | | 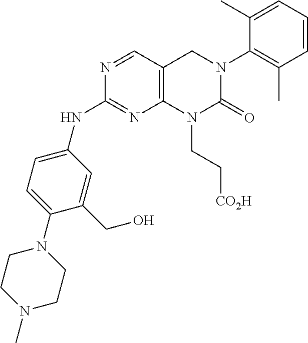 [SMILES: Cc1cccc(C)c1N1Cc2cnc(Nc3ccc(N4CCN(C)CC4)c(CO)c3)nc2N(CCC(=O)O)C1=O] |
| | SLT-053 | | SLT-020 |
| | 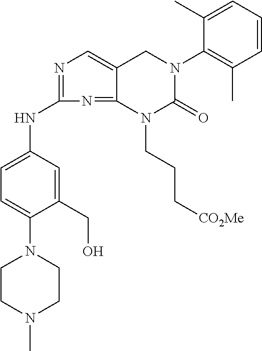 [SMILES: COC(=O)CCCN1C(=O)N(c2c(C)cccc2C)Cc2cnc(Nc3ccc(N4CCN(C)CC4)c(CO)c3)nc21] | | 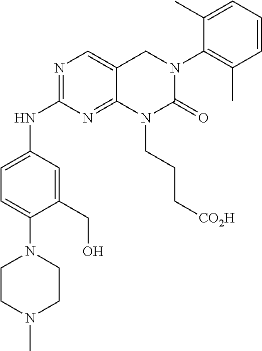 [SMILES: Cc1cccc(C)c1N1Cc2cnc(Nc3ccc(N4CCN(C)CC4)c(CO)c3)nc2N(CCCC(=O)O)C1=O] |
| | SLT-019 | | SLT-025 |
| | 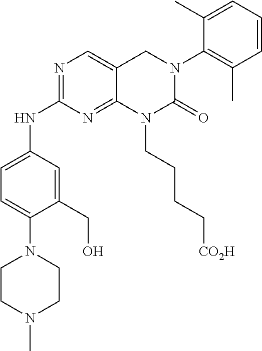 [SMILES: Cc1cccc(C)c1N1Cc2cnc(Nc3ccc(N4CCN(C)CC4)c(CO)c3)nc2N(CCCCC(=O)O)C1=O] | | 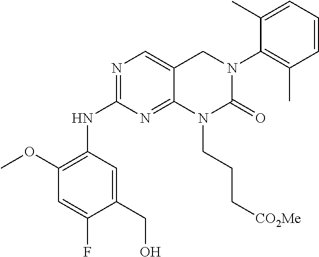 [SMILES: COC(=O)CCCN1C(=O)N(c2c(C)cccc2C)Cc2cnc(Nc3cc(CO)c(F)cc3OC)nc21] |
| | SLT-027 | | SLT-030 |
| | 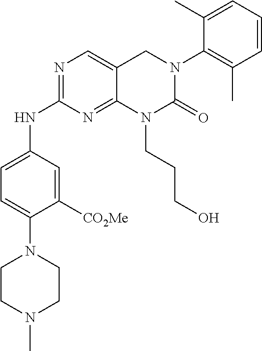 [SMILES: COC(=O)c1cc(Nc2ncc3c(n2)N(CCCO)C(=O)N(c2c(C)cccc2C)C3)ccc1N1CCN(C)CC1] | | 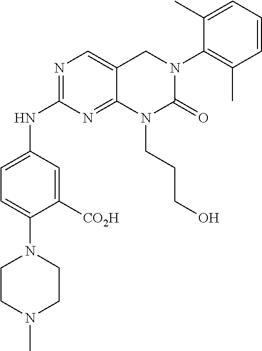 [SMILES: Cc1cccc(C)c1N1Cc2cnc(Nc3ccc(N4CCN(C)CC4)c(C(=O)O)c3)nc2N(CCCO)C1=O] |
| | SLT-039 | | SLT-040 |
| | 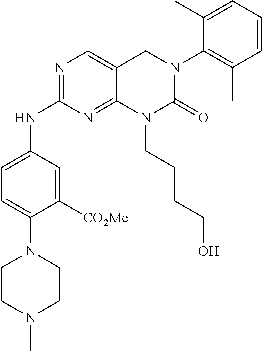 [SMILES: COC(=O)c1cc(Nc2ncc3c(n2)N(CCCCO)C(=O)N(c2c(C)cccc2C)C3)ccc1N1CCN(C)CC1] | | 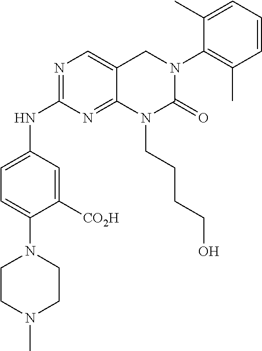 [SMILES: Cc1cccc(C)c1N1Cc2cnc(Nc3ccc(N4CCN(C)CC4)c(C(=O)O)c3)nc2N(CCCCO)C1=O] |
| | SLT-034 | | SLT-035 |
| | 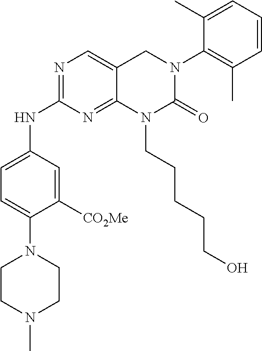 [SMILES: COC(=O)c1cc(Nc2ncc3c(n2)N(CCCCCO)C(=O)N(c2c(C)cccc2C)C3)ccc1N1CCN(C)CC1] | | 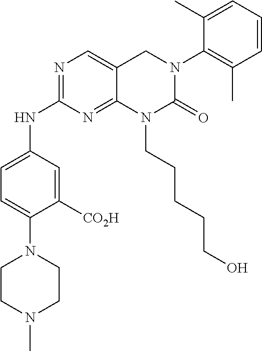 [SMILES: Cc1cccc(C)c1N1Cc2cnc(Nc3ccc(N4CCN(C)CC4)c(C(=O)O)c3)nc2N(CCCCCO)C1=O] |
| | SLT-036 | | SLT-037 |
| | 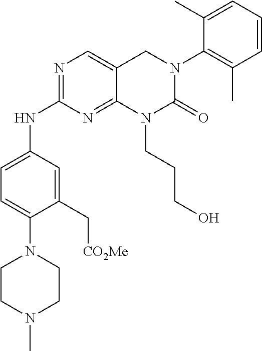 [SMILES: COC(=O)Cc1cc(Nc2ncc3c(n2)N(CCCO)C(=O)N(c2c(C)cccc2C)C3)ccc1N1CCN(C)CC1] | | 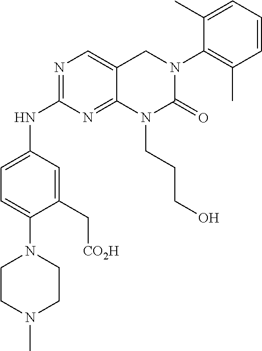 [SMILES: Cc1cccc(C)c1N1Cc2cnc(Nc3ccc(N4CCN(C)CC4)c(CC(=O)O)c3)nc2N(CCCO)C1=O] |
| | SLT-045 | | SLT-044 |
| | 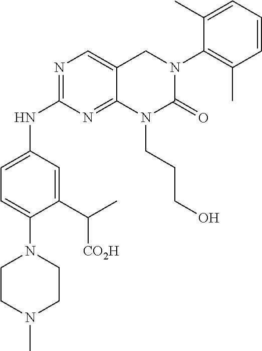 [SMILES: Cc1cccc(C)c1N1Cc2cnc(Nc3ccc(N4CCN(C)CC4)c(C(C)C(=O)O)c3)nc2N(CCCO)C1=O] | | 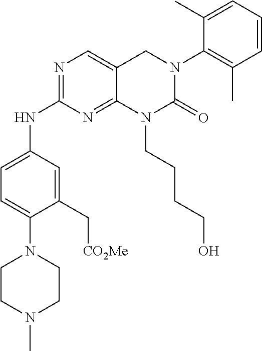 [SMILES: COC(=O)Cc1cc(Nc2ncc3c(n2)N(CCCCO)C(=O)N(c2c(C)cccc2C)C3)ccc1N1CCN(C)CC1] |
| | SLT-066 | | SLT-054 |
| | 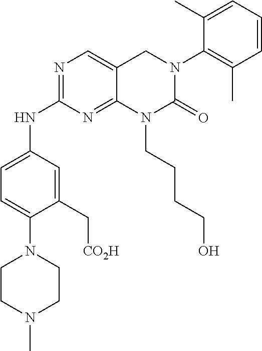 [SMILES: Cc1cccc(C)c1N1Cc2cnc(Nc3ccc(N4CCN(C)CC4)c(CC(=O)O)c3)nc2N(CCCCO)C1=O] | | 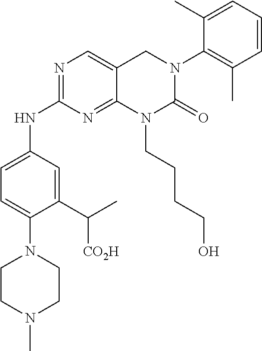 [SMILES: Cc1cccc(C)c1N1Cc2cnc(Nc3ccc(N4CCN(C)CC4)c(C(C)C(=O)O)c3)nc2N(CCCCO)C1=O] |
| | SLT-057 | | SLT-065 |
| | 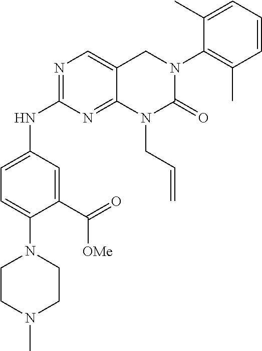 [SMILES: C=CCN1C(=O)N(c2c(C)cccc2C)Cc2cnc(Nc3ccc(N4CCN(C)CC4)c(C(=O)OC)c3)nc21] | | 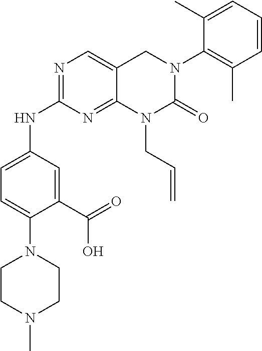 [SMILES: C=CCN1C(=O)N(c2c(C)cccc2C)Cc2cnc(Nc3ccc(N4CCN(C)CC4)c(C(=O)O)c3)nc21] |
| | 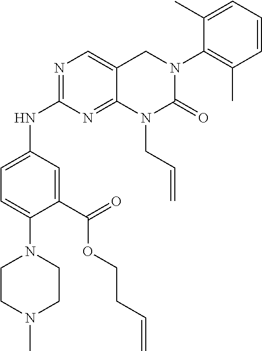 [SMILES: C=CCCOC(=O)c1cc(Nc2ncc3c(n2)N(CC=C)C(=O)N(c2c(C)cccc2C)C3)ccc1N1CCN(C)CC1] | | 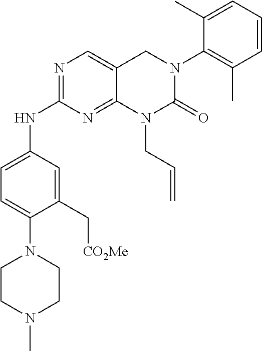 [SMILES: C=CCN1C(=O)N(c2c(C)cccc2C)Cc2cnc(Nc3ccc(N4CCN(C)CC4)c(CC(=O)OC)c3)nc21] |
| | 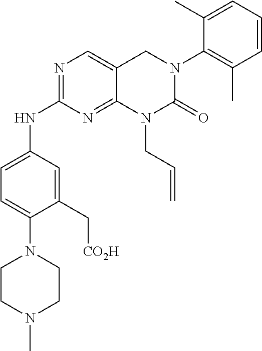 [SMILES: C=CCN1C(=O)N(c2c(C)cccc2C)Cc2cnc(Nc3ccc(N4CCN(C)CC4)c(CC(=O)O)c3)nc21] | | 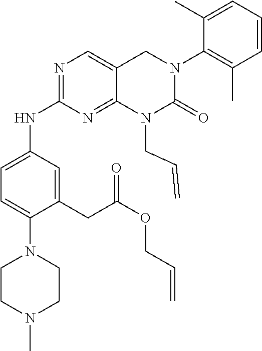 [SMILES: C=CCOC(=O)Cc1cc(Nc2ncc3c(n2)N(CC=C)C(=O)N(c2c(C)cccc2C)C3)ccc1N1CCN(C)CC1] |
| | 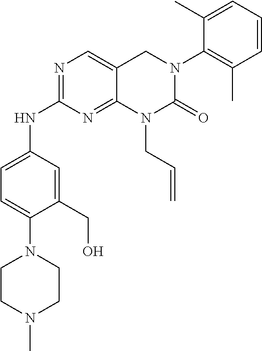 [SMILES: C=CCN1C(=O)N(c2c(C)cccc2C)Cc2cnc(Nc3ccc(N4CCN(C)CC4)c(CO)c3)nc21] | | 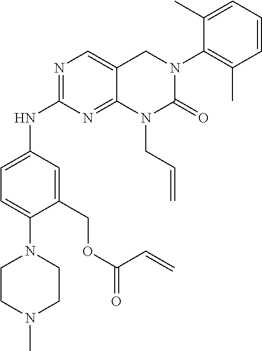 [SMILES: C=CCN1C(=O)N(c2c(C)cccc2C)Cc2cnc(Nc3ccc(N4CCN(C)CC4)c(COC(=O)C=C)c3)nc21] |
| | 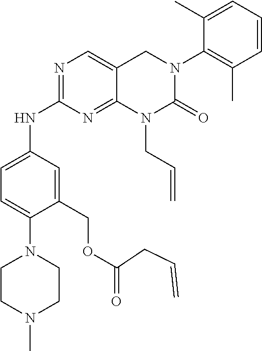 [SMILES: C=CCC(=O)OCc1cc(Nc2ncc3c(n2)N(CC=C)C(=O)N(c2c(C)cccc2C)C3)ccc1N1CCN(C)CC1] | | 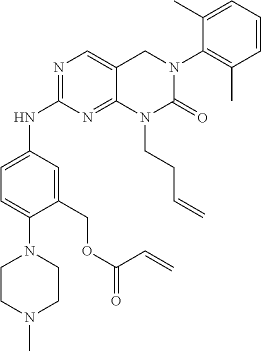 [SMILES: C=CCCN1C(=O)N(c2c(C)cccc2C)Cc2cnc(Nc3ccc(N4CCN(C)CC4)c(COC(=O)C=C)c3)nc21] |

In embodiments, R^{2A} is halogen. In embodiments, R^{2A} is —F. In embodiments, R^{2A} is —Cl. In embodiments, R^{2A} is —Br. In embodiments, R^{2A} is —I.

In embodiments, exemplary compounds of Formula (III-a) or (IV-a) having R^{2A} of halogen are presented in Table 4.

TABLE 4

| Compound | Structure | Compound | Structure |
|---|---|---|---|
| SLT-030 | 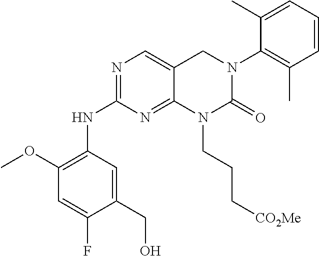 [SMILES: COC(=O)CCCN1C(=O)N(c2c(C)cccc2C)Cc2cnc(Nc3cc(CO)c(F)cc3OC)nc21] | SLT-031 | 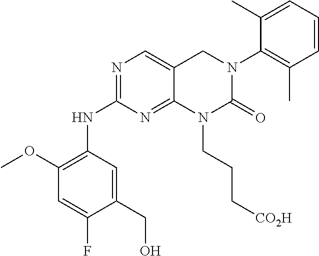 [SMILES: COc1cc(F)c(CO)cc1Nc1ncc2c(n1)N(CCCC(=O)O)C(=O)N(c1c(C)cccc1C)C2] |
| | SLT-030 | | SLT-031 |
| SLT-049 | 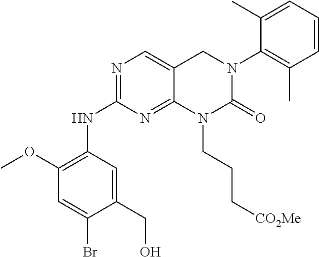 [SMILES: COC(=O)CCCN1C(=O)N(c2c(C)cccc2C)Cc2cnc(Nc3cc(CO)c(Br)cc3OC)nc21] | | |
| | SLT-049 | | |

In embodiments, each R^{2B} and R^{2C} is independently hydrogen, or —OR^{2F}. In embodiments, R^{2F} is hydrogen or unsubstituted C₁-C₄ alkyl. In embodiments, R^{2B} is hydrogen, —OCH₃, or —OCH₂CH₃. In embodiments, R^{2C} is hydrogen, —OCH₃, or —OCH₂CH₃.

In embodiments, R^{2B} is hydrogen and R^{2C} is —OCH₃. In embodiments, R^{2B} is —OCH₃ and R^{2C} is hydrogen. In embodiments, R^{2B} and R^{2C} are hydrogen. In embodiments, R^{2B} and R^{2C} are —OCH₃.

In embodiments, R³ is hydrogen. In embodiments, R³ is —CH₃.

### Pharmaceutical Compositions

In an aspect, pharmaceutical compositions are also provided that comprise one or more of the present compounds, including one or more compounds of Formulae (I), (II), (III), and (IV) and a pharmaceutically acceptable excipient. The pharmaceutical compositions may include one or more compounds of Formulae (I), (II), (III), and (IV) in a therapeutically effective amount (e.g., a therapeutically effective amount).

In addition, the pharmaceutical composition of the present invention may be manufactured with additional pharmaceutically acceptable carrier for each formulation. The type of the carrier that can be used in the present invention is not particularly limited, any carrier conventionally used in the area of industry and pharmaceutically acceptable may be used.

Saline, sterilized water, IV fluids, buffer saline, albumin injection solution, dextrose solution, maltodextrin solution, glycerol, ethanol are non-limiting examples of the usable carriers. These carriers may be used alone or in combination of two or more. The carrier may include a non-naturally occurring carrier. If necessary, other conventionally used additives like an antioxidant, and/or a buffer.

A pharmaceutical composition suitably may be in the form of a spray or liquid wash or other formulation for topical application such as a lotion, cream, ointment, paste, gel, foam, or any other physical form as a carrier generally known for topical administration. Such thickened topical formulations are particularly advantageous because the formulations adhere to the area of the skin on which the material is placed, thus allowing a localized high concentration of one or more of the present compounds to be introduced to the particular area. For example, paraffin- and lanolin-based creams are generally known in the art. Other thickeners, such as polymer thickeners, may be used. The formulations may also comprise one or more of the following: water, preservatives, active surfactants, emulsifiers, anti-oxidants, or solvents.

Pharmaceutical composition may be formulated for a variety of other administration routes such as nasal, oral, parenteral, intramuscular, intra-articular, intravenous, subcutaneous, or transdermal administration. Suitable pharmaceutical compositions may be formulated with for example a diluent, a dispersant, a surfactant, a bonding agent, a lubricant to make an injection solution like aqueous solution, or as a suspension, emulsion, and as pills, capsules, granules or tablets, and the like.

As discussed, kits are also provided. For instance, one or more compounds disclosed herein including any one of Formulae (I), (II), (III), and (IV) suitably can be packaged in suitable containers labeled, for example, for use as a therapy to treat a subject suffering from pigmentation disorders, unevenness in skin tone, hypopigmentation, inflammatory dermatoses including rosacea, vitiligo or other skin-related disorders or diseases. In addition, an article of manufacture or kit further may include, for example, packaging materials, instructions for use, delivery devices, for treating or monitoring the specified condition.

The kit may also include a legend (e.g., a printed label or insert or other medium describing the product's use (e.g., an audio- or videotape)). The legend can be associated with the container (e.g., affixed to the container) and can describe the manner in which the compositions therein should be administered (e.g., the frequency and route of administration), indications therefor, and other uses. The compositions can be ready for administration (e.g., present in dose-appropriate units), and may include one or more additional pharmaceutically acceptable adjuvants, carriers or other diluents and/or an additional therapeutic agent. Alternatively, the compositions for example can be provided in a concentrated form with a diluent and instructions for dilution.

As discussed, methods are provided for treating a disease or disorder, such as an inflammatory disease or disorder, associated with salt-inducible kinases (SIK) by administering a compound, or a pharmaceutical composition including a compound to a subject. Preferably, the compound may have a structure of any one of Formulae (I), (II), (III), and (IV). Inflammatory disorders associated with aberrant expression of salt-inducible kinases include but are not limited to, ulcerative colitis, rheumatoid arthritis, psoriasis rosacea and systemic lupus erythematosus, osteoporosis; hyperproliferative diseases, prostate cancer, and ovarian cancer (Darling N.J., and Cohen P, Nuts and bolts of the salt-inducible kinases (SIKs), Biochem J. (2021) Apr. 16; 478(7):1377-1397; Sun Z, Jiang Q, Li J and Guo J. The potent roles of salt-inducible kinases (SIKs) in metabolic homeostasis and tumorigenesis. Signal Transduction and Targeted Therapy (2020) 5:150. The method may suitably include administering an effective amount (e.g., therapeutically effective amount) of the compound alone or together with one additional compound including, but not limited to, a retinoid, (Adapalene 6-[3-(1-adamantyl)-4-methoxyphenyl]-2-naphthoic acid), retinoic acid, alpha hydroxy acid, lactic acid, beta hydroxy acid, salicylic acid, a corticosteroid (hydrocortisone, clobetasole propionate), a Vitamin D3 derivative (Calcitriol 1,25-dihydroxycholecalciferol) an aryl hydrocarbon receptor modulator (Tapinarof 3,5-dihydroxy-4-isopropyl-trans-stilbene), a Janus kinase inhibitor (Ruxolitinib (3R)-3-Cyclopentyl-3-[4-(7H-pyrrolo[2,3-d]pyrimidin-4-yl)pyrazol-1-yl]propanenitrile), a phosphodiesterase-4 inhibitor (Crisaborole 4-[(1-hydroxy-1,3-dihydro-2,1-benzoxaborol-5-yl)oxy]benzonitrile), a cell cycle checkpoint kinase 1 and 2 inhibitor (Prexasertib 5-((5-(2-(3-Aminopropoxy)-6-methoxyphenyl)-1H-pyrazol-3-yl)amino)-2-pyrazinecarbonitrile), and combinations thereof, wherein the two separate compositions are administered concomitantly or sequentially, in either order.

In preferred aspects, the methods are to treat or prevent pigmentation disorders, unevenness in skin tone, hypopigmentation, inflammatory dermatoses including rosacea, vitiligo or other skin-related diseases or disorders suitably for a subject that is suffering from or susceptible to such diseases or disorders. As discussed, the subject suitably may be a male or female human.

The subject may be suffering from or susceptible to vitiligo, which is a disorder characterized by the appearance on the skin of white patches associated with a pigmentation defect.

The subject may be suffering from or susceptible to any of erythematotelangiectatic rosacea (subtype 1 rosacea), papulopustular rosacea (subtype 2 rosacea), phymatous rosacea (subtype 3 rosacea) and/or ocular rosacea (subtype 4 rosacea). In some embodiments, the treatment methods may further comprise a step of identifying and selecting the subject suffering from rosacea, including a particular sub-type of rosacea, or other skin-related disease or disorder. In subjects suffering from erythematotelangiectatic rosacea (subtype 1 rosacea), a subject may exhibit redness and flushing of skin and visible blood vessels. In subjects suffering from papulopustular rosacea (subtype 2 rosacea), a subject may exhibit redness, swelling and acne-like breakouts. In subjects suffering from phymatous rosacea (subtype 3), a subject may exhibit thickening of facial or other skin and the skin may develop a bumpy texture. In subjects suffering from ocular rosacea (subtype 4), a subject may exhibit bloodshot and watery eyes, eyes that feel gritty, dry, itchy eyes, diminished vision. The identified and selected subject then may be treated with a therapeutic compound or composition as disclosed herein.

In some embodiments, the treatment methods may provide methods for increasing skin pigmentation and/or reducing the risk of skin cancer in a subject in need thereof. The present disclosure provides methods for increasing skin pigmentation for cosmetic purposes. In certain embodiments, provided herein are methods of increasing the appearance of skin darkening in a subject in need thereof using the described compounds (e.g., via topical administration of the described compounds).

In certain embodiments, the present disclosure provides methods of increasing the appearance of skin pigmentation in a subject, the methods comprising administering topically to the subject's skin a compound having a structure of any one of Formulae (I), (II), (III), and (IV) or a pharmaceutically acceptable salt, solvate, hydrate, tautomer, or stereoisomer thereof, or a pharmaceutical composition thereof.

In certain embodiments, the present disclosure provides methods of treating polymorphic light eruption (e.g., sun hypersensitivity). The present disclosure provides methods of inducing eumelanin synthesis. The present disclosure provides methods of inducing melanosomal maturation, export, and localization.

In certain embodiments, provided are methods of reversibly increasing skin pigmentation and/or reducing the risk of skin cancer in a subject in need thereof, the method comprising administering topically to the subject's skin an effective amount of a compound having a structure of any one of Formulae (I), (II), (III), and (IV), or a pharmaceutical composition thereof. In certain embodiments, provided is a method of increasing skin pigmentation and/or reducing the risk of skin cancer by topically administering the compounds described herein to a subject's skin on a body part. In certain embodiments, the body part is the face of the subject. In certain embodiments, the body part is the neck of the subject. In certain embodiments, the body part is the chest of the subject. In certain embodiments, the body part is the back of the subject. In certain embodiments, the skin on the body part is skin on the arms of the subject. In certain embodiments, the skin on the body part is skin on the legs of the subject. In certain embodiments, the skin is on the torso of the subject.

In certain embodiments, the present invention relates to the cosmetic and/or dermatological use of a compound having a structure of any one of Formulae (I), (II), (III), and (IV), or a pharmaceutical composition thereof, for coloring and/or pigmenting the skin and/or body hair and/or head hair. Applications are suitable for improving pigmentation imperfections and disorders, for instance the appearance of white hairs in human beings (canities or natural whitening of hair) which can be either a visible manifestation of the aging process (senile canities), or linked to a genetic predisposition. The pigmentation of head hair and of body hair requires the presence of melanocytes in the bulb of the hair follicle. It is now accepted that canities is associated with a decrease in the amount of melanin in the hair shaft. Since maintaining a constant coloration of the head of hair is a sizeable aspiration, it is therefore desirable to be able to combat the appearance of these visible signs of aging, i.e. to maintain or re-establish the coloration of body hair and/or of head hair.

In certain embodiments, provided are methods of increasing cellular DNA stability or repair in the skin tissue of a subject in need thereof, comprising administering to the subject a compound having a structure of any one of Formulae (I), (II), (III), and (IV), or a pharmaceutical composition thereof, in an amount sufficient to decrease apoptosis and/or thymine dimer formation in the cellular DNA of the skin tissue, thereby increasing cellular DNA stability or repair in the skin tissue of the subject. Compounds described herein are administered prior to UV exposure to increase DNA stability and accordingly following UV exposure to increase DNA repair. Methods of assessing DNA stability and repair are known in the art to include, for example, immunostaining of thymine dimers and TUNEL assay.

### Methods of Producing

Provided herein is a method of producing a compound having the structure of formula (I) or (II),

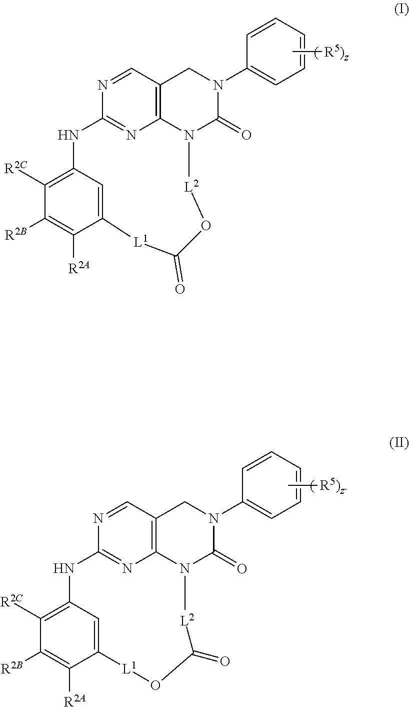

L¹, L², R^{2A}, R^{2B}, R^{2C}, R⁵ and z are as described herein.

In embodiments, the method includes providing a compound having the structure of formula (III) or (IV),

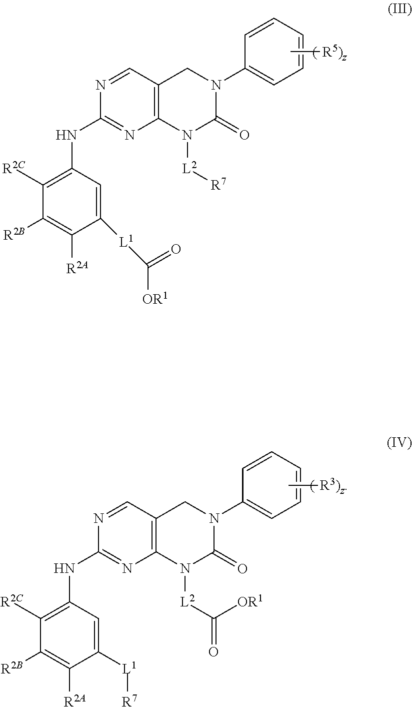

L¹, L², R¹, R^{2A}, R^{2B}, R^{2C}, R⁵, R⁷ and z are as described herein.

In embodiments, the compounds of Formula (III) or (IV) may be provided in a reaction vessel.

In embodiments, after providing the compounds of Formula (III) or (IV), the method may further include treating the compounds of (III) or (IV) with a coupling reagent. In embodiments, the coupling reagent may include EDC hydrochloride, but it is not limited thereto.

In embodiments, after providing the compounds of Formula (III) or (IV), the method may further include heat-treating, or elevating the temperature in the reaction vessel. In embodiments, during the heat-treating, the temperature in the reaction vessel may range from about 40 to about 80° C., from about 45 to about 70° C., or from about 50 to about 60° C.

In embodiments, the method may further include purifying the reaction product obtained in the reaction vessel.

In an aspect, provided is a method for treating a subject suffering from or susceptible to a skin-related disorder or disease. The method includes administering to the subject an effective amount of a compound or composition as described herein.

In embodiments, the subject is identified as suffering from a skin-related disorder or disease and the compound or composition in administered to the identified subject.

In an aspect, provided is a method for treating a subject suffering from or susceptible to rosacea, comprising, administering to the subject an effective amount of a compound or composition as described herein.

In embodiments, the subject is identified as suffering from rosacea and the compound or composition is administered to the identified subject.

In embodiments, the subject is suffering from erythematotelangiectatic rosacea (subtype 1), papulopustular rosacea (subtype 2), phymatous rosacea (subtype 3) and/or ocular rosacea (subtype 4).

In embodiments, the subject has been identified as suffering from erythematotelangiectatic rosacea (subtype 1), papulopustular rosacea (subtype 2), phymatous rosacea (subtype 3) and/or ocular rosacea (subtype 4) and the compound or composition in administered to the identified subject.

In an aspect, provided is a method of increasing pigmentation in a tissue of a subject, said method comprising administering to the subject a compound or composition of as disclosed herein, including Formulae (I-a) and/or Formulae (II-a), in an amount sufficient to increase melanin production, thereby increasing pigmentation in the tissue of the subject.

In embodiments, the tissue of the subject is skin or hair.

In an aspect, provided is a method of increasing cellular DNA stability or repair in the skin tissue of a subject in need thereof. The method includes administering to the subject a compound or composition as described herein in an amount sufficient to decrease apoptosis and/or thymine dimer formation in the cellular DNA of the skin tissue, thereby increasing cellular DNA stability or repair in the skin tissue of the subject.

### EXAMPLES

### Example 1: Synthesis of Anilines

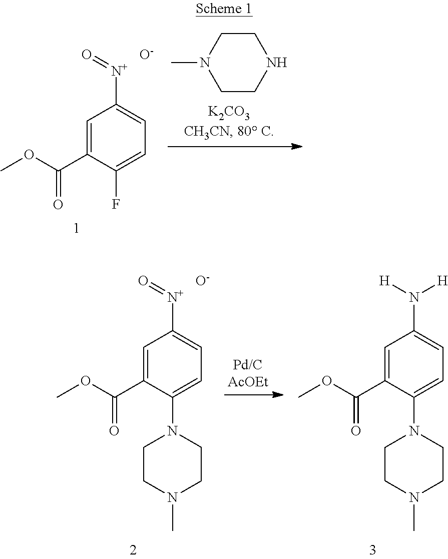

### methyl 2-(4-methylpiperazin-1-yl)-5-nitrobenzoate (2)

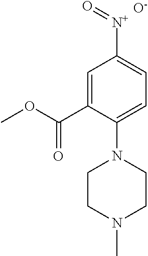

Methyl 2-fluoro-5-nitrobenzenecarboxylate (20.13 g, 119.7 mmol) was dissolved in MeCN (200.0 mL), then potassium carbonate (20.14 g, 143.6 mmol) was added followed by N-methyl piperazine (16.0 mL, 143.6 mmol) and the reaction crude was heated at 80° C. for 16 h. Thereafter, the solvent was removed under vacuum and the obtained residue was purified by flash column chromatography on silica (DCM:MeOH 100:0 to 90:10) to yield pure product (23.4 g, Yield: 70%, yellow solid). LC-MS (ESI+) m/z: 279.8, RT: 0.78 min (TACC50).

### Methyl 5-amino-2-(4-methylpiperazin-1-yl)benzoate (3)

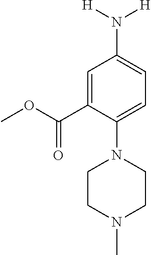 [SMILES: COC(=O)c1cc(N)ccc1N1CCN(C)CC1]

methyl 2-(4-methylpiperazin-1-yl)-5-nitrobenzoate (23.43 g, 83.89 mmol) was dissolved in AcOEt (250 mL). The reaction flask was purged (vacuum and N2 gas cycles). Then, Pd/C (2.3 g) was added and the reaction was subjected to H2 atmosphere at room temperature for 16 h. Thereafter, the reaction crude was filtered over celite and rinsed with AcOEt. Finally, the solvent was removed under vacuum to yield pure product (21.0 g, Yield: quant., brown pale solid). LC-MS (ESI+) m/z: 249.9, RT: 0.197 min (TACC50).

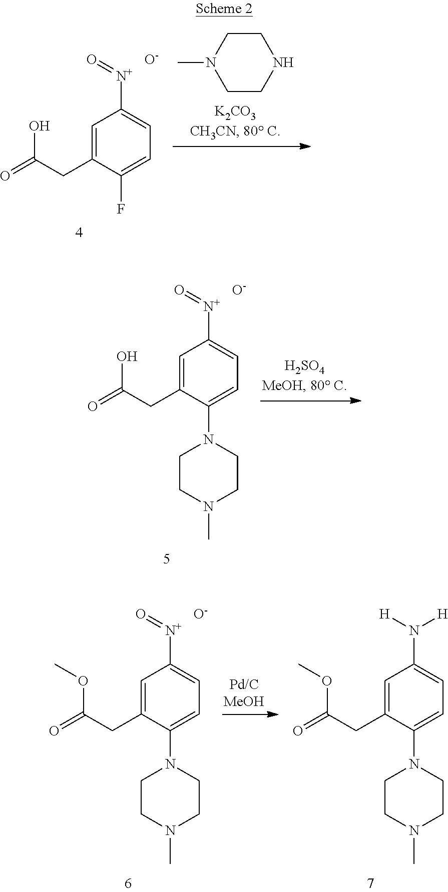

### [2-(4-methylpiperazin-1-yl)-5-nitrophenyl]acetic acid (5)

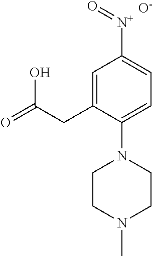

(2-fluoro-5-nitrophenyl)acetic acid (11.0 g, 55.24 mmol) was placed in a sealed tube and dissolved in 120 mL of CH₃CN. Then N-methylpiperazine (15.37 mL, 138.1 mmol) and potassium carbonate (9.29 g, 66.29 mmol) were added and the reaction was stirred at 80° C. for 16 h. Thereafter, the reaction was filtered to remove solids and solvent was removed under vacuum to yield a residue which was used without further purification. LC-MS (ESI+) m/z: 279.8, RT: 0.69 min (TACC50).

### methyl 2-(2-(4-methylpiperazin-1-yl)-5-nitrophenyl)acetate (6)

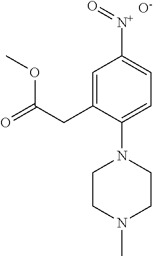

Sulfuric acid (5.0 mL) was added to a solution of [2-(4-methylpiperazin-1-yl)-5-nitrophenyl]acetic acid (9.0 g, 32.2 mmol) in MeOH (100.0 mL) and it was heated at 80° C. for 2 h. The reaction was cooled to room temperature and solvent was removed under vacuum. Then, the reaction crude was diluted with AcOEt and washed with water. Aqueous phase was extracted several times with CHCl3:iPrOH 1:1 mixture. The combined organic layers were dried over MgSO4, filtered and concentrated under vacuum. The obtained residue was purified by flash column chromatography on silica (DCM:MeOH 100:0 to 90:10) to yield pure product (8.19 g, Yield: 87%). LC-MS (ESI+) m/z: 293.9, RT: 0.77 min (TACC50).

### methyl [5-amino-2-(4-methylpiperazin-1-yl)phenyl]acetate (7)

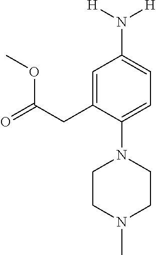 [SMILES: COC(=O)Cc1cc(N)ccc1N1CCN(C)CC1]

Methyl 2-(2-(4-methylpiperazin-1-yl)-5-nitrophenyl)acetate (8.19 g, 27.92 mmol) was dissolved in MeOH (85.0 mL). The reaction flask was purged (vacuum and N2 gas cycles). Then, Pd/C (0.82 g) was added and the reaction was subjected to H2 atmosphere at room temperature for 16 h. Thereafter, the reaction crude was filtered over celite and rinsed with MeOH. Finally, the solvent was removed under vacuum to yield pure product (7.40 g, Yield: quant., brown solid). LC-MS (ESI+) m/z: 263.9, RT: 0.21 min (TACC50).

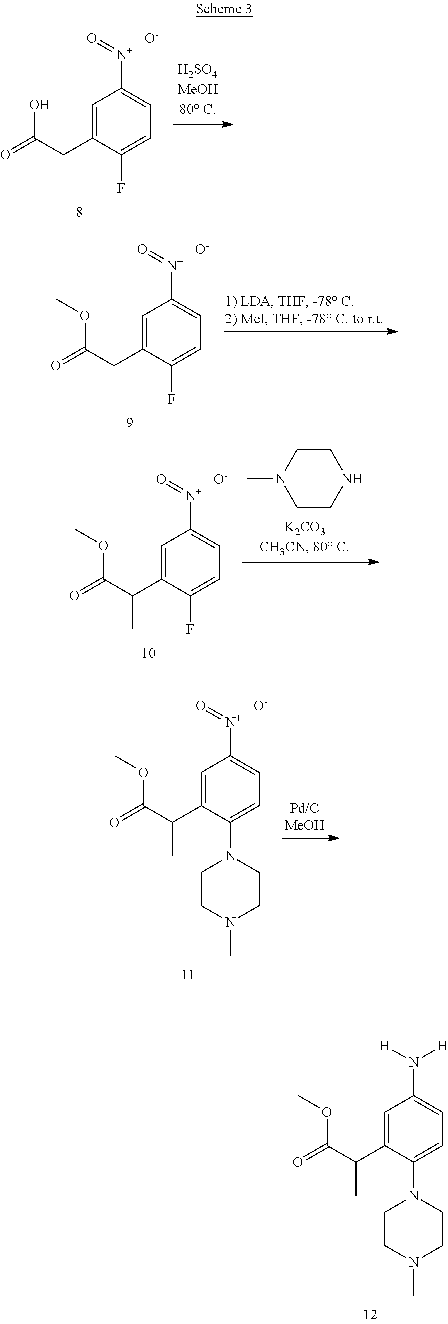

### methyl 2-(2-fluoro-5-nitrophenyl)acetate (9)

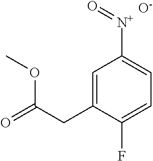

Sulfuric acid (15.5 mL) was added to a solution of 2-fluoro-5-nitrophenylacetic acid (25.0 g, 125.54 mmol) in MeOH (500 mL) and it was heated at 80° C. for 4 hours. The reaction was cooled to room temperature and the solvent was removed under vacuum. The obtained residue was diluted with AcOEt, washed with water, dried over MgSO4, filtered and concentrated under vacuum to yield the pure product (25.25 g, Yield: 94%, brown oil) which was used without further purification. LC-MS (ESI−) m/z: 214.0, RT: 1.26 min (TACC50).

### methyl 2-(2-fluoro-5-nitrophenyl)propanoate (10)

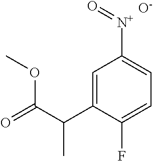

A solution of diisopropylamine (9.43 mL, 67.08 mmol) in anhydrous THF (170 mL) was cooled to −78° C. Then, BuLi 2.5 M (24.8 mL, 61.92 mmol) was dropwise added and the reaction was stirred at −78° C. for 15 min. Thereafter, a solution of methyl 2-(2-fluoro-5-nitrophenyl)acetate (11.00 g, 51.6 mmol) in anhydrous in THF (10.0 mL) was slowly added and it was stirred at −78° C. for 30 minutes. Finally, methyl iodide (3.2 mL, 50.66 mmol) was added and stirred at −78° C. for 10 minutes. After the addition, the reaction was stirred at room temperature for 1 hour. Thereafter, the reaction crude was treated with an aqueous solution of NH4Cl (sat.), diluted with AcOEt and washed with an aqueous solution of NH4Cl (sat.). The combined organic layers were dried over MgSO4, filtered and concentrated under vacuum. The obtained residue was purified by flash column chromatography on silica (Heptane:AcOEt 100:0 to 70:30) to yield the pure product (5.49 g, Yield: 47%, orange oil). LC-MS (ESI−) m/z: 228.0, RT: 1.36 min (TACC50).

### 2-[2-(4-Methyl-piperazin-1-yl)-5-nitro-phenyl]-propionic acid methyl ester (11)

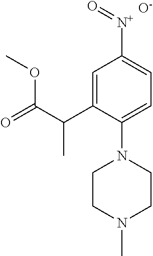

Methyl 2-(2-fluoro-5-nitrophenyl)propanoate (5.49 g, 24.17 mmol) and K2CO3 (4.07 g, 29.00 mmol) were placed in a sealed tube and dissolved in anhydrous ACN (60.0 mL). Then, N-methylpiperazine (6.72 mL, 60.41 mmol) was added and the reaction was stirred at 80° C. for 16 h. Thereafter, the solvent was removed under vacuum, the obtained residue was diluted with AcOEt and washed with water. The combined organic layers were dried over MgSO4, filtered and concentrated under vacuum. The obtained residue was purified by flash column chromatography on silica (DCM:MeOH 100:0 to 90:10) to yield pure product (5.24 g, Yield: 71%, orange solid). LC-MS (ESI−) m/z: 308.0, RT: 0.84 min (TACC50).

### 2-[5-Amino-2-(4-methyl-piperazin-1-yl)-phenyl]-propionic acid methyl ester (12)

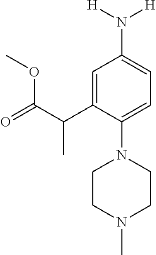 [SMILES: COC(=O)C(C)c1cc(N)ccc1N1CCN(C)CC1]

2-[2-(4-Methyl-piperazin-1-yl)-5-nitro-phenyl]-propionic acid methyl ester (5.24 g, 17.05 mmol) was dissolved in MeOH (50.0 mL). The reaction flask was purged (vacuum and N2 gas cycles). Then, Pd/C (0.54 g) was added and the reaction was subjected to H2 atmosphere at room temperature for 16 h. Thereafter, the reaction crude was filtered over celite and rinsed with MeOH. Finally, the solvent was removed under vacuum to yield pure product (4.37 g, Yield: 92%). LC-MS (ESI+) m/z: 278.0, RT: 0.20 min (TACC50).

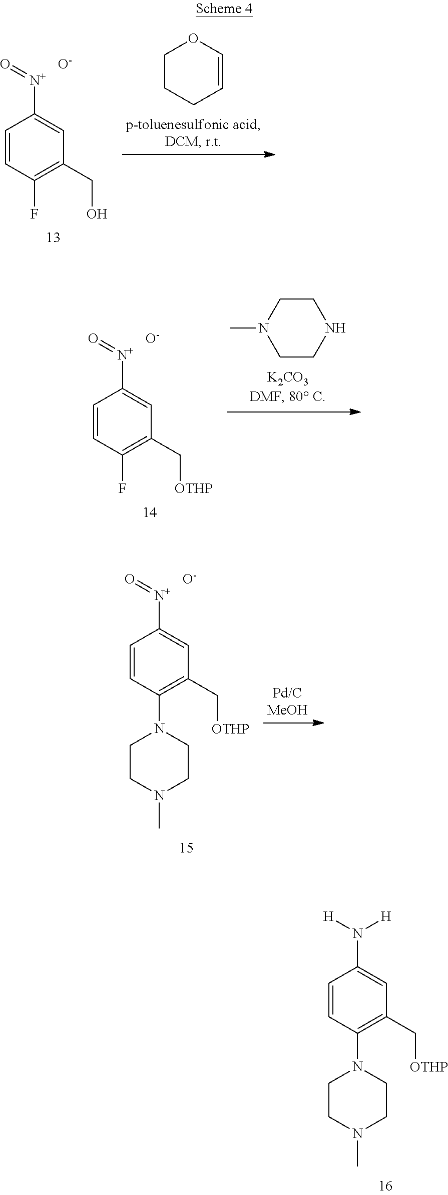

### 2-[4-(Tetrahydro-pyran-2-yloxy)-but-2-enyl]-isoindole-1,3-dione (14)

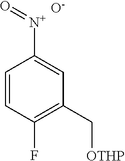

3,4-Dihydro-2H-pyran (19.07 mL, 208.6 mmol) was added to a solution of 2-(4-Hydroxy-but-2-enyl)-isoindole-1,3-dione (11.90 g, 69.54 mmol) in DCM (180.0 mL). Then, Toluene-4-sulfonic acid monohydrate (13.23 g, 69.54 mmol) was added portionwise and the reaction was stirred at room temperature for 16 h. Thereafter, the reaction crude was washed with sat. aqueous NaHCO3. The combined organic layers were dried over MgSO4, filtered and concentrated under vacuum. The obtained residue was purified by flash column chromatography on silica (Heptane:AcOEt 100:0 to 70:30) to yield pure product (9.609 g, Yield: 54%, yellow oil). LC-MS (ESI+): RT: 1.49 min (TACC50).

### 1-methyl-4-(4-nitro-2-{[(oxan-2-yl)oxy]methyl}phenyl)piperazine (15)

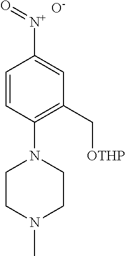

N-methylpiperazine (7.32 mL, 65.80 mmol) and potassium carbonate (6.329 g, 45.13 mmol) were added to a solution of 2-[4-(Tetrahydro-pyran-2-yloxy)-but-2-enyl]-isoindole-1,3-dione (9.60 g, 37.61 mmol) in DMF (115.0 mL) and it was stirred at 80° C. for 16 h. Solvent was removed under vacuum and the obtained residue was diluted with DCM and washed with water. The combined organic layers were dried over MgSO4, filtered and concentrated under vacuum. The obtained residue was purified by flash column chromatography on silica (DCM:MeOH 100:0 to 90:10) to yield the pure product (12.04 g, Yield: 95%, orange oil). LC-MS (ESI+) m/z: 336.1, RT: 0.888 min (TACC50).

### 4-(4-Methyl-piperazin-1-yl)-3-(tetrahydro-pyran-2-yloxymethyl)-phenylamine (16)

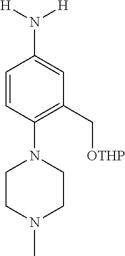

1-methyl-4-(4-nitro-2-{[(oxan-2-yl)oxy]methyl}phenyl)piperazine (8.45 g, 25.21 mmol) was dissolved in MeOH (80.0 mL). The reaction flask was purged (vacuum and N2 gas cycles). Then, Pd/C (0.84 g) was added and the reaction was subjected to H2 atmosphere at room temperature for 16 h. Thereafter, the reaction crude was filtered over celite and rinsed with MeOH. Finally, the solvent was removed under vacuum to yield pure product (7.07, Yield: 92%, brown oil). LC-MS (ESI+m/z: 306.0 RT: 0.32 min TACC50).

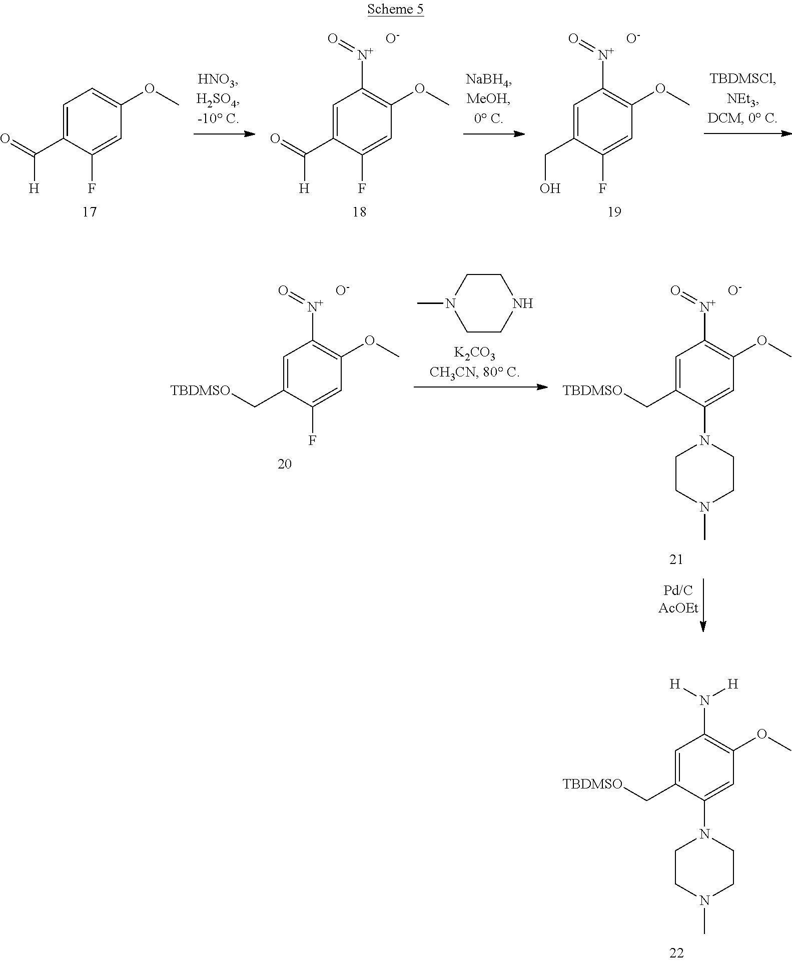

### 2-Fluoro-4-methoxy-5-nitro-benzaldehyde (18)

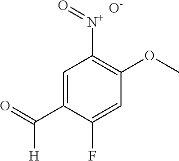

2-Fluoro-4-methoxy-benzaldhyde (10.0 g, 64.9 mmol) was added to a stirred solution of concentrated sulfuric acid (70.0 mL) at −10° C. The reaction was maintained at −10° C. while nitric acid (71.364 mmol 65%, thus 6.9 mL) was added dropwise. Further stirring was allowed for 2 h at this temperature before the mixture was poured into crushed ice. The resulting precipitate was collected by filtration, dissolved in dichloromethane and washed with saturated aqueous NaHCO3 solution. The organic layer was dried over MgSO4, filtered and the solvent was removed under vacuum to yield pure product (11.58 g, Yield: 90%, yellow solid). GC-MS m/z: 199.0, RT: 9.30 min.

### (2-Fluoro-4-methoxy-5-nitro-phenyl)-methanol (19)

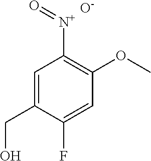

Sodium borohydride (4.593 g, 121.4 mmol) was added portionwise to a stirring solution of 2-Fluoro-4-methoxy-5-nitro-benzaldehyde (12.09 g, 60.71 mmol) in methanol at 0° C. After 2 hours, the methanol was removed under vacuum. The residue was treated with cold water and extracted with dicloromethane. The combined organic layers were washed with brine, dried over MgSO4 and concentrated under vacuum to yield the product which was used without further purification (10.75 g, Yield: 88%, orange oil). LC-MS (ESI+) m/z: 201.8, RT: 0.93 min (TACC50).

### tert-Butyl-(2-fluoro-4-methoxy-5-nitro-benzyloxy)-dimethyl-silane (20)

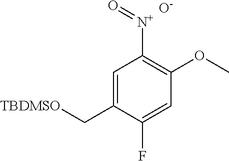

Triethylamine (33.96 mL, 249.05 mmol) was added to an iced-cold solution of (2-Fluoro-4-methoxy-5-nitro-phenyl)-methanol (20.04 g, 99.62 mmol) in anhydrous DCM (300 mL), the mixture was stirred 5 min at 0° C., then tert-butylchlorodimethylsilane (18.0 g, 119.55 mmol) was added portionwise at 0° C. The reaction mixture was stirred at r.t for 16 h. The reaction crude was diluted with DCM and washed with water. Combined organic layers were dried over MgSO4, filtered and concentrated under vacuum. The obtained residue was purified by flash column chromatography on silica (Heptane:AcOEt 100:0 to 70:30) to afford pure product (9.558 g, Yield: 30%). LC-MS (ESI+) m/z: 280/254, RT: 1.89 min (TACC50).

### 1-(2-(((tert-butyldimethylsilyl)oxy)methyl)-5-methoxy-4-nitrophenyl)-4-methylpiperazine (21)

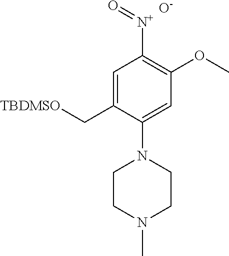

Tert-Butyl-(2-fluoro-4-methoxy-5-nitro-benzyloxy)-dimethyl-silane (9.56 g, 30.3 mmol), 1-methylpiperazine (4.05 mL, 36.4 mmol) and K2CO3 (5.03 g, 36.4 mmol) were dissolved in ACN (90 mL) and stirred at 80° C. for 16 h. The solvent was removed under vacuum and the obtained residue was diluted with AcOEt and washed with water. Combined organic layers were dried over MgSO4, filtered and concentrated under vacuum. The obtained residue was purified by flash column chromatography on silica (DCM:MeOH from 100:0 to 50% 90:10) to yield pure product (2.408 g, Yield: 20%). LC-MS (ESI+) m/z: 395.8, RT: 1.31 min (TACC50).

### 5-(((tert-butyldimethylsilyl)oxy)methyl)-2-methoxy-4-(4-methylpiperazin-1-yl)aniline (22)

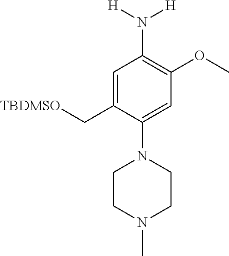 [SMILES: COc1cc(N2CCN(C)CC2)c(CO[Si](C)(C)C(C)(C)C)cc1N]

1-[2-(tert-Butyl-dimethyl-silanyloxymethyl)-5-methoxy-4-nitro-phenyl]-4-methyl-piperazine (2.408 g, 6.087 mmol) was dissolved in AcOEt (20.0 mL). The reaction flask was purged (vacuum and N2 gas cycles). Then, Pd/C (0.240 g) was added and the reaction was subjected to H2 atmosphere for 16 h at room temperature. Thereafter, the reaction crude was filtered over celite and rinsed with AcOEt. Finally, the solvent was removed under vacuum to yield pure product (2.181 g, Yield: 98%, Brown oil). LC-MS (ESI+) m/z: 365.9, RT: 0.929 min (TACC50).

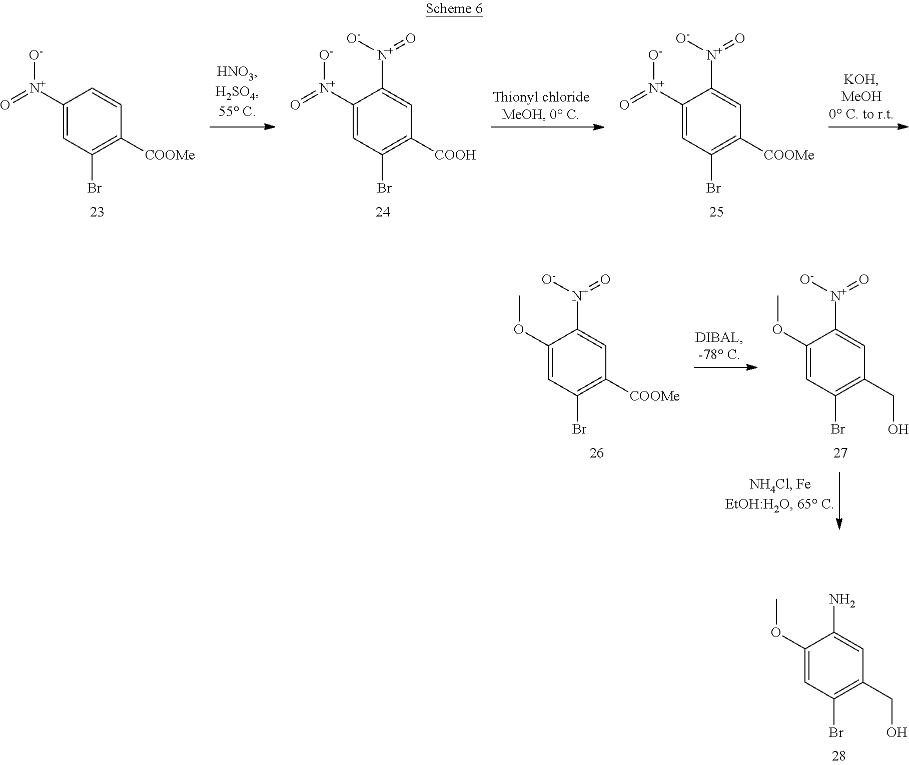 [SMILES: COC(=O)c1ccc([N+](=O)[O-])cc1Br ; O=C(O)c1cc([N+](=O)[O-])c([N+](=O)[O-])cc1Br ; COC(=O)c1cc([N+](=O)[O-])c([N+](=O)[O-])cc1Br ; COC(=O)c1cc([N+](=O)[O-])c(OC)cc1Br ; COc1cc(Br)c(CO)cc1[N+](=O)[O-] ; COc1cc(Br)c(CO)cc1N]

### 2-bromo-4,5-dinitrobenzoic acid (24)

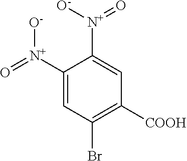 [SMILES: O=C(O)c1cc([N+](=O)[O-])c([N+](=O)[O-])cc1Br]

Methyl 2-bromo-4-nitrobenzoate (9.66 g, 37.147 mmol) was added to an iced-cold solution of H2SO4 (30.0 mL). Then HNO3 (15.0 mL) was added dropwise. The reaction was allowed to reach room temperature and it was heated at 65° C. for 16 h. Thereafter, the reaction was cooled to room temperature and poured into ice-cold water. The obtained solid was collected by filtration, washed with water and dried under vacuum to yield pure product (6.41 g, Yield: 59%, Yellow solid). LC-MS (ESI+) RT: 0.21 min (TACC50).

### methyl 2-bromo-4,5-dinitrobenzoate (25)

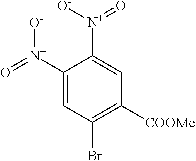 [SMILES: COC(=O)c1cc([N+](=O)[O-])c([N+](=O)[O-])cc1Br]

Thionyl chloride (12.1 mL, 166.31 mmol) was added dropwise to an iced-cold solution of 2-bromo-4,5-dinitrobenzoic acid (4.84 g, 16.63 mmol) in MeOH (50.0 mL). The reaction was stirred at 0° C. for 30 min and then heated at reflux for 16 h. Thereafter, the solvent was removed under vacuum and excess of thionyl chloride removed by co-evaporation with DCM. The obtained residue (5.07 g, Yield: 99%) was used without further purification.

### methyl 2-bromo-4-methoxy-5-nitrobenzoate (26)

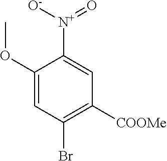 [SMILES: COC(=O)c1cc([N+](=O)[O-])c(OC)cc1Br]

A solution of KOH (1.86 g, 33.2 mmol) in MeOH (10.0 mL) was added dropwise to an iced-cold solution of methyl 2-bromo-4,5-dinitrobenzoate (5.06 g, 16.6 mmol) in MeOH (60.0 mL). Then the reaction was stirred at room temperature for 4 h. Thereafter, the solvent was removed under vacuum and cold water was added to the obtained residue (100 mL). The formed precipitate was collected by filtration, washed with water and dried under vacuum to yield pure product (3.95 g, Yield: 82%, white solid). LC-MS (ESI+) m/z: 290.0 RT: 1.49 min (TACC50).

### (2-bromo-4-methoxy-5-nitrophenyl)methanol (27)

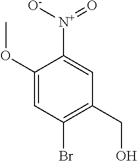

Methyl 2-bromo-4-methoxy-5-nitrobenzoate (2.749 g, 9.477 mmol) was dissolved in anhydrous DCM (20.0 mL) and cooled to −78° C. Then DIBAL (18.9 mmol, 1M solution in toluene, so 19.0 mL) was added and the reaction was stirred at −78° C. for 1 h. Thereafter, the reaction was quenched by the addition of aqueous NaOH solution and filtered through a plug of celite. The filtrate was extracted with DCM and washed with brine. Combined organic layers were dried over MgSO4, filtered and concentrated under vacuum to yield the product which was used without further purification (1.00 g, Yield: 40%, yellow solid). GC-MS (ESI+) m/z: 262.9 RT: 11.307 min.

### (5-amino-2-bromo-4-methoxyphenyl)methanol (28)

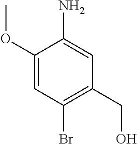

(2-bromo-4-methoxy-5-nitrophenyl)methanol (3.12 g, 11.90 mmol), ammonium chloride (6.369 g, 119.1 mmol) and iron (3.325 g, 59.53 mmol) were placed in a sealed tube and dissolved in EtOH:H2O mixture (50.0 mL, 4:1). Then the reaction was heated at 65° C. for 16 h. Thereafter, the reaction crude was filtered through a plug of celite and rinsed with EtOH. The solvent was removed under vacuum and the obtained residue was diluted with AcOEt and neutralized with an aqueous solution of sat. NaHCO3. The combined organic layers were washed with brine, dried over MgSO4, filtered and concentrated under vacuum to yield pure product (2.52 g, Yield: 91%, brown solid) which was used without further purification. LC-MS (ESI+) m/z: 232.0 RT: 0.66 min (TACC50).

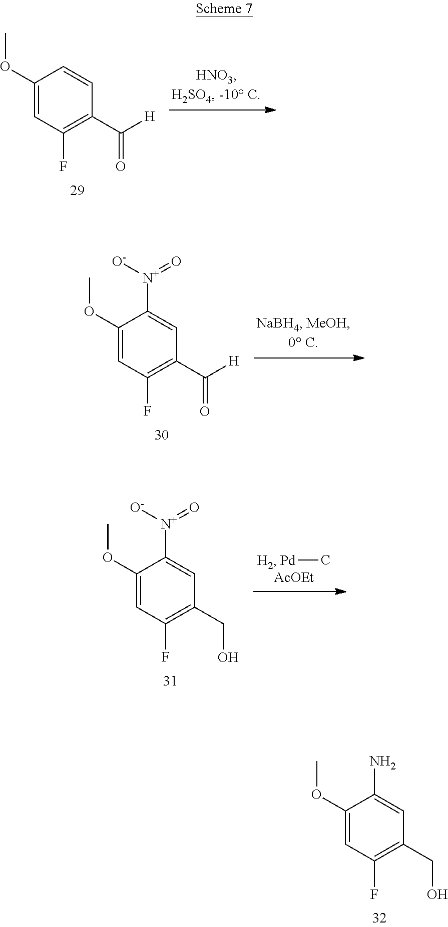

### 2-Fluoro-4-methoxy-5-nitro-benzaldehyde (30)

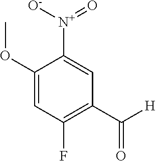

2-Fluoro-4-methoxy-benzaldhyde (5.0 g, 32.438 mmol) was added over concentrated sulfuric acid (35.0 ml) at 0° C. Then, the solution was maintained at −10° C. while nitric acid (35.682 mmol 65%, thus 3.5 mL) was added dropwise. Further stirring was allowed for 2 h at this temperature before the mixture was poured into crushed ice. The resulting precipitate was collected by filtration, dissolved in dichloromethane and washed with saturated aqueous NaHCO3 solution. The organic layer was dried MgSO4 and concentrated under vacuum to yield pure product (5.48 g, Yield: 85%, yellow solid).

### (2-Fluoro-4-methoxy-5-nitro-phenyl)-methanol (31)

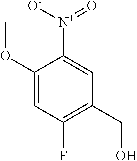

Sodium borohydride (2.454 g, 64.880 mmol) was portionwise added to an iced-cold solution of 2-Fluoro-4-methoxy-5-nitro-benzaldehyde (5.48 g, 32.44 mmol) in methanol (80.0 mL). After 2 hours, solvent was removed under vacuum and the obtained residue was treated with cold water and finally extracted with dicloromethane. The combined organic layers were dried with MgSO4, filtered and concentrated under vacuum. The obtained residue was purified by flash column chromatography on silica (heptane:AcOEt 100:0 to 50:50) to yield pure product (1.71 g, Yield: 26%, yellow oil). LC-MS (ESI+) m/z: 201.9 RT: 1.05 min (TACC50).

### (5-amino-2-fluoro-4-methoxyphenyl)methanol (32)

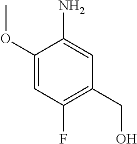

(2-Fluoro-4-methoxy-5-nitro-phenyl)-methanol (1.710 g, 8.501 mmol) was dissolved in AcOEt (30.0 mL). The reaction was subjected to cycles of vacuum-N2 atmosphere. Then Pd on carbon (0.170 g,) was added and the reaction subjected to H2 atmosphere for 16 h. The reaction crude was filtered through a plug of celite and rinsed with AcOEt. Finally, the solvent was removed under vacuum to yield pure product (0.856 g, Yield: 59%, brown pale solid). LC-MS (ESI+) m/z: 172.0 RT: 0.25 min (TACC50).

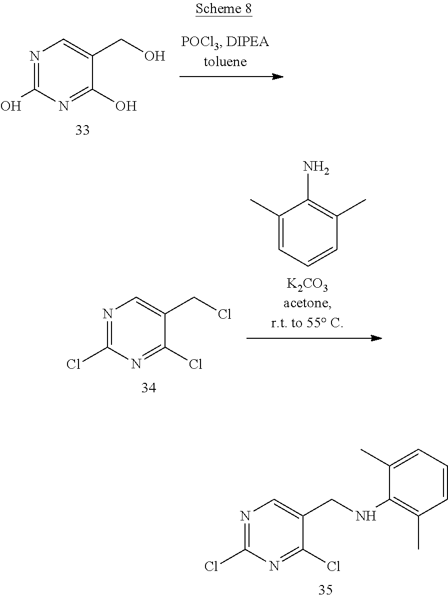

### 2,4-dichloro-5-(chloromethyl)pyrimidine (34)

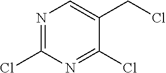

DIPEA (92.0 mL, 527.72 mmol) was slowly added via addition funnel to an iced-cold solution of 5-Hydroxymethyl-1H-pyrimidine-2,4-dione (25.00 g, 175.90 mmol) and phosphorus(V) oxychloride (82.0 mL, 879.54 mmol) in toluene (75.0 mL). After completion of the addition, the cooling bath was removed and the mixture was heated very slowly at 115° C. for 1 h, and then at 125° C. for 3 h. Once the reaction was completed, the reaction mixture was cooled to rt and carefully added using an addition funnel into an iced-cold bi-phasic mixture of water (250 mL) and AcOEt (250 mL). Finally, the mixture was stirred for 45 min at 0° C. and then it was extracted with toluene. The combined organic layers were dried over MgSO4, filtered and concentrated under vacuum. The obtained residue was purified by flash column chromatography on silica (Heptane/AcOEt 100:0 to 50:50) to yield pure product (24.72 g, Yield: 71%, pale yellow oil). LC-MS (ESI+) m/z: 198.1, RT: 1.41 min (TACC50).

### (2,4-Dichloro-pyrimidin-5-ylmethyl)-(2,6-dimethyl-phenyl)-amine (35)

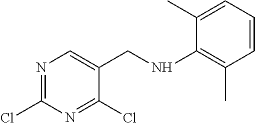 [SMILES: Cc1cccc(C)c1NCc1cnc(Cl)nc1Cl]

Sodium iodide (1.139 g, 7.597 mmol) was dissolved in acetone (20 mL) and the mixture was stirred until clear solution was obtained. Then 2,4-Dichloro-5-chloromethyl-pyrimidine (1.500 g, 7597 mmol) was added under N2 atmosphere and the mixture was stirred at rt for 15 min and 45 min at 60° C. The mixture was cooled and diluted with acetone (40 mL). Then, 2,6-dimethyl-phenylamine (0.939 mL, 7.597 mmol) and potassium carbonate (3.150 g, 22.791 mmol) were added and the reaction was heated at 55° C. for 12 hours. After that time, the reaction mixture was poured into cold water and extracted with AcOEt. The organic layer was washed with an aqueous sodium bisulphite solution and brine, dried over MgSO4, filtered and concentrated under vacuum. The obtained residue was purified by flash column chromatography on silica (heptane/EtOAc from 100:0 to 85:15) to yield pure product (1.677 g, Yield: 87%, white solid). LC-MS (ESI+) m/z: 282.0, RT: 1.82 min (TACC50).

### Example 2: Synthesis of Type A Macrocycles

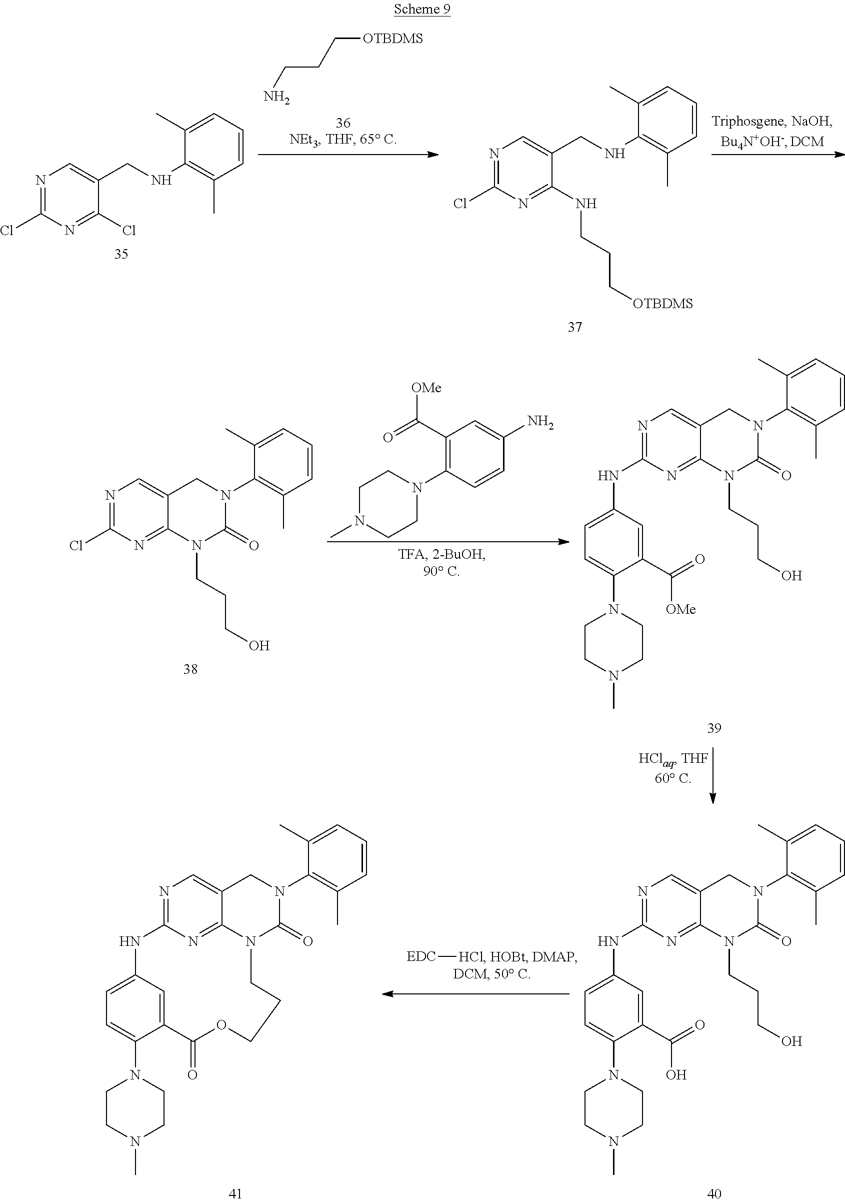

### 3-(tert-Butyl-dimethyl-silanyloxy)-propylamine (36)

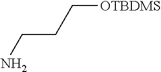 [SMILES: CC(C)(C)[Si](C)(C)OCCCN]

3-amino-1-propanol (5.00 mL, 66.57 mmol) and tert-Butyldimethylsilyl chloride (12.04 g, 79.88 mmol) were dissolved in anhydrous DCM (170 mL) and cooled to 0° C. Then anhydrous triethylamine (15.77 mL, 113.17 mmol) was added and the reaction was allowed to reach room temperature and stirred for 16 h. After that time, the reaction mixture was washed with water. The organic layer was dried over MgSO4, filtered and concentrated under vacuum to yield pure product which was used without further purification. LC-MS (ESI+) m/z: 190.0, RT: 0.97 min (TACC50).

### [3-(tert-Butyl-dimethyl-silanyloxy)-propyl]-{2-chloro-5-[(2,6-dimethyl-phenylamino)-methyl]-pyrimidin-4-yl}-amine (37)

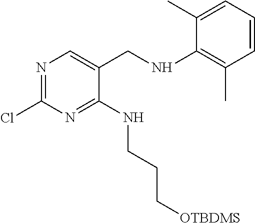 [SMILES: Cc1cccc(C)c1NCc1cnc(Cl)nc1NCCCO[Si](C)(C)C(C)(C)C]

3-(tert-Butyl-dimethyl-silanyloxy)-propylamine (12.54 g, 66.23 mmol) and triethylamine (9.48 mL, 68.04 mmol) were added to a solution of N-((2,4-dichloropyrimidin-5-yl)methyl)-2,6-dimethylaniline (12.8 g, 45.36 mmol) in anhydrous THF (130.0 mL) and the mixture was stirred at 65° C. for 16 h. Thereafter, the reaction crude was diluted with AcOEt and washed with brine. The organic layer was dried over MgSO4, filtered and concentrated under vacuum. The obtained residue was purified by flash column chromatography on silica (Heptane:AcOEt 100:0 to 70:30) to yield pure product (13.24 g, Yield: 67%, colorless oil). LC-MS (ESI+) m/z: 436.0, RT: 2.05 min (TACC50).

### 7-Chloro-3-(2,6-dimethyl-phenyl)-1-(3-hydroxy-propyl)-3,4-dihydro-1H-pyrimido[4,5-d]pyrimidin-2-one (38)

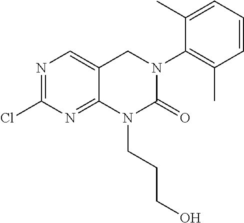 [SMILES: Cc1cccc(C)c1N1Cc2cnc(Cl)nc2N(CCCO)C1=O]

Triphosgene (4.51 g, 15.20 mmol) was added to a solution of [3-(tert-Butyl-dimethyl-silanyloxy)-propyl]-(2-chloro-5-[(2,6-dimethyl-phenylamino)-methyl]-pyrimidin-4-yl)-amine (8.82 g, 20.27 mmol) in DCM (60.0 mL) and it was stirred at room temperature for 1 h. Then an aq. solution of sodium hydroxide (8.11 g, 202.72 mmol, 4.9 M) and tetrabutylammonium hydroxide (2.63 mmol 55% in aq. solution, thus 1.31 mL) was added at 0° C. The reaction was allowed to reach room temperature and stirred at room temperature for 16 h. Thereafter, the reaction crude was diluted with DCM and washed with water. The organic layer was dried over MgSO4, filtered and concentrated under vacuum. The obtained residue was purified by flash column chromatography on silica (Heptane:AcOEt from 100:0 to 30:70) to yield the pure product (6.04 g, Yield: 86%, white solid). LC-MS (ESI+) m/z: 348.0, RT: 1.19 min (TACC50).

### Methyl 5-((6-(2,6-dimethylphenyl)-8-(3-hydroxypropyl)-7-oxo-5,6,7,8-tetrahydropyrimido[4,5-d]pyrimidin-2-yl)amino)-2-(4-methylpiperazin-1-yl)benzoate (39)

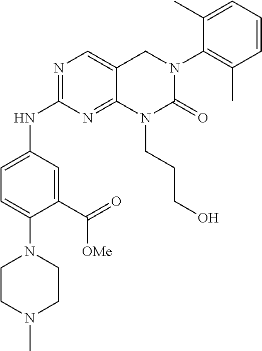 [SMILES: COC(=O)c1cc(Nc2ncc3c(n2)N(CCCO)C(=O)N(c2c(C)cccc2C)C3)ccc1N1CCN(C)CC1]

7-Chloro-3-(2,6-dimethyl-phenyl)-1-(3-hydroxy-propyl)-3,4-dihydro-1H-pyrimido[4,5-d]pyrimidin-2-one (0.480 g, 1.384 mmol) and methyl 5-amino-2-(4-methylpiperazino)benzenecarboxylate (0.459 g, 1.841 mmol) were placed in a sealed tube and dissolved in anhydrous 2-BuOH (20.0 mL). Then 3A MS were added followed by anhydrous trifluoroacetic acid (0.213 mL, 2.768 mmol) and the reaction was stirred at 90° C. for 16 h. Thereafter, the reaction was diluted with water and treated with aq. solution of NaHCO3 (2.768 mmol). The aqueous phase was extracted with CHCl3/iPrOH mixture (1:1). Finally, the combined organic layers were dried over MgSO4, filtered and concentrated under vacuum. The obtained residue was purified by flash column chromatography on silica (DCM:MeOH 100:0 to 90:10) to yield pure product (0.64 g, Yield: 83%). LC-MS (ESI+) m/z: 561.2, RT: 0.52 min (TACC50).

### 5-((6-(2,6-dimethylphenyl)-8-(3-hydroxypropyl)-7-oxo-5,6,7,8-tetrahydroprimido[4,5-d]pyrimidin-2-yl)amino)-2-(4-methylpiperazin-1-yl)benzoic acid (40)

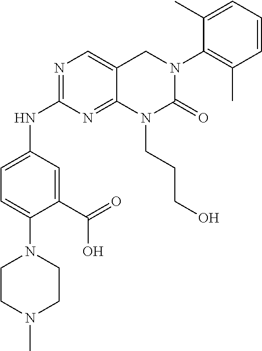 [SMILES: Cc1cccc(C)c1N1Cc2cnc(Nc3ccc(N4CCN(C)CC4)c(C(=O)O)c3)nc2N(CCCO)C1=O]

Methyl 5-((6-(2,6-dimethylphenyl)-8-(3-hydroxypropyl)-7-oxo-5,6,7,8-tetrahydropyrimido[4,5-d]pyrimidin-2-yl)amino)-2-(4-methylpiperazin-1-yl)benzoate was dissolved in THF (4.0 mL) and treated with HCl (5.36 mmol, 6 M aq solution, so 0.89 mL) and it was stirred at 60° C. for 16 h. Thereafter, solvent was removed under vacuum and the obtained residue was purified by reverse phase chromatography (from 95% [aq. 0.1% HCOOH]-5% ACN to 63% [aq. 0.1% HCOOH]-37% ACN) to yield pure product (0.440 g, Yield: 75%). LC-MS (ESI+) m/z: 547.3, RT: 2.094 min (VILLA).

### 1³-(2,6-dimethylphenyl)-3⁴-(4-methylpiperazin-1-yl)-1¹,1²,1³,1⁴-tetrahydro-5-oxa-2-aza-1(7,1)-pyrimido[4,5-d]pyrimidina-3(1,3)-benzenacyclooctaphane-1²,4-dione (41)

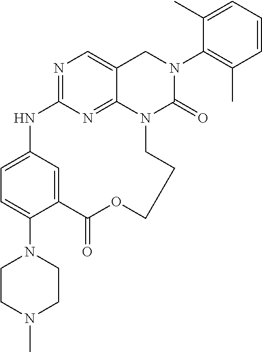 [SMILES: Cc1cccc(C)c1N1Cc2cnc3nc2N(CCCOC(=O)c2cc(ccc2N2CCN(C)CC2)N3)C1=O]

5-((6-(2,6-dimethylphenyl)-8-(3-hydroxypropyl)-7-oxo-5,6,7,8-tetrahydropyrimido[4,5-d]pyrimidin-2-yl)amino)-2-(4-methylpiperazin-1-yl)benzoic acid (0.870 g, 1.598 mmol) and DMAP (0.488 g, 3.995 mmol) were placed in a sealed tube and dissolved in anhydrous DCM (200 mL). Then 3A MS were added followed by EDC hydrochloride (0.306 g, 1.598 mmol) and 1-hydroxybenzotriazole hydrate (0.324 g, 2.397 mmol) and the reaction was heated at 50° C. for 16 h. Thereafter, the reaction crude was washed with an aqueous solution of Rochelle salt. The organic layer was dried over MgSO4, filtered and concentrated under vacuum. The obtained residue was purified flash column chromatography on silica (deactivated with DCM:NH₃ 1%) (DCM:MeOH 100:0 to 90:10) to yield pure product (0.250 g, Yield: 30%, yellow pale solid). LC-MS (ESI+) m/z: 528.2, RT: 2.354 min (VILL_J). 1H NMR (400 MHz, CDCl3) δ 9.93 (d, J=2.7 Hz, 1H), 7.97 (s, 1H), 7.20-7.08 (m, 4H), 7.00 (dd, J=8.6, 2.7 Hz, 1H), 6.88 (d, J=8.6 Hz, 1H), 4.83-4.33 (m, broad signal, 4H), 4.28-3.92 (m, broad signal 2H), 3.25 (s, 4H), 2.72 (s, 4H), 2.42 (s, 3H), 2.25 (s, 6H), 0.86 (m, 2H).

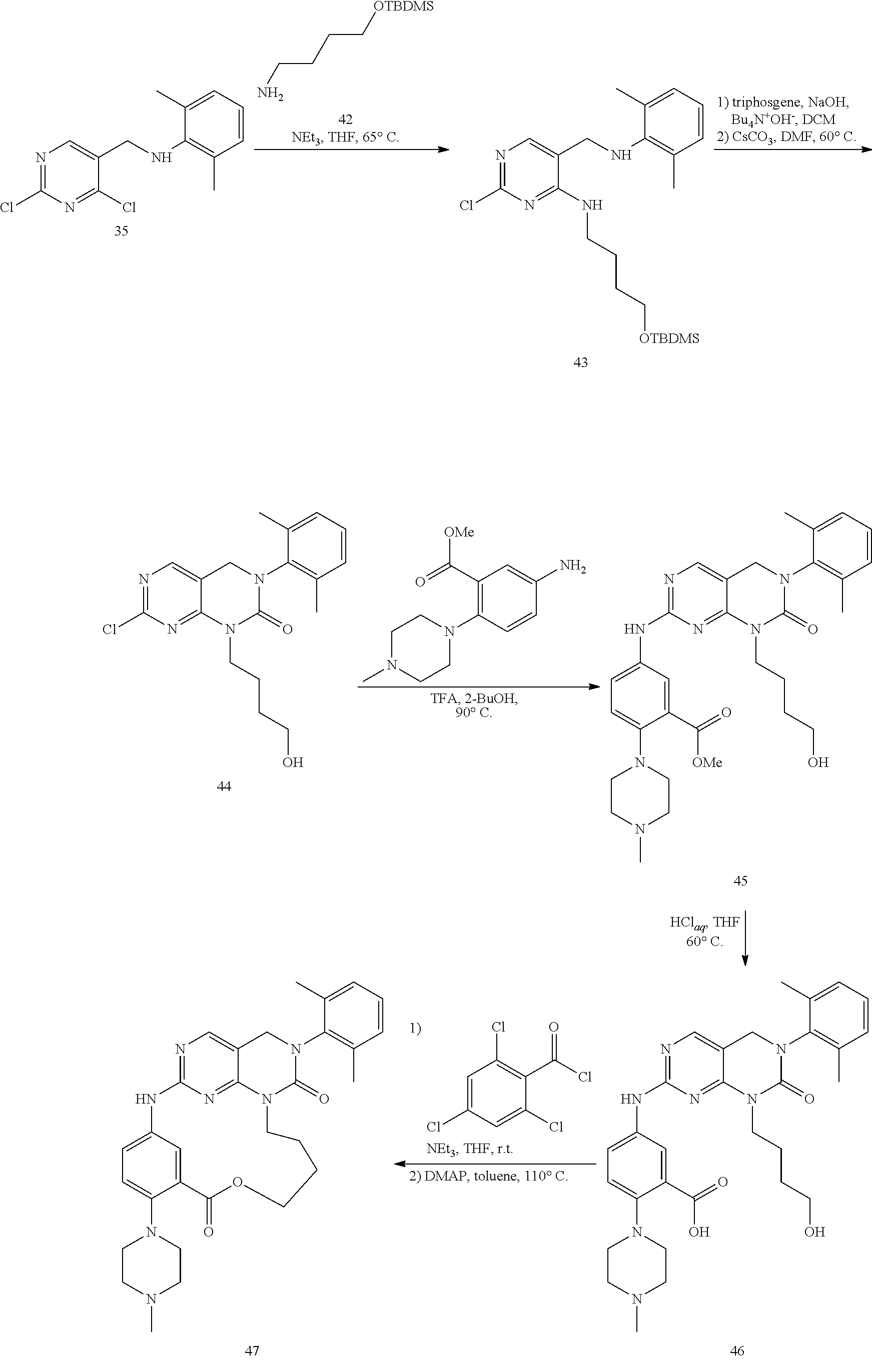

### 4-(tert-Butyl-dimethyl-silanyloxy)-butylamine (42)

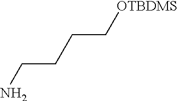 [SMILES: CC(C)(C)[Si](C)(C)OCCCCN]

4-amino-1-butanol (5.00 mL, 56.09 mmol) and tert-Butyldimethylsilyl chloride (8.88 g, 58.9 mmol) were dissolved in anhydrous DCM (100 mL) and cooled to 0° C. Then anhydrous triethylamine (10.16 mL, 72.92 mmol) was added and the reaction was allowed to reach room temperature and stirred for 16 h. After that time, the reaction mixture was washed with water. The organic layer was dried over MgSO4, filtered and concentrated under vacuum to yield pure product (11.40 g, Yield: quant.) which was used without further purification. LC-MS (ESI+) m/z: 204.0, RT: 0.901 min (TACC50).

### [4-(tert-Butyl-dimethyl-silanyloxy)-butyl]-{2-chloro-5-[(2,6-dimethyl-phenylamino)-methyl]-pyrimidin-4-yl}-amine (43)

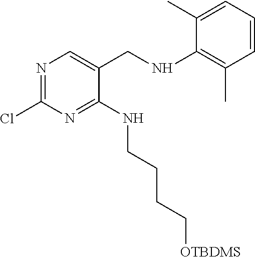 [SMILES: Cc1cccc(C)c1NCc1cnc(Cl)nc1NCCCCO[Si](C)(C)C(C)(C)C]

4-(tert-Butyl-dimethyl-silanyloxy)-butylamine (11.05 g, 54.33 mmol) and triethylamine (7.78 mL, 55.82 mmol) were added to a solution of (2,4-Dichloro-pyrimidin-5-ylmethyl)-(2,6-dimethyl-phenyl)-amine (10.508 g, 37.21 mmol) in anhydrous THF (130.0 mL) and the mixture was stirred at 65° C. for 16 h. Thereafter, the reaction crude was diluted with AcOEt and washed with brine. The organic layer was dried over MgSO4, filtered and concentrated under vacuum. The obtained residue was purified by flash column chromatography on silica (Heptane:AcOEt from 100:0 to 70:30) to yield pure product (16.70 g, Yield: quant., colorless oil). LC-MS (ESI+) m/z: 450.0, RT: 2.14 min (TACC50).

### 7-Chloro-3-(2,6-dimethyl-phenyl)-1-(4-hydroxy-butyl)-3,4-dihydro-1H-pyrimido[4,5-d]pyrimidin-2-one (44)

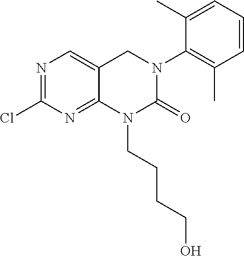 [SMILES: Cc1cccc(C)c1N1Cc2cnc(Cl)nc2N(CCCCO)C1=O]

Triphosgene (8.28 g, 27.91 mmol) was added to a solution of [4-(tert-Butyl-dimethyl-silanyloxy)-butyl]-{2-chloro-5-[(2,6-dimethyl-phenylamino)-methyl]-pyrimidin-4-yl}-amine (16.7 g, 37.21 mmol) in DCM (100 mL) and it was stirred at room temperature for 1 h. Then an aq. solution of sodium hydroxide (14.89 g, 372.12 mmol, 4.9 M) and tetrabutylammonium hydroxide (4.84 mmol 55% in aq. solution, thus 2.4 mL) was added at 0° C. The reaction was allowed to reach room temperature and stirred at room temperature for 16 h. Thereafter, the reaction crude was diluted with DCM and washed with water. The organic layer was dried over MgSO4, filtered and concentrated under vacuum. The obtained residue was dissolved in DMF (100.0 mL) and cessium carbonate (12.25 g, 37.58 mmol) was added. The reaction mixture was heated at 60° C. for 16 h. Thereafter, the reaction crude was diluted with DCM and washed with brine. The organic layer was dried over MgSO4, filtered and concentrated under vacuum. The obtained residue was purified by flash column chromatography on silica (Heptane:AcOEt from 100:0 to 70:30) to yield pure product (3.43 g, Yield: 26%., white solid). LC-MS (ESI+) m/z: 360.0, RT: 1.21 min (TACC50).

### 5-[6-(2,6-Dimethyl-phenyl)-8-(4-hydroxy-butyl)-7-oxo-5,6,7,8-tetrahydro-pyrimido[4,5-d]pyrimidin-2-ylamino]-2-(4-methyl-piperazin-1-yl)-benzoic acid methyl ester (45)

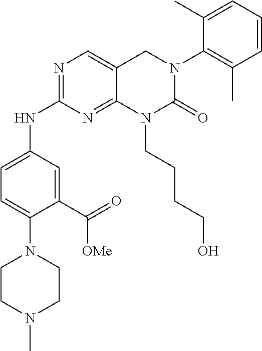 [SMILES: COC(=O)c1cc(Nc2ncc3c(n2)N(CCCCO)C(=O)N(c2c(C)cccc2C)C3)ccc1N1CCN(C)CC1]

7-Chloro-3-(2,6-dimethyl-phenyl)-1-(4-hydroxy-butyl)-3,4-dihydro-1H-pyrimido[4,5-d]pyrimidin-2-one (0.5 g, 1.386 mmol) and methyl 5-amino-2-(4-methylpiperazino)-benzenecarboxylate (0.459 g, 1.843 mmol) were placed in a sealed tube with and dissolved in anhydrous 2-BuOH (4.0 mL). Then 3A MS were added followed by anhydrous TFA (0.316 mL, 2.772 mmol) and the reaction was stirred at 90° C. for 16 h. Thereafter, the reaction was diluted with water and treated with aq. solution of NaHCO3 (2.772 mmol). The aqueous phase was extracted with CHCl3/iPrOH mixture (1:1). Finally, the combined organic layers were dried over MgSO4, filtered and concentrated under vacuum. The obtained residue was purified by flash column chromatography on silica (DCM:MeOH 100:0 to 90:10) to yield pure product (0.371 g, Yield. 47%, yellow solid). LC-MS (ESI+) m/z: 574.8 RT: 1.12 min (TACC50).

### 5-((6-(2,6-dimethylphenyl)-8-(4-hydroxybutyl)-7-oxo-5,6,7,8-tetrahydropyrimido[4,5-d]pyrimidin-2-yl)amino)-2-(4-methylpiperazin-1-yl)benzoic acid (46)

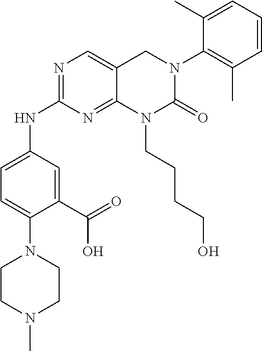 [SMILES: Cc1cccc(C)c1N1Cc2cnc(Nc3ccc(N4CCN(C)CC4)c(C(=O)O)c3)nc2N(CCCCO)C1=O]

5-[6-(2,6-Dimethyl-phenyl)-8-(4-hydroxy-butyl)-7-oxo-5,6,7,8-tetrahydro-pyrimido[4,5-d]pyrimidin-2-ylamino]-2-(4-methyl-piperazin-1-yl)-benzoic acid methyl ester (0.516 g, 0.899 mmol) was dissolved in THF (4.0 mL) and treated with HCl (4.49 mmol, 6 M aq solution, so 0.75 mL) and it was stirred at 60° C. for 16 h. Thereafter, solvent was removed under vacuum and the obtained residue was purified by reverse phase chromatography (from 95% [aq. 0.1% HCOOH]-5% ACN to 63% [aq. 0.1% HCOOH]-37% ACN) to yield pure product (0.338 g, Yield: 67%, white solid). LC-MS (ESI+) m/z: 560.2, RT: 0.67 min (TACC50).

### 1³-(2,6-dimethylphenyl)-3⁴-(4-methylpiperazin-1-yl)-1¹,1²,1³,1⁴-tetrahydro-5-oxa-2-aza-1(7,1)-pyrimido[4,5-d]pyrimidina-3(1,3)-benzenacyclononaphane-1²,4-dione (47)

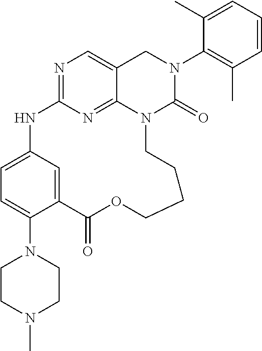 [SMILES: Cc1cccc(C)c1N1Cc2cnc3nc2N(CCCCOC(=O)c2cc(ccc2N2CCN(C)CC2)N3)C1=O]

5-((6-(2,6-dimethylphenyl)-8-(4-hydroxybutyl)-7-oxo-5,6,7,8-tetrahydropyrimido[4,5-d]pyrimidin-2-yl)amino)-2-(4-methylpiperazin-1-yl)benzoic acid (0.350 g, 0.625 mmol) was dissolved in anhydrous THF (36.0 mL). Then triethylamine (0.436 mL, 3.125 mmol) and 2,4,6-trichlorobenzoyl chloride (0.489 mL, 3.125 mmol) were added and the reaction was stirred at room temperature for 16 h. After that time, the solvent was removed under vacuum and the obtained residue was dissolved in anhydrous DMF (5.0 mL) and diluted with anhydrous toluene (106.0 mL). Then it was dropwise added to a refluxing solution of DMAP (0.458 g, 3.750 mmol)) in anhydrous toluene (14.0 mL, final concentration 0.005 mmol/mL, total volume 125.0 mL) via addition funnel and it was stirred at that temperature for 16 h. Thereafter the solvent was removed under vacuum and the obtained residue was purified by reverse phase chromatography (from 59% [aq. 25 mM NH4HCO3]-41% ACN to 17% [aq. 25 mM NH4HCO3]-83% ACN) to yield pure product (0.074, Yield: 22%, white solid). LC-MS (ESI+) m/z: 542.2, RT: 2.459 min (VILL_J). 1H NMR (400 MHz, CDCl3) δ 9.60 (s, 1H), 7.96 (s, 1H), 7.21-7.10 (series of m, 4H), 7.02-6.97 (series of m, 2H), 4.50 (s, 2H), 4.33 (t, J=5.8 Hz, 2H), 4.18-4.12 (m, 2H), 3.29 (broad signal, 4H), 2.91 (broad signal, 4H), 2.55 (s, 3H), 2.26 (s, 6H), 2.14-2.05 (m, 2H), 1.87-1.79 (m, 2H).

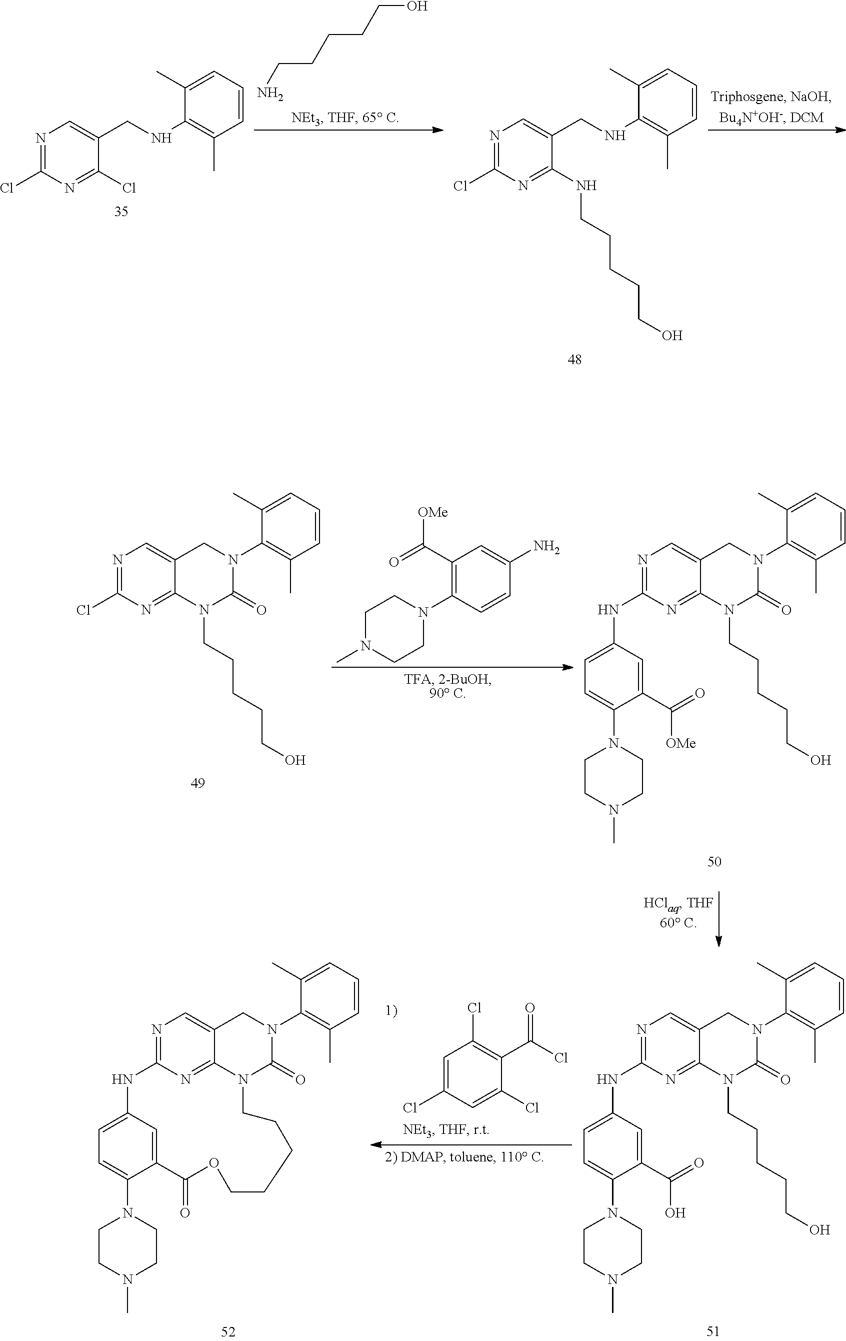

### 5-{2-Chloro-5-[(2,6-dimethyl-phenylamino)-methyl]pyrimidin-4-ylamino}-pentan-1-ol (48)

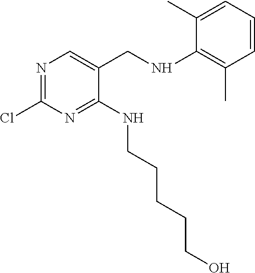 [SMILES: Cc1cccc(C)c1NCc1cnc(Cl)nc1NCCCCCO]

5-amino-1-pentanol (1.29 g, 12.48 mmol) and triethylamine (1.74 mL, 12.48 mmol) were added to a solution of N-((2,4-dichloropyrimidin-5-yl)methyl)-2,6-dimethylaniline (3.200 g, 11.341 mmol) in anhydrous THF (32.0 mL) and the mixture was stirred at 65° C. for 16 h. Thereafter, the reaction crude was diluted with AcOEt and washed with brine. The organic layer was dried over MgSO4, filtered and concentrated under vacuum. The obtained residue was purified by flash column chromatography on silica (DCM:MeOH from 100:0 to 19:1) to yield pure product (2.49 g, Yield: 63%, orange oil). LC-MS (ESI+) m/z: 348.9, RT: 1.47 min (TACC50).

### 7-Chloro-3-(2,6-dimethyl-phenyl)-1-(5-hydroxy-pentyl)-3,4-dihydro-1H-pyrimido[4,5-d]pyrimidin-2-one (49)

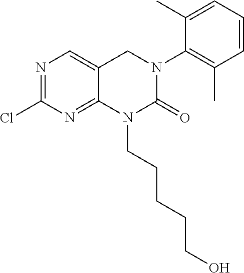 [SMILES: Cc1cccc(C)c1N1Cc2cnc(Cl)nc2N(CCCCCO)C1=O]

Triphosgene (B, 3.183 g, 10.727 mmol) was added to a solution of 5-{2-Chloro-5-[(2,6-dimethyl-phenylamino)-methyl]-pyrimidin-4-ylamino}-pentan-1-ol (2.495 g, 7.151 mmol) in DCM (22.0 mL) and it was stirred at room temperature for 1 h. Then an aq. solution of sodium hydroxide (5.721 g, 143.02 mmol, 4.9 M) and tetrabutylammonium hydroxide (0.930 mmol 55% in aq. solution, thus 0.441 mL) was added at 0° C. The reaction was allowed to reach room temperature and stirred at room temperature for 16 h. Thereafter, the reaction crude was diluted with DCM and washed with water. The organic layer was dried over MgSO4, filtered and concentrated under vacuum. The obtained residue was purified by flash column chromatography on silica (DCM:MeOH from 100:0 to 90:10) to yield pure product (0.953 g, Yield: 36%, yellow solid). LC-MS (ESI+) m/z: 374.9, RT: 1.42 min (TACC50)

### 5-[6-(2,6-Dimethyl-phenyl)-8-(5-hydroxy-pentyl)-7-oxo-5,6,7,8-tetrahydro-pyrimido[4,5-d]pyrimidin-2-ylamino]-2-(4-methyl-piperazin-1-yl)-benzoic acid methyl ester (50)

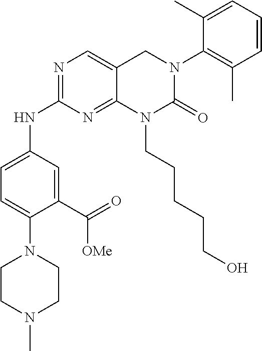 [SMILES: COC(=O)c1cc(Nc2ncc3c(n2)N(CCCCCO)C(=O)N(c2c(C)cccc2C)C3)ccc1N1CCN(C)CC1]

7-Chloro-3-(2,6-dimethyl-phenyl)-1-(5-hydroxy-pentyl)-3,4-dihydro-1H-pyrimido[4,5-d]pyrimidin-2-one (0.50 g, 1.334 mmol) and methyl 5-amino-2-(4-methylpiperazino)benzenecarboxylate (0.442 g, 1.774 mmol) were placed in a sealed tube and dissolved in anhydrous 2-BuOH (20.0 mL). Then 3A MS were added followed by anhydrous trifluoroacetic acid (0.20 mL, 2.67 mmol) and the reaction was stirred at 90° C. for 16 h. Thereafter, the reaction was diluted with water and treated with aq. solution of NaHCO3 (2.67 mmol). The aqueous phase was extracted with CHCl3/iPrOH mixture (1:1). Finally, the combined organic layers were dried over MgSO4, filtered and concentrated under vacuum. The obtained residue was purified by flash column chromatography on silica (DCM:MeOH 100:0 to 90:10) to yield pure product (0.420 g, Yield: 54%, yellow solid). LC-MS (ESI+) m/z: 587.8, RT: 2.308 min (TACC50).

### 5-[6-(2,6-Dimethyl-phenyl)-8-(5-hydroxy-pentyl)-7-oxo-5,6,7,8-tetrahydro-pyrimido[4,5-d]pyrimidin-2-ylamino]-2-(4-methyl-piperazin-1-yl)-benzoic acid (51)

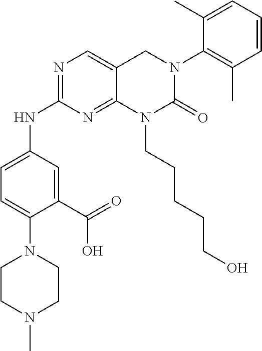 [SMILES: Cc1cccc(C)c1N1Cc2cnc(Nc3ccc(N4CCN(C)CC4)c(C(=O)O)c3)nc2N(CCCCCO)C1=O]

5-[6-(2,6-Dimethyl-phenyl)-8-(5-hydroxy-pentyl)-7-oxo-5,6,7,8-tetrahydro-pyrimido[4,5-d]pyrimidin-2-ylamino]2-(4-methyl-piperazin-1-yl)-benzoic acid methyl ester (0.45 g, 0.771 mmol) was dissolved in THF (3 mL) and treated with HCl (3.855 mmol, 6 M aq solution, so 0.643 mL) and it was stirred at 60° C. for 16 h. Thereafter, solvent was removed under vacuum and the obtained residue was purified by reverse phase chromatography (from 95% [aq. 0.1% HCOOH]-5% ACN to 63% [aq. 0.1% HCOOH]-37% ACN) to yield pure product (0.296 g, Yield: 67%, white solid). LC-MS (ESI+): m/z: 575.0, RT: 2.217 min (VILLA).

### 1³-(2,6-dimethylphenyl)-3⁴-(4-methylpiperazin-1-yl)-1¹,1²,1³,1⁴-tetrahydro-5-oxa-2-aza-1(7,1)-pyrimido[4,5-d]pyrimidina-3(1,3) benzenacyclodecaphane-1²,4-dione (52)

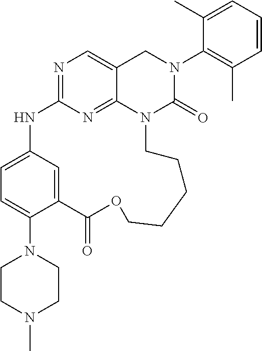 [SMILES: Cc1cccc(C)c1N1Cc2cnc3nc2N(CCCCCOC(=O)c2cc(ccc2N2CCN(C)CC2)N3)C1=O]

5-[6-(2,6-Dimethyl-phenyl)-8-(5-hydroxy-pentyl)-7-oxo-5,6,7,8-tetrahydro-pyrimido[4,5-d]pyrimidin-2-ylamino]2-(4-methyl-piperazin-1-yl)-benzoic acid (0.130 g, 0.227 mmol) was dissolved in anhydrous THF (13.0 mL). Then triethylamine (0.16 mL, 1.135 mmol) and 2,4,6-trichlorobenzoyl chloride (0.18 mL, 1.135 mmol) were added and the reaction was stirred at room temperature for 16 h. After that time, the solvent was removed under vacuum and the obtained residue was dissolved in anhydrous DMF (5.0 mL) and diluted with anhydrous toluene (35.0 mL). Then it was dropwise added to a refluxing solution of DMAP (0.166 g, 1.362 mmol) in anh. toluene (5.0 mL, final concentration 0.005 mmol/mL, total volume 45.0 mL) via addition funnel and it was stirred at that temperature for 16 h. Thereafter the solvent was removed under vacuum and the obtained residue was purified by reverse phase chromatography (from 59% [aq. 25 mM NH4HCO3]-41% ACN to 17% [aq. 25 mM NH4HCO3]-83% ACN) to yield pure product (0.0066 g, Yield: 6%, white solid). LC-MS (ESI+): m/z: 556.3176, RT: 3.187 min (VILLA_2T). ¹H NMR (400 MHz, CDCl₃) δ 9.06 (d, J=2.5 Hz, 1H), 7.87 (s, 1H), 7.13-6.99 (series of m, 5H), 6.92 (dd, J=8.7, 2.7 Hz, 1H), 4.42 (s, 2H), 4.30-4.25 (m, 2H), 4.06-3.95 (m, 2H), 3.15 (broad signal, 4H), 2.81 (broad signal, 4H), 2.47 (s, 3H), 2.19 (s, 6H), 2.03-1.95 (m, J=15.2, 7.2 Hz, 2H), 1.82-1.75 (m, 2H), 1.67-1.59 (m, J=12.6, 6.3 Hz, 2H).

### Type B Macrocycles

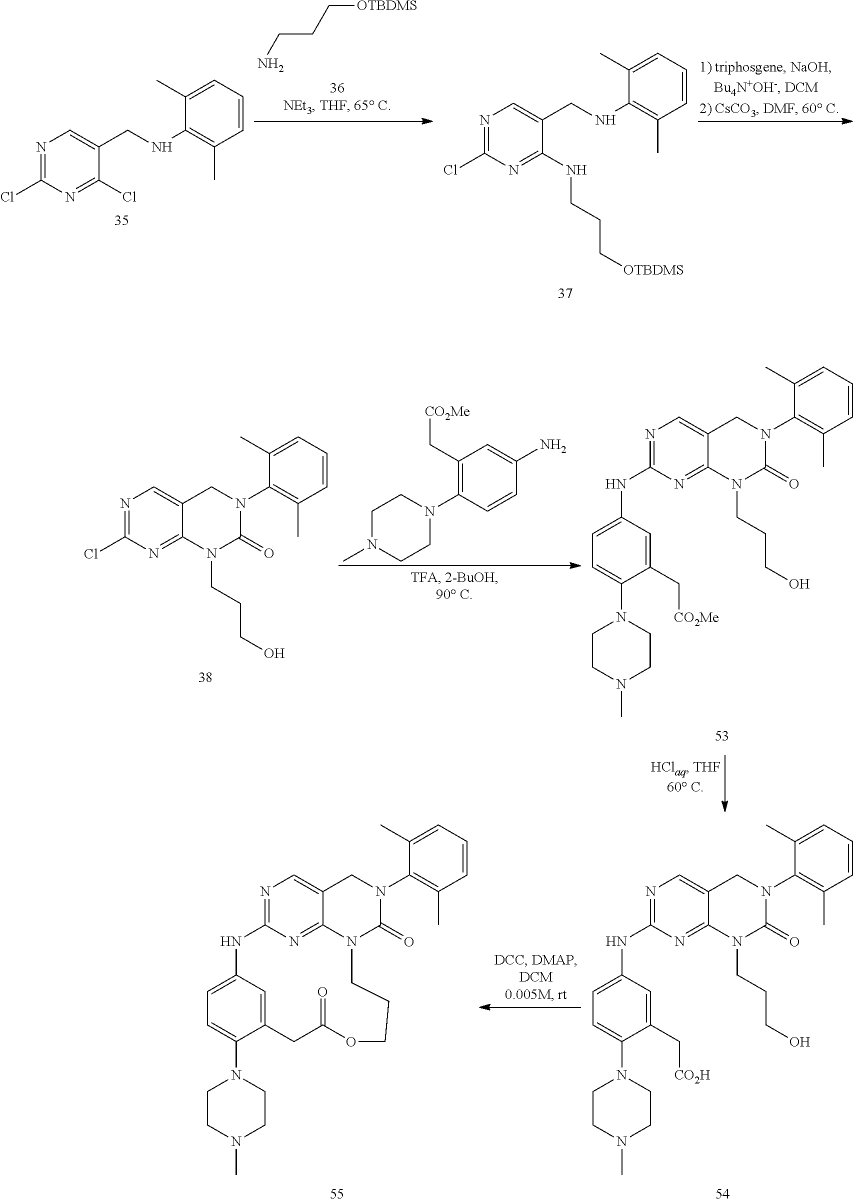

Previous intermediates (35-38) have already been described in the experimental section.

### [5-[6-(2,6-Dimethyl-phenyl)-8-(3-hydroxy-propyl)-7-oxo-5,6,7,8-tetrahydro-pyrimido[4,5-d]pyrimidin-2-ylamino]-2-(4-methyl-piperazin-1-yl)-phenyl]-acetic acid methyl ester (53)

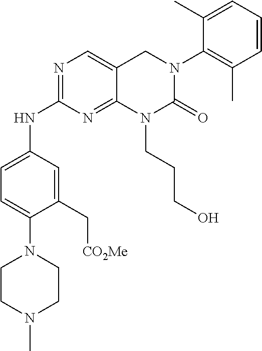 [SMILES: COC(=O)Cc1cc(Nc2ncc3c(n2)N(CCCO)C(=O)N(c2c(C)cccc2C)C3)ccc1N1CCN(C)CC1]

7-Chloro-3-(2,6-dimethyl-phenyl)-1-(3-hydroxy-propyl)-3,4-dihydro-1H-pyrimido[4,5-d]pyrimidin-2-one (0.277 g, 0.799 mmol) and methyl [5-amino-2-(4-methylpiperazin-1-yl)phenyl]acetate (0.280 g, 1.063 mmol) were placed in a sealed tube and dissolved in anhydrous 2-BuOH (3.0 mL). Then 3A MS were added followed by anhydrous trifluoroacetic acid (0.122 mL, 1.6 mmol) and the reaction was stirred at 90° C. for 16 h. Thereafter, the reaction was diluted with water and treated with aq. solution of NaHCO3 (1.6 mmol). The aqueous phase was extracted with CHCl3/iPrOH mixture (1:1). Finally, the combined organic layers were dried over MgSO4, filtered and concentrated under vacuum. The obtained residue was purified by flash column chromatography on silica (DCM:MeOH 100:0 to 90:10) to yield pure product (0.449 g, Yield: 98%, brown pale solid). LC-MS (ESI+): m/z: 575.3, RT: 2.211 min (VILLA).

### [5-[6-(2,6-Dimethyl-phenyl)-8-(3-hydroxy-propyl)-7-oxo-5,6,7,8-tetrahydro-pyrimido[4,5-d]pyrimidin-2-ylamino]-2-(4-methyl-piperazin-1-yl)-phenyl]-acetic acid (54)

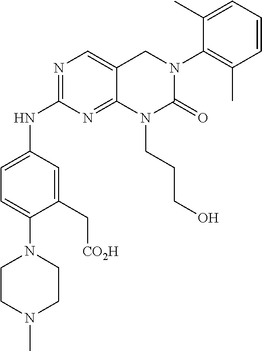 [SMILES: Cc1cccc(C)c1N1Cc2cnc(Nc3ccc(N4CCN(C)CC4)c(CC(=O)O)c3)nc2N(CCCO)C1=O]

[5-[6-(2,6-Dimethyl-phenyl)-8-(3-hydroxy-propyl)-7-oxo-5,6,7,8-tetrahydro-pyrimido[4,5-d]pyrimidin-2-ylamino]-2-(4-methyl-piperazin-1-yl)-phenyl]-acetic acid methyl ester (0.370 g, 0.645 mmol) was dissolved in THF (3.0 mL) and treated with HCl (3.22 mmol, 6 M solution, 0.538 mL) at 60° C. for 5 hours. Thereafter, solvent was removed under vacuum and the obtained residue was purified by reverse phase chromatography (from 95% [aq. 25 mM NH4HCO3]-5% ACN to 63% [aq. 25 mM NH4HCO3]-37% ACN) to yield pure product (0.099 g, Yield: 27%, white solid). LC-MS (ESI+): m/z: 561.3, RT: 2.065 min (VILLA).

### 1³-(2,6-dimethylphenyl)-3⁴-(4-methylpiperazin-1-yl)-1¹,1²,1³,1⁴-tetrahydro-6-oxa-2-aza-1(7,1-pyrimido[4,5-d]pyrimidina-3(1,3)-benzenacyclononaphane-1²,5-dione (55)

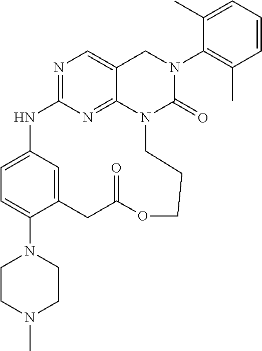 [SMILES: Cc1cccc(C)c1N1Cc2cnc3nc2N(CCCOC(=O)Cc2cc(ccc2N2CCN(C)CC2)N3)C1=O]

[5-[6-(2,6-Dimethyl-phenyl)-8-(3-hydroxy-propyl)-7-oxo-5,6,7,8-tetrahydro-pyrimido[4,5-d]pyrimidin-2-ylamino]-2-(4-methyl-piperazin-1-yl)-phenyl]-acetic acid was dissolved in anhydrous DMF (2.0 mL) and diluted with anhydrous DCM (63.0 mL). 3A molecular shieves were added followed by DMAP (0.060 g, 0.492 mmol) and DCC (0.081 g, 0.394 mmol) and the reaction was stirred at room temperature for 2 h. Then, solvent was removed under vacuum and the obtained residue was dissolved in ACN, formed solids were removed by filtration. The obtained filtrate was concentrated under vacuum rendering a residue which was purified by reverse phase chromatograhy (from 70% 65 mM [NH4OAc+ACN (90:10)]-30% ACN to 27% [NH4OAc+ACN (90:10)]-73% ACN) to yield the pure product (0.0384 g, Yield. 21%, white solid). LC-MS (ESI+): m/z: 542.3, RT: 2.489 min (VILL-J). 1H NMR (400 MHz, CDCl3) δ 8.37 (d, J=2.4 Hz, 1H), 7.92 (s, 1H), 7.63-7.54 (broad signal, 1H), 7.19-7.10 (m, 3H), 7.10-7.05 (m, 1H), 6.80 (dd, J=8.5, 2.5 Hz, 1H), 4.47 (s, 2H), 4.40-4.32 (m, 2H), 4.29-4.21 (m, 2H), 3.84 (s, 2H), 2.93 (t, J=4.7 Hz, 4H), 2.61 (broad signal, 4H), 2.37 (s, 3H), 2.24 (s, 6H), 2.13-2.04 (m, 2H).

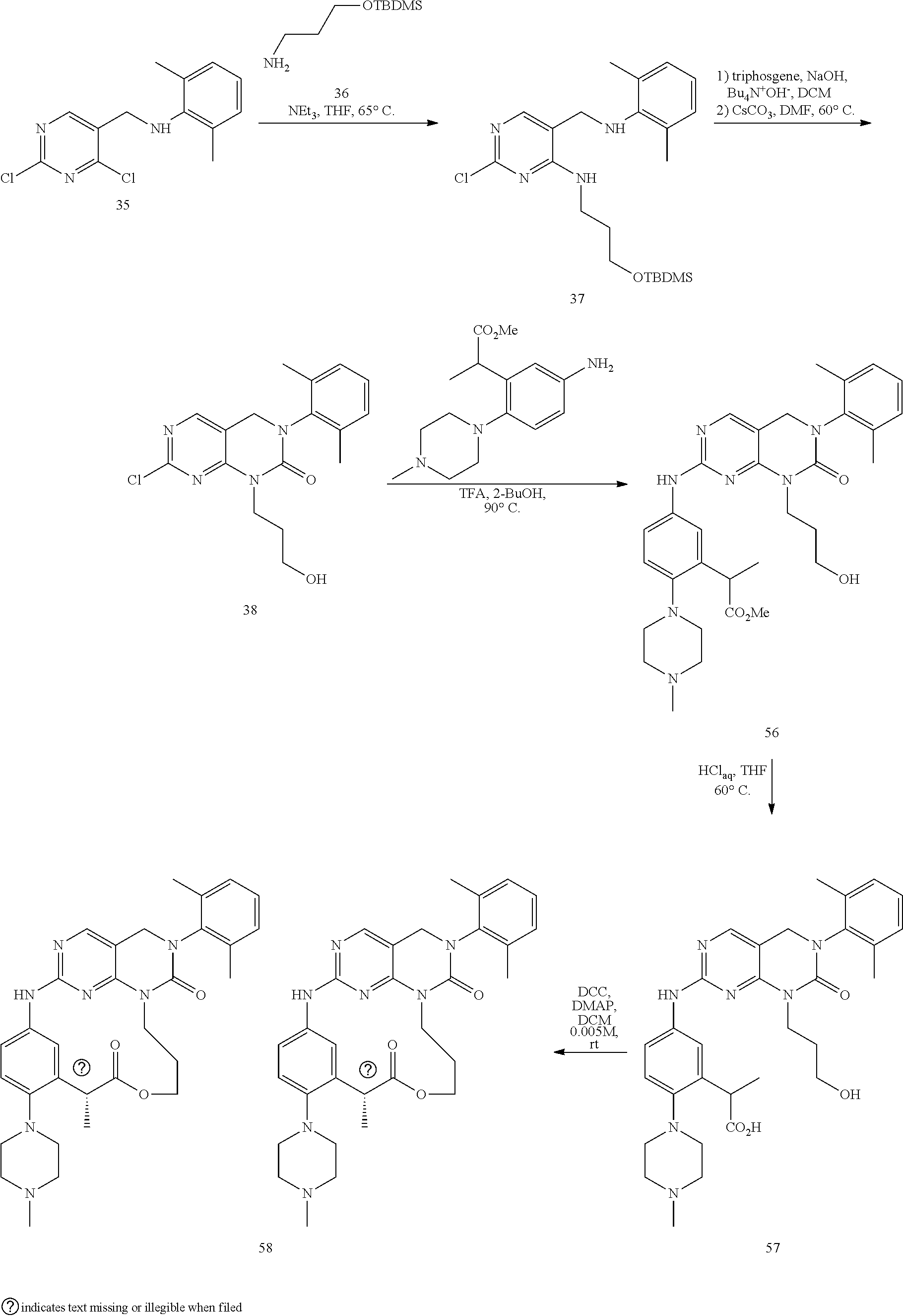

Previous intermediates (35-38) have already been described in the experimental section.

### 2-[5-[6-(2,6-Dimethyl-phenyl)-8-(3-hydroxy-propyl)-7-oxo-5,6,7,8-tetrahydro-pyrimido[4,5-d]pyrimidin-2-ylamino]-2-(4-methyl-piperazin-1-yl)-phenyl]-propionic acid methyl ester (56) (as racemic mixture)

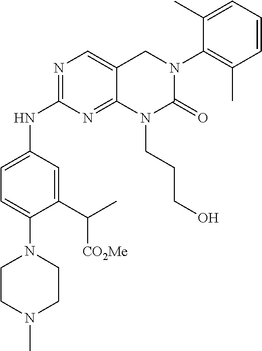 [SMILES: COC(=O)C(C)c1cc(Nc2ncc3c(n2)N(CCCO)C(=O)N(c2c(C)cccc2C)C3)ccc1N1CCN(C)CC1]

7-Chloro-3-(2,6-dimethyl-phenyl)-1-(3-hydroxy-propyl)-3,4-dihydro-1H-pyrimido[4,5-d]pyrimidin-2-one (0.969 g, 2.79 mmol) and 2-[5-Amino-2-(4-methyl-piperazin-1-yl)-phenyl]-propionic acid methyl ester (1.03 g, 3.72 mmol) were placed in a sealed tube and dissolved in anhydrous 2-BuOH (8.0 mL). Then 3A MS were added followed by anhydrous trifluoroacetic acid (0.428 mL, 5.6 mmol) and the reaction was stirred at 90° C. for 16 h. Thereafter, the reaction was diluted with water and treated with aq. solution of NaHCO3 (5.6 mmol). The aqueous phase was extracted with CHCl3/iPrOH mixture (1:1). Finally, the combined organic layers were dried over MgSO4, filtered and concentrated under vacuum. The obtained residue was purified by flash column chromatography on silica (DCM:MeOH from 100:0 to 90:10) to yield pure product (1.33 g, Yield: 81%, brown solid). LC-MS (ESI+): m/z: 588.3 RT: 1.178 min (TACC50).

### 2-[5-[6-(2,6-Dimethyl-phenyl)-8-(3-hydroxy-propyl)-7-oxo-5,6,7,8-tetrahydro-pyrimido[4,5-d]pyrimidin-2-ylamino]-2-(4-methyl-piperazin-1-yl)-phenyl]-propionic acid (57) (as racemic mixture)

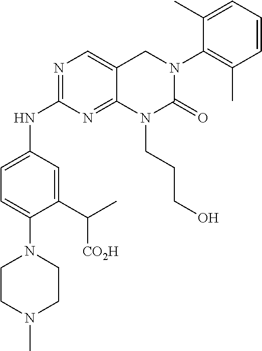 [SMILES: Cc1cccc(C)c1N1Cc2cnc(Nc3ccc(N4CCN(C)CC4)c(C(C)C(=O)O)c3)nc2N(CCCO)C1=O]

2-[5-[6-(2,6-Dimethyl-phenyl)-8-(3-hydroxy-propyl)-7-oxo-5,6,7,8-tetrahydro-pyrimido[4,5-d]pyrimidin-2-ylamino]-2-(4-methyl-piperazin-1-yl)-phenyl]-propionic acid methyl ester (0.8 g, 1.37 mmol) was dissolved in THF (4.0 mL) and treated with HCl (6.9 mmol, 6M aqueous solution, so 1.2 mL). The reaction was stirred at 60° C. for 16 h. Thereafter, solvent was removed under vacuum and the obtained residue was used without further purification. LC-MS (ESI+): m/z: 575.3, RT. 2.175 min (VILLA).

### 1³-(2,6-dimethylphenyl)-4-methyl-3⁴-(4-methylpiperazin-1-yl)-1¹,1²,1³,1⁴-tetrahydro-6-oxa-2-aza-1(7,1)-pyrimido[4,5-d]pyrimidina-3(1,3)-benzenacyclononaphane-1²,5-dione (58)

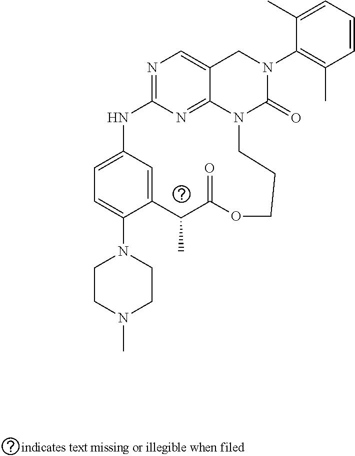 [SMILES: Cc1cccc(C)c1N1Cc2cnc3nc2N(CCCOC(=O)[C@H](C)c2cc(ccc2N2CCN(C)CC2)N3)C1=O]

2-[5-[6-(2,6-Dimethyl-phenyl)-8-(3-hydroxy-propyl)-7-oxo-5,6,7,8-tetrahydro-pyrimido[4,5-d]pyrimidin-2-ylamino]-2-(4-methyl-piperazin-1-yl)-phenyl]-propionic acid (0.393 g, 0.685 mmol) was dissolved in anhydrous DMF (3.0 mL) and diluted with anhydrous DCM (130.0 mL). 3A molecular shieves were added followed by DMAP (0.125 g, 1.03 mmol) and DCC (0.170 g, 0.82 mmol)) and the reaction was stirred at room temperature for 16 h. Then, solvent was removed under vacuum and the obtained residue was purified by flash column chromatography on silica (deactivated with DCM:NH3 1%) (DCM:MeOH from 100:0 to 90:10) to yield pure product. SFC purification was performed on a Jasco SFC prep system using an i-Cellulose-C column (Regis Technologies) 100 mm long×4.6 mm I.D. 3 μm particle size, on isocratic mode at 2.5 ml/min of CO2 (60%)-Ethanol (40%)+0.1% Diethylamine at 35° C., BPR 100 Bar to render pure enantiomers.

Enantiomer F1 (0.0291 g): LC-MS (ESI+): m/z: 557.3 RT: 2.450 min (VILLA). 99% e.e. (RT: 8.142 min).

Enantiomer F2 (0.0311 g): LC-MS (ESI+): m/z: 557.3 RT: 2.390 min (VILLA). 98% e.e (RT: 8.473 min).

1H NMR (400 MHz, DMSO) δ 9.56 (s, 1H), 8.14 (s, 1H), 8.10 (s, JH), 7.19-7.12 (m, 4H), 7.04 (dd, J=8.6, 2.4 Hz, 1H), 4.54 (d, J=14.5 Hz, 1H), 4.40 (d, J=14.4 Hz, 2H), 4.34 (q, J=6.9 Hz, 1H), 4.17 (t, J=10.2 Hz, 1H), 4.11-4.01 (m, 2H), 2.98-2.91 (m, 2H), 2.73-2.66 (m, 2H), 2.48-2.40 (m, 4H), 2.23 (s, 3H), 2.20 (s, 3H), 2.13 (s, 3H), 1.99-1.88 (m, 1H), 1.86-1.75 (m, 1H), 1.31 (d, J=6.9 Hz, 3H).

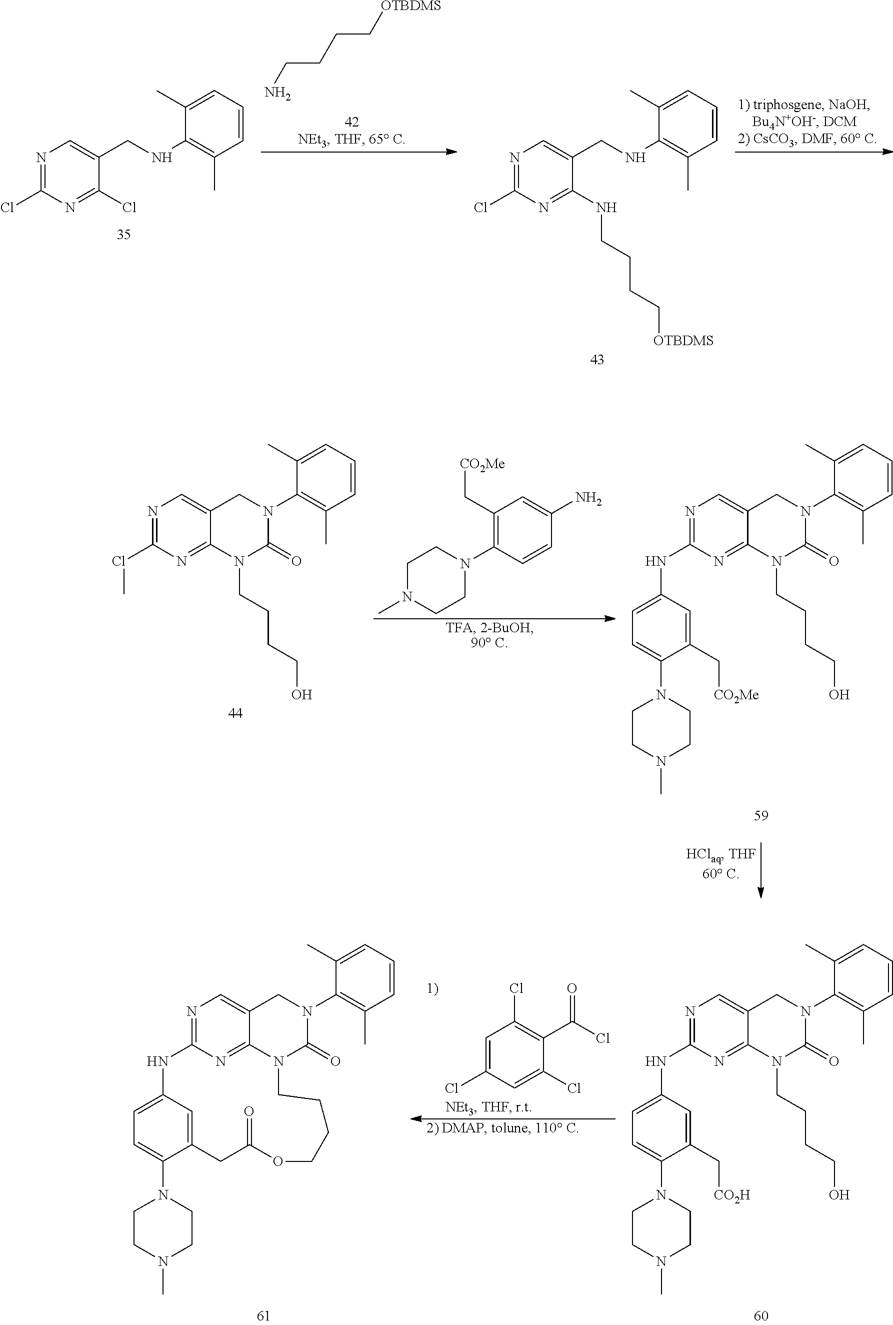

Previous intermediates (35-44) have already been described in the experimental section.

### [5-[6-(2,6-Dimethyl-phenyl)-8-(4-hydroxy-butyl)-7-oxo-5,6,7,8-tetrahydro-pyrimido[4,5-d]pyrimidin-2-ylamino]-2-(4-methyl-piperazin-1-yl)-phenyl]-acetic acid methyl ester (59)

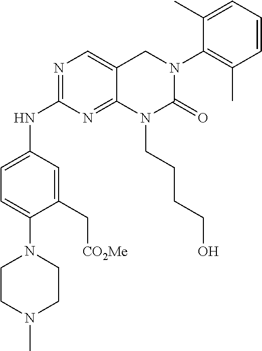 [SMILES: COC(=O)Cc1cc(Nc2ncc3c(n2)N(CCCCO)C(=O)N(c2c(C)cccc2C)C3)ccc1N1CCN(C)CC1]

7-Chloro-3-(2,6-dimethyl-phenyl)-1-(4-hydroxy-butyl)-3,4-dihydro-1H-pyrimido[4,5-d]pyrimidin-2-one (1.0 g, 2.771 mmol) and methyl [5-amino-2-(4-methylpiperazin-1-yl)phenyl] acetate (0.970 g, 3.685 mmol) were placed in a sealed tube and dissolved in anhydrous 2-BuOH (20.0 mL). Then 3A MS were added followed by anhydrous trifluoroacetic acid (0.63 mL, 5.54 mmol) and the reaction was stirred at 90° C. for 16 h. Thereafter, the reaction was diluted with water and treated with aq. solution of NaHCO3 (5.54 mmol). The aqueous phase was extracted with CHCl3/iPrOH mixture (1:1). Finally, the combined organic layers were dried over MgSO4, filtered and concentrated under vacuum. The obtained residue was purified by flash column chromatography on silica (DCM:MeOH from 100:0 to 90:10) to yield pure product (1.047 g, Yield: 64%, brown pale solid). LC-MS (ESI+): m/z: 588.3 RT: 2.290 min (VILLA). 2-(5-((6-(2,6-dimethylphenyl)-8-(4-hydroxybutyl)-7-oxo-5,6,7,8-tetrahydropyrimido[4,5-d]pyrimidin-2-yl)amino)-2-(4-methylpiperazin-1-yl)phenyl)acetic acid (60)

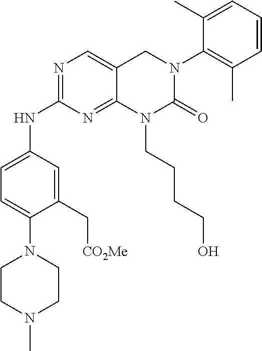 [SMILES: COC(=O)Cc1cc(Nc2ncc3c(n2)N(CCCCO)C(=O)N(c2c(C)cccc2C)C3)ccc1N1CCN(C)CC1]

[5-[6-(2,6-Dimethyl-phenyl)-8-(4-hydroxy-butyl)-7-oxo-5,6,7,8-tetrahydro-pyrimido[4,5-d]pyrimidin-2-ylamino]-2-(4-methyl-piperazin-1-yl)-phenyl]-acetic acid methyl ester (0.598 g, 1.02 mmol) was dissolved in THF (4.0 mL) and treated with HCl (5.08 mmol, 6M aqueous solution, so 0.845 mL). The reaction was stirred at 60° C. for 16 h. Thereafter, solvent was removed under vacuum and the obtained residue was purified by reverse phase chromatography (from 95% [aq. 0.1% HCOOH]-5% ACN to 63% [aq. 0.1% HCOOH]-37% ACN) to yield pure product (0.186 g, Yield: 32%). LC-MS (ESI+): m/z: 575.3, RT: 2.096 min (VILLA).

### 1³-(2,6-dimethylphenyl)-3⁴-(4-methylpiperazin-1-yl)-1¹,1²,1³,1⁴-tetrahydro-6-oxa-2-aza-1(7,1)-pyrimido[4,5-d]pyrimidina-3(1,3)-benzenacyclodecaphane-1²,5-dione (61)

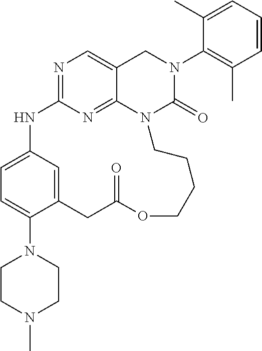 [SMILES: Cc1cccc(C)c1N1Cc2cnc3nc2N(CCCCOC(=O)Cc2cc(ccc2N2CCN(C)CC2)N3)C1=O]

2-(5-((6-(2,6-dimethylphenyl)-8-(4-hydroxybutyl)-7-oxo-5,6,7,8-tetrahydropyrimido[4,5-d]pyrimidin-2-yl)amino)-2-(4-methylpiperazin-1-yl)phenyl)acetic acid (0.160 g, 0.279 mmol) was dissolved in anhydrous THF (12.0 mL). Then triethylamine (0.086 mL, 0.614 mmol) and 2,4,6-trichlorobenzoyl chloride (0.087 mL, 0.558 mmol) were added and the reaction was stirred at room temperature for 16 h. After that time, the solvent was removed under vacuum and the obtained residue was dissolved in anhydrous DMF (2.0 mL) and diluted with anhydrous toluene (4.0 mL). Then it was dropwise added to a refluxing solution of DMAP (0.205 g, 1.674 mmol) in anhydrous toluene (50.0 mL, final concentration 0.005 mmol/mL, total volume 56.0 mL) via addition funnel and it was stirred at that temperature for 16 h. Thereafter the solvent was removed under vacuum and the obtained residue was purified by reverse phase chromatography (from 47% [65 mM NH4OAc+ACN (90:10)]-53% ACN to 18% [65 mM NH4OAc+ACN (90:10)]-82% ACN) to yield pure product (0.020 g, Yield: 13%). LC-MS (ESI+): m/z: 556.5, RT: 2.494 min (VILLA). 1H NMR (400 MHz, DMSO) δ 9.47 (s, 1H), 8.12 (s, 1H), 7.77 (s, 1H), 7.35 (d, J=8.1 Hz, 1H), 7.20-7.13 (m, 3H), 7.09 (d, J=8.6 Hz, 1H), 4.49 (s, 2H), 4.07 (t, J=5.8 Hz, 2H), 3.99 (t, J=6.6 Hz, 2H), 3.58 (s, 2H), 2.70-2.63 (m, 4H), 2.37-2.27 (m, 4H), 2.18 (s, 6H), 2.11 (s, 3H), 1.79-1.62 (m, 4H).

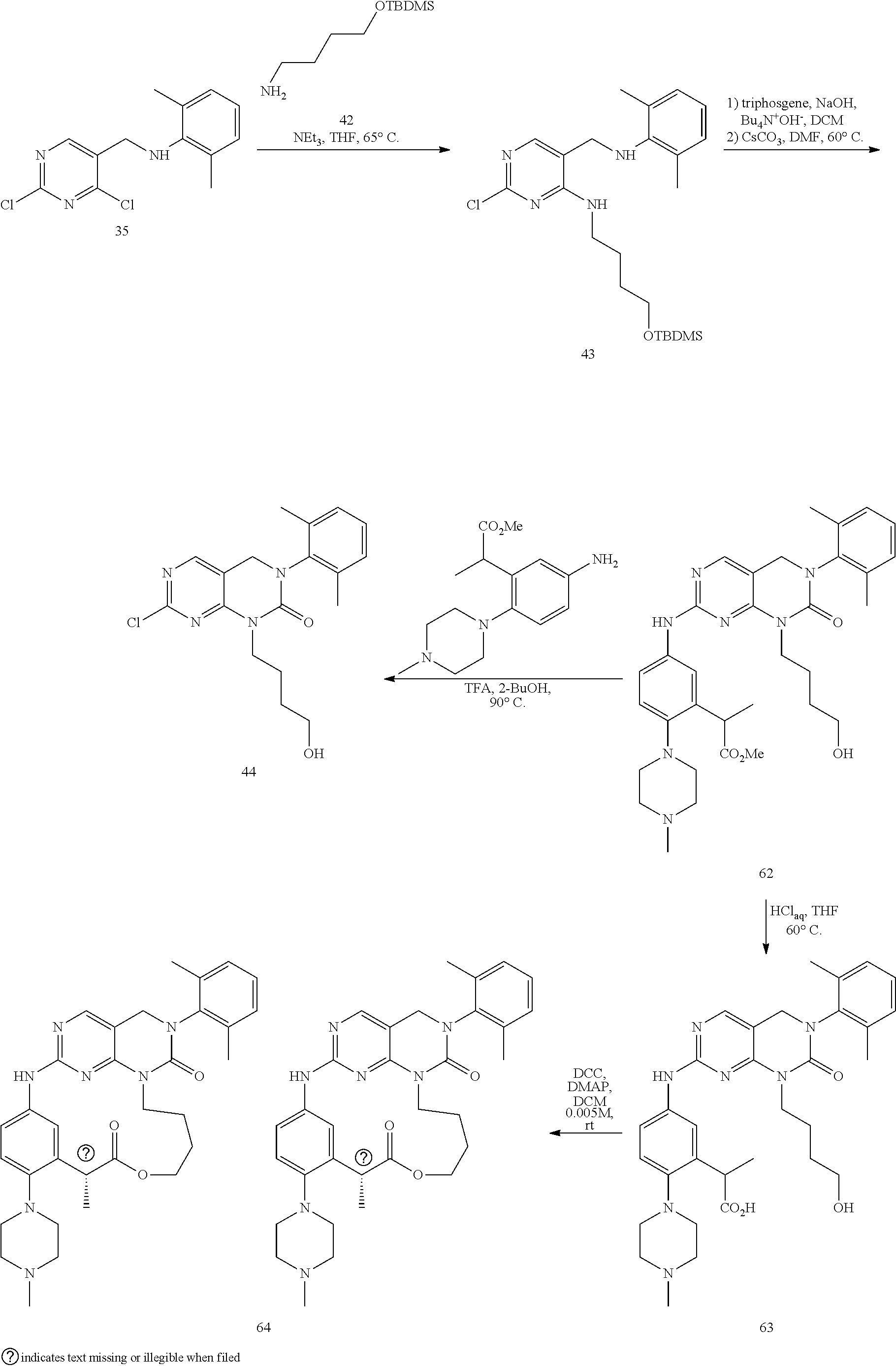

Previous intermediates (35-44) have already been described in the experimental section.

### 2-[5-[6-(2,6-Dimethyl-phenyl)-8-(4-hydroxy-butyl)-7-oxo-5,6,7,8-tetrahydro-pyrimido[4,5-d]pyrimidin-2-ylamino]-2-(4-methyl-piperazin-1-yl)-phenyl]-propionic acid methyl ester (62) (as racemic mixture)

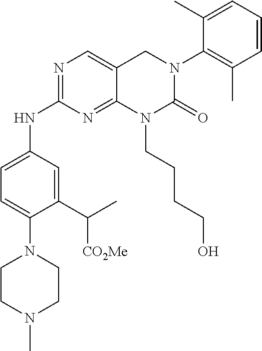 [SMILES: COC(=O)C(C)c1cc(Nc2ncc3c(n2)N(CCCCO)C(=O)N(c2c(C)cccc2C)C3)ccc1N1CCN(C)CC1]

7-Chloro-3-(2,6-dimethyl-phenyl)-1-(4-hydroxy-butyl)-3,4-dihydro-1H-pyrimido[4,5-d]pyrimidin-2-one (1.40 g, 3.88 mmol) and 2-[5-Amino-2-(4-methyl-piperazin-1-yl)-phenyl]-propionic acid methyl ester (1.43 g, 5.16 mmol) were placed in a sealed tube and dissolved in anhydrous 2-BuOH (20.0 mL). Then 3A MS were added followed by anhydrous trifluoroacetic acid (0.59 mL, 7.76 mmol) and the reaction was stirred at 90° C. for 16 h. Thereafter, the reaction was diluted with water and treated with aq. solution of NaHCO3 (7.76 mmol). The aqueous phase was extracted with CHCl3/iPrOH mixture (1:1). Finally, the combined organic layers were dried over MgSO4, filtered and concentrated under vacuum. The obtained residue was purified by flash column chromatography on silica (DCM:MeOH from 100:0 to 90:10) to yield pure product (1.24 g, Yield: 53%, yellow oil). LC-MS (ESI+): m/z: 601.7, RT: 2.148 min (VILLA).

### 2-[5-[6-(2,6-Dimethyl-phenyl)-8-(4-hydroxy-butyl)-7-oxo-5,6,7,8-tetrahydro-pyrimido[4,5-d]pyrimidin-2-ylamino]-2-(4-methyl-piperazin-1-yl)-phenyl]-propionic acid (63)

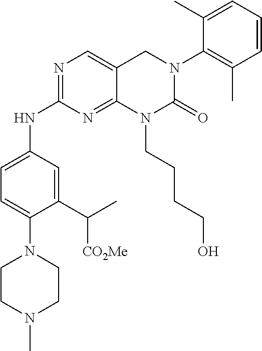 [SMILES: COC(=O)C(C)c1cc(Nc2ncc3c(n2)N(CCCCO)C(=O)N(c2c(C)cccc2C)C3)ccc1N1CCN(C)CC1]

2-[5-[6-(2,6-Dimethyl-phenyl)-8-(4-hydroxy-butyl)-7-oxo-5,6,7,8-tetrahydro-pyrimido[4,5-d]pyrimidin-2-ylamino]-2-(4-methyl-piperazin-1-yl)-phenyl]-propionic acid methyl ester (0.99 g, 1.65 mmol) was dissolved in THF (10.0 mL) and treated with HCl (8.22 mmol, 6 M aqueous solution, so 1.37 mL). The reaction was stirred at 60° C. for 16 h. Thereafter, solvent was removed under vacuum and the obtained residue was used without further purification. LC-MS (ESI+): m/z: 588.0, RT: 1.57 min (TACC50).

### 1³-2,6-dimethylphenyl)-4-methyl-3⁴-(4-methylpiperazin-1-yl)-1¹,1²,1³,1⁴-tetrahydro-6-oxa-2-aza-1(7,1)-pyrimido[4,5-d]pyrimidina-3(1,3)-benzenacyclodecaphane-12,5-dione (64)

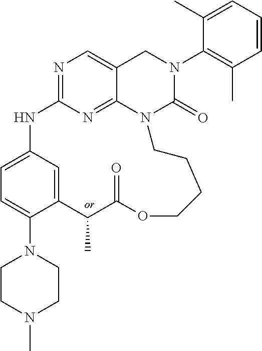 [SMILES: Cc1cccc(C)c1N1Cc2cnc3nc2N(CCCCOC(=O)[C@H](C)c2cc(ccc2N2CCN(C)CC2)N3)C1=O]

2-[5-[6-(2,6-Dimethyl-phenyl)-8-(4-hydroxy-butyl)-7-oxo-5,6,7,8-tetrahydro-pyrimido[4,5-d]pyrimidin-2-ylamino]-2-(4-methyl-piperazin-1-yl)-phenyl]-propionic acid (0.965 g, 1.645 mmol) was dissolved in anhydrous DMF (2.0 mL) and diluted with anhydrous DCM (220 mL). 3A molecular shieves were added followed by DMAP (0.30 g, 2.47 mmol) and DCC (0.41 g, 1.97 mmol) and the reaction was stirred at room temperature for 16 h. The reaction was recharged with DMAP (C, 0.15 g, 1.24 mmol) and DCC (B, 0.41 g, 1.97 mmol) and it was stirred at room temperature for further 16 h. Then, solvent was removed under vacuum. The obtained residue was dissolved in ACN, formed solids were removed by filtration. The obtained filtrate was concentrated under vacuum rendering a residue which was purified by flash column chromatography on silica (deactivated with DCM:NH3 1%) (DCM:MeOH from 100:0 to 90:10) to yield pure product. SFC purification was performed on a Jasco SFC prep system using an i-Cellulose-C column (Regis Technologies) 100 mm long×4.6 mm I.D. 3 μm particle size, on isocratic mode at 2.5 ml/min of CO2 (60%)-2-propanol (40%)+0.1% Diethylamine at 35° C., BPR 100 Bar to render pure enantiomers.

Enantiomer F1 (0.0338 g): LC-MS (ESI+): m/z: 571.3 RT: 2.442 min (VILLA), 99% e.e (RT: 7.697 min)

Enantiomer F2 (0.0354 g): LC-MS (ESI+): m/z: 571.3 RT: 2.443 min (VILLA), 97% e.e (RT: 7.960 min)

1H NMR (400 MHz, DMSO) δ 9.46 (s, 1H), 8.10 (s, 1H), 8.06 (d, J=2.4 Hz, 1H), 7.19-7.12 (m, 4H), 7.02 (dd, J=8.6, 2.4 Hz, 1H), 4.47 (series of m, 3H), 4.34-4.27 (m, 1H), 4.11-4.02 (m, 1H), 3.97-3.90 (m, 1H), 3.85 (dd, J=16.0, 8.4 Hz, 1H), 3.09-3.00 (m, 2H), 2.67 (broad signal, 2H), 2.44 (broad signal, 4H), 2.23 (s, 3H), 2.18 (s, 3H), 2.17 (s, 3H), 1.88-1.80 (m, 1H), 1.73-1.60 (m, 3H), 1.31 (d, J=7.1 Hz, 3H).

### Type C Macrocycles

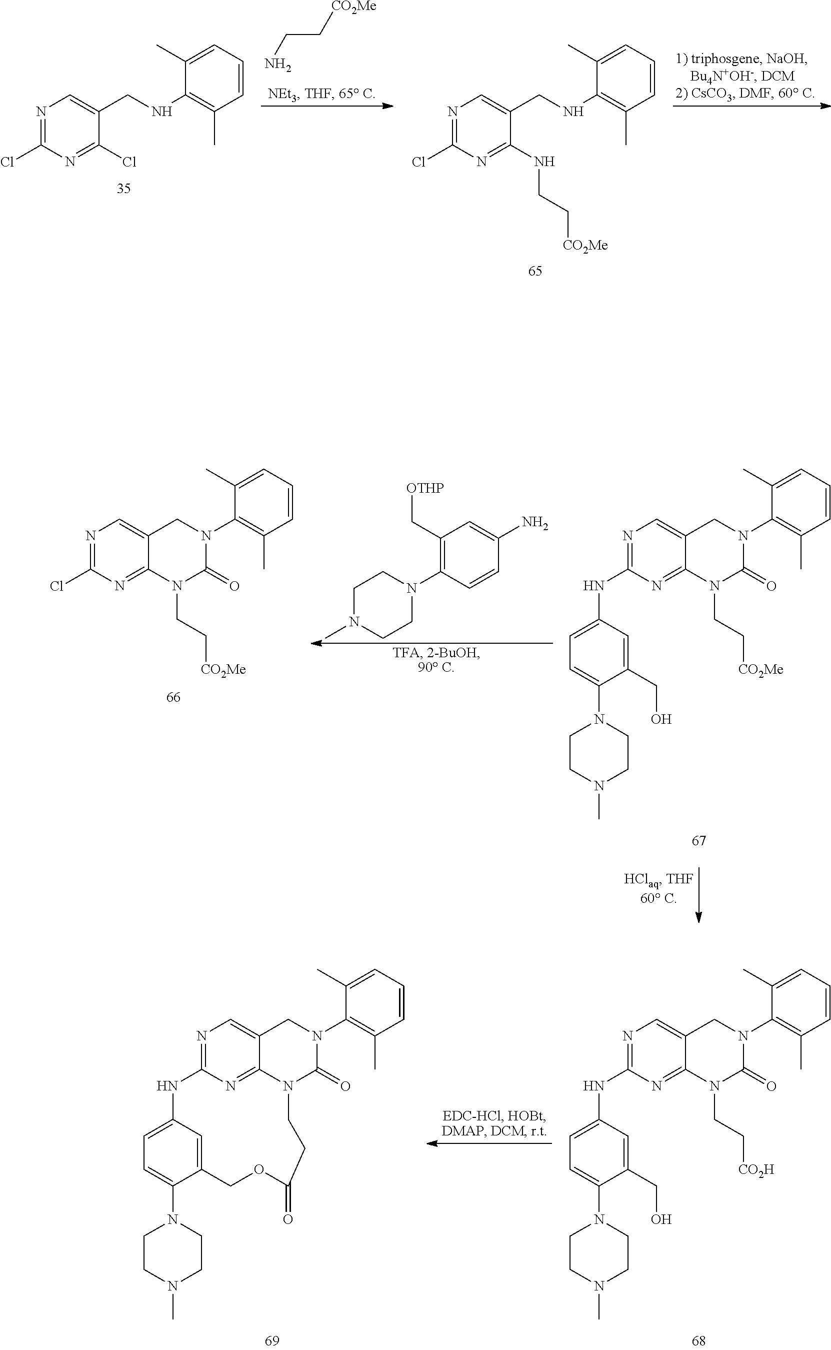

### Methyl 3-({2-chloro-5-[(2,6-dimethylanilino)methyl]pyrimidin-4-yl}amino)propanoate (65)

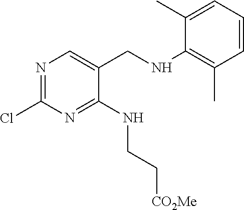 [SMILES: COC(=O)CCNc1nc(Cl)ncc1CNc1c(C)cccc1C]

Methyl 3-aminopropanoate hydrochloride (4.91 g, 35.44 mmol) and triethylamine (7.41 mL, 53.16 mmol) were added to a solution of N-((2,4-dichloropyrimidin-5-yl)methyl)-2,6-dimethylaniline (4.61 g, 16.34 mmol) in anhydrous THF (50.0 mL) and the mixture was stirred at 65° C. for 16 h. Thereafter, the reaction crude was diluted with AcOEt and washed with brine. The organic layer was dried over MgSO4, filtered and concentrated under vacuum. The obtained residue was purified by flash column chromatography on silica (heptane/EtOAc from 100:0 to 70:30) to yield pure product (5.12 g, Yield: 83%, yellow oil). LC-MS (ESI+): m/z: 348.9, RT: 1.616 min (TACC50).

### methyl 3-(7-chloro-3-(2,6-dimethylphenyl)-2-oxo-3,4-dihydropyrimido[4,5-d]pyrimidin-1(2H)-yl)propanoate (66)

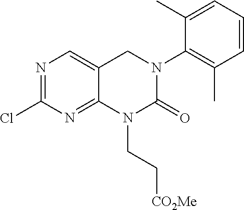 [SMILES: COC(=O)CCN1C(=O)N(c2c(C)cccc2C)Cc2cnc(Cl)nc21]

Triphosgene (6.53 g, 22.01 mmol) was added to a solution of methyl 3-({2-chloro-5-[(2,6-dimethylanilino)methyl]pyrimidin-4-yl}amino)propanoate (5.12 g, 14.68 mmol) in DCM (45.0 mL) and it was stirred at room temperature for 1 h. Then an aq. solution of sodium hydroxide (11.74 g, 293.54 mmol, 4.9 M) and tetrabutylammonium hydroxide (1.908 mmol 55% in aq. solution, so 0.9 mL) was added at 0° C. The reaction was allowed to reach room temperature and stirred at room temperature for 16 h. Thereafter, the solvent was removed under vacuum and the obtained residue was dissolved in DCM and washed with water. The organic layer was dried over MgSO4, filtered and concentrated under vacuum. The obtained residue was dissolved in DMF (40.0 mL) and cessium carbonate (3.11 g, 16.05 mmol) was added. The reaction mixture was heated at 60° C. for 16 h. Thereafter, the reaction crude was diluted with DCM and washed with brine. The organic layer was dried over MgSO4, filtered and concentrated under vacuum. The obtained residue was purified by flash column chromatography on silica (Heptane:AcOEt 100:0 to 80:20) to yield pure product (3.054 g, Yield: 56%, yellow solid). LC-MS (ESI+) m/z: 374.8, RT: 1.58 min (TACC50).

### 3-{3-(2,6-Dimethyl-phenyl)-7-[4-(4-methyl-piperazin-1-yl)-3-(tetrahydro-pyran-2-yloxymethyl)-phenylamino]-2-oxo-3,4-dihydro-2H-pyrimido[4,5-d]pyrimidin-1-yl}-propionic acid methyl ester 67)

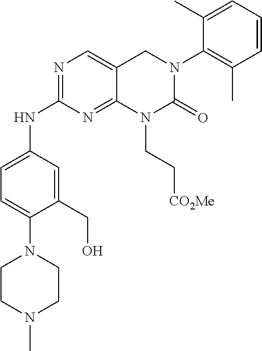 [SMILES: COC(=O)CCN1C(=O)N(c2c(C)cccc2C)Cc2cnc(Nc3ccc(N4CCN(C)CC4)c(CO)c3)nc21]

3-[7-Chloro-3-(2,6-dimethyl-phenyl)-2-oxo-3,4-dihydro-2H-pyrimido[4,5-d]pyrimidin-1-yl]-propionic acid methyl ester (0.5 g, 1.33 mmol) and 4-(4-Methyl-piperazin-1-yl)-3-(tetrahydro-pyran-2-yloxymethyl)-phenylamine (0.54 g, 1.77 mmol) were placed in a sealed tube and dissolved in anhydrous 2-BuOH (10.0 mL). Then 3A Ms were added followed by trifluoroacetic acid (0.2 mL, 2.66 mmol) and the reaction was stirred at 90° C. for 16 h. Thereafter, the reaction was diluted with water and treated with aq. solution of NaHCO3 (2.66 mmol). The aqueous phase was extracted with CHCl3/iPrOH mixture (1:1). Finally, the combined organic layers were dried over MgSO4, filtered and concentrated under vacuum. The obtained residue was purified by flash column chromatography on silica (DCM:MeOH 100:0 to 90:10) to yield pure product (0.50 g, Yield: 67%, brown pale solid). LC-MS (ESI+): m/z: 560.9 RT: 1.124 min (TACC50).

### 3-{3-(2,6-Dimethyl-phenyl)-7-[3-hydroxymethyl-4-(4-methyl-piperazin-1-yl)-phenylamino]-2-oxo-3,4-dihydro-2H-pyrimido[4,5-d]pyrimidin-1-yl}-propionic acid (68)

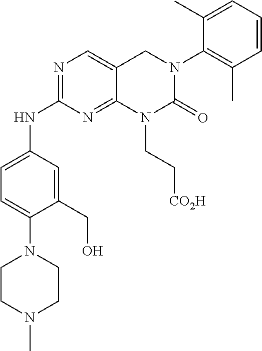 [SMILES: Cc1cccc(C)c1N1Cc2cnc(Nc3ccc(N4CCN(C)CC4)c(CO)c3)nc2N(CCC(=O)O)C1=O]

3-{3-(2,6-Dimethyl-phenyl)-7-[3-hydroxymethyl-4-(4-methyl-piperazin-1-yl)-phenylamino]-2-oxo-3,4-dihydro-2H-pyrimido[4,5-d]pyrimidin-1-yl}-propionic acid methyl ester (0.5 g, 0.89 mmol) was dissolved in THF (3 mL) and treated with HCl (4.46 mmol, 6 M aq solution, so 0.743 mL) and it was stirred at 60° C. for 3 h. Thereafter, solvent was removed under vacuum and the obtained residue was purified by reverse phase chromatography (from 95% [aq. 0.1% HCOOH]-5% CAN, to 63% [aq. 0.1% HCOOH]-37% ACN) to yield pure product (0.308 g, Yield: 63%, white solid). LC-MS (ESI+) m/z: 546.2, RT: 2.029 min (VILLA).

### 1³-(2,6-dimethylphenyl)-3⁴-(4-methylpiperazin-1-yl)-1¹,1²,1³,1⁴-tetrahydro-5-oxa-2-aza-1(7,1)-pyrimido[4,5-d]pyrimidina-3(1,3)-benzenacyclooctaphane-1²,6-dione (69)

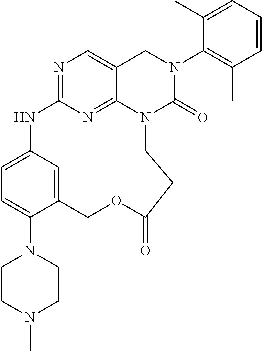 [SMILES: Cc1cccc(C)c1N1Cc2cnc3nc2N(CCC(=O)OCc2cc(ccc2N2CCN(C)CC2)N3)C1=O]

3-{3-(2,6-Dimethyl-phenyl)-7-[3-hydroxymethyl-4-(4-methyl-piperazin-1-yl)-phenylamino]-2-oxo-3,4-dihydro-2H-pyrimido[4,5-d]pyrimidin-1-yl}-propionic acid (1.00 g, 1.849 mmol) and DMAP (0.565 g, 4.622 mmol) were dissolved in anhydrous DCM (230 mL). Then 3A MS were added followed by EDC hydrochloride (0.354 g, 1.849 mmol) and 1-hydroxybenzotriazole hydrate (0.375 g, 2.773 mmol) and the reaction was stirred at room temperature for 16 h. Thereafter, the reaction crude was washed with an aqueous solution of Rochelle salt. The organic layer was dried over MgSO4, filtered and concentrated under vacuum. The obtained residue was purified flash column chromatography on silica (deactivated with DCM:NH3 1%) (DCM:MeOH 100:0 to 90:10) to yield pure product (0.320 g, Yield: 33%, white solid). LC-MS (ESI+) m/z: 528.2, RT. 2.383 min (VILLJ). 1H NMR (400 MHz, CDCl3) δ 8.24 (d, J=2.5 Hz, 1H), 7.96 (s, 1H), 7.47 (s, 1H), 7.19-7.10 (m, 3H), 7.01 (d, J=8.4 Hz, 1H), 6.82 (dd, J=8.4, 2.6 Hz, 1H), 5.37 (s, 2H), 4.46 (s, 2H), 4.29 (t, J=6.1 Hz, 2H), 2.95 (t, J=4.3 Hz, 4H), 2.60 (s, 4H), 2.37 (s, 3H), 2.26 (s, 6H).

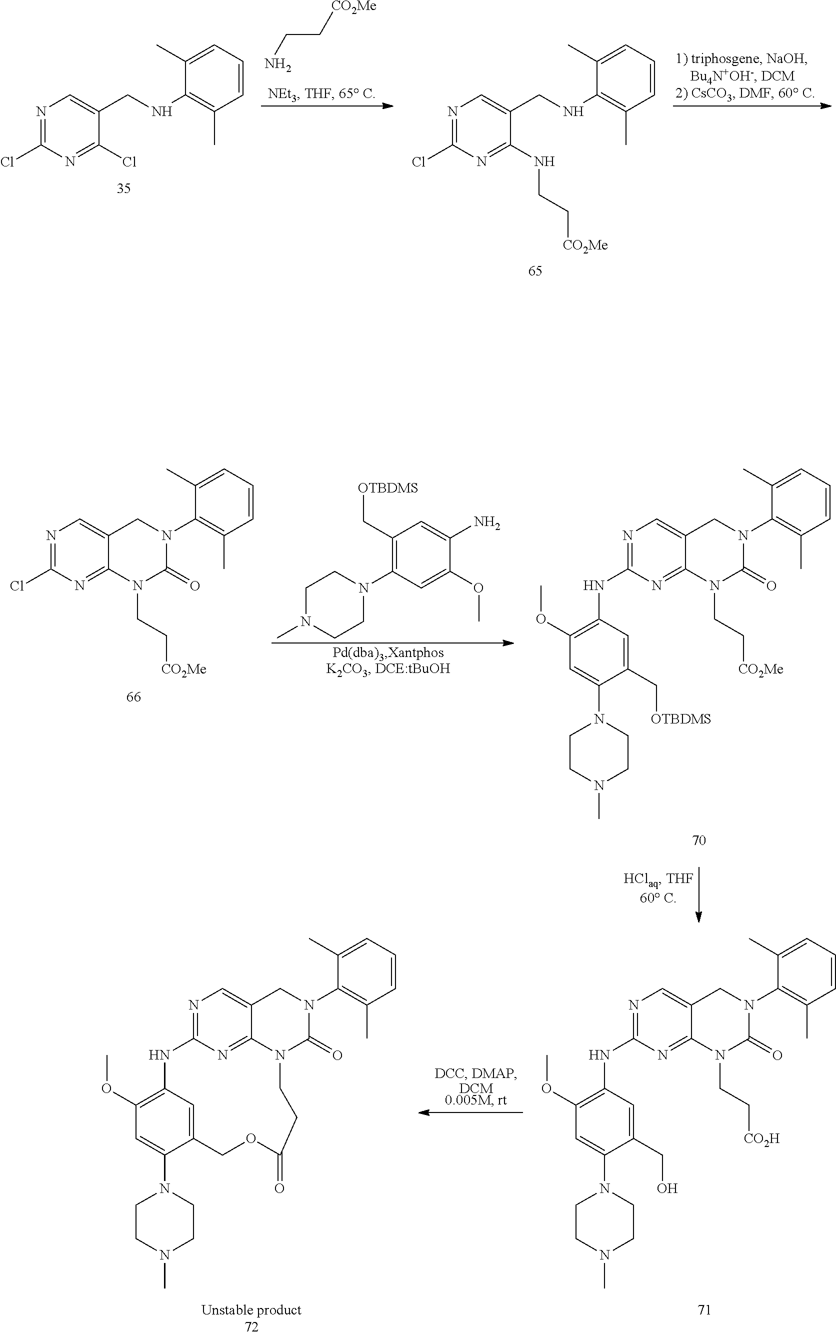

Previous intermediates (35-66) have already been described in the experimental section.

### methyl 3-(7-((5-(((tert-butyldimethylsilyl)oxy)methyl)-2-methoxy-4-(4-methylpiperazin-1-yl)phenyl)amino)-3-(2,6-dimethylphenyl)-2-oxo-3,4-dihydropyrimido[4,5-d]pyrimidin-1(2H)-yl)propanoate (70)

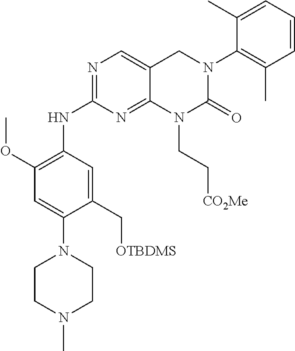 [SMILES: COC(=O)CCN1C(=O)N(c2c(C)cccc2C)Cc2cnc(Nc3cc(CO[Si](C)(C)C(C)(C)C)c(N4CCN(C)CC4)cc3OC)nc21]

methyl 3-[7-chloro-3-(2,6-dimethylphenyl)-2-oxo-3,4-dihydropyrimido[4,5-d]pyrimidin-1(2H)-yl]propanoate (1.80 g, 4.802 mmol), 5-(tert-Butyl-dimethyl-silanyloxymethyl)-2-methoxy-4-(4-methyl-piperazin-1-yl)-phenylamine (1.843 g, 5.04 mmol) and K2CO3 (1.00 g, 7.20 mmol) were placed in a sealed tube and dissolved in a tBuOH:DCE mixture (7:1, 32.0 mL). The reaction was purged with N2 and then, Pd2dba3 (0.659 g, 0.720 mmol) and XantPhos (0.391 g, 0.72 mmol) were added. The mixture was purged with N2 again and the reaction was stirred at 85° C. for 16 h. Then, the reaction crude was filtered over celite and rinsed with DCM. The filtrate was washed with water, dried over MgSO4, filtered and concentrated under vacuum. The obtained residue was purified by reverse phase chromatography (from 32% [25 mM NH4HCO3]-68% ACN to 4% [25 mM NH4HCO3]-96% ACN) to yield pure product (0.610 g, Yield: 18%, brown solid). LC-MS (ESI+): m/z: 704.3, R.T: 1.61 min (TACC50).

### 3-(3-(2,6-dimethylphenyl)-7-((5-(hydroxymethyl)-2-methoxy-4-(4-methylpiperazin-1-yl)phenyl)amino)-2-oxo-3,4-dihydropyrimido[4,5-d]pyrimidin-1(2H)-yl)propanoic acid (71)

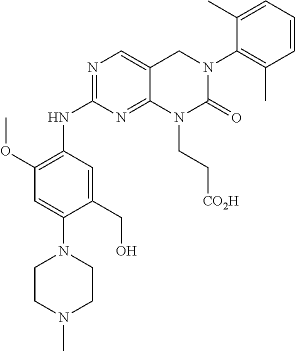 [SMILES: COc1cc(N2CCN(C)CC2)c(CO)cc1Nc1ncc2c(n1)N(CCC(=O)O)C(=O)N(c1c(C)cccc1C)C2]

Methyl 3-(7-((5-(((tert-butyldimethylsilyl)oxy)methyl)-2-methoxy-4-(4-methylpiperazin-1-yl)phenyl)amino)-3-(2,6-dimethylphenyl)-2-oxo-3,4-dihydropyrimido[4,5-d]pyrimidin-1(2H)-yl)propanoate (0.612 g, 0.871 mmol) was dissolved in THF (10.0 mL) and treated with HCl (4.355 mmol, 6 M aqueous solution, so 0.73 mL) and it was heated at 60° C. for 2 h. Thereafter, solvent was removed under vacuum and the obtained residue was used without further purification. LC-MS (ESI+): m/z: 576.30 RT: 0.68 min (TACC50).

### 1³-2,6-dimethylphenyl)-3⁶-methoxy-3⁴-(4-methylpiperazin-1-yl)-1¹,1²,1³,1⁴-tetrahydro-5-oxa-2-aza-1(7,1)-pyrimido[4,5-d]pyrimidina-3(1,3)-benzenacyclooctaphane-1²,6-dione (72)

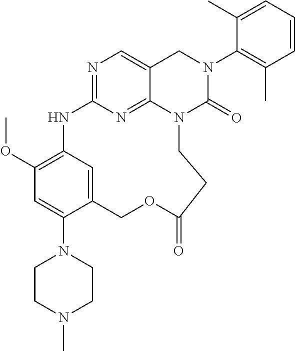 [SMILES: COc1cc(N2CCN(C)CC2)c2cc1Nc1ncc3c(n1)N(CCC(=O)OC2)C(=O)N(c1c(C)cccc1C)C3]

3-(3-(2,6-dimethylphenyl)-7-((5-(hydroxymethyl)-2-methoxy-4-(4-methylpiperazin-1-yl)phenyl)amino)-2-oxo-3,4-dihydropyrimido[4,5-d]pyrimidin-1(2H)-yl)propanoic acid (0.871 g, 1.513 mmol) was dissolved in anhydrous DMF (2.0 mL) and diluted with anhydrous DCM (118.0 mL). 3A molecular shieves were added followed by DMAP (0.277 g, 2.269 mmol) and DCC (0.375 g, 1.816 mmol) and the reaction was stirred at room temperature for 16 h. Then, solvent was removed under vacuum and the obtained residue was purified by reverse phase chromatography (from 81%-19% ACN to 45% [aq. 25 mM NH4HCO3]-55% ACN) to yield the product. This product turned out to be unstable and decomposed spontaneously to a mixture of open intermediate:macrocycle.

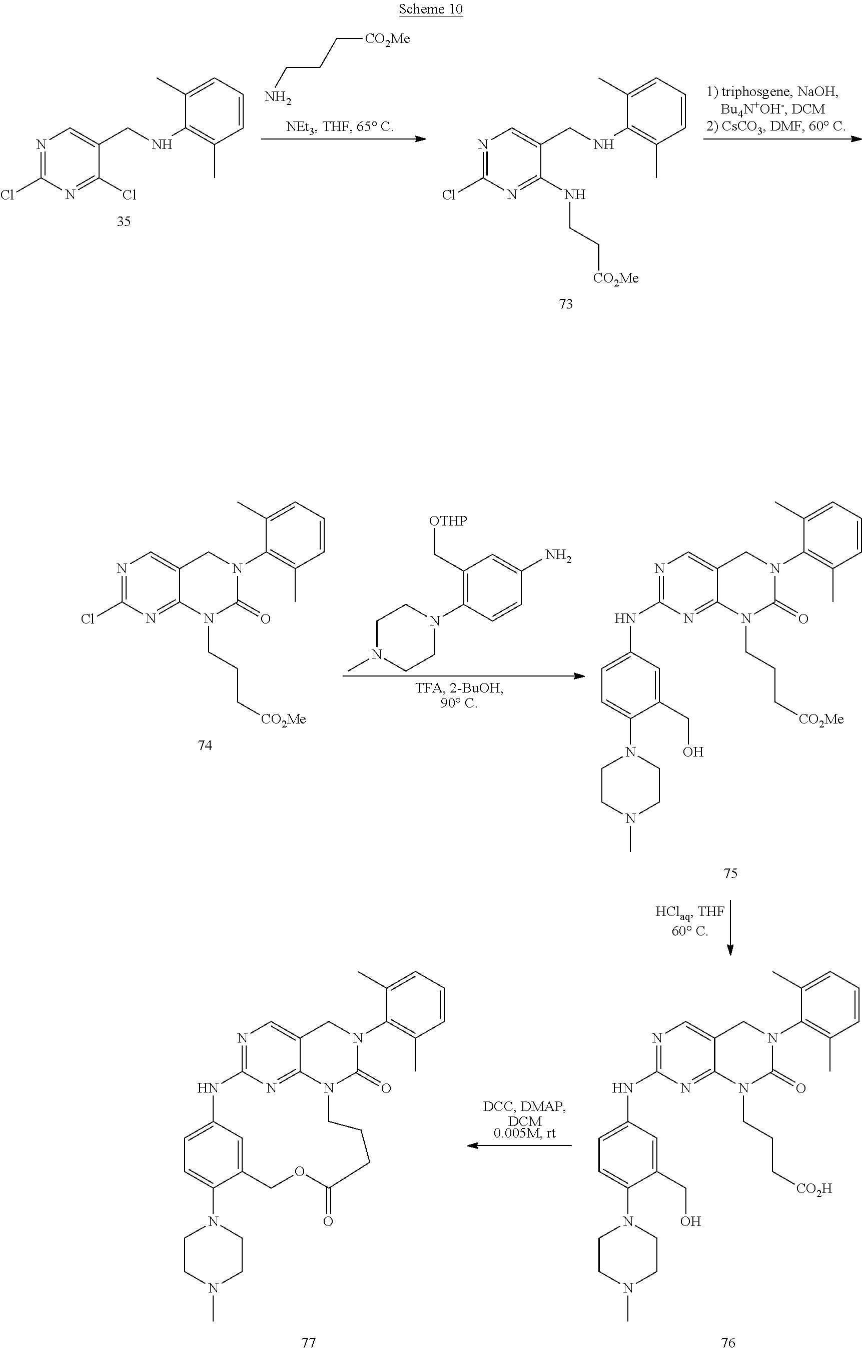

### methyl 4-({2-chloro-5-[(2,6-dimethylanilino)methyl]pyrimidin-4-yl}amino)butanoate (73)

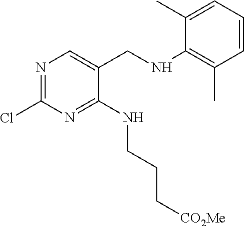 [SMILES: COC(=O)CCCNc1nc(Cl)ncc1CNc1c(C)cccc1C]

Methyl 4-Aminobutyrate hydrochloride (2.418 g, 15.95 mmol) and triethylamine (4.446 mL, 31.89 mmol) were added to a solution of N-((2,4-dichloropyrimidin-5-yl)methyl)-2,6-dimethylaniline (3.00 g, 10.63 mmol) in anhydrous THF (32.0 mL) and the mixture was stirred at 65° C. for 16 h. Thereafter, the reaction crude was diluted with AcOEt and washed with brine. The organic layer was dried over MgSO4, filtered and concentrated under vacuum. The obtained residue was purified by flash column chromatography on silica (heptane/EtOAc from 100:0 to 70:30) to yield pure product (3.48 g, Yield: 90%, yellow oil). LC-MS (ESI+): m/z: 362.8 RT: 1.616 min (TACC50).

### methyl 4-[7-chloro-3-(2,6-dimethylphenyl)-2-oxo-3,4-dihydropyrimido[4,5-d]pyrimidin-1(2H)-yl]butanoate (74)

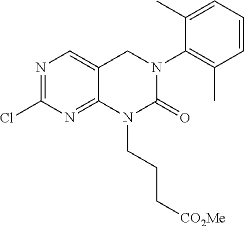 [SMILES: COC(=O)CCCN1C(=O)N(c2c(C)cccc2C)Cc2cnc(Cl)nc21]

Triphosgene (4.26 g, 14.34 mmol) was added to a solution of methyl 4-({2-chloro-5-[(2,6-dimethylanilino)methyl]pyrimidin-4-yl}amino)butanoate (3.47 g, 9.563 mmol) in DCM (29.0 mL) and it was stirred at room temperature for 1 h. Then an aq. solution of sodium hydroxide (7.65 g, 191.26 mmol, 4.9 M) and tetrabutylammonium hydroxide (1.243 mmol 55% in aq. solution, so 0.58 mL) was added at 0° C. The reaction was allowed to reach room temperature and stirred at room temperature for 16 h. Thereafter, the reaction crude was diluted with DCM and washed with water. The organic layer was dried over MgSO4, filtered and concentrated under vacuum. The obtained residue was dissolved in DMF (29.0 mL) and cessium carbonate (2.031 g, 10.47 mmol) was added. The reaction mixture was heated at 60° C. for 16 h. Thereafter, the reaction crude was diluted with DCM and washed with brine. The organic layer was dried over MgSO4, filtered and concentrated under vacuum. The obtained residue was purified by flash column chromatography on silica (Heptane:AcOEt 100:0 to 50:50) to yield pure product (2.21 g, Yield: 60%, yellow solid). LC-MS (ESI+): m/z: 388.8 RT: 1.652 min (TACC50).

### 4-{3-(2,6-Dimethyl-phenyl)-7-[4-(4-methyl-piperazin-1-yl)-3-(tetrahydro-pyran-2-yloxymethyl)-phenylamino]-2-oxo-3,4-dihydro-2H-pyrimido[4,5-d]pyrimidin-1-yl}-butyric acid methyl ester (75)

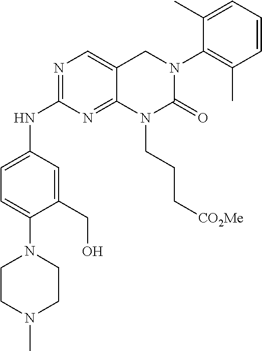 [SMILES: COC(=O)CCCN1C(=O)N(c2c(C)cccc2C)Cc2cnc(Nc3ccc(N4CCN(C)CC4)c(CO)c3)nc21]

4-[7-Chloro-3-(2,6-dimethyl-phenyl)-2-oxo-3,4-dihydro-2H-pyrimido[4,5-d]pyrimidin-1-yl]-butyric acid methyl ester (1.0 g, 2.57 mmol) and 4-(4-Methyl-piperazin-1-yl)-3-(tetrahydro-pyran-2-yloxymethyl)-phenylamine (1.05 g, 3.4 mmol) were placed in a sealed tube and dissolved in anhydrous 2-BuOH (20.0 mL). Then 3A MS were added followed by anhydrous TFA (0.4 mL, 5.14 mmol) and the reaction was stirred at 90° C. for 16 h. Thereafter, the reaction was diluted with water and treated with aq. solution of NaHCO3 (5.14 mmol). The aqueous phase was extracted with CHCl3/iPrOH mixture (1:1). Finally, the combined organic layers were dried over MgSO4, filtered and concentrated under vacuum. The obtained residue was purified by flash column chromatography on silica (DCM:MeOH 100:0 to 90:10) to yield pure product (0.947 g, Yield: 64%, brown pale solid). LC-MS (ESI+): m/z: 574.2 RT: 2.256 min (VILL-J).

### 4-{3-(2,6-Dimethyl-phenyl)-7-[3-hydroxymethyl-4-(4-methyl-piperazin-1-yl)-phenylamino]-2-oxo-3,4-dihydro-2H-pyrimido[4,5-d]pyrimidin-1-yl}-butyric acid (76)

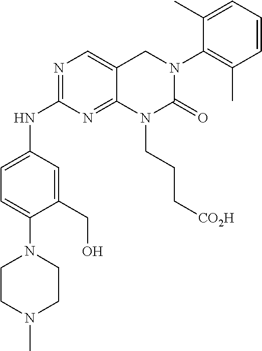 [SMILES: Cc1cccc(C)c1N1Cc2cnc(Nc3ccc(N4CCN(C)CC4)c(CO)c3)nc2N(CCCC(=O)O)C1=O]

4-{3-(2,6-Dimethyl-phenyl)-7-[3-hydroxymethyl-4-(4-methyl-piperazin-1-yl)-phenylamino]-2-oxo-3,4-dihydro-2H-pyrimido[4,5-d]pyrimidin-1-yl}-butyric acid methyl ester (0.90 g, 1.57 mmol) was dissolved in THF (5.0 mL) and treated with HCl (7.84 mmol, 6 M aqueous solution, so 1.3 mL) at 60° C. for 3 h. Thereafter, solvent was removed under vacuum and the obtained residue was purified by reverse phase chromatography (from 95% [aq. 0.1% HCOOH]-5% ACN to 63% [aq. 0.1% HCOOH]-37% ACN) to yield pure product (0.520 g, Yield: 59%, white solid). LC-MS (ESI+): m/z: 560.2 RT: 2.017 min (VILLA).

### 1³-(2,6-dimethylphenyl)-3⁴-(4-methylpiperazin-1-yl)-1¹,1², 1³,1⁴-tetrahydro-5-oxa-2-aza-1(7,1)-pyrimido[4,5-d]pyrimidina-3(1,3)-benzenacyclononaphane-1²,6-dione (77)

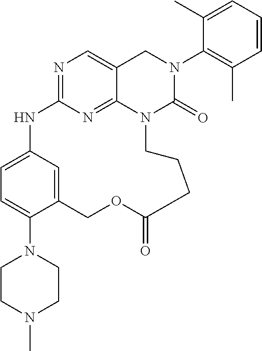 [SMILES: Cc1cccc(C)c1N1Cc2cnc3nc2N(CCCC(=O)OCc2cc(ccc2N2CCN(C)CC2)N3)C1=O]

4-{3-(2,6-Dimethyl-phenyl)-7-[3-hydroxymethyl-4-(4-methyl-piperazin-1-yl)-phenylamino]-2-oxo-3,4-dihydro-2H-pyrimido[4,5-d]pyrimidin-1-yl}-butyric acid (0.114 g, 0.204 mmol) was dissolved in anhydrous DMF (3.0 mL) and diluted in anhydrous DCM (35.0 mL). The reaction mixture was cooled to 0° C. and DCC (0.051 g, 0.245 mmol), DMAP (0.050 g, 0.408 mmol) and 3A MS were sequentially added. The reaction was allowed to reach room and it was stirred for 16 h. The reaction crude was concentrated under vacuum and the obtained residue was dissolved in ACN. Formed solids were removed by filtration. The filtrate was concentrated under vacuum and the obtained residue was purified by reverse phase chromatography (from 59% [aq. 25 mM NH4HCO3]-41% ACN to 17% [aq. 25 mM NH4HCO3]-83% ACN) to yield pure product (0.0101 g, Yield: 9%, white solid). LC-MS (ESI+): m/z: 542.1 RT: 2.501 min (VILL-J). 1H NMR (400 MHz, CDCl3) δ 8.48 (d, J=1.9 Hz, 1H), 7.95 (s, 1H), 7.42 (s, 1H), 7.19-7.05 (m, 4H), 6.78 (dd, J=8.4, 2.3 Hz, 1H), 5.42 (s, 2H), 4.48 (s, 2H), 4.20-4.12 (m, 2H), 2.92 (t, J=4.6 Hz, 4H), 2.55 (series of m, 6H), 2.36 (s, 3H), 2.25 (s, 6H), 2.20 (broad signal, 2H).

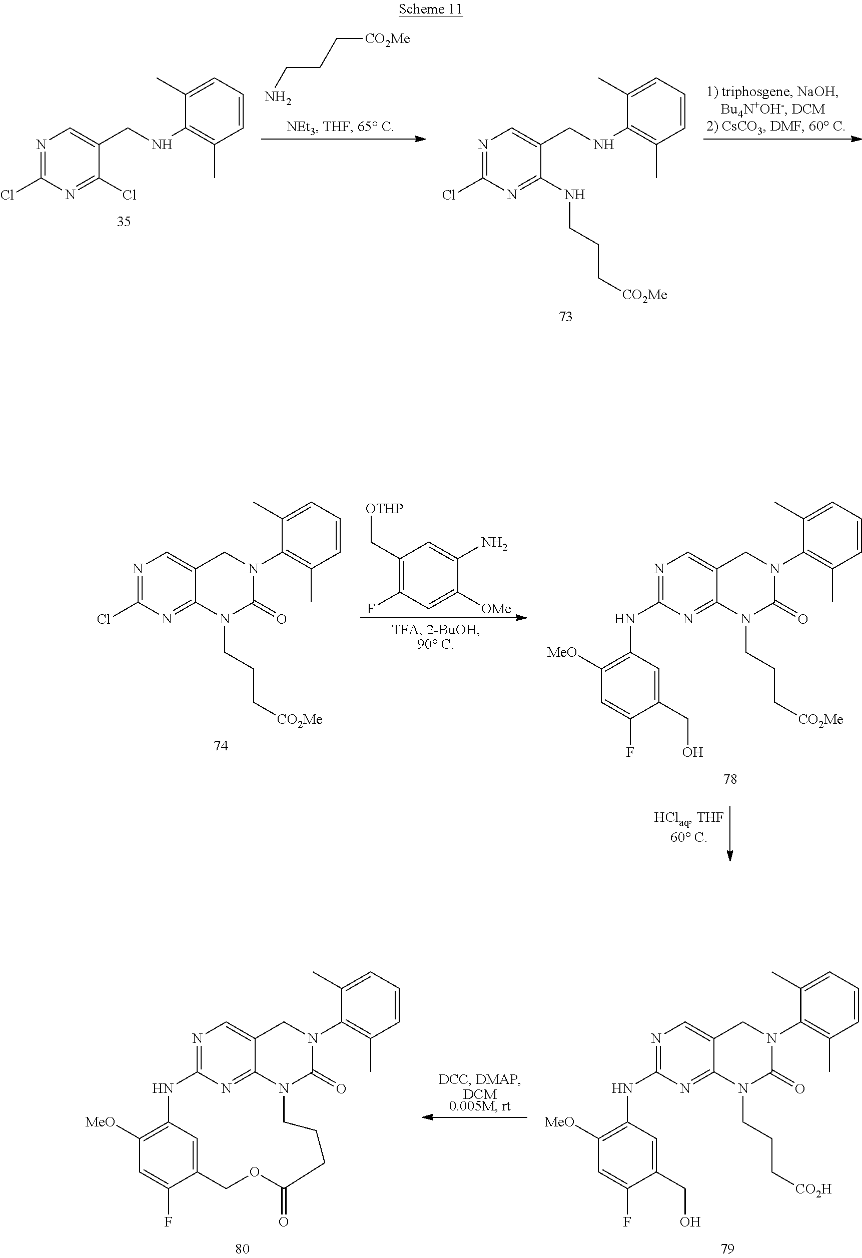

Previous intermediates (35-74) have already been described in the experimental section.

### Methyl 4-(3-(2,6-dimethylphenyl)-7-((4-fluoro-5-(hydroxymethyl)-2-methoxyphenyl)amino)-2-oxo-3,4-dihydropyrimido[4,5-d]pyrimidin-1(2H)-yl)butanoate (78)

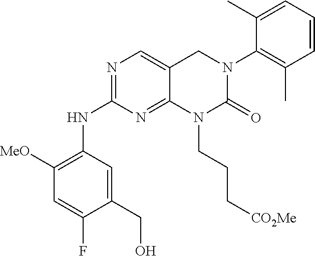 [SMILES: COC(=O)CCCN1C(=O)N(c2c(C)cccc2C)Cc2cnc(Nc3cc(CO)c(F)cc3OC)nc21]

Methyl 4-((2-chloro-5-(((chlorocarbonyl)(2,6-dimethylpheynl)amino)methyl)pyrimidin-4-yl)amino)butanoate (1.300 g, 3.343 mmol) and (5-amino-2-fluoro-4-methoxyphenyl)methanol (0.761 g, 0.761 mmol) were placed in a sealed tube and dissolved in anhydrous 2-BuOH (50.0 mL). Then 3A MS were added followed by anhydrous TFA (0.512 mL, 6.686 mmol) and the reaction was stirred at 90° C. for 16 h. Thereafter, the reaction was diluted with water and treated with aq. solution of NaHCO3 (6.686 mmol). The aqueous phase was extracted with CHCl3/iPrOH mixture (1:1). Finally, the combined organic layers were dried over MgSO4, filtered and concentrated under vacuum. The obtained residue was purified by flash column chromatography on silica (heptane:AcOEt from 70:30 to 0:100) to yield pure product (1.452 g, Yield: 83%, yellow pale solid). LC-MS (ESI+): m/z: 523.8 RT: 1.450 min (TACC50).

### 4-(3-(2,6-dimethylphenyl)-7-((4-fluoro-5-(hydroxymethyl)-2-methoxyphenyl)amino)-2-oxo-3,4-dihydropyrimido[4,5-d]pyrimidin-1(2H)-yl)butanoic acid (79)

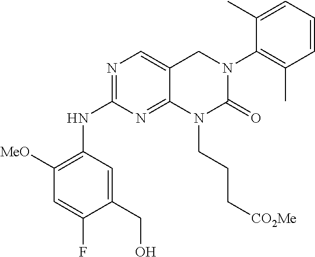 [SMILES: COC(=O)CCCN1C(=O)N(c2c(C)cccc2C)Cc2cnc(Nc3cc(CO)c(F)cc3OC)nc21]

Methyl 4-(3-(2,6-dimethylphenyl)-7-((4-fluoro-5-(hydroxymethyl)-2-methoxyphenyl)amino)-2-oxo-3,4-dihydropyrimido[4,5-d]pyrimidin-1(2H)-yl)butanoate (0.639 g, 1.220 mmol) was dissolved in THF (5.0 mL) and treated with HCl (6.10 mmol, 6 M aqueous solution, so 0.22 mL) and it was heated at 60° C. for 16 h. Thereafter, solvent was removed under vacuum and the obtained residue was purified by reverse phase chromatography (from 90% [aq. 0.1% HCOOH]-10% ACN to 54% [aq. 0.1% HCOOH]-46% ACN) to yield pure product (0.386 g, Yield: 62%, yellow pale solid). LC-MS (ESI+): m/z: 510.2 RT: 1.00 min (TACC50).

### 1³-(2,6-dimethylphenyl)-3⁴-fluoro-3⁶-methoxy-1¹,1²,1³,1⁴-tetrahydro-5-oxa-2-aza-1(7,1)-pyrimido[4,5-d]pyrimidina-3(1,3)-benzenacyclononaphane-1²,6-dione (80)

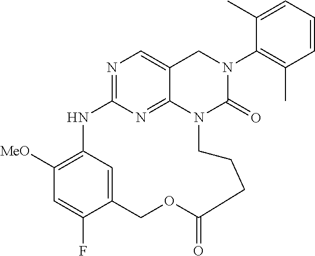 [SMILES: COc1cc(F)c2cc1Nc1ncc3c(n1)N(CCCC(=O)OC2)C(=O)N(c1c(C)cccc1C)C3]

4-(3-(2,6-dimethylphenyl)-7-((4-fluoro-5-(hydroxymethyl)-2-methoxyphenyl)amino)-2-oxo-3,4-dihydropyrimido [4,5-d]pyrimidin-1(2H)-yl)butanoic acid (0.316 g, 0.62 mmol) was dissolved in anhydrous DMF (10.0 mL) and diluted with anhydrous DCM (80.0 mL). 3A molecular shieves were added followed by DMAP (0.114 g, 0.930 mmol) and DCC (0.154 g, 0.744 mmol) and the reaction was stirred at room temperature for 16 h. Then, solvent was removed under vacuum and the obtained residue was purified by flash column chromatography on silica (deactivated with DCM:NH3 1%) (DCM:MeOH from 100:0 to 90:10). The obtained product was recrystallized in ACN to yield pure product (0.174 g, Yield: 57%, white solid). LC-MS (ESI+): m/z: 492.2 RT: 4.047 min (VILLA). 1H NMR (400 MHz, CDCl3) δ 8.46 (d, J=8.1 Hz, 1H), 7.95 (s, 1H), 7.71 (s, 1H), 7.20-7.10 (m, 3H), 6.65 (d, J=11.1 Hz, 1H), 5.37 (s, 2H), 4.49 (s, 2H), 4.19-4.12 (m, 2H), 3.89 (s, 3H), 2.57-2.50 (m, 2H), 2.25 (s, 6H), 2.23-2.15 (m, 2H).

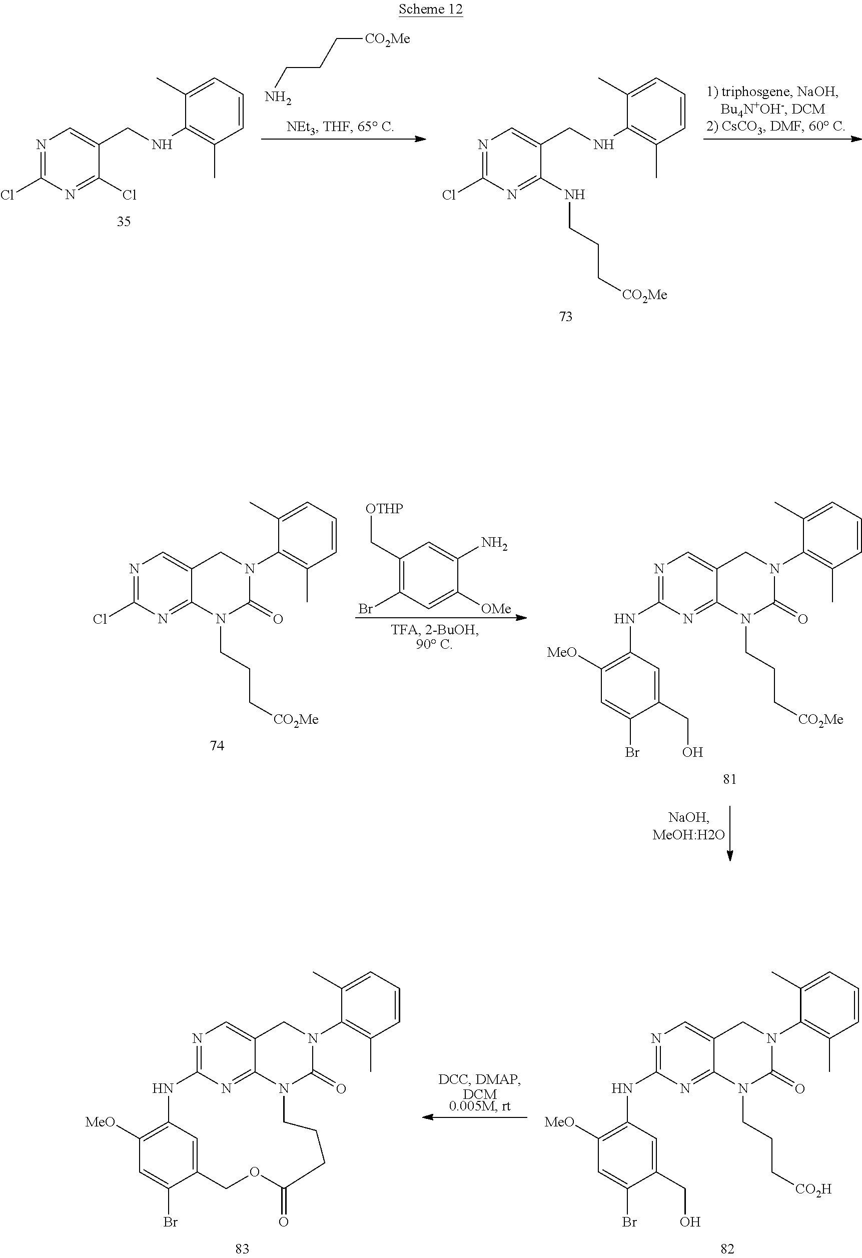

Previous intermediates (35-74) have already been described in the experimental section.

### Methyl 4-(7-((4-bromo-5-(hydroxymethyl)-2-methoxyphenyl)amino)-3-(2,6-dimethylphenyl)-2-oxo-3,4-dihydropyrimido[4,5-d]pyrimidin-1(2H)-yl)butanoate (81)

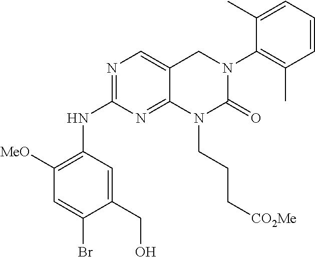 [SMILES: COC(=O)CCCN1C(=O)N(c2c(C)cccc2C)Cc2cnc(Nc3cc(CO)c(Br)cc3OC)nc21]

Methyl 4-(7-chloro-3-(2,6-dimethylphenyl)-2-oxo-3,4-dihydropyrimido[4,5-d]pyrimidin-1(2H)-yl)butanoate (1.2 g, 3.086 mmol) and (5-amino-2-bromo-4-methoxyphenyl)methanol (1.074 g, 4.629 mmol) were placed in a sealed tube and dissolved in anhydrous of 2-butanol (20.0 mL). Then 3A MS and anhydrous TFA (0.704 mL, 6.172 mmol) were added and the reaction was stirred at 90° C. for 16 h. Thereafter, the reaction was diluted with water and treated with aq. solution of NaHCO3 (6.172 mmol). The aqueous phase was extracted with CHCl3/iPrOH mixture (1:1). Finally, the combined organic layers were dried over MgSO4, filtered and concentrated under vacuum. The obtained residue was purified by flash column chromatography on silica (heptane:AcOEt from 100:0 to 70:30) to yield pure product (1.258 g, Yield: 70%, brown pale solid). LC-MS (ESI+): m/z: 585.0 RT: 1.66 min (TACC50).

### 4-[7-(4-Bromo-5-hydroxymethyl-2-methoxy-phenylamino)-3-(2,6-dimethyl-phenyl)-2-oxo-3,4-dihydro-2H-pyrimido[4,5-d]pyrimidin-1-yl]-butyric acid (82)

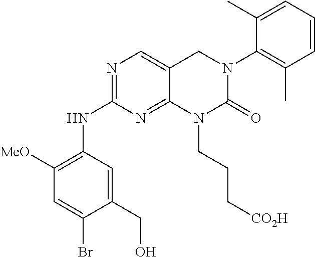 [SMILES: COc1cc(Br)c(CO)cc1Nc1ncc2c(n1)N(CCCC(=O)O)C(=O)N(c1c(C)cccc1C)C2]

4-[7-(4-Bromo-5-hydroxymethyl-2-methoxy-phenylamino)-3-(2,6-dimethyl-phenyl)-2-oxo-3,4-dihydro-2H-pyrimido[4,5-d]pyrimidin-1-yl]-butyric acid methyl ester (0.7 g, 1.2 mmol) was dissolved in a MeOH:H2O mixture (5:1, 12.0 mL) and treated with an aqueous solution of sodium hydroxide (0.4 mL, 6.0 mmol) and the reaction was stirred at room temperature for 3 hours. Then, solvent was removed under vacuum and the obtained residue was used without further purification.

### 3⁴-bromo-13-(2,6-dimethylphenyl)-3⁶-methoxy-1¹,1²,1³,1⁴-tetrahydro-5-oxa-2-aza-1(7,1)-pyrimido[4,5-d]pyrimidina-3(1,3)-benzenacyclononaphane-1²,6-dione (83)

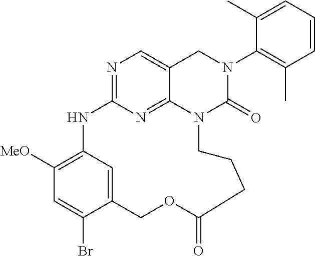 [SMILES: COc1cc(Br)c2cc1Nc1ncc3c(n1)N(CCCC(=O)OC2)C(=O)N(c1c(C)cccc1C)C3]

4-[7-(4-Bromo-5-hydroxymethyl-2-methoxy-phenylamino)-3-(2,6-dimethyl-phenyl)-2-oxo-3,4-dihydro-2H-pyrimido[4,5-d]pyrimidin-1-yl]-butyric acid (0.68 g, 1.19 mmol) was dissolved in anhydrous DMF (10.0 mL) and diluted with anhydrous DCM (230.0 mL). 3A molecular shieves were added followed by DMAP (0.220 g, 1.797 mmol) and DCC (0.297 g, 1.438 mmol) and the reaction was stirred at room temperature for 16 h. Then, solvent was removed under vacuum and the obtained residue was purified by flash column chromatography on silica (Heptane:AcOEt from 100:0 to 50:50) to yield pure product (0.131 g, Yield: 20%, brown pale solid). LC-MS (ESI+): m/z: 554.2366 RT: 6.123 min (VILLA-2T). 1H NMR (400 MHz, DMSO) δ 8.41 (s, 1H), 8.15 (s, 1H), 8.03 (s, 1H), 7.28 (s, 1H), 7.19-7.14 (m, 3H), 5.75 (s, 1H), 5.18 (s, 2H), 4.52 (s, 2H), 4.00-3.93 (m, 3H), 3.91 (s, 3H), 2.18 (s, 6H), 2.05 (t, J=12.0 Hz, 2H).

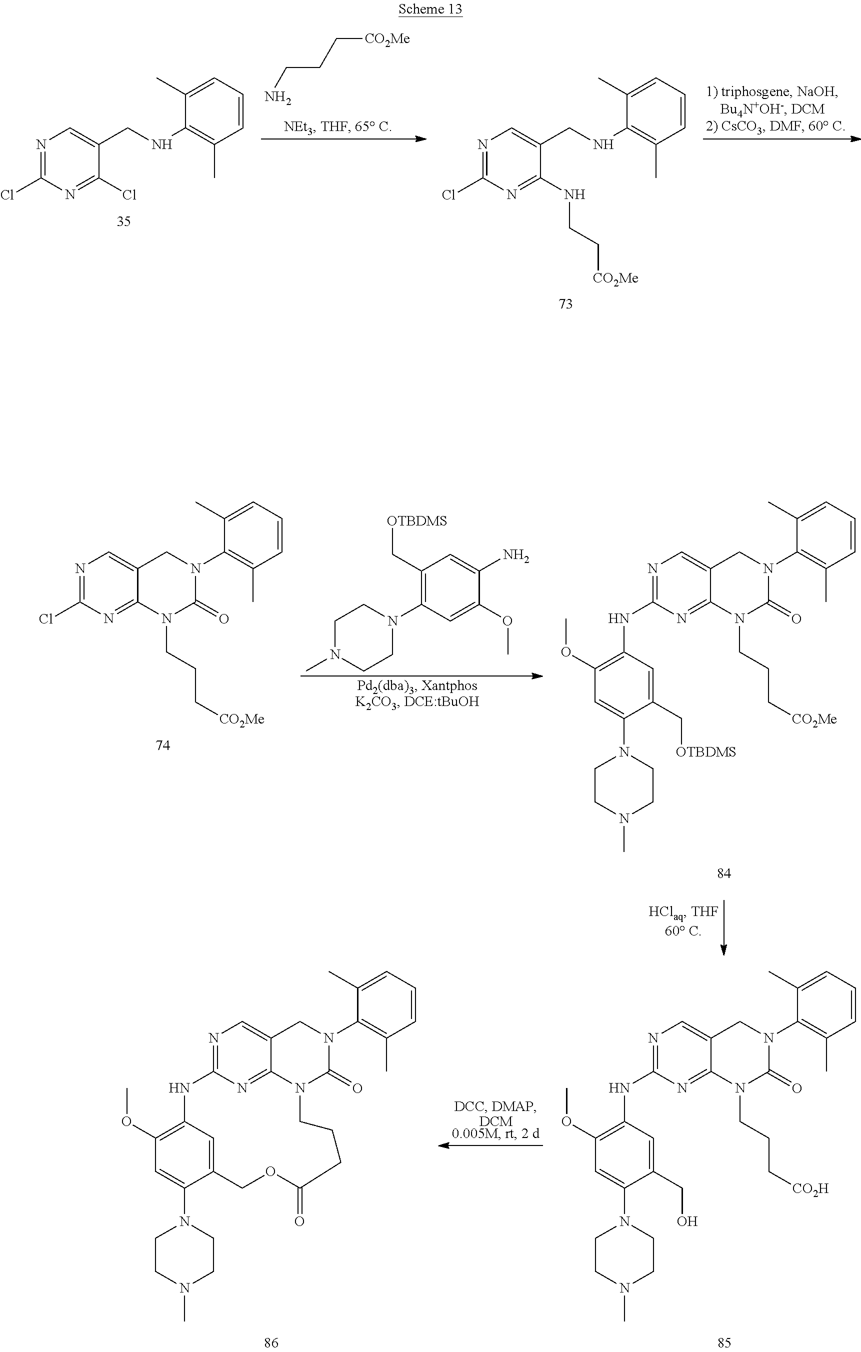

Previous intermediates (35-74)) have already been described in the experimental section.

### 4-[7-[5-(tert-Butyl-dimethyl-silanyloxymethyl)-2-methoxy-4-(4-methyl-piperazin-1-yl)-phenylamino]-3-(2,6-dimethyl-phenyl)-2-oxo-3,4-dihydro-2H-pyrimido[4,5-d]pyrimidin-1-yl]-butyric acid methyl ester (84)

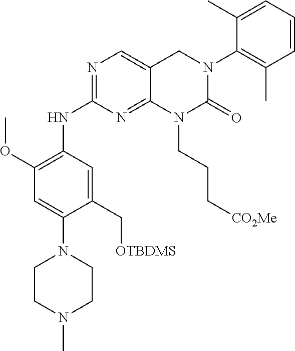 [SMILES: COC(=O)CCCN1C(=O)N(c2c(C)cccc2C)Cc2cnc(Nc3cc(CO[Si](C)(C)C(C)(C)C)c(N4CCN(C)CC4)cc3OC)nc21]

4-[7-Chloro-3-(2,6-dimethyl-phenyl)-2-oxo-3,4-dihydro-2H-pyrimido[4,5-d]pyrimidin-1-yl]-butyric acid methyl ester (1.30 g, 3.34 mmol), 5-(tert-Butyl-dimethyl-silanyloxymethyl)-2-methoxy-4-(4-methyl-piperazin-1-yl)-phenylamine (1.28 g, 3.51 mmol) and K2CO3 (0.700 g, 5.02 mmol) were placed in a sealed tube and dissolved in a tBuOH:DCE mixture (7.1, 16.0 mL). The reaction was purged with N2 and then, Pd2dba3 (0.460 g, 0.500 mmol) and XantPhos (0.270 g, 0.500 mmol) were added. The mixture was purged with N2 again and the reaction was stirred at 85° C. for 16 h. Then, the reaction crude was filtered over celite and rinsed with DCM. The filtrate was washed with water, dried over MgSO4, filtered and concentrated under vacuum. The obtained residue was purified by reverse phase chromatography (from 32% [25 mM NH4HCO3]-68% ACN to 4% [25 mM NH4HCO3]-96% ACN) to yield pure product (0.150 g, Yield: 6%, brown pale solid). LC-MS (ESI+): m/z: 717.9 RT: 4.383 min (TACC50).

### 4-(3-(2,6-dimethylphenyl)-7-((5-(hydroxymethyl)-2-methoxy-4-(4-methylpiperazin-1-yl)phenyl)amino)-2-oxo-3,4-dihydropyrimido[4,5-d]pyrimidin-1(2H)-yl)butanoic acid (85)

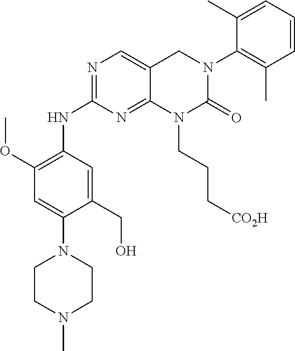 [SMILES: COc1cc(N2CCN(C)CC2)c(CO)cc1Nc1ncc2c(n1)N(CCCC(=O)O)C(=O)N(c1c(C)cccc1C)C2]

4-[7-[5-(tert-Butyl-dimethyl-silanyloxymethyl)-2-methoxy-4-(4-methyl-piperazin-1-yl)-phenylamino]-3-(2,6-dimethyl-phenyl)-2-oxo-3,4-dihydro-2H-pyrimido[4,5-d]pyrimidin-1-yl]-butyric acid methyl ester (0.30 g, 0.42 mmol) was dissolved in THF (5.0 mL) and treated with HCl (2.09 mmol, 6 M aqueous solution, so 0.35 mL) and it was heated at 60° C. for 2 h. Thereafter, solvent was removed under vacuum and the obtained residue was used without further purification. LC-MS (ESI+): m/z: 590.0 RT: 2.76 min (TACC50).

### 1³-(2,6-dimethylphenyl)-3⁶-methoxy-3⁴-(4-methylpiperazin-1-yl)-1¹,1²,1³,1⁴-tetrahydro-5-oxa-2-aza-1(7,1)-pyrimido[4,5-d]pyrimidina-3(1,3)-benzenacyclononaphane-1²,6-dione (86)

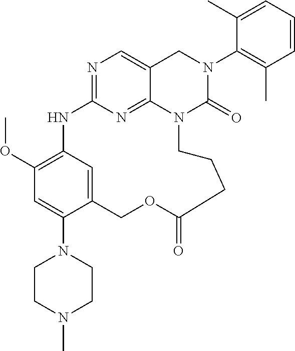 [SMILES: COc1cc(N2CCN(C)CC2)c2cc1Nc1ncc3c(n1)N(CCCC(=O)OC2)C(=O)N(c1c(C)cccc1C)C3]

4-(3-(2,6-dimethylphenyl)-7-((5-(hydroxymethyl)-2-methoxy-4-(4-methylpiperazin-1-yl)phenyl)amino)-2-oxo-3,4-dihydropyrimido[4,5-d]pyrimidin-1(2H)-yl)butanoic acid (0.210 g, 0.348 mmol) was dissolved in anhydrous DMF (2.0 mL) and diluted with anhydrous DCM (50.0 mL). 3A molecular shieves were added followed by DMAP (0.064 g, 0.522 mmol) and DCC (0.086 g, 0.418 mmol) and the reaction was stirred at room temperature for 16 h. Then, solvent was removed under vacuum and the obtained residue was purified by reverse phase chromatography (from 32% [25 mM NH4HCO3]-68% ACN to 4% [25 mM NH4HCO3]-96% ACN) to yield pure product (0.012 g, Yield: 6%, white solid). LC-MS (ESI+): m/z: 572.3 RT: 2.544 min (VILLA). 1H NMR (400 MHz, DMSO-d6) δ 8.16 (s, 1H), 8.04 (s, 1H), 7.82 (s, 1H), 7.02-7.16 (m, 3H), 6.77 (s, 1H), 5.19 (s, 2H), 4.43 (s, 2H), 3.85-3.90 (m, 2H), 3.82 (s, 3H), 2.75-2.82 (m, 4H), 2.37-2.42 (m, 6H)*, 2.17 (s, 3H), 2.11 (s, 6H), 1.96 (broad signal, 2H). *overlapped by solvent signal.

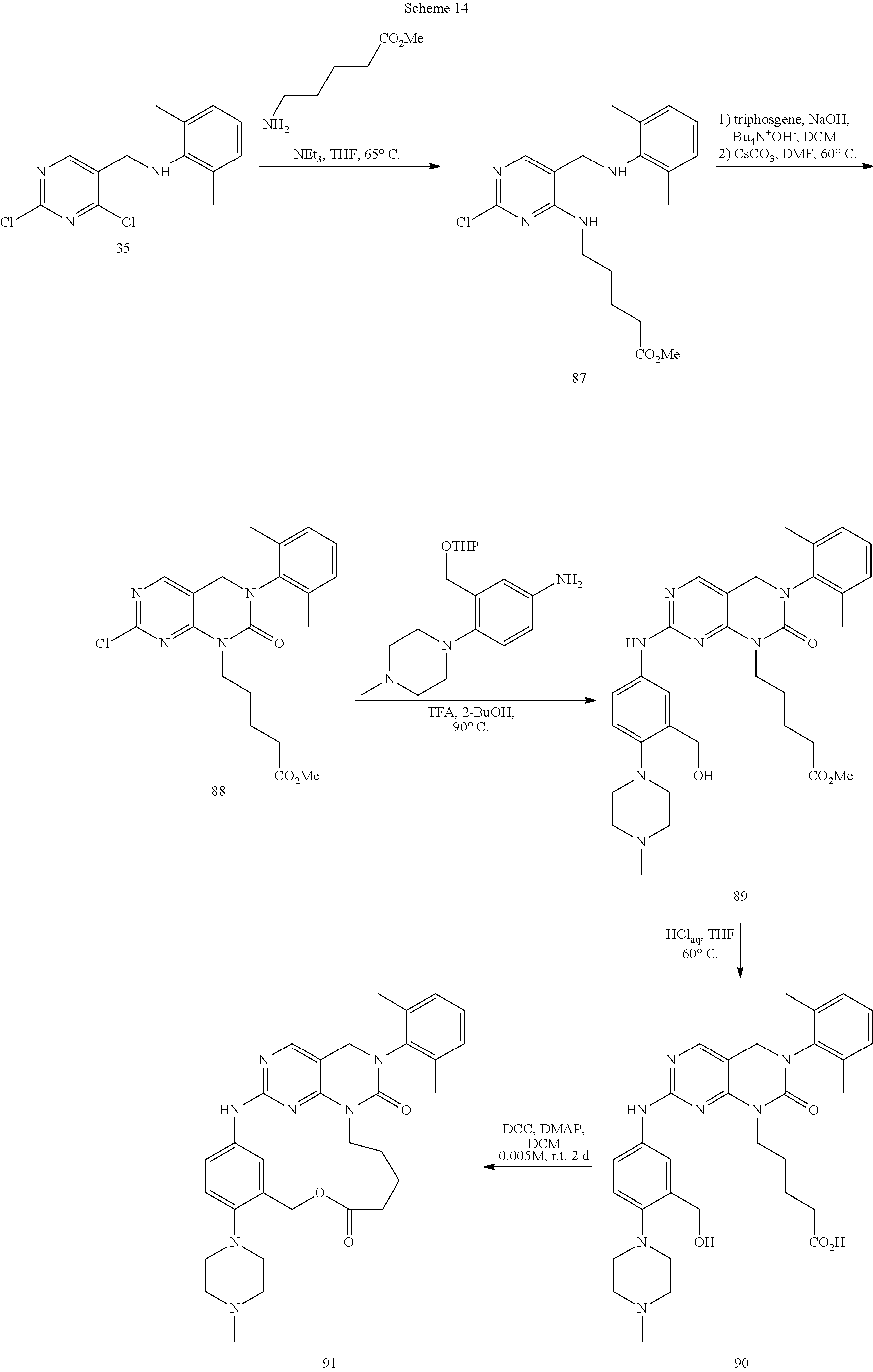

### Methyl 5-(2-chloro-5-(((2,6-dimethylphenyl)amino)methyl)pyrimidin-4-yl)pentanoate (87)

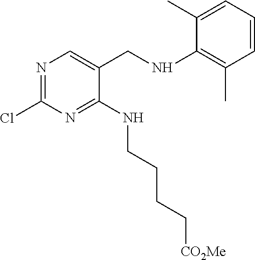 [SMILES: COC(=O)CCCCNc1nc(Cl)ncc1CNc1c(C)cccc1C]

Methyl-5-aminopentanoate hydorchloride (3.12 g, 18.61 mmol) and triethylamine (5.18 mL, 37.21 mmol) were added to a solution of N-((2,4-dichloropyrimidin-5-yl)methyl)-2,6-dimethylaniline (3.50 g, 12.40 mmol) in anhydrous THF (37.0 mL) and the mixture was stirred at 65° C. for 16 h. Thereafter, the reaction crude was diluted with AcOEt and washed with brine. The organic layer was dried over MgSO4, filtered and concentrated under vacuum. The obtained pure product (4.60 g, Yield: 98%, yellow oil) was used without further purification. LC-MS (ESI+): m/z: 376.9 RT: 1.709 min (TACC50).

### 5-[7-Chloro-3-(2,6-dimethyl-phenyl)-2-oxo-3,4-dihydro-2H-pyrimido[4,5-d]pyrimidin-1-y]-pentanoic acid methyl ester (88)

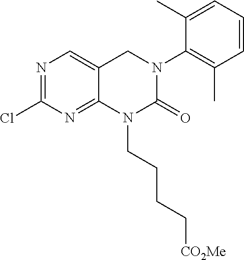 [SMILES: COC(=O)CCCCN1C(=O)N(c2c(C)cccc2C)Cc2cnc(Cl)nc21]

Triphosgene (5.433 g, 18.31 mmol) was added to a solution of methyl 5-(2-chloro-5-(((2,6-dimethylphenyl)amino)methyl)pyrimidin-4-yl)pentanoate (4.600 g, 12.205 mmol) in DCM (35.0 mL) and it was stirred at room temperature for 1 h. Then an aq. solution of sodium hydroxide (9.76 g, 244.10 mmol, 4.9 M) and tetrabutylammonium hydroxide (1.587 mmol 55% in aq. solution, so 0.75 mL) was added at 0° C. The reaction was allowed to reach room temperature and stirred at room temperature for 16 h. Thereafter, the reaction crude was diluted with DCM and washed with water. The organic layer was dried over MgSO4, filtered and concentrated under vacuum. The obtained residue was dissolved in DMF (35.0 mL) and cessium carbonate (4.04 g, 12.402 mmol) was added. The reaction mixture was heated at 60° C. for 16 h. Thereafter, the reaction crude was diluted with DCM and washed with brine. The organic layer was dried over MgSO4, filtered and concentrated under vacuum. The obtained residue was purified by flash column chromatography on silica (Heptane:AcOEt 100:0 to 80:20) to yield pure product (2.626 g, Yield: 53%, yellow solid). LC-MS (ESI+): m/z: 402.0 RT: 1.623 min (TACC50).

### Methyl 5-(3-(2,6-dimethylphenyl)-7-((3-(hydroxymethyl)-4-(4-methylpiperazin-1-yl)phenyl)amino)-2-oxo-3,4-dihydropyrimido[4,5-d]pyrimidin-1(2H)-yl)pentanoate (89)

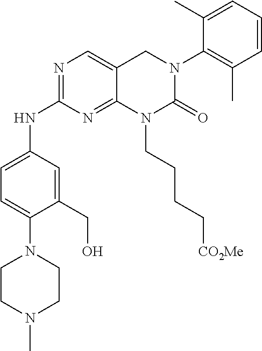 [SMILES: COC(=O)CCCCN1C(=O)N(c2c(C)cccc2C)Cc2cnc(Nc3ccc(N4CCN(C)CC4)c(CO)c3)nc21]

5-[7-Chloro-3-(2,6-dimethyl-phenyl)-2-oxo-3,4-dihydro-2H-pyrimido[4,5-d]pyrimidin-1-yl]-pentanoic acid methyl ester (0.965 g, 2.395 mmol) and 4-(4-Methyl-piperazin-1-yl)-3-(tetrahydro-pyran-2-yloxymethyl)-phenylamine (B, 0.973 g, 3.185 mmol) were placed in a sealed tube and dissolved in anhydrous 2-BuOH (20.0 mL). Then 3A MS were added followed by anhydrous TFA (0.369 mL, 4.790 mmol) and the reaction was stirred at 90° C. for 16 h. Thereafter, the reaction was diluted with water and treated with aq. solution of NaHCO3 (4.79 mmol). The aqueous phase was extracted with CHCl3/iPrOH mixture (1:1). Finally, the combined organic layers were dried over MgSO4, filtered and concentrated under vacuum. The obtained residue was purified by flash column chromatography on silica (DCM:MeOH 100:0 to 90:10) to yield pure product (0.595 g, Yield: 42%, brown pale solid). LC-MS (ESI+): m/z: 587.0 RT: 1.359 min (TACC50).

### 5-{3-(2,6-Dimethyl-phenyl)-7-[3-hydroxymethyl-4-(4-methyl-piperazin-1-yl-phenylamino]-2-oxo-3,4-dihydro-2H-pyrimido[4,5-d]pyrimidin-1-yl}-pentanoic acid (90)

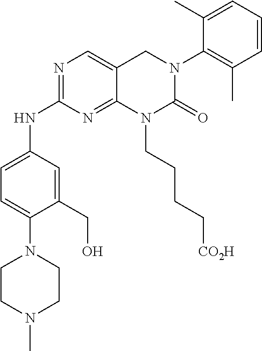 [SMILES: Cc1cccc(C)c1N1Cc2cnc(Nc3ccc(N4CCN(C)CC4)c(CO)c3)nc2N(CCCCC(=O)O)C1=O]

Methyl 5-(3-(2,6-dimethylphenyl)-7-((3-(hydroxymethyl)-4-(4-methylpiperazin-1-yl)phenyl)amino)-2-oxo-3,4-dihydropyrimido[4,5-d]pyrimidin-1(2H)-yl)pentanoate (0.595 g, 1.012 mmol) was dissolved in THF (3.0 mL) and treated with HCl (5.06 mmol, 6 M aqueous solution, so 0.84 mL) and it was heated at 60° C. for 2 h. Thereafter, solvent was removed under vacuum and the obtained residue was purified by reverse phase chromatography (from 95% [aq. 0.1% HCOOH]-5% ACN to 63% [aq. 0.1% HCOOH]-37% ACN) to yield pure product (0.395 g, Yield: 68%, white solid). LC-MS (ESI+): m/z: 574.2 RT: 2.127 min (VILLA).

### 1³-(2,6-dimethylphenyl)-3⁴-(4-methylpiperazin-1-yl)-1¹,1²,1³,1⁴-tetrahydro-5-oxa-2-aza-1(7,1)-pyrimido[4,5-d]pyrimidina-3(1,3)-benzenacyclodecaphane-1²,6-dione (91)

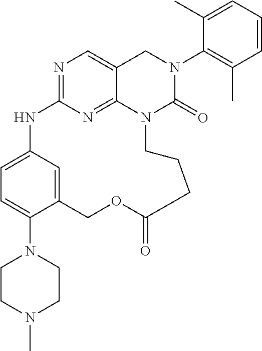 [SMILES: Cc1cccc(C)c1N1Cc2cnc3nc2N(CCCC(=O)OCc2cc(ccc2N2CCN(C)CC2)N3)C1=O]

5-{3-(2,6-Dimethyl-phenyl)-7-[3-hydroxymethyl-4-(4-methyl-piperazin-1-yl)-phenylamino]-2-oxo-3,4-dihydro-2H-pyrimido[4,5-d]pyrimidin-1-yl}-pentanoic acid (0.353 g, 0.615 mmol) was dissolved in anhydrous DMF (10.0 mL) and diluted with anhydrous DCM (70.0 mL). 3A molecular shieves were added followed by DMAP (0.1130 g, 0.922 mmol) and DCC (0.152 g, 0.738 mmol) and the reaction was stirred at room temperature for 16 h. The reaction was recharged with DMAP (0.1130 g, 0.922 mmol) and DCC (0.152 g, 0.738 mmol) and the reaction was stirred at room temperature for further 16 h. Then, solvent was removed under vacuum and the obtained residue was dissolved in ACN. Formed solids were removed by filtration. The filtrate was concentrated under vacuum and the obtained residue was purified by reverse phase chromatography (from 59% [aq. 25 mM NH4HCO3]-41% ACN to 17% [aq. 25 mM NH4HCO3]-83% ACN) to yield pure product (0.0488 g, Yield: 14%, white solid). LC-MS (ESI+): m/z: 556.2 RT: 2.624 min (VILL_J). 1H NMR (400 MHz, CDCl3) δ 8.10 (d, J=2.2 Hz, 1H), 7.92 (s, 1H), 7.18-7.07 (m, 5H), 6.84 (dd, J=8.5, 2.4 Hz, 1H), 5.37 (s, 2H), 4.47 (s, 2H), 4.03-3.97 (m, 2H), 2.93 (t, J=4.6 Hz, 4H), 2.59 (s, 4H), 2.53-2.48 (m, 2H), 2.36 (s, 3H), 2.25 (s, 6H), 1.97-1.87 (m, 2H), 1.84-1.76 (m, 2H).

### Example 3: Synthesis of Macrocycles Synthesized by RCM

For the ring closing metathesis reaction, the commonly known as Zhan Catalyst-1B was employed (CAS: 918870-76-5) which corresponds to Dichloro1,3-bis(2,4,6-trimethylphenyl)-2-imidazolidinylidene5-(dimethylamino)sulfonyl-2-(1-methylethoxy-O)phenylmethylene-Cruthenium(II) structure.

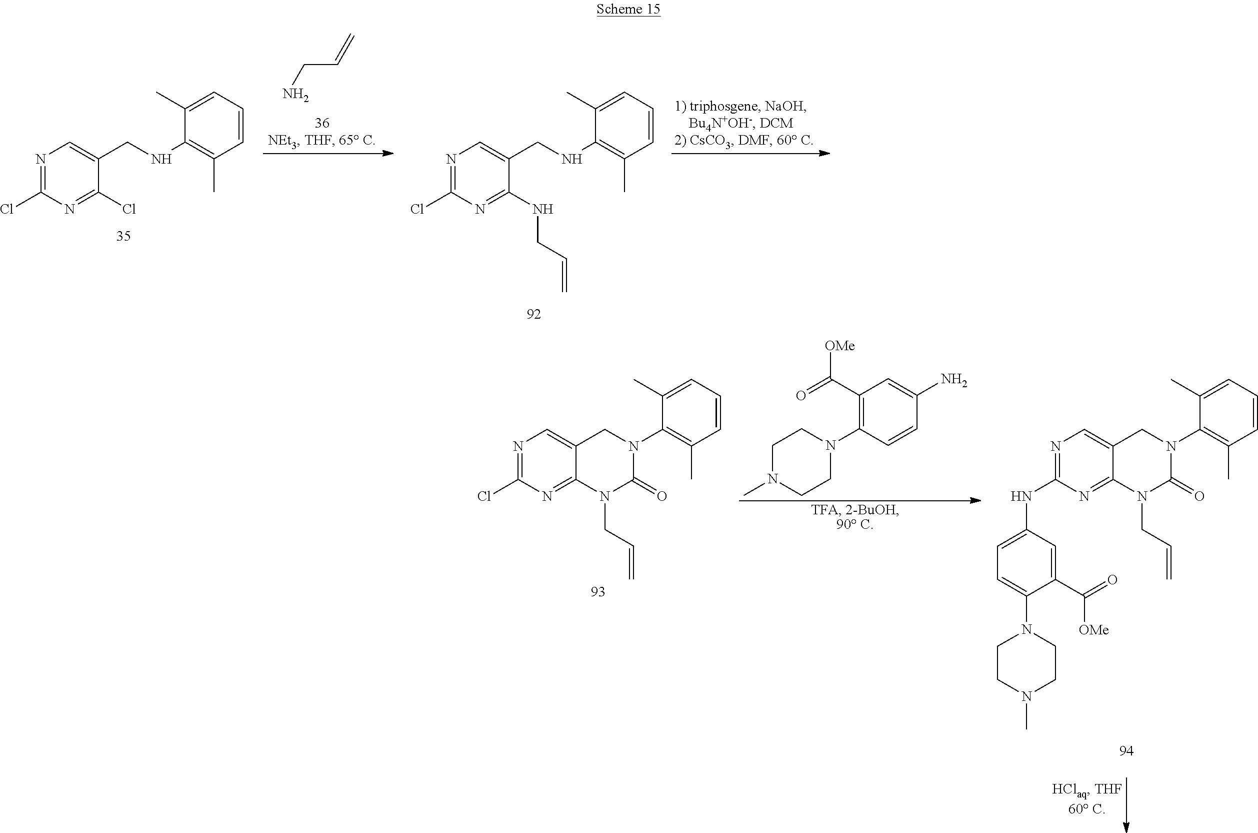

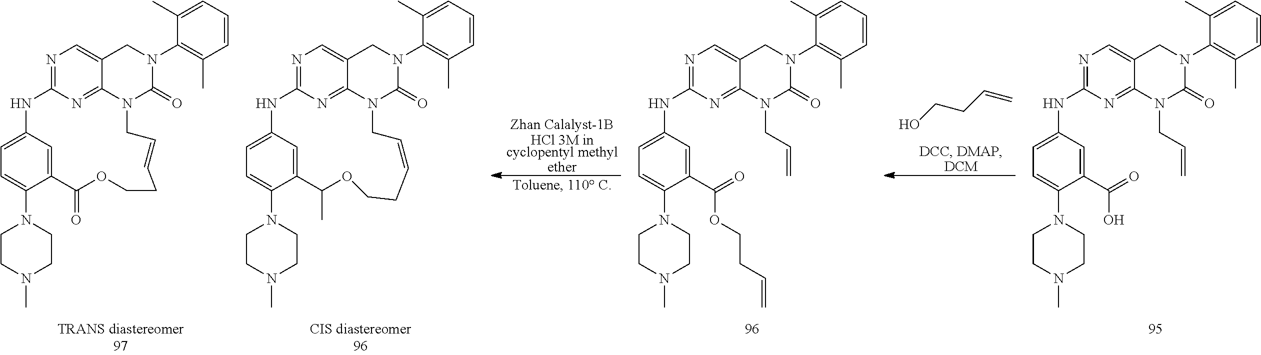

### Allyl-{2-chloro-5-[(2,6-dimethyl-phenylamino)-methyl]-pyrimidin-4-yl}-amine (92)

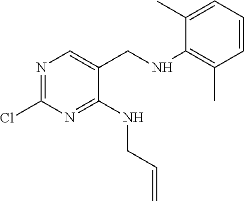 [SMILES: C=CCNc1nc(Cl)ncc1CNc1c(C)cccc1C]

Allyl amine (1.60 g, 21.26 mmol) and triethylamine (2.96 mL, 21.26 mmol) were added to a solution of N-((2,4-dichloropyrimidin-5-yl)methyl)-2,6-dimethylaniline (4.0 g, 14.18 mmol) in anhydrous THF (45.0 mL) and the mixture was stirred at 65° C. for 16 h. Thereafter, the reaction crude was diluted with AcOEt and washed with brine. The organic layer was dried over MgSO4, filtered and concentrated under vacuum. The obtained pure product (4.29 g, Yield: quantitative, yellow oil) was used without further purification. LC-MS (ESI+): m/z: 303.0 RT: 1.76 min (TACC50).

### 1-Allyl-7-chloro-3-(2,6-dimethyl-phenyl)-3,4-dihydro-1H-pyrimido[4,5-d]pyrimidin-2-one (93)

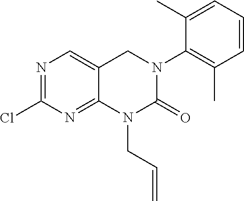 [SMILES: C=CCN1C(=O)N(c2c(C)cccc2C)Cc2cnc(Cl)nc21]

Triphosgene (6.30 g, 21.26 mmol) was added to a solution of Allyl-{2-chloro-5-[(2,6-dimethyl-phenylamino)-methyl]-pyrimidin-4-yl}-amine (4.29 g, 14.2 mmol) in DCM (45.0 mL) and it was stirred at room temperature for 1 h. Then an aq. solution of sodium hydroxide (11.30 g, 283.5 mmol, 4.9 M) and tetrabutylammonium hydroxide (1.84 mmol 55% in aq. solution, so 0.90 mL) was added at 0° C. The reaction was allowed to reach room temperature and stirred at room temperature for 16 h. Thereafter, the reaction crude was diluted with DCM and washed with water. The organic layer was dried over MgSO4, filtered and concentrated under vacuum. The obtained residue was dissolved in DMF (40.0 mL) and cessium carbonate (4.66 g, 14.32 mmol) was added. The reaction mixture was heated at 60° C. for 16 h. Thereafter, the reaction crude was diluted with DCM and washed with brine. The organic layer was dried over MgSO4, filtered and concentrated under vacuum. The obtained residue was purified by flash column chromatography on silica (Heptane:AcOEt 100:0 to 0:100) to yield pure product (2.80 g, Yield: 60%, white solid). LC-MS (ESI+): m/z: 329.1 RT: 1.648 min (TACC50).

### Methyl 5-((8-allyl-6-(2,6-dimethylphenyl)-7-oxo-5,6,7,8-tetrahydropyrimido[4,5-d]pyrimidin-2-yl)amino)-2-(4-methylpiperazin-1-yl)benzoate (94)

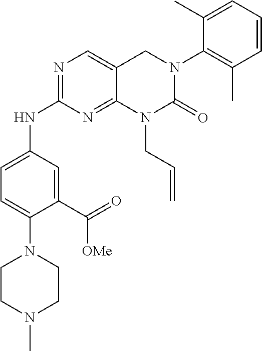 [SMILES: C=CCN1C(=O)N(c2c(C)cccc2C)Cc2cnc(Nc3ccc(N4CCN(C)CC4)c(C(=O)OC)c3)nc21]

1-Allyl-7-chloro-3-(2,6-dimethyl-phenyl)-3,4-dihydro-1H-pyrimido[4,5-d]pyrimidin-2-one (1.05 g, 3.193 mmol) and methyl 5-amino-2-(4-methylpiperazin-1-yl)benzoate (1.114 g, 4.47 mmol) were placed in a sealed tube and dissolved in anhydrous 2-BuOH (20.0 mL). Then 3A MS were added followed by anhydrous trifluoroacetic acid (0.728 mL, 6.386 mmol) and the reaction was stirred at 90° C. for 16 h. Thereafter, the reaction was diluted with water and treated with aq. solution of NaHCO3 (6.386 mmol). The aqueous phase was extracted with CHCl3/iPrOH mixture (1:1). Finally, the combined organic layers were dried over MgSO4, filtered and concentrated under vacuum. The obtained residue was purified by flash column chromatography on silica (Deactivated with DCM:1% NH3) (DCM:MeOH from 100:0 to 90:10) to yield pure product (1.37 g, Yield: 79%, yellow solid) LC-MS (ESI+): m/z: 542.0 RT: 1.15 min (TACC50).

### 5-[8-Allyl-6-(2,6-dimethyl-phenyl)-7-oxo-5,6,7,8-tetrahydro-pyrimido[4,5-d]pyrimidin-2-ylamino]-2-(4-methyl-piperazin-1-yl)-benzoic acid (95)

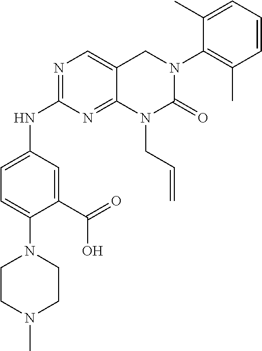 [SMILES: C=CCN1C(=O)N(c2c(C)cccc2C)Cc2cnc(Nc3ccc(N4CCN(C)CC4)c(C(=O)O)c3)nc21]

Methyl 5-((8-allyl-6-(2,6-dimethylphenyl)-7-oxo-5,6,7,8-tetrahydropyrimido[4,5-d]pyrimidin-2-yl)amino)-2-(4-methylpiperazin-1-yl)benzoate (1.29 g, 2.38 mmol) was dissolved in THF (8.0 mL) and treated with HCl (11.92 mmol, 6 M aqueous solution, so 1.98 mL) and it was heated at 60° C. for 4 h. Thereafter, solvent was removed under vacuum and the obtained product was used without further purification. LC-MS (ESI+): m/z: 528.3 RT: 0.814 min (TACC50).

### 5-[8-Allyl-6-(2,6-dimethyl-phenyl)-7-oxo-5,6,7,8-tetrahydro-pyrimido[4,5-d]pyrimidin-2-ylamino]-2-(4-methyl-piperazin-1-yl)-benzoic acid but-3-enyl ester (96)

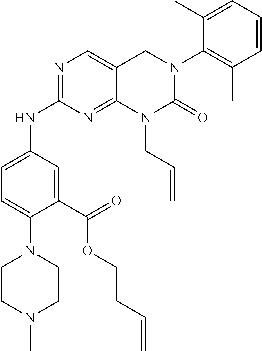 [SMILES: C=CCCOC(=O)c1cc(Nc2ncc3c(n2)N(CC=C)C(=O)N(c2c(C)cccc2C)C3)ccc1N1CCN(C)CC1]

3-buten-1-ol (0.3 mL, 3.57 mmol), DMAP (0.30 g, 2.38 mmol) and DCC (0.37 mg, 1.8 mmol) were sequentially added to a solution of 5-[8-Allyl-6-(2,6-dimethyl-phenyl)-7-oxo-5,6,7,8-tetrahydro-pyrimido[4,5-d]pyrimidin-2-ylamino]-2-(4-methyl-piperazin-1-yl)-benzoic acid (0.7 g, 1.19 mmol) in a DCM:DMF mixture (5:1, 5.0 mL) and the reaction was stirred at room temperature for 16 h. Thereafter, solvent was removed under vacuum and the obtained residue was purified by flash column chromatography on silica (DCM:MeOH from 100:0 to 90:10) to yield pure product (0.52 g, Yield: 75%, yellow solid). LC-MS (ESI+): m/z: 582.2 RT: 1.481 min (TACC50).

### (E)-1³-(2,6-dimethylphenyl)-3⁴-(4-methylpiperazin-1-yl)-1¹,1²,1³,1⁴-tetrahydro-5-oxa-2-aza-1(7,1)-pyrimido[4,5-d]pyrimidina-3(1,3)-benzenacyclodecaphan-8-ene-1²,4-dione (97) and (Z)-1³-(2,6-dimethylphenyl)-3⁴-(4-methylpiperazin-1-yl)-1¹,1²,1³,1⁴-tetrahydro-5-oxa-2-aza-1(7,1)-pyrimido[4,5-d]pyrimidina-3(1,3)-benzenacyclodecaphan-8-ene-1²,4-dione (98)

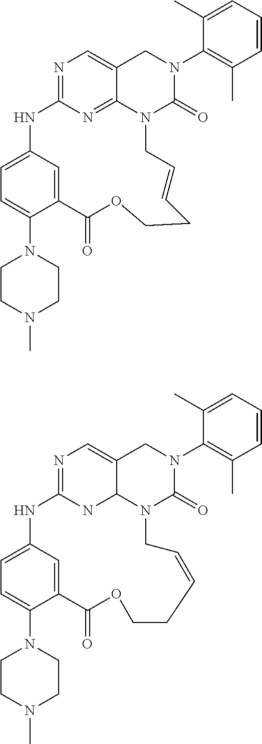

5-[8-Allyl-6-(2,6-dimethyl-phenyl)-7-oxo-5,6,7,8-tetrahydro-pyrimido[4,5-d]pyrimidin-2-ylamino]-2-(4-methyl piperazin-1-yl)-benzoic acid but-3-enyl ester (0.517 g, 0.889 mmol) was placed in a sealed tube and dissolved in anhydrous toluene (100 mL). Then HCl (0.889 mmol 3M in cyclopentyl methyl ether, so 0.30 mL) was added. The reaction crude was degassed by bubbling N2 through the solution, followed by the addition of a solution of Zhan catalyst (0.178 mmol, 0.131 g) in anhydrous toluene (18.0 mL). The reaction crude was degassed again by bubbling

N2 through the solution and heated at 110° C. for 16 h. Thereafter, solvent was removed under vacuum and the obtained residue was purified by reverse phase chromatography (from 32% [65 mM NH4OAc+ACN (90:10)]-68% ACN to 4% [65 mM NH4OAc+ACN (90:10)]-96% ACN) to yield both diastereomers.

Product A, TRANS diastereomer (18.2 mg, white solid). 1H NMR (400 MHz, DMSO) δ 9.64 (s, 1H), 8.54 (d, J=2.6 Hz, 1H), 8.15 (s, 1H), 7.18 (series of m, 4H), 7.06 (d, J=8.8 Hz, 1H), 5.67 (dt, J=15.9, 4.3 Hz, 1H), 5.59-5.49 (m, 1H), 4.50 (broad signal, 4H), 4.32 (broad signal, 2H), 2.91 (s, 4H), 2.45-2.35 (m, 6H), 2.20 (s, 3H), 2.18 (s, 6H). LC-MS (ESI+): m/z: 555.3 RT: 2.40 min (VILLA).

Product B, CIS diastereomer (18.5 mg, yellow pale solid). 1H NMR (400 MHz, DMSO) δ 9.69 (s, 1H), 8.79 (d, J=2.7 Hz, 1H), 8.14 (s, 1H), 7.29 (dd, J=8.8, 2.7 Hz, 1H), 7.17 (series of m, 3H), 7.07 (d, J=8.9 Hz, 1H), 5.46 (dt, J=10.0, 8.0 Hz, 1H), 5.37 (dt, J=11.0, 3.5 Hz, 1H), 4.81 (s, 2H), 4.52 (s, 2H), 4.32-4.27 (m, 2H), 2.95-2.90 (m, 4H), 2.44 (s, 6H), 2.22 (s, 3H), 2.19 (s, 6H). LC-MS (ESI+): m/z: 555.3 RT: 2.496 min (VILLA).

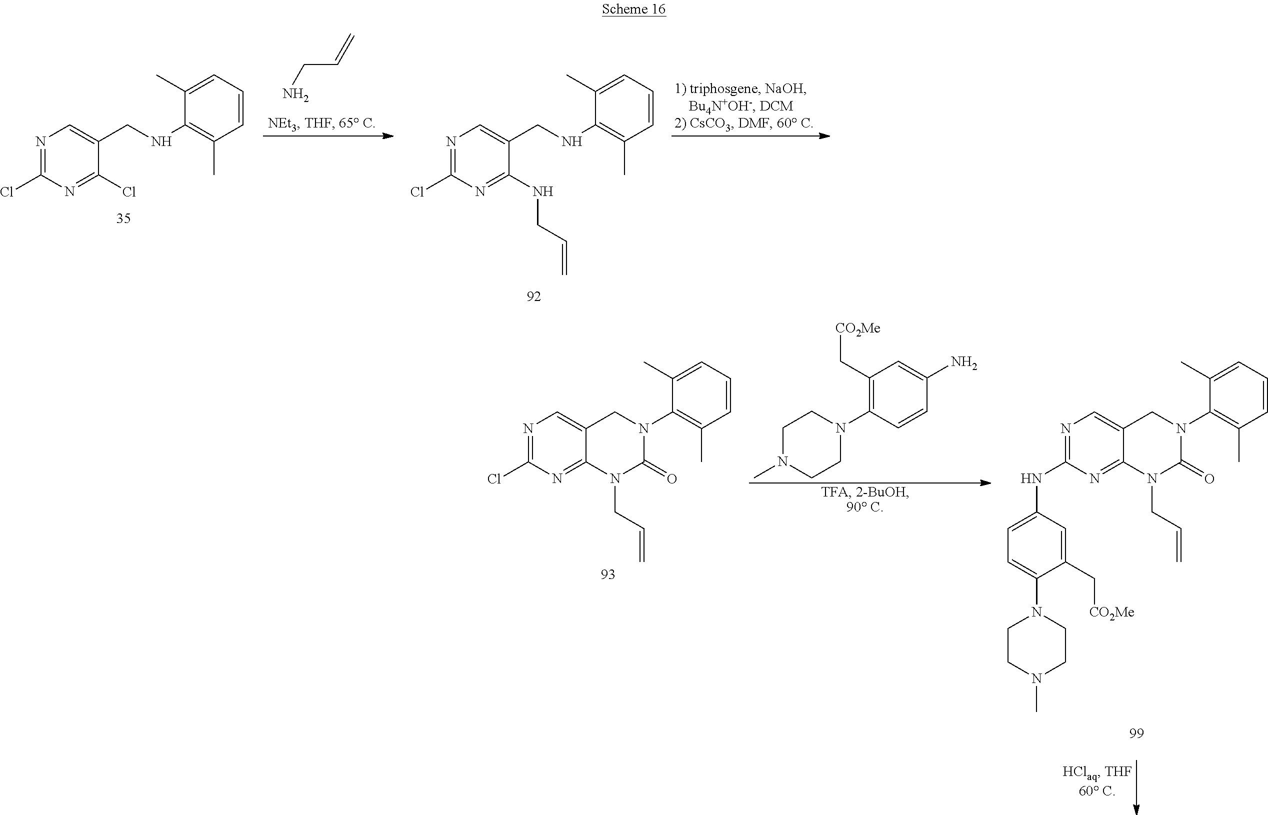

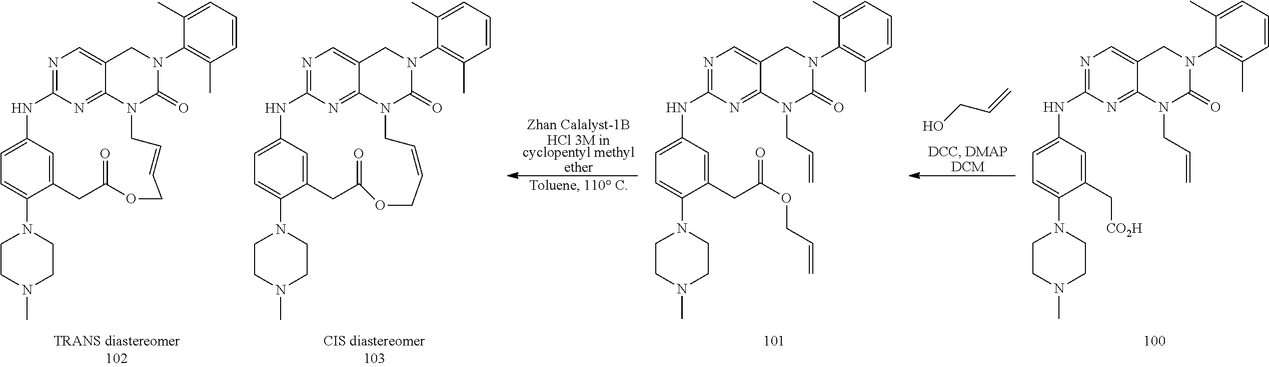

Previous intermediates (35-93) have already been described in the experimental section.

### Methyl 2-(5-((8-allyl-6-(2,6-dimethylphenyl)-7-oxo-5,6,7,8-tetrahydroprimido[4,5-d]pyrimidin-2-yl)amino)-2-(4-methylpiperazin-1-yl)phenyl)acetate (99)

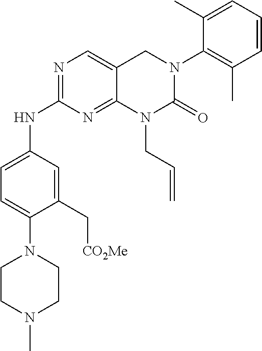 [SMILES: C=CCN1C(=O)N(c2c(C)cccc2C)Cc2cnc(Nc3ccc(N4CCN(C)CC4)c(CC(=O)OC)c3)nc21]

1-Allyl-7-chloro-3-(2,6-dimethyl-phenyl)-3,4-dihydro-1H-pyrimido[4,5-d]pyrimidin-2-one (1.05 g, 3.193 mmol) and methyl 2-(5-amino-2-(4-methylpiperazin-1-yl)phenyl)acetate (1.177 g, 4.47 mmol) were placed in a sealed tube and dissolved in anhydrous 2-BuOH (20.0 mL). Then 3A MS were added followed by trifluoroacetic acid (0.728 mL, 6.386 mmol) and the reaction was stirred at 90° C. for 16 h. Thereafter, the reaction was diluted with water and treated with aq. solution of NaHCO3 (6.386 mmol). The aqueous phase was extracted with CHCl3/iPrOH mixture (1:1). Finally, the combined organic layers were dried over MgSO4, filtered and concentrated under vacuum. The obtained residue was purified by flash column chromatography on silica (Deactivated with DCM:1% NH3) (DCM:MeOH from 100:0 to 90:10) to yield pure product (1.20 g, Yield: 68%, yellow solid). LC-MS (ESI+): m/z: 556.0 RT: 1.15 min (TACC50).

### [5-[8-Allyl-6-(2,6-dimethyl-phenyl)-7-oxo-5,6,7,8-tetrahydro-pyrimido[4,5-d]pyrimidin-2-ylamino]-2-(4-methyl-piperazin-1-yl)-phenyl]-acetic acid (100)

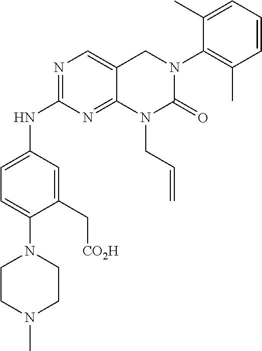 [SMILES: C=CCN1C(=O)N(c2c(C)cccc2C)Cc2cnc(Nc3ccc(N4CCN(C)CC4)c(CC(=O)O)c3)nc21]

Methyl 2-(5-((8-allyl-6-(2,6-dimethylphenyl)-7-oxo-5,6,7,8-tetrahydropyrimido[4,5-d]pyrimidin-2-yl)amino)-2-(4-methylpiperazin-1-yl)phenyl)acetate (1.14 g, 2.052 mmol) was dissolved in THF (8.0 mL) and treated with HCl (10.26 mmol, 6 M aqueous solution, so 1.71 mL) and it was heated at 60° C. for 4 h. Thereafter, solvent was removed under vacuum and the obtained product was used without further purification. LC-MS (ESI+): m/z: 542.0 RT: 1.16 min (TACC50).

### [5-[8-Allyl-6-(2,6-dimethyl-phenyl)-7-oxo-5,6,7,8-tetrahydro-pyrimido[4,5-d]pyrimidin-2-ylamino]-2-(4-methyl-piperazin-1-yl)-phenyl]-acetic acid allyl ester (101)

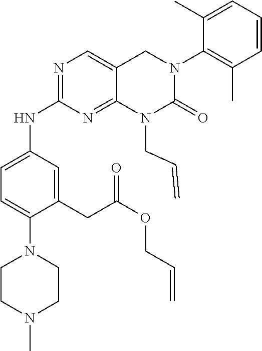 [SMILES: C=CCOC(=O)Cc1cc(Nc2ncc3c(n2)N(CC=C)C(=O)N(c2c(C)cccc2C)C3)ccc1N1CCN(C)CC1]

Allyl alcohol (0.420 mL, 6.15 mmol), DMAP (0.501 g, 4.10 mmol) and DCC (0.634 g, 6.15 mmol) were sequentially added to a solution of [5-[8-Allyl-6-(2,6-dimethyl-phenyl)-7-oxo-5,6,7,8-tetrahydro-pyrimido[4,5-d]pyrimidin-2-ylamino]-2-(4-methyl-piperazin-1-yl)-phenyl]-acetic acid (1.11 g, 2.05 mmol) in a DCM:DMF mixture (4:1, 80.0 mL) and the reaction was stirred at room temperature for 16 h. Thereafter, solvent was removed under vacuum and the obtained residue was purified by flash column chromatography on silica (DCM:MeOH from 100:0 to 90:10) to yield pure product (0.337 g, Yield: 28%, yellow pale solid). LC-MS (ESI+): m/z: 582.2 RT: 1.25 min (TACC50).

### (E)-1³-(2,6-dimethylphenyl)-3⁴-(4-methylpiperazin-1-yl)-1¹,1²,1³,1⁴-tetrahydro-6-oxa-2-aza-1(7,1)-pyrimido[4,5-d]pyrimidina-3(1,3)-benzenacyclodecaphan-8-ene-1²,5-dione (102) and (Z)-1³-(2,6-dimethylphenyl)-3⁴-(4-methylpiperazin-1-yl)-1¹,1²,1³,1⁴-tetrahydro-6-oxa-2-aza-1(7,1)-pyrimido[4,5-d]pyrimidina-3(1,3)-benzenacyclodecaphan-8-ene-1²,5-dione (103)

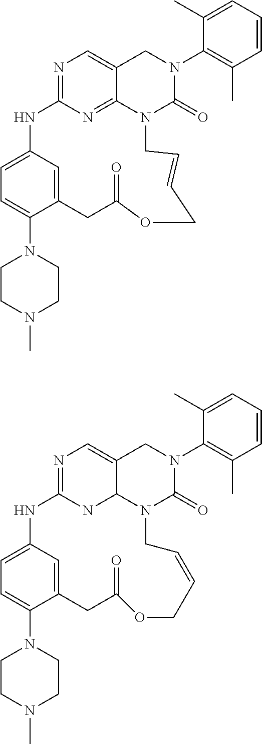

[5-[8-Allyl-6-(2,6-dimethyl-phenyl)-7-oxo-5,6,7,8-tetrahydro-pyrimido[4,5-d]pyrimidin-2-ylamino]-2-(4-methyl-piperazin-1-yl)-phenyl]-acetic acid allyl ester (0.337 g, 0.579 mmol) was placed in a sealed tube and dissolved in anhydrous DMF (3.0 mL) and diluted with toluene (80.0 mL). Then HCl (0.579 mmol 3M in cyclopentyl methyl ether, so 0.19 mL) was added. The reaction crude was degassed by bubbling N2 through the solution, followed by the addition of a solution of Zhan catalyst (0.085 g, 0.116 mmol) in anhydrous toluene (30.0 mL). The reaction crude was degassed again by bubbling N2 through the solution and heated at 110° C. for 16 h. Thereafter, solvent was removed under vacuum and the obtained residue was purified by reverse phase chromatography (from 59% [aq. 25 mM NH4HCO3]-41% ACN to 17% [aq. 25 mM NH4HCO3]-83% ACN) to yield both diastereomers.

Product A, TRANS diastereomer (9.0 mg, white solid). 1H NMR (400 MHz, CDCl3) δ 7.94 (s, 1H), 7.50 (d, J=2.5 Hz, 1H), 7.13 (series of m, 4H), 6.95 (dd, J=8.5, 2.5 Hz, 1H), 6.79 (s, 1H), 6.11 (dt, J=15.8, 6.2 Hz, 1H), 5.74 (dt, J=15.7, 4.0 Hz, 1H), 4.67 (d, J=2.5 Hz, 2H), 4.46 (s, 2H), 4.36 (d, J=6.0 Hz, 2H), 3.84 (s, 2H), 3.04 (broad signal, 4H), 2.68-2.55 (m, 4H), 2.37 (s, 3H), 2.25 (s, 6H). LC-MS (ESI+): m/z: 554.3 RT: 2.665 min (VILLA).

Product B, CIS diastereomer (10.2 mg, white solid). 1H NMR (400 MHz, CDCl3) δ 8.21 (d, J=2.5 Hz, 1H), 7.96 (s, 1H), 7.13 (series of m, 5H), 6.76 (dd, J=8.5, 2.6 Hz, 1H), 5.84-5.76 (m, 1H), 5.61 (dt, J=10.9, 4.3 Hz, 1H), 5.11-5.07 (m, 2H), 4.68 (d, J=7.4 Hz, 2H), 4.49 (s, 2H), 3.87 (s, 2H), 2.96 (t, J=4.7 Hz, 4H), 2.59 (broad signal, 4H), 2.36 (s, 3H), 2.26 (s, 6H). LC-MS (ESI+): m/z: 554.3 RT: 2.833 min (VILLA).

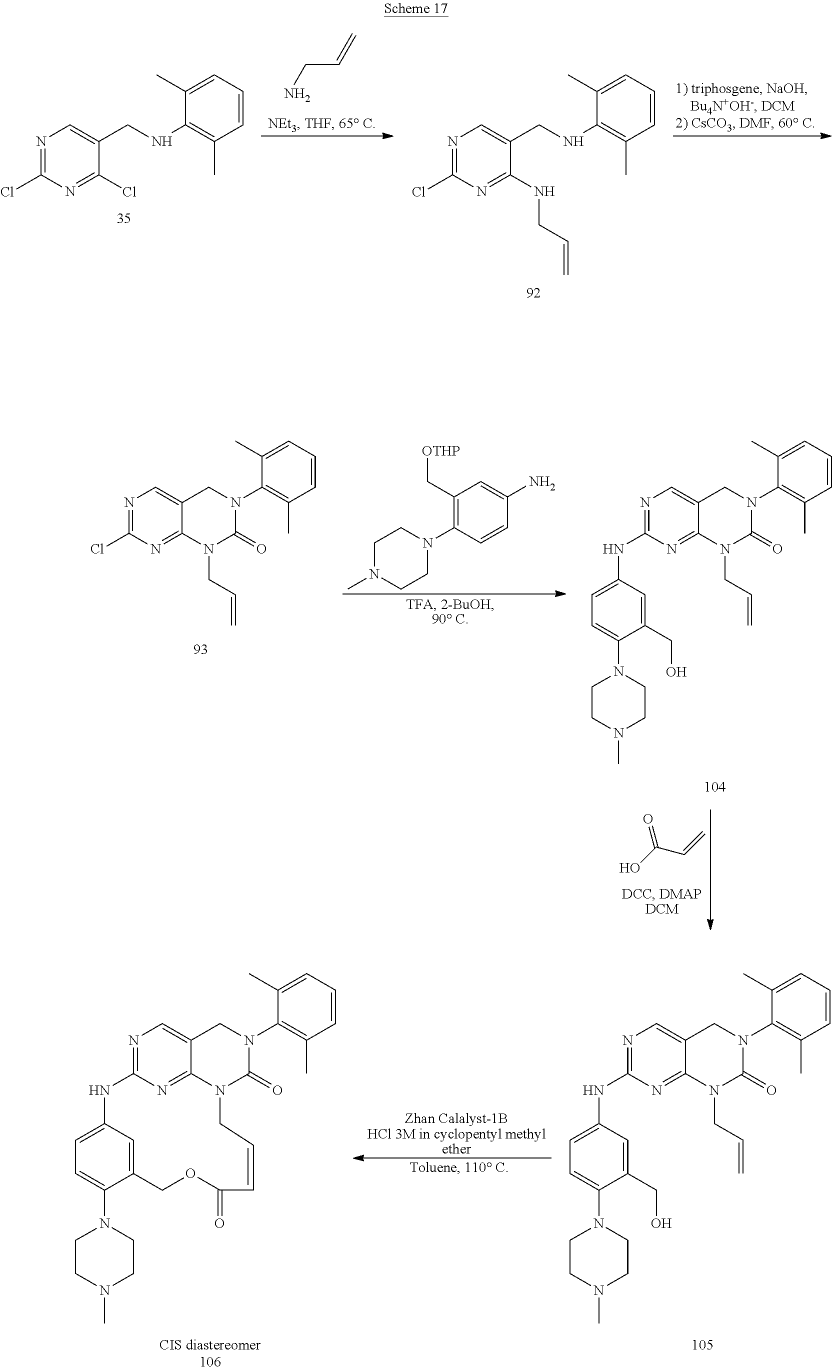

Previous intermediates (35-93)) have already been described in the experimental section.

### 3-(2,6-Dimethyl-phenyl)-7-[3-hydroxymethyl-4-(4-methyl-piperazin-1-yl)-phenylamino]-1-propyl-3,4-dihydro-1H-pyrimido[4,5-d]pyrimidin-2-one (104)

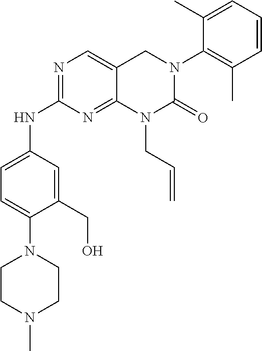 [SMILES: C=CCN1C(=O)N(c2c(C)cccc2C)Cc2cnc(Nc3ccc(N4CCN(C)CC4)c(CO)c3)nc21]

7-Chloro-3-(2,6-dimethyl-phenyl)-1-propyl-3,4-dihydro-1H-pyrimido[4,5-d]pyrimidin-2-one (2.5 g, 7.6 mmol) and 4-(4-Methyl-piperazin-1-yl)-3-(tetrahydro-pyran-2-yloxymethyl)-phenylamine (3.25 g, 10.64 mmol) were placed in a sealed tube and dissolved in anhydrous 2-BuOH (20.0 mL). Then 3A MS were added followed by trifluoroacetic acid (1.20 mL, 15.2 mmol)) and the reaction was stirred at 90° C. for 16 h. Thereafter, the reaction was diluted with water and treated with aq. solution of NaHCO3 (15.2 mmol). The aqueous phase was extracted with CHCl3/iPrOH mixture (1:1). Finally, the combined organic layers were dried over MgSO4, filtered and concentrated under vacuum. The obtained residue was purified by flash column chromatography on silica (DCM:MeOH from 100:0 to 90:10) to yield pure product (2.17 g, Yield: 56%, yellow solid). LC-MS (ESI+): m/z: 514.2 RT: 1.095 min (TACC50).

### Acrylic acid 5-[8-allyl-6-(2,6-dimethyl-phenyl)-7-oxo-5,6,7,8-tetrahydro-pyrimido[4,5-d]pyrimidin-2-ylamino]-2-(4-methyl-piperazin-1-yl)-benzyl ester (105)

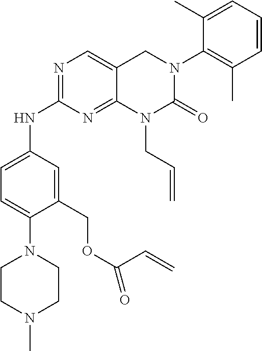 [SMILES: C=CCN1C(=O)N(c2c(C)cccc2C)Cc2cnc(Nc3ccc(N4CCN(C)CC4)c(COC(=O)C=C)c3)nc21]

DMAP (0.52 g, 4.22 mmol), DCC (1.31 g, 6.34 mmol) and acrylic acid (0.58 mL, 8.45 mmol) were sequentially added to a solution of 1-Allyl-3-(2,6-dimethyl-phenyl)-7-[3-hydroxymethyl-4-(4-methyl-piperazin-1-yl)-phenylamino]-3,4-dihydro-1H-pyrimido[4,5-d]pyrimidin-2-one (2.17 g, 4.22 mmol) in anhydrous DCM (50.0 mL) at 0° C. The reaction was allowed to reach room temperature and stirred at that temperature for 16 h. Thereafter, solvent was removed under vacuum and the obtained residue was purified by flash column chromatography on silica (DCM:MeOH from 100:0 to 90:10) to yield pure product (0.996 g, Yield: 42% yield, yellow solid). LC-MS (ESI+): m/z: 568.2 RT: 1.343 min (TACC50).

### (Z)-1³-(2,6-dimethylphenyl)-3⁴-(4-methylpiperazin-1-yl)-1¹,1²,1³,1⁴-tetrahydro-5-oxa-2-aza-1(7,1)-pyrimido[4,5-d]pyrimidina-3(1,3)-benzenacyclononaphan-7-ene-1²,6-dione (106)

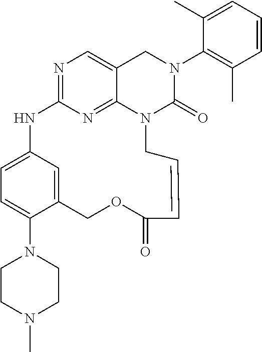 [SMILES: Cc1cccc(C)c1N1Cc2cnc3nc2N(C/C=C\C(=O)OCc2cc(ccc2N2CCN(C)CC2)N3)C1=O]

Acrylic acid 5-[8-allyl-6-(2,6-dimethyl-phenyl)-7-oxo-5,6,7,8-tetrahydro-pyrimido[4,5-d]pyrimidin-2-ylamino]-2-(4-methyl-piperazin-1-yl)-benzyl ester (0.450 g, 0.793 mmol) was placed in a sealed tube and dissolved in anhydrous toluene (140.0 mL). Then HCl (0.793 mmol 3M in cyclopentyl methyl ether, so 0.26 mL) was added. The reaction crude was degassed by bubbling N2 through the solution, followed by the addition of a solution of Zhan catalyst (0.117 g, 0.159 mmol) in anhydrous toluene (20.0 mL). The reaction crude was degassed again by bubbling N2 through the solution and heated at 110° C. for 16 h. Thereafter, solvent was removed under vacuum and the obtained residue was purified by reverse phase chromatography (from 59% [aq. 25 mM NH4HCO3]-41% ACN to 17% [aq. 25 mM NH4HCO3]-83% ACN) to yield pure product as a single CIS diastereomer (0.0154 g, yellow pale solid). 1H NMR (400 MHz, DMSO) δ 9.55 (s, 1H), 8.11 (s, 1H), 7.74 (d, J=2.1 Hz, 1H), 7.17 (series of m, 3H), 7.11 (d, J=8.6 Hz, 1H), 7.02 (dd, J=8.6, 2.3 Hz, 1H), 6.31 (d, J=11.6 Hz, 1H), 6.19 (dt, J=11.7, 6.0 Hz, 1H), 5.39 (s, 2H), 4.90 (d, J=5.1 Hz, 2H), 4.50 (s, 2H), 2.81 (t, J=4.5 Hz, 4H), 2.47 (broad signal, 4H), 2.23 (s, 3H), 2.21 (s, 6H). LC-MS (ESI+): m/z: 540.2 RT: 2.656 min (VILLA).

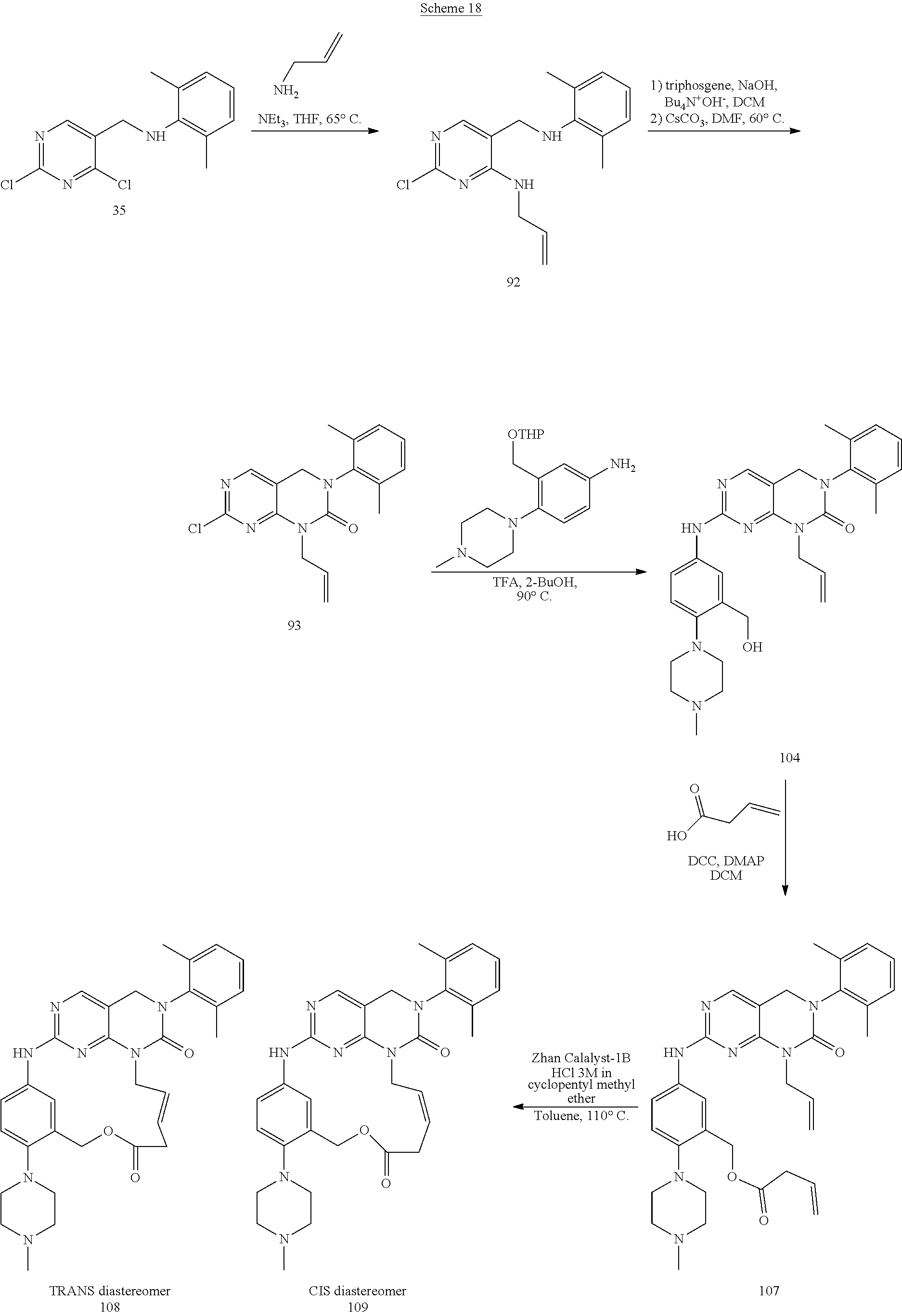

Previous intermediates (35-104) have already been described in the experimental section.

### But-3-enoic acid 5-[8-allyl-6-(2,6-dimethyl-phenyl)-7-oxo-5,6,7,8-tetrahydro-pyrimido[4,5-d]pyrimidin-2-ylamino]-2-(4-methyl-piperazin-1-yl)-benzyl ester (107)

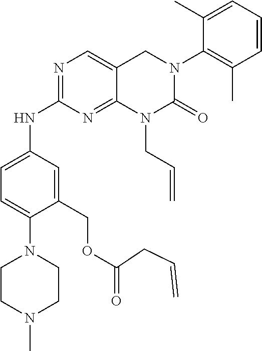 [SMILES: C=CCC(=O)OCc1cc(Nc2ncc3c(n2)N(CC=C)C(=O)N(c2c(C)cccc2C)C3)ccc1N1CCN(C)CC1]

DMAP (0.036 g, 0.292 mmol), DCC (0.145 g, 0.701 mmol) and 3-butenoic acid (0.060 mL, 0.701 mmol) were sequentially added to an ice-cold solution of 1-allyl-3-(2,6-dimethylphenyl)-7-((3-(hydroxymethyl)-4-(4-methylpiperazin-1-yl)phenyl)amino)-3,4-dihydropyrimido[4,5-d]pyrimidin-2(1H)-one (0.30 g, 0.584 mmol) in anhydrous DCM (5.0 mL). The reaction was allowed to reach room temperature and stirred at that temperature for 16 h. Thereafter, the reaction crude was diluted with DCM and washed with brine. Combined organic layers were dried over MgSO4, filtered and concentrated under vacuum. The obtained residue was purified by flash column chromatography on silica (DCM:MeOH from 100:0 to 9:1) to yield pure product (0.285 g, Yield: 84%, yellow pale solid). LC-MS (ESI+): m/z: 582.3 RT: 1.30 min (TACC50).

### (E)-1³-(2,6-dimethylphenyl)-34-(4-methylpiperazin-1-yl)-1¹,1²,1³,1⁴-tetrahydro-5-oxa-2-aza-1(7,1)-pyrimido[4,5-d]pyrimidina-3(1,3)-benzenacyclodecaphan-8-ene-1²,6-dione (108) and (Z)-1³-(2,6-dimethylphenyl)-3⁴-(4-methylpiperazin-1-yl)-1¹,1²,1³,1⁴-tetrahydro-5-oxa-2-aza-1(7,1)-pyrimido[4,5-d]pyrimidina-3(1,3)-benzenacyclodecaphan-8-ene-1²,6-dione (109)

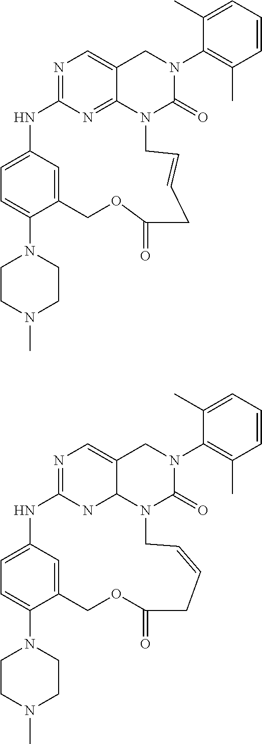

But-3-enoic acid 5-[8-allyl-6-(2,6-dimethyl-phenyl)-7-oxo-5,6,7,8-tetrahydro-pyrimido[4,5-d]pyrimidin-2-ylamino]-2-(4-methyl-piperazin-1-yl)-benzyl ester (0.225 g, 0.387 mmol) was placed in a sealed tube and dissolved in anhydrous toluene (72.0 mL). Then HCl (0.387 mmol 3M in cyclopentyl methyl ether, so 0.129 mL) was added. The reaction crude was degassed by bubbling N2 through the solution, followed by the addition of a solution of Zhan catalyst (0.057 g, 0.077 mmol) in anhydrous toluene (4.0 mL). The reaction crude was degassed again by bubbling N2 through the solution and heated at 110° C. for 16 h. Thereafter, solvent was removed under vacuum and the obtained residue was purified by reverse phase chromatography (from 50% [25 mM NH4HCO3]-50% ACN to 25% [25 mM NH4HCO3]-75% ACN) to yield both diastereomers.

Product A, TRANS diastereomer (12.1 mg, white solid). 1H NMR (400 MHz, CDCl3) δ 7.96 (s, 1H), 7.81 (s, 1H), 7.18-7.08 (m, 4H), 6.94 (s, 1H), 6.86 (dd, J=8.5, 2.3 Hz, 1H), 6.02 (dt, J=15.7, 4.5 Hz, 1H), 5.76-5.64 (m, 1H), 5.36 (s, 2H), 4.56 (s, 2H), 4.50 (s, 2H), 3.10 (d, J=6.8 Hz, 2H), 2.95-2.89 (m, 4H), 2.61 (broad signal, 4H), 2.38 (s, 3H), 2.26 (s, 6H). LC-MS (ESI+): m/z: 555.3 RT: 2.303 min (VILLA).

Product B, CIS diastereomer (9.3 mg, white solid). 1H NMR (400 MHz, CDCl3) δ 8.52 (d, J=2.1 Hz, 1H), 7.97 (s, 1H), 7.15 (series of m, 5H), 6.79 (dd, J=8.5, 2.4 Hz, 1H), 5.69-5.60 (m, 1H), 5.59-5.51 (m, 1H), 5.33 (s, 2H), 5.09-5.03 (m, 2H), 4.51 (s, 2H), 3.21 (d, J=8.3 Hz, 2H), 2.93-2.87 (m, 4H), 2.62 (broad signal, 4H), 2.38 (s, 3H), 2.26 (s, 6H). LC-MS (ESI+): m/z: 555.3 RT: 2.444 min (VILLA).

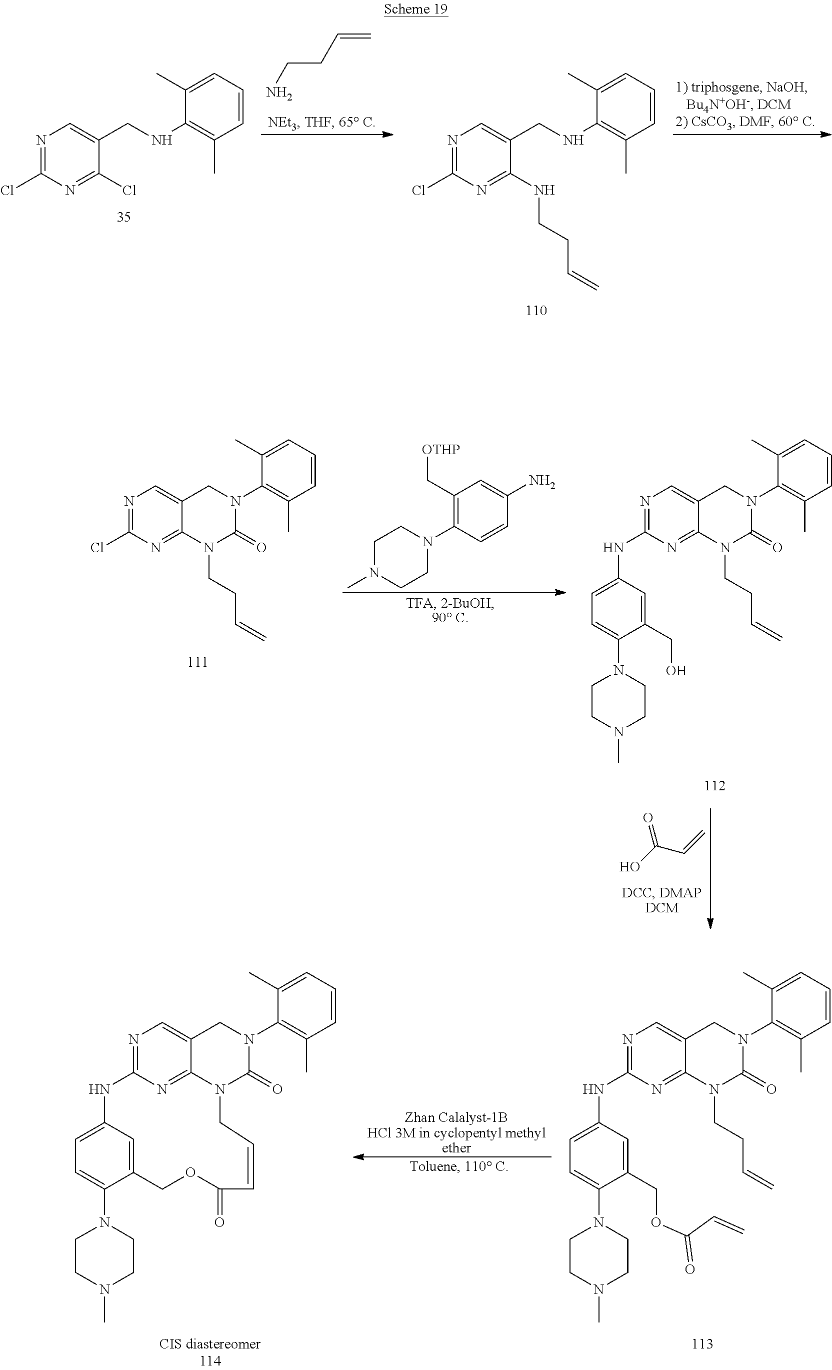

Previous intermediates (35) have already been described in the experimental section.

### Butyl-{2-chloro-5-[(2,6-dimethyl-phenylamino)-methyl]-pyrimidin-4-yl}-amine (110)

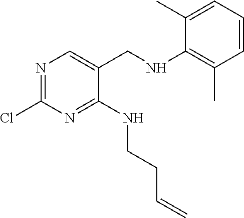 [SMILES: C=CCCNc1nc(Cl)ncc1CNc1c(C)cccc1C]

Anhydrous triethylamine (2.96 mL, 21.26 mmol) was added to a solution of (2,4-Dichloro-pyrimidin-5-ylmethyl)-(2,6-dimethyl-phenyl)-amine (4.00 g, 14.2 mmol) and 3-butenylamine (2.0 g, 21.26 mmol) in anhydrous THF (45.0 mL). The reaction crude was stirred at 60° C. for 16 h. Thereafter, the reaction crude was diluted with AcOEt and washed with brine. The organic layer was dried over MgSO4, filtered and concentrated under vacuum. The obtained residue was purified by flash column chromatography on silica (Heptane:AcOEt from 100:0 to 50:50) to yield pure product (4.22 g, Yield: 94%, yellow oil). LC-MS (ESI+): m/z: 317.1 RT: 1.841 min (TACC50).

### 1-Allyl-7-chloro-3-(2,6-dimethyl-phenyl)-3,4-dihydro-1H-pyrimido[4,5-d]pyrimidin-2-one (111)

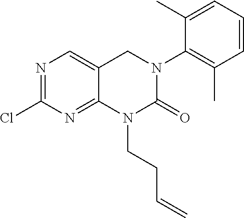 [SMILES: C=CCCN1C(=O)N(c2c(C)cccc2C)Cc2cnc(Cl)nc21]

Triphosgene (5.90 g, 19.9 mmol) was added to a solution of Butyl-{2-chloro-5-[(2,6-dimethyl-phenylamino)-methyl]-pyrimidin-4-yl}-amine (4.20 g, 13.26 mmol) in DCM (45.0 mL) and it was stirred at room temperature for 1 h. Then an aq. solution of sodium hydroxide (10.60 g, 265.12 mmol, 4.9 M) and tetrabutylammonium hydroxide (1.70 mmol 55% in aq. solution, so 0.85 mL) was added at 0° C. The reaction was allowed to reach room temperature and stirred at room temperature for 16 h. Thereafter, the reaction crude was diluted with DCM and washed with water. The organic layer was dried over MgSO4, filtered and concentrated under vacuum. The obtained residue was dissolved in DMF (40.0 mL) and cessium carbonate (4.36 g, 13.39 mmol) was added. The reaction mixture was heated at 60° C. for 16 h. Thereafter, the reaction crude was diluted with DCM and washed with brine. The organic layer was dried over MgSO4, filtered and concentrated under vacuum. The obtained residue was purified by flash column chromatography on silica (Heptane:AcOEt 100:0 to 60:40) to yield pure product (2.61 g, Yield: 57%, yellow solid). LC-MS (ESI+): m/z: 343.1 RT. 1.786 min (TACC50).

### 1-Butyl-3-(2,6-dimethyl-phenyl)-7-[3-hydroxymethyl-4-(4-methyl-piperazin-1-yl)-phenylamino]-3,4-dihydro-1H-pyrimido[4,5-d]pyrimidin-2-one (112)

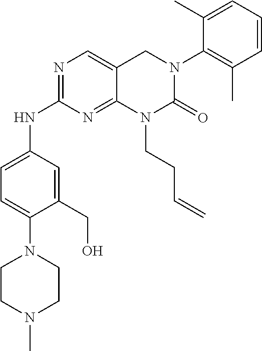 [SMILES: C=CCCN1C(=O)N(c2c(C)cccc2C)Cc2cnc(Nc3ccc(N4CCN(C)CC4)c(CO)c3)nc21]

1-Allyl-7-chloro-3-(2,6-dimethyl-phenyl)-3,4-dihydro-1H-pyrimido[4,5-d]pyrimidin-2-one (1.00 g, 2.92 mmol) and 4-(4-Methyl-piperazin-1-yl)-3-(tetrahydro-pyran-2-yloxymethyl)-phenylamine (1.25 g, 4.08 mmol) were placed in a sealed tube and dissolved in anhydrous 2-BuOH (10.0 mL). Then 3A MS were added followed by anhydrous trifluoroacetic acid (0.45 mL, 5.8 mmol) and the reaction was stirred at 90° C. for 16 h. Thereafter, the reaction was diluted with water and treated with aq. solution of NaHCO3 (5.8 mmol). The aqueous phase was extracted with CHCl3/iPrOH mixture (1:1). Finally, the combined organic layers were dried over MgSO4, filtered and concentrated under vacuum. The obtained residue was purified by flash column chromatography on silica (DCM:MeOH from 100:0 to 90:10) to yield pure product (1.03 g, Yield: 67%, yellow solid). LC-MS (ESI+): m/z: 528.2 RT: 1.009 min (TACC50).

### Acrylic acid 5-[8-butyl-6-(2,6-dimethyl-phenyl)-7-oxo-5,6,7,8-tetrahydro-pyrimido[4,5-d]pyrimidin-2-ylamino]-2-(4-methyl-piperazin-1-yl)-benzyl ester (113)

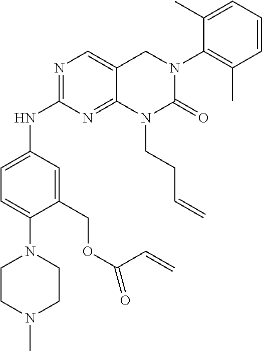 [SMILES: C=CCCN1C(=O)N(c2c(C)cccc2C)Cc2cnc(Nc3ccc(N4CCN(C)CC4)c(COC(=O)C=C)c3)nc21]

DMAP (0.24 g, 1.95 mmol), DCC (0.60 g, 2.93 mmol) and acrylic acid (0.27 mL, 3.90 mmol) were sequentially added to an ice-cold solution of 1-Butyl-3-(2,6-dimethyl-phenyl)-7-[3-hydroxymethyl-4-(4-methyl-piperazin-1-yl)-phenylamino]-3,4-dihydro-H-pyrimido[4,5-d]pyrimidin-2-one (1.03 g, 1.95 mmol) in anhydrous DCM (12.0 mL). The reaction was allowed to reach room temperature and stirred at that temperature for 16 h. Thereafter, the reaction crude was diluted with DCM and washed with brine. Combined organic layers were dried over MgSO4, filtered and concentrated under vacuum. The obtained residue was purified by flash column chromatography on silica (DCM:MeOH from 100:0 to 90:10) to yield pure product (0.67 g, Yield: 59%, white solid). LC-MS (ESI+): m/z: 582.2 RT: 1.316 min (TACC50).

### (Z)-1³-(2,6-dimethylphenyl)-3⁴-(4-methylpiperazin-1-yl)-1¹,1²,1³,1⁴-tetrahydro-5-oxa-2-aza-1(7,1)-pyrimido[4,5-d]pyrimidina-3(1,3)-benzenacyclodecaphan-7-ene-1²,6-dione (114)

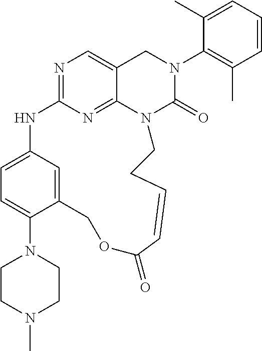 [SMILES: Cc1cccc(C)c1N1Cc2cnc3nc2N(CC/C=C\C(=O)OCc2cc(ccc2N2CCN(C)CC2)N3)C1=O]

Acrylic acid 5-[8-butyl-6-(2,6-dimethyl-phenyl)-7-oxo-5,6,7,8-tetrahydro-pyrimido[4,5-d]pyrimidin-2-ylamino]-2-(4-methyl-piperazin-1-yl)-benzyl ester (0.20 g, 0.344 mmol) was placed in a sealed tube and dissolved in anhydrous toluene (64.0 mL). Then HCl (0.344 mmol, 3M in cyclopentyl methyl ether, so 0.115 mL) was added. The reaction crude was degassed by bubbling N2 through the solution, followed by the addition of a solution of Zhan catalyst (0.051 g, 0.069 mmol) in anhydrous toluene (5.0 mL). The reaction crude was degassed again by bubbling N2 through the solution and heated at 110° C. for 16 h. Thereafter, the solvent removed under vacuum and the obtained residue was purified by reverse phase chromatography (from 59% [aq. 25 mM NH4HCO3]-41% ACN to 17% [aq. 25 mM NH4HCO3]-83% ACN) to yield pure product as a single CIS diastereomer (0.017 g, brown pale solid). 1H NMR (400 MHz, DMSO) δ 9.62 (s, 1H), 8.54 (d, J=2.0 Hz, 1H), 8.14 (s, 1H), 7.20-7.15 (m, 3H), 7.10 (dt, J=8.7, 5.6 Hz, 2H), 6.33 (dt, J=11.7, 8.9 Hz, 1H), 5.89 (d, J=11.9 Hz, 1H), 5.32 (s, 2H), 4.49 (s, 2H), 3.95-3.89 (m, 2H), 2.95 (dd, J=14.0, 8.3 Hz, 2H), 2.79 (t, J=4.5 Hz, 4H), 2.48-2.42 (m, 3H), 2.24 (s, 3H), 2.18 (s, 6H). LC-MS (ESI+): m/z: 555.3 RT: 2.504 min (VILLA).

### Experimental Procedure

### HPLC_MS Method Description

TACC50: Agilent 1260 Infinity (Quat. Pump) DAD LC/MS G6120 (G1948B) instrument. Column Thermo Scientific Accucore C18 (50×4.6 mm, 2.6 μm). Mobile phases: A. 0.1% HCOOH in H2O B: CH₃CN. From 90% A to 10% A in 1.5 min, held for 0.9 min, to 95% A in 0.1 min. Flow 3 mL/min, 30° C.

VILLA: Agilent 1100 HPLC DAD LC/MS G1956A instrument. Column YMC-pack ODS-AQ C18 (50×4.6 mm, 3 μm). Mobile phases: A: 0.1% HCOOH in H2O B: CH3CN. From 95% A to 5% A in 4.8 min, held for 1.0 min, to 95% A in 0.2 min. Flow 2.6 mL/min, 35° C.

VILL-J: Agilent 1100 HPLC DAD LC/MS G1956A instrument. Column Waters-XBridge C18 (50×4.6 mm, 3.5 μm). Mobile phases: A: 40 mM NH4OAc in H2O+5% CH3CN B: CH3CN. From 95% A to 0% A in 4.5 min, held for 1.0 min, to 95% A in 0.5 min. Flow 2.6 mL/min, 50° C.

VILLA-2T: Agilent 1260 Infinity DAD TOF-LC/MS G6224A instrument. Column: YMC-pack ODS-AQ C18 (50×4.6 mm, 3 μm). Mobile phases: A: 0.1% HCOOH in H2O B: CH3CN. From 95% A to 5% A in 4.8 min, held for 1.0 min, to 95% A in 0.2 min. Flow 2.6 mL/min, 35° C.

### Example 4: SIK1, 2 and 3 Kinase Activity

SIK1, 2 and 3 kinase activity was assessed using a Eurofins Cerep S.A. (‘Eurofins’) KinaseProfiler. All compounds were prepared to 50× final assay concentration in 100% DMSO. This working stock of the compound was added to the assay well as the first component in the reaction, followed by the remaining components as detailed in the general assay protocols below. In the standard KinaseProfiler service, there is no pre-incubation step between the compound and the kinase prior to initiation of the reaction. The positive control well contains all components of the reaction, except the compound of interest; however, DMSO (at a final concentration of 2%) is included in these wells to control for solvent effects. The blank wells contain all components of the reaction, with a reference inhibitor replacing the compound of interest. This abolishes kinase activity and establishes the base-line (0% kinase activity remaining).

SIK (h) was incubated with 8 mM MOPS pH 7.0, 0.2 mM EDTA, 100 μM AMARAASAAALARRR, 10 mM Magnesium acetate and [γ-33P]-ATP (specific activity and concentration as required). The reaction was initiated by the addition of the Mg/ATP mix. After incubation for 40 minutes at room temperature, the reaction was stopped by the addition of phosphoric acid to a concentration of 0.5%, 10 μL of the reaction was then spotted onto a P30 filtermat and washed four times for 4 minutes in 0.425% phosphoric acid and once in methanol prior to drying and scintillation counting.

SIK2 (h) was incubated with 8 mM MOPS pH 7.0, 0.2 mM EDTA, 100 μM KKKVSRSGLYRSPSMPENLNRPR, 10 mM Magnesium acetate and [γ-33P-ATP](specific activity and concentration as required). The reaction was initiated by the addition of the Mg/ATP mix. After incubation for 40 minutes at room temperature, the reaction was stopped by the addition of phosphoric acid to a concentration of 0.5%, 10 μL of the reaction was then spotted onto a P30 filtermat and washed four times for 4 minutes in 0.425% phosphoric acid and once in methanol prior to drying and scintillation counting.

SIK3 (h) was incubated with 8 mM MOPS pH 7.0, 0.2 mM EDTA, 100 μM KKKVSRSGLYRSPSMPENLNRPR, 10 mM Magnesium acetate and [γ-33P-ATP](specific activity and concentration as required). The reaction was initiated by the addition of the Mg/ATP mix. After incubation for 40 minutes at room temperature, the reaction is stopped by the addition of phosphoric acid to a concentration of 0.5%, 10 LL of the reaction was then spotted onto a P30 filtermat and washed four times for 4 minutes in 0.425% phosphoric acid and once in methanol prior to drying and scintillation counting.

Results are depicted in the following Table 5:

TABLE 5

| Macrocycle Precursor | IC50 nM | | |
|---|---|---|---|
| Compound | SIK1 | SIK2 | SIK3 |
| SLT-016 | 5 | 7 | 20 |
| SLT-017 | 5 | 5 | 21 |
| SLT-018 | 3 | 2 | 7 |
| SLT-019 | 2 | 3 | 8 |
| SLT-020 | 5 | 10 | 43 |
| SLT-021 | 2 | 2 | 8 |
| SLT-022 | 4 | 13 | 26 |
| SLT-024 | 4 | 5 | 11 |
| SLT-025 | 3 | 6 | 21 |
| SLT-027 | 2 | 5 | 21 |
| SLT-028 | 4 | 8 | 34 |

Results are depicted in the following Table 6:

TABLE 6

| Macrocycle | IC50 nM | | |
|---|---|---|---|
| Compound | SIK1 | SIK2 | SIK3 |
| SLT-023 | 3 | 3 | 18 |
| SLT-026 | 2 | 2 | 3 |
| SLT-033 | | 0.8 | |
| SLT-038 | | 1 | |
| SLT-042 | 2 | 0.7 | 2 |
| SLT-048 | | 0.6 | |
| SLT-058 | 26 | 201 | 339 |
| SLT-059 | 286 | 250/>500 | |
| SLT-061 | | 2 | |
| SLT-062 | | 0.8 | |
| SLT-068 | | 17 | |
| SLT-070 | | 2 | |
| SLT-071 | | 3 | |
| SLT-073 | | 0.7 | |
| SLT-079 | | 1 | |
| SLT-080 | | 3 | |
| SLT-081 | | 1 | |
| SLT-091 | 12 | 30 | 44 |

Macrocycle compounds show potent inhibitory activity against the Salt Induced Kinases. Ring size and substitution of the aromatic moieties can affect the potency, as evidenced by SLT-058 and SLT-059.

### Example 5: Cell-Based Kinase Activity

Cell-based kinase activity was determined using a PathHunter detection system. PathHunter cell lines were expanded from freezer stocks according to standard procedures. Cells were seeded in a total volume of 20 μL into white walled, 384-well microplates and incubated for the appropriate time prior to testing. For agonist determination, cells were incubated with sample to induce response. Intermediate dilution of sample stocks was performed to generate 5× sample in assay buffer. 5 μL of 5× sample was added to cells and incubated at room temperature for 3 hours. Vehicle concentration was 1%. Assay signal was generated through a single addition of 12.5 or 15 μL (50% v/v) of PathHunter Detection reagent cocktail for agonist and antagonist assays respectively, followed by a one hour incubation at room temperature. Microplates were read following signal generation with a PerkinElmer Envision™ instrument for chemilumine-scent signal detection. Compound activity was analyzed using CBIS data analysis suite (ChemInnovation, CA). For agonist mode assays, percentage activity was calculated using the following formula: % Activity=100%×(mean RLU of test sample−mean RLU of vehicle control)/(mean MAX RLU control ligand −mean RLU of vehicle control).

Results are depicted in the following Table 7:

TABLE 7

| Compound | EC50 SIK1 (nM) |
|---|---|
| SLT-023 | 86.6 |
| SLT-026 | 3.24 |
| SLT-027 | 408 |
| SLT-033 | 6.08 |
| SLT-038 | 4.37 |
| SLT-041 | 10.5 |
| SLT-042 | 2.02 |
| SLT-048 | 2.58 |

### Example 6: Cell-Based SIK1 Activity of SLT-048 and SLT-026

SIK1 activity was determined using a NanoBRET Target Engagement Intracellular Kinase Assay for screening of compounds binding to SIK1. The NanoBRET target engagement assay employs an energy transfer technique designed to measure molecular proximity in living cells. The assay measures the apparent affinity of test compounds by competitive displacement of the NanoBRET tracer, reversibly bound to a NanoLuc luciferase-kinase fusion construct in cells. The intracellular binding affinity and selectivity are physiologically relevant and fundamental to the pharmacological mechanism of the compounds. HEK293 cells transiently expressing a NanoLuc-SIK1 Fusion Vector were seeded into the wells of 384-well plates. The cells were pre-treated with the NanoBRET Tracer K-4 and then treated with reference compound Dasatinib for 1 hour. The BRET signal was measured on an Envision 2104 Multilabel Reader. IC50 value was calculated and the IC50 curve was plotted using the GraphPad Prism 4 program based on a sigmoidal dose-response equation. The IC50 (M) for the reference compound Dasatinib was 3.188e⁻⁹.

Next, SIK1 activity was determined for SLT-026 and SLT-048. HEK293 cells were transfected with 1 μg SIK1-NanoLuc fusion vector and 9 μg transfection carrier DNA. The transfected cells were treated with customer compounds (starting at 10 μM, 10-dose with 3-fold dilution) and reference compound (starting at 1 μM, 10-dose with 3-fold dilution). SIK1 target engagement was measured by NanoBRET assay. Curve fits were performed only when the percent of NanoBret signal at the highest concentration of compounds was less than 55%.

IC50 (M) values for SLT-048 and SLT-026 were 1.63e⁻¹⁰ and 4.25e⁻¹¹, respectively (Dasatinib was 2.77e⁻⁹). SLT-048 and SLT-026 demonstrated increased SIK1 inhibitory activity compared to controls (Dasatinib).

### Example 7: Evaluation of the Effects of SLT-023 and SLT-048 on the Pigmentation of B16 4A5 Cells In Culture

The present study aimed to evaluate the pro-pigmenting properties of SLT-023 and SLT-048 compounds in B16 4A5 cells. Firstly, a dose determination study was performed by testing 5 concentrations of each compound on cell viability using an MTS assay. Secondly, the tanning properties of the test compounds, applied at 3 concentrations in culture medium, were studied through melanin content measurement after chemical extraction. In parallel, the dosage of proteins using the Bradford method was performed in order to normalize the melanin content to whole cell proteins.

The study was carried out on B16 4A5 mouse melanoma cells (ECACC; 94042254)—melanin producing. The cells were cultivated as a monolayer with DMEM medium supplemented with 20% M199, 10% FBS and penicillin and streptomycin and grown in a cell incubator at 37° C. and 5% CO₂.

B16 4A5 cells were seeded in 24-well plates at 20,000 cells/well, 24 h before the beginning of the treatment with test compounds at 5 concentrations, in triplicates (n=3). Then cells were incubated for 72 h with SLT-023 and SLT-048 at 5 concentrations (5, 0.7, 0.1, 0.0146 and 0.002 μg/mL), 72 h with SDS 0.05% or were left untreated. Additionally, DMSO at 0.1%, used for solubilization of the test compounds, was tested in parallel. The treatments were performed in culture medium containing only 1% of FBS. At the end of the treatments, cell viability was evaluated with a MTS assay (3-(4,5-dimethylthiazol-2-yl)-5-(3-carboxy-methoxyphenyl)-2-(4-sulfophenyl-2H-tetrazolium).

B16-4A5 melanocytes were seeded at 100,000 cells/well in 6-well plates 24 h before being treated for 72 h with SLT-023 (1, 0.3 and 0.1 μg/mL), and SLT-048 (3, 1 and 0.3 μg/mL) Additionally, DMSO at 0.06% 72 h and theophylline (Sigma; T1633) at 2 mM 72 h were used for the test compounds solubilization and positive control of pigmentation, respectively. The treatments were performed in culture medium+1% FBS.

After 72 h of treatment, B16-4A5 cells were lysed with 1M NaOH and the total protein content was quantified by Bradford method. Melanin content was determined after heating the samples for 1 hour at 80° C. The absorbance reading was done at 490 nm and compared to a synthetic melanin standard curve. These results were normalised to the amount of protein present in each sample measured using the Bradford method.

The effects of the compounds on B16 4A5 viability was assessed. To this purpose, cells were incubated during 72 h with 5 concentrations of the compounds, were left untreated, treated with SDS or with DMSO 0.1%. After 72 h of incubation, B16 4A5 viability was assessed by a MTS assay with 0.01<p-values<0.05 considered as significant (* or $), 0.001<p-values<0.01 as highly significant (** or $$) and p-values<0.001 as very highly significant (*** or $$$).

Since the solvent of SLT-023 and SLT-048 was DMSO, the viability of cells incubated with these compounds was compared with DMSO 0.1% control. The viability of cells incubated with SDS 0.05% was strongly and significantly diminished compared to untreated cells, which ensures for the robustness of the experiment. The viability of cells incubated with 5 μg/mL of SLT-023 and SLT-048 was slightly diminished. The cell viability of cells incubated with 0.7 μg/mL of SLT-023 was slightly decreased.

Next, the effects of the compounds on B16 4A5 melanin production were assessed. For this purpose, cells were incubated 72 h with 3 concentrations of the compounds, were left untreated, treated with theophylline 2 mM (positive control of melanin production) or with DMSO 0.06%. After 72 h incubation of B16 4A5, the intracellular melanin content was assessed.

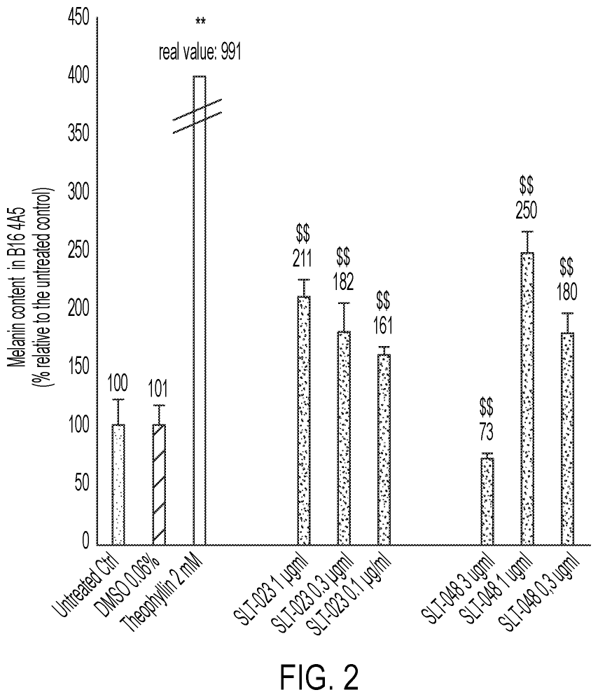

At the end of the 72 h of treatment, cells were lysed with NaOH and the melanin content was determined by spectrophotometry after 1 h heating at 80° C. The quantity of proteins present in each sample was determined using Bradford method. The melanin content was then normalized to the quantity of proteins. A statistical analysis (Student t-test) was performed on the normalized data. The effects of the test compounds SLT-023 and SLT-048 were compared to the DMSO 0.06% condition with 0.01<p-values<0.05 considered as significant (* or $), 0.001<p-values<0.01 as highly significant (** or $$) and p-values<0.001 as very highly significant (*** or $$$) (FIG. 2).

The compound SLT-023 (1, 0.3 and 0.1 μg/ml) significantly increased the production of melanin at every dose, and in a dose-dependent manner. SLT-048 at 1 and 0.3 μg/ml was also able to significantly increase the production of melanin within the cells after 72 h of treatment.

In conclusion, SLT-048 at 1 and 0.3 μg/ml was able to increase the production of melanin (respectively 250% and 180% in comparison with the 0.06% DMSO used as solvent). SLT-023 significantly increased the production of melanin in B16 4A5 cells at any doses, and in a dose-dependent manner.

### Example 8: Assessment of SLT-023, -026, -042 and -048, on Pigmentation Using Living Human Skin Explants Ex Vivo

The aim of the study was to evaluate effects of different products on pigmentation (pro-pigmentation effects) using living human skin explants. This activity was evaluated by controlling cell viability after Masson's trichrome staining and visualization of melanin after Fontana-Masson staining.

All the tested products were diluted in the vehicle noted as E (Ethanol 65%, Propylene glycol 25%, Transcutol 10%). The diluted solutions were stored at 4° C. within the study period. 36 human skin explants of an average diameter of 11 mm (+1 mm) were prepared on an abdoplasty coming from a 53-year-old Caucasian woman (reference: P2462-AB53) with a III phototype. The explants were kept in survival in BEM culture medium (BIO-EC's Explants Medium) at 37° C. in a humid, 5%-CO2 atmosphere. Study is performed on human skin tissue, obtained from surgical residues in full respect with the Declaration of Helsinki and the article L. 1243-4 of the French Public Health Code. The latter does not require any prior authorization by an ethics committee for sampling and using surgical wastes.

The excipient E and the tested products were applied topically at a rate of 2 μL per 1 cm² explant (˜2 mg/cm²) and spread and gently rubbed for 10 seconds using a small spatula on day 0 (DO), D3, D5, D7 and D10. The control explants T did not receive any treatment except the renewal of culture medium. The culture medium was half renewed (1 ml per well) on D3, D5, D7 and D10. On D0, D3, D4, D5, D6, D7 and D10, the culture media of the irradiated explants (UV) were replaced by HBSS (Hank's Balanced Saline Solution; 1 ml per explant). Then, the explants of the “UV” batch were irradiated using a UV simulator Vibert Lourmat RMX 3W with a dose of 2.25 J/cm² of UVA (with 6-8% of UVB) corresponding to 0.5 MED (minimal erythemal dose) on a skin with a III phototype. At the end of the UV irradiation, the explants form the batch TUV were put back in 2 mL of BEM medium. On D0, the 3 explants from the batch TO were collected and cut in two parts. Half was fixed in buffered formalin solution and half was frozen at −80° C. On D10, 3 explants from the concerned batches were collected and treated in the same way than in DO. According to the dispositions mentioned in the study plan, the days of treatments, irradiations and sampling were adjusted to the schedule of working days. After fixation for 24 hours in buffered formalin, the samples were dehydrated and impregnated in paraffin using a Leica PEARL dehydration automat. The samples were embedded using a Leica EG 1160 embedding station. 5-μm-thick sections were made using a Leica RM 2125 Minot-type microtome, and the sections were mounted on Superfrost® histological glass slides. The frozen samples were cut into 7-μm-thick sections using a Leica CM 3050 cryostat. Sections were then mounted on Superfrost®, plus silanized glass slides. The microscopical observations were realized using a Leica DMLB or Olympus BX43 microscope. Pictures were digitized with a numeric DP72 Olympus camera with CellSens storing software.

The cell viability of the epidermal and dermal structures was assessed by microscopical observation of formalin-fixed paraffin-embedded (FFPE) skin sections after Masson's trichrome staining, Goldner variant. Cell viability was very slightly decreased among samples treated with 1% SLT-023, -026, -042 and -048.

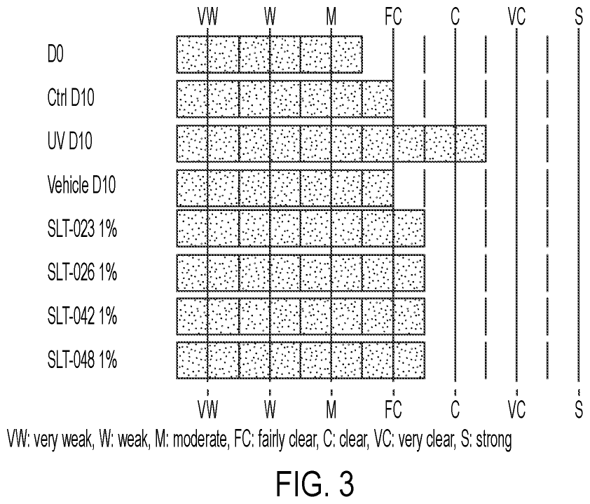

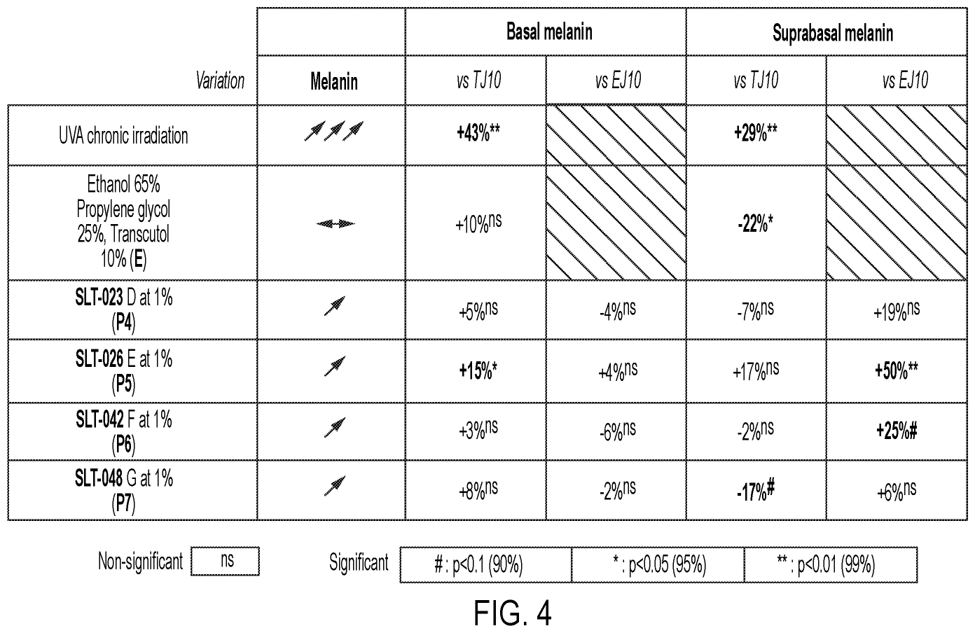

Further analysis to quantify the melanin in basal and suprabasal layers was undertaken. Melanin was visualized after silver impregnation according to Masson's Fontana staining method on FFPE skin sections. The staining was assessed by microscopical observation (FIG. 3) and by image analysis (FIG. 4). On day 0 and day 10, macroscopic photos were taken using the Nikon D5300 camera.

The UV-induced increase of melanin vs untreated explants (UVA) validated the experiment as a positive control. The chronic UVA/B irradiations induced a significant increase of 43%** in the basal layer and a significant increase of 29% in the suprabasal layers. The vehicle “Ethanol 65%, Propylene glycol 25%, Transcutol 10%” (E) induced no significant variation in the basal layer and a significant decrease of 22%* in the suprabasal layers. The product SLT-026, E at 1% (P5) induced a significant increase of 50%** in the suprabasal layers. The product SLT-042, F at 1% (P6) induced a significant increase of 25% # in the suprabasal layers.

In conclusion, SLT-023, SLT-026, SLT-042 and SLT-048 increased melanin production in living human skin explants.

### Example 9: Assessment of SLT-048 on Pigmentation Using Living Human Skin Explants Ex Vivo

The aim of this study was to evaluate the effects of SLT-048 on pigmentation (pro-pigmentation effects) using living human skin explants in survival medium.

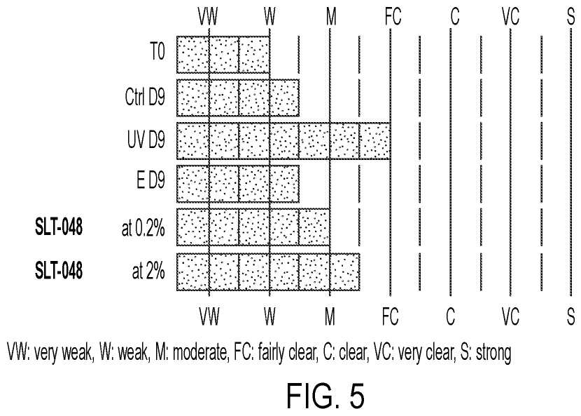

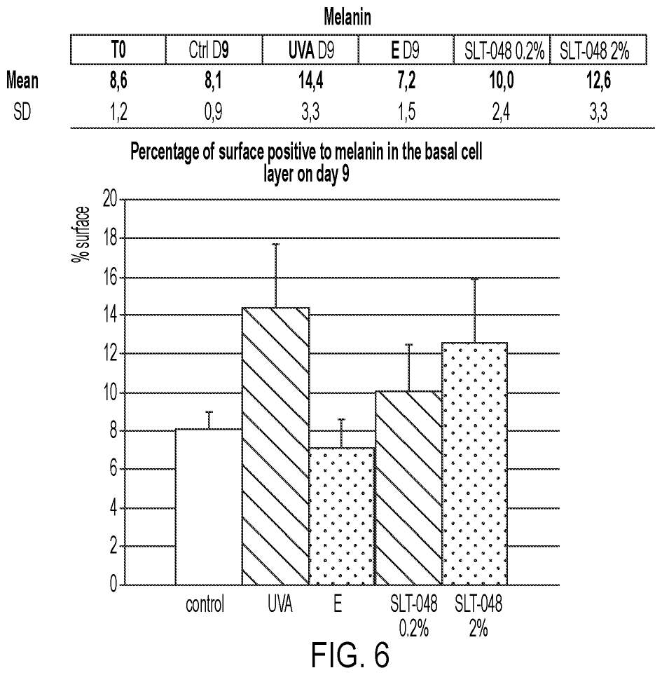

All the tested products were diluted in the vehicle (Ethanol 70%, Propylene glycol 30%). 24 human skin explants of an average diameter of 1 mm (±1 mm) were prepared on an abdoplasty coming from a 43-year-old Caucasian woman (reference: P2511-AB43) with a II phototype. The explants were kept in survival in BEM culture medium (BIO-EC's Explants Medium) at 37° C. in a humid, 5%-CO₂ atmosphere. Products were applied topically at a rate of 2 μL per 1 cm² explant (2 mg/cm²) and spread and gently rubbed for 10 seconds using a small spatula on day 0 (D0), D2, D5 and D7. The control explants did not receive any treatment except the renewal of culture medium. The culture medium was half renewed (1 ml per well) on D2, D5 and D7. After fixation for 24 hours in buffered formalin, the samples were dehydrated and impregnated in paraffin using a Leica PEARL dehydration automat. The samples were embedded using a Leica EG 1160 embedding station. 5-μm-thick sections were made using a Leica RM 2125 Minot-type microtome, and the sections were mounted on Superfrost® histological glass slides. The frozen samples were cut into 7-μm-thick sections using a Leica CM 3050 cryostat. Sections were then mounted on Superfrost® plus silanized glass slides. The microscopical observations were realized using a Leica DMLB or Olympus BX43 microscope. Pictures were digitized with a numeric DP72 Olympus camera with CellSens storing software. Melanin was visualized after silver impregnation according to Masson's Fontana staining method on FFPE skin sections. The staining was assessed by microscopical observation (FIG. 5) and by image analysis (FIG. 6).

The UV-induced increase of melanin vs untreated explants (UVA) validated the experiment as a positive control. 0.2% SLT-048 produced a slight increase of melanin and 2% SLT-048 produced a moderate increase of melanin.

UV irradiation significantly increased melanin content (+78%) as a positive control. 0.2% SLT-048 produced 10.0 (+24%) and 2% SLT-048 produced 12.6 (+56%) of surface occupied by melanin in the basal cell layer of the epidermis vs the vehicle E 7.9.

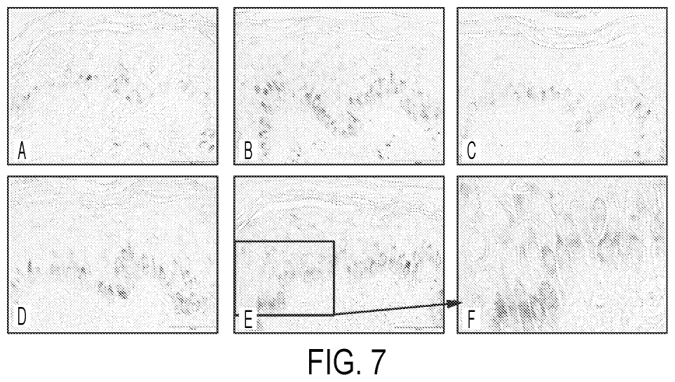

Melanin visualized after silver impregnation according to Masson's Fontana staining method on FFPE skin sections is illustrated in FIG. 7.

The melanin content was increased by 0.2% SLT-048 and highly increased by 2% SLT-048: keratinocyte nuclear capping by melanin was also detected. The melanin increase was high in the basal layers of the epidermis and to a lesser extent in the suprabasal layers.

Following topical application, SLT-048 significantly increased the epidermal melanin expression in an Ex Vivo skin explant model in a dose-dependent manner.

### Example 10: Assessment of SLT-045 on Pigmentation Using Living Human Skin Explants Ex Vivo

The aim of this study was to evaluate the effects of SLT-045 on pigmentation (pro-pigmentation effects) using living human skin explants in survival medium.

SLT-045 (3, 7, 15, 21 mM; 5 μL/cm2) in vehicle (44.9% DMSO/37.5% EtOH/17.4% propylene glycol) was applied topically applied using a Pasteur pipette, each day for 6 days using triplicate skin samples (8 mm punch).

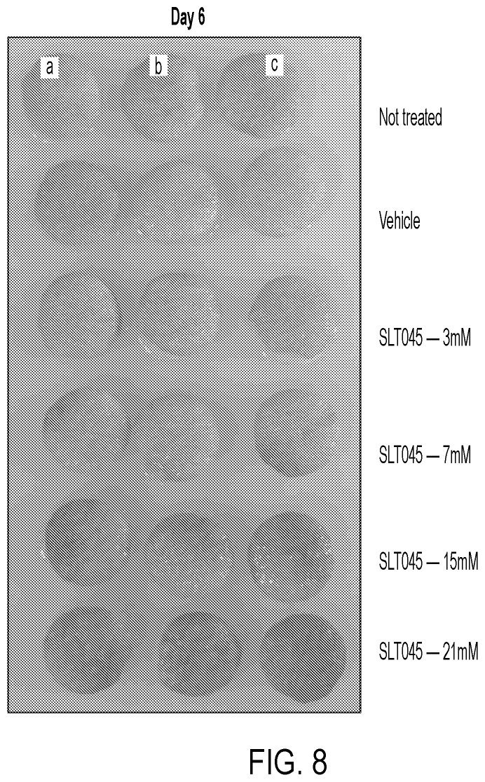

SLT-045 15 mM and 21 mM topically applied ex vivo for 6 days induced a visual skin pigmentation as illustrated in FIG. 8.

What is claimed:

1. A compound having a structure of Formula (I) or (II), 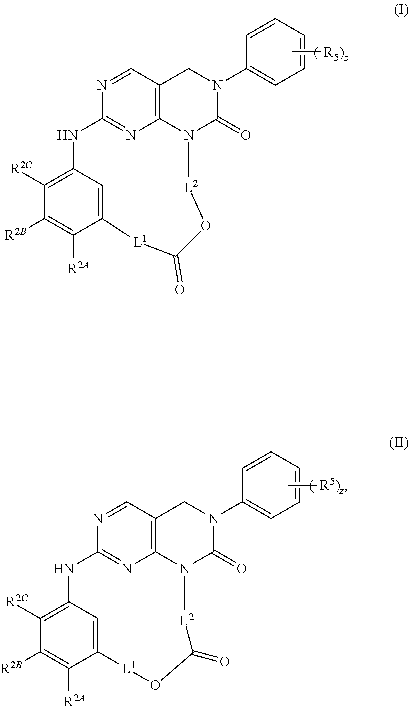
wherein:
each L¹ and L² is independently a bond, substituted or unsubstituted alkylene, or substituted or unsubstituted alkenylene;
each R^{2A}, R^{2B} and R^{2C} is independently hydrogen, halogen, —CX²₃, —CHX²₂, —CH₂X², —OCX², —OCH₂X², —OCHX²₂, —OR^{2F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is independently halogen, —CX⁵₃, —CHX⁵₂, —CH₂X⁵, —OCX⁵₃, —OCH₂X⁵, —OCHX⁵₂, —OR^{5F}, or substituted or unsubstituted C₁-C₄ alkyl;
z is an integer of 0 to 5;
each X² and X⁵ is independently —F, —Br, —Cl, or —I; and
each R^{2F} and R^{5F} is hydrogen, or substituted or unsubstituted alkyl.

2. The compound of claim 1, wherein z is 2.

3. The compound of any one of claims 1 to 2, wherein R⁵ is independently unsubstituted C₁-C₄ alkyl.

4. The compound of any one of claims 1 to 3, wherein the compound has the structure of Formula (I-a) or (II-a),

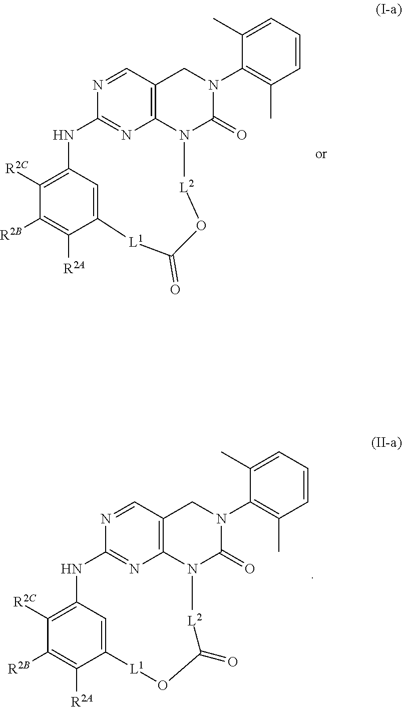

5. The compound of any one of claims 1 to 4, wherein R^{2A} is substituted or unsubstituted heterocycloalkyl, or substituted or unsubstituted aryl.

6. The compound of any one of claims 1 to 5, wherein R^{2A} is substituted or unsubstituted piperazinyl.

7. The compound of any one of claims 1 to 4, wherein the compound has the structure of Formula (I-b) or (II-b), 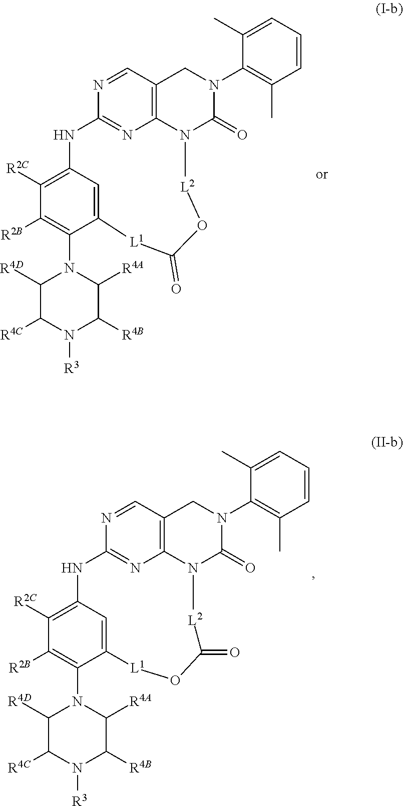
wherein:
R³ is hydrogen or substituted or unsubstituted alkyl;
Each R^{4A}, R^{4B}, R^{4C}, and R^{4D} is independently hydrogen, halogen, —CX⁴₃, —CHX⁴₂, —CH₂X⁴, —OCX⁴₃, —OCH₂X⁴, —OCHX⁴₂, —OR^{4F}, or substituted or unsubstituted alkyl;
X⁴ is independently —F, —Br, —Cl, or —I; and
R^{4F} is hydrogen, or substituted or unsubstituted alkyl.

8. The compound of claim 7, wherein the compound has the structure of formula (I-c), 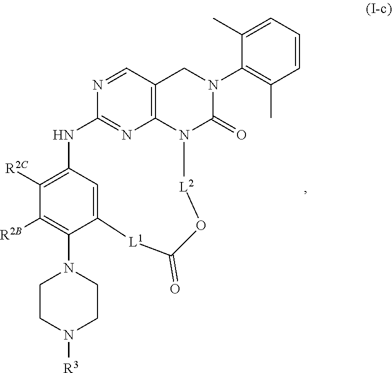
wherein:
L¹ is a bond, or R⁶-substituted or unsubstituted C₁-C₄ alkylene;
L² is a substituted or unsubstituted C₂-C₅ alkylene, or substituted or unsubstituted C₂-C₅ alkenylene; and
R⁶ is halogen, or unsubstituted C₁-C₄ alkylene.

9. The compound of claim 7, wherein the compound has the structure of formula (II-c), 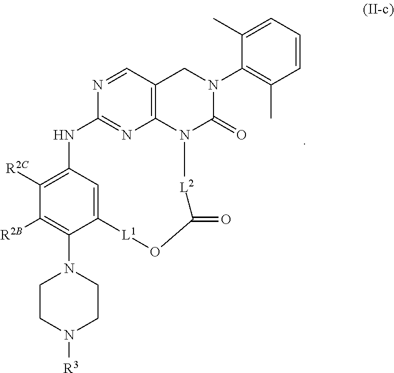
wherein:
L¹ is a R⁶-substituted or unsubstituted C₁-C₄ alkylene;
L² is a substituted or unsubstituted C₂-C₅ alkylene, or substituted or unsubstituted C₂-C₅ alkenylene; and
R⁶ is halogen, or unsubstituted C₁-C₄ alkylene.

10. The compound of any one of claims 1 to 4, wherein R^{2A} is halogen.

11. The compound of any one of claims 1 to 10, wherein each R^{2B} and R^{2C} is independently hydrogen, or —OR^{2F}, and R^{2F} is hydrogen or unsubstituted C₁-C₄ alkyl.

12. The compound of any one of claims 1 to 11, wherein R^{2′} is hydrogen and R^{2C} is —OCH₃.

13. The compound of any one of claims 1 to 12, wherein R³ is —CH₃.

14. The compound of claim 1, wherein the compound is

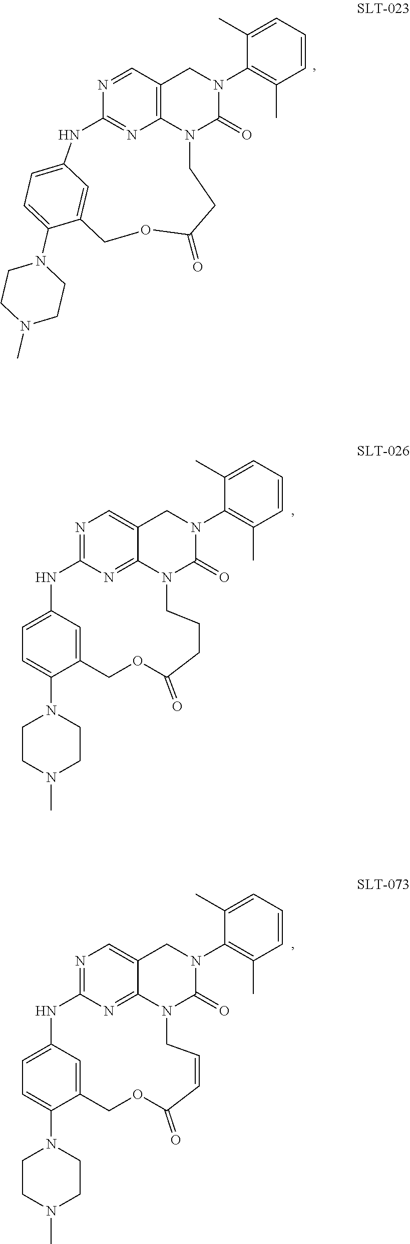 [SMILES: Cc1cccc(C)c1N1Cc2cnc3nc2N(CCC(=O)OCc2cc(ccc2N2CCN(C)CC2)N3)C1=O ; Cc1cccc(C)c1N1Cc2cnc3nc2N(CCCC(=O)OCc2cc(ccc2N2CCN(C)CC2)N3)C1=O ; Cc1cccc(C)c1N1Cc2cnc3nc2N(C/C=C\C(=O)OCc2cc(ccc2N2CCN(C)CC2)N3)C1=O]

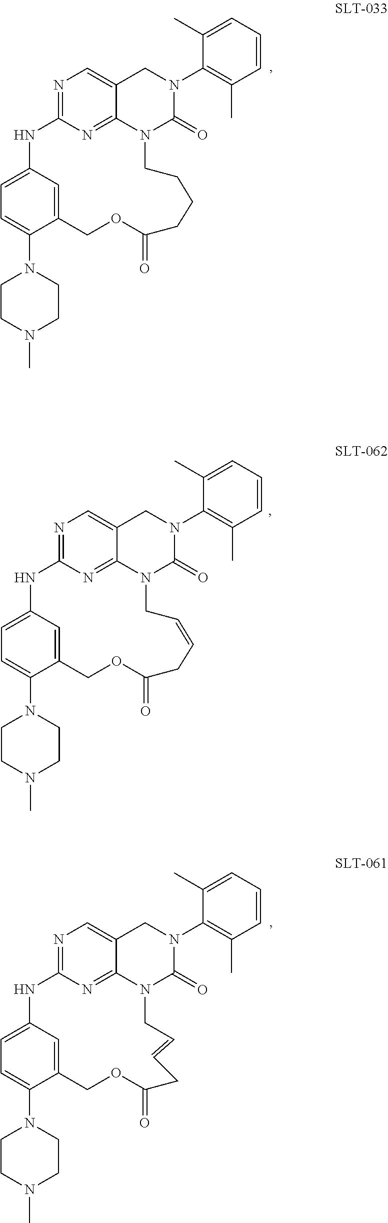 [SMILES: Cc1cccc(C)c1N1Cc2cnc3nc2N(CCCCC(=O)OCc2cc(ccc2N2CCN(C)CC2)N3)C1=O ; Cc1cccc(C)c1N1Cc2cnc3nc2N(C/C=C\CC(=O)OCc2cc(ccc2N2CCN(C)CC2)N3)C1=O ; Cc1cccc(C)c1N1Cc2cnc3nc2N(C/C=C/CC(=O)OCc2cc(ccc2N2CCN(C)CC2)N3)C1=O]

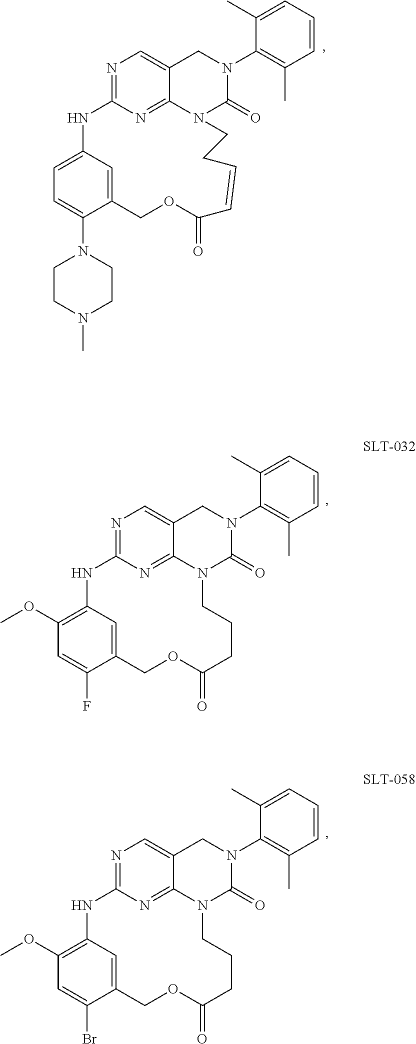 [SMILES: Cc1cccc(C)c1N1Cc2cnc3nc2N(CC/C=C\C(=O)OCc2cc(ccc2N2CCN(C)CC2)N3)C1=O ; COc1cc(F)c2cc1Nc1ncc3c(n1)N(CCCC(=O)OC2)C(=O)N(c1c(C)cccc1C)C3 ; COc1cc(Br)c2cc1Nc1ncc3c(n1)N(CCCC(=O)OC2)C(=O)N(c1c(C)cccc1C)C3]

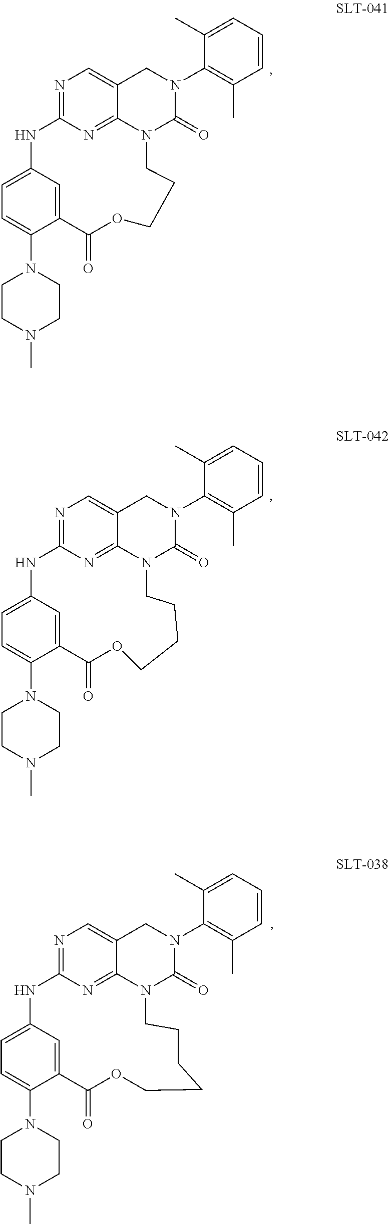 [SMILES: Cc1cccc(C)c1N1Cc2cnc3nc2N(CCCOC(=O)c2cc(ccc2N2CCN(C)CC2)N3)C1=O ; Cc1cccc(C)c1N1Cc2cnc3nc2N(CCCCOC(=O)c2cc(ccc2N2CCN(C)CC2)N3)C1=O ; Cc1cccc(C)c1N1Cc2cnc3nc2N(CCCCCOC(=O)c2cc(ccc2N2CCN(C)CC2)N3)C1=O]

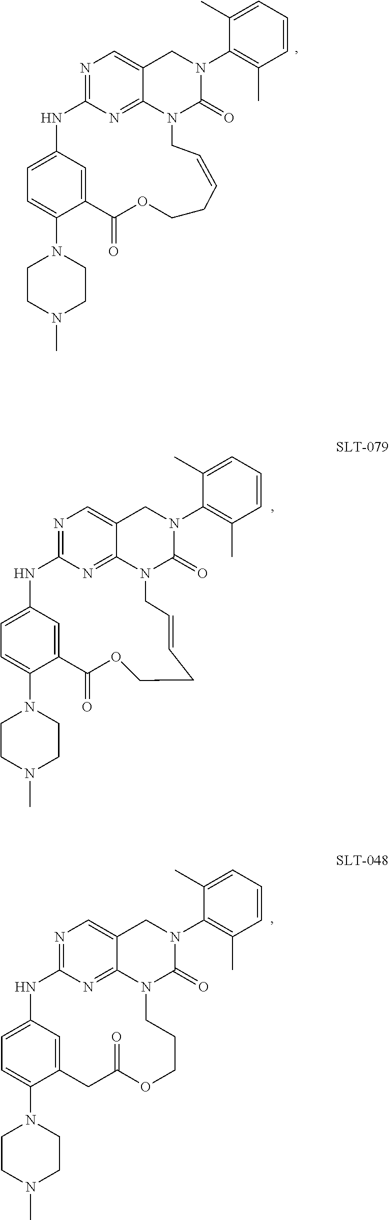 [SMILES: Cc1cccc(C)c1N1Cc2cnc3nc2N(C/C=C\CCOC(=O)c2cc(ccc2N2CCN(C)CC2)N3)C1=O ; Cc1cccc(C)c1N1Cc2cnc3nc2N(C/C=C/CCOC(=O)c2cc(ccc2N2CCN(C)CC2)N3)C1=O ; Cc1cccc(C)c1N1Cc2cnc3nc2N(CCCOC(=O)Cc2cc(ccc2N2CCN(C)CC2)N3)C1=O]

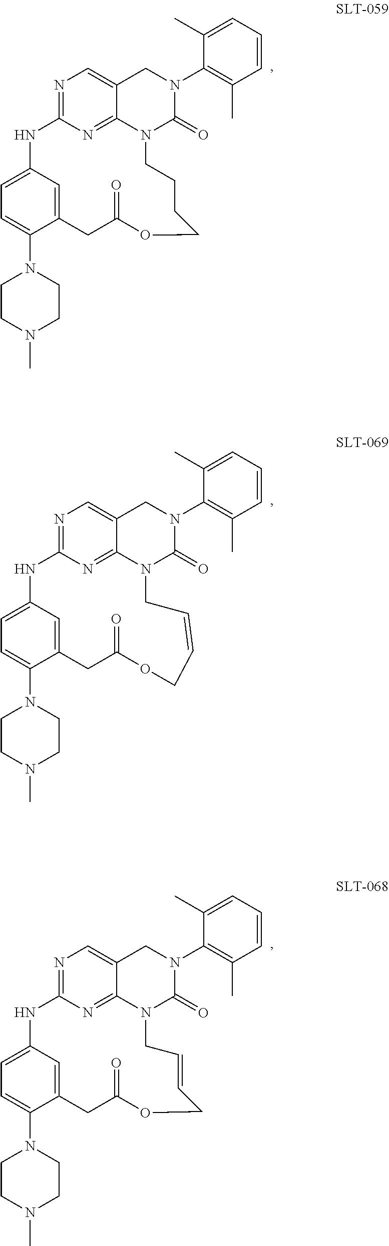 [SMILES: Cc1cccc(C)c1N1Cc2cnc3nc2N(CCCCOC(=O)Cc2cc(ccc2N2CCN(C)CC2)N3)C1=O ; Cc1cccc(C)c1N1Cc2cnc3nc2N(C/C=C\COC(=O)Cc2cc(ccc2N2CCN(C)CC2)N3)C1=O ; Cc1cccc(C)c1N1Cc2cnc3nc2N(C/C=C/COC(=O)Cc2cc(ccc2N2CCN(C)CC2)N3)C1=O]

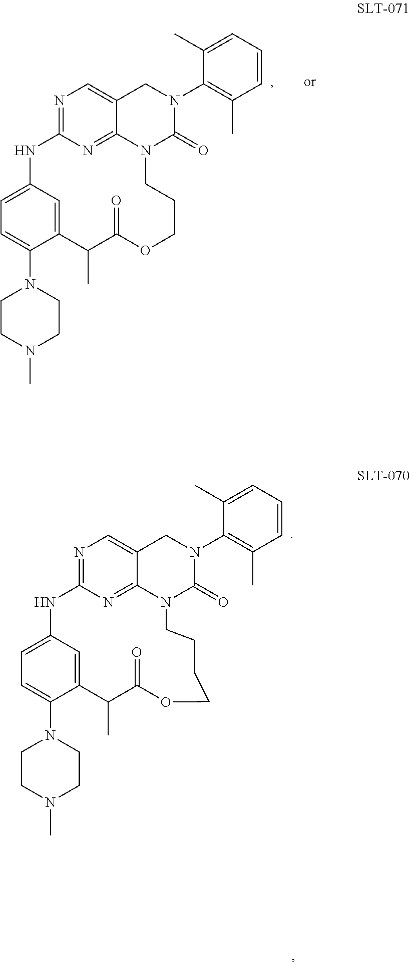 [SMILES: Cc1cccc(C)c1N1Cc2cnc3nc2N(CCCOC(=O)C(C)c2cc(ccc2N2CCN(C)CC2)N3)C1=O ; Cc1cccc(C)c1N1Cc2cnc3nc2N(CCCCOC(=O)C(C)c2cc(ccc2N2CCN(C)CC2)N3)C1=O]

15. A compound having a structure of Formula (III) or (IV), 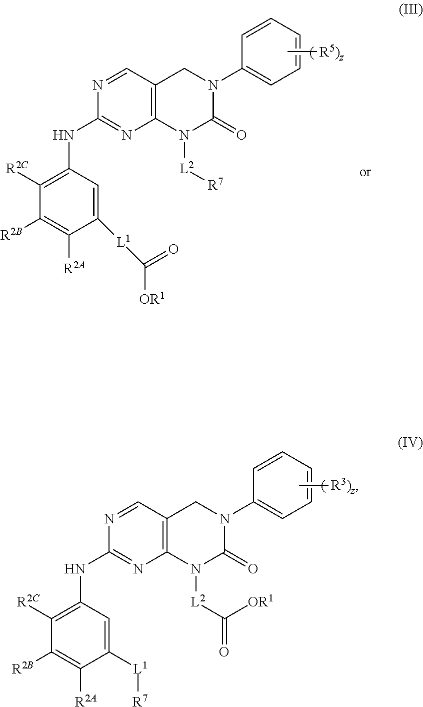
wherein:
R¹ is independently a hydrogen, substituted or unsubstituted alkyl, or substituted or unsubstituted alkenyl;
each L¹ and L² is independently a bond, substituted or unsubstituted alkylene, or substituted or unsubstituted alkenylene:
each R^{2A}, R^{2B} and R^{2C} is independently hydrogen, halogen, —CX²₃, —CHX²₂, —CH₂X², —OCX²₃, —OCH₂X², —OCHX²₂, —OR^{2F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is independently halogen, —CX⁵₃, —CHX⁵₂, —CH₂X⁵, —OCX⁵₃, —OCH₂X⁵, —OCHX⁵₂, —OR^{5F}, or substituted or unsubstituted C₁-C₄ alkyl;
z is an integer of 0 to 5;
R⁷ is —OR^{7F}, or substituted or unsubstituted alkenyl;
each X² and X⁵ is independently —F, —Br, —Cl, or —I; and
each R^{2F}, R^{5F} and OR^{7F} is independently hydrogen, or substituted or unsubstituted alkyl.

16. The compound of claim 15, wherein R¹ is hydrogen, unsubstituted C₁-C₄ alkyl, or unsubstituted C₁-C₄ alkenyl.

17. The compound of any one of claims 15 to 16, wherein z is 2.

18. The compound of any one of claims 15 to 17, wherein each R⁵ is unsubstituted C₁-C₄ alkyl.

19. The compound of any one of claims 15 to 18, wherein the compound has the structure of Formula (III-a) or (IV-a),

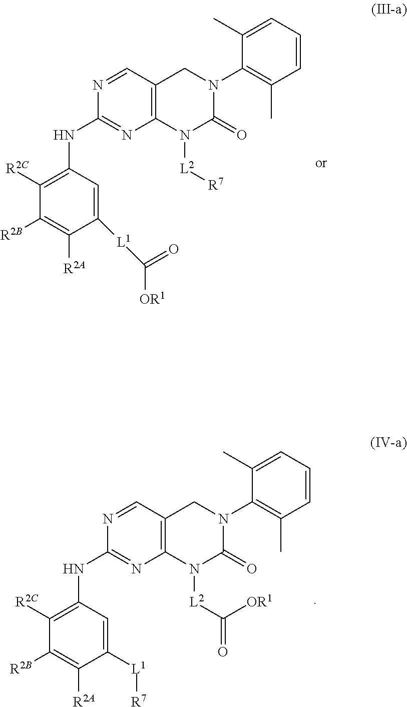

20. The compound of any one of claims 15 to 19, wherein R^{2A} is substituted or unsubstituted heterocycloalkyl, or substituted or unsubstituted aryl.

21. The compound of any one of claims 15 to 20, wherein R^{2A} is substituted or unsubstituted piperazinyl.

22. The compound of any one of claims 15 to 21, wherein the compound has the structure of Formula (III-b) or (IV-b), 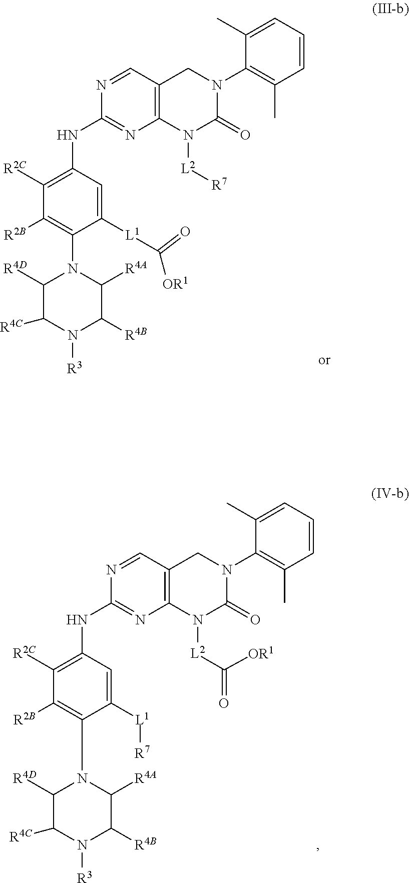
wherein:
R³ is hydrogen or substituted or unsubstituted alkyl;
Each R^{4A}, R^{4B}, R^{4C}, and R^{4D} is independently hydrogen, halogen, —CX⁴₃, —CHX⁴₂, —CH₂X⁴, —OCX⁴₃, —OCH₂X⁴, —OCHX⁴₂, —OR^{4F}, or substituted or unsubstituted alkyl;
R⁷ is —OH, or unsubstituted C₁-C₄ alkenyl;
X⁴ is independently —F, —Br, —Cl, or —I; and
R^{4F} is hydrogen, or substituted or unsubstituted alkyl.

23. The compound of claim 22, wherein the compound has the structure of formula (III-c), 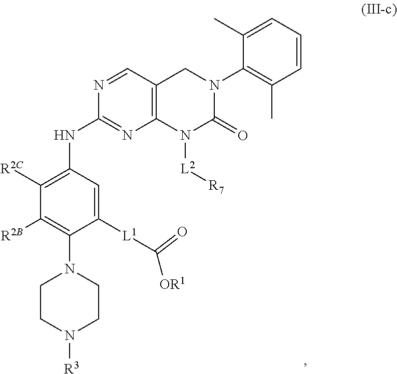
wherein:
L¹ is a bond, or R⁶-substituted or unsubstituted C₁-C₄ alkylene;
L² is a substituted or unsubstituted C₂-C₅ alkylene, or substituted or unsubstituted C₂-C₅ alkenylene; and
R⁶ is halogen, or unsubstituted C₁-C₄ alkylene.

24. The compound of claim 22, wherein the compound has the structure of formula (IV-c), 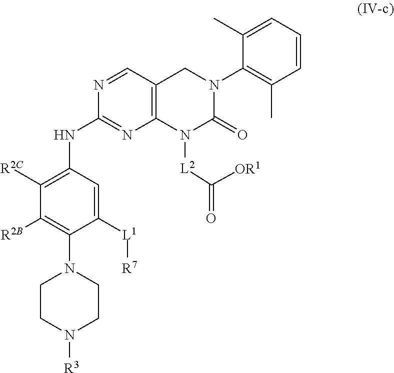
wherein:
L¹ is a R⁶-substituted or unsubstituted C₁-C₄ alkylene;
L² is a substituted or unsubstituted C₂-C₅ alkylene, or substituted or unsubstituted C₂-C₅ alkenylene; and
R⁶ is halogen, or unsubstituted C₁-C₄ alkylene.

25. The compound of any one of claims 15 to 24, R⁷ is —OH, or unsubstituted C₁-C₄ alkenyl.

26. The compound of any one of claims 15 to 19, wherein R^{2A} is halogen.

27. The compound of any one of claims 15 to 26, wherein each R^{2B} and R^{2C} is independently hydrogen, or —OR^{2F}, and R^{2F} is hydrogen or unsubstituted C₁-C₄ alkyl.

28. The compound of any one of claims 15 to 27, wherein R^{2B} is hydrogen and R^{2C} is —OCH₃.

29. The compound of any one of claims 15 to 28, wherein R³ is —CH₃.

30. The compound of claim 15, wherein the compound is

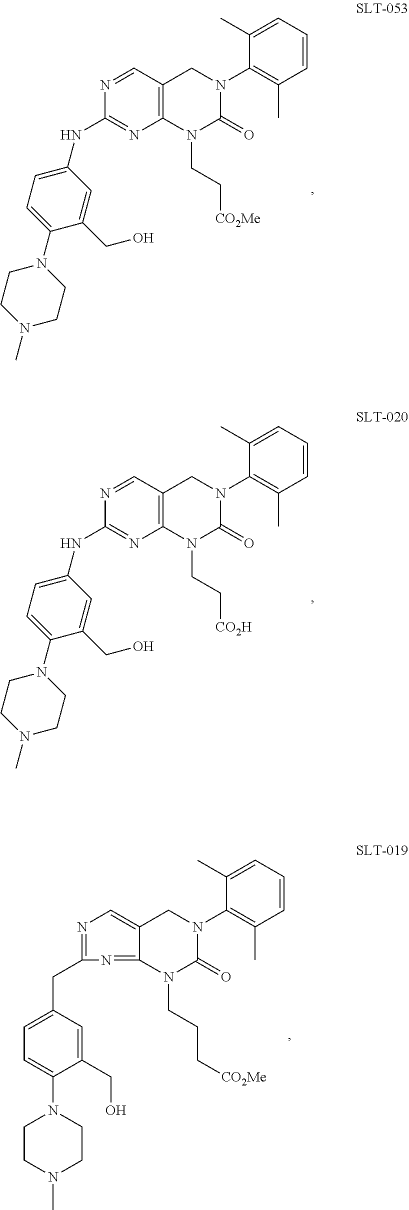 [SMILES: COC(=O)CCN1C(=O)N(c2c(C)cccc2C)Cc2cnc(Nc3ccc(N4CCN(C)CC4)c(CO)c3)nc21 ; Cc1cccc(C)c1N1Cc2cnc(Nc3ccc(N4CCN(C)CC4)c(CO)c3)nc2N(CCC(=O)O)C1=O ; COC(=O)CCCN1C(=O)N(c2c(C)cccc2C)Cc2cnc(Cc3ccc(N4CCN(C)CC4)c(CO)c3)nc21]

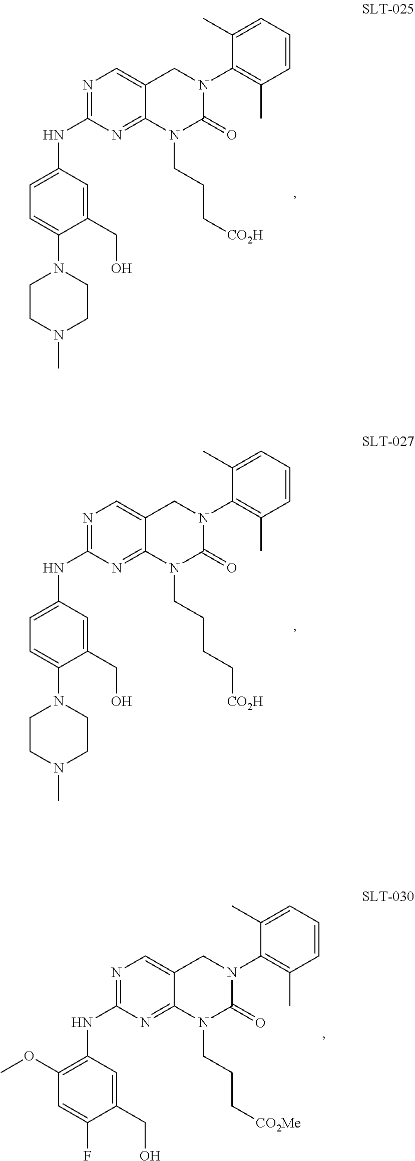 [SMILES: Cc1cccc(C)c1N1Cc2cnc(Nc3ccc(N4CCN(C)CC4)c(CO)c3)nc2N(CCCC(=O)O)C1=O ; Cc1cccc(C)c1N1Cc2cnc(Nc3ccc(N4CCN(C)CC4)c(CO)c3)nc2N(CCCCC(=O)O)C1=O ; COC(=O)CCCN1C(=O)N(c2c(C)cccc2C)Cc2cnc(Nc3cc(CO)c(F)cc3OC)nc21]

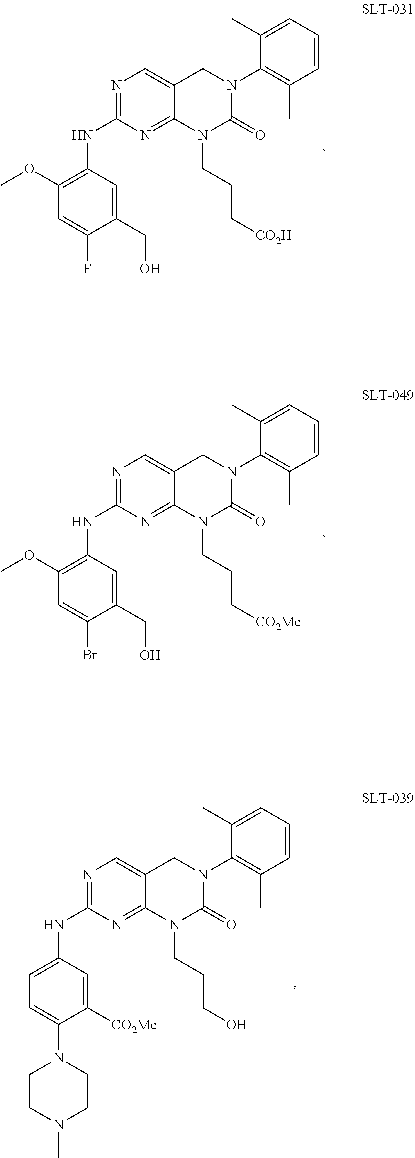 [SMILES: COc1cc(F)c(CO)cc1Nc1ncc2c(n1)N(CCCC(=O)O)C(=O)N(c1c(C)cccc1C)C2 ; COC(=O)CCCN1C(=O)N(c2c(C)cccc2C)Cc2cnc(Nc3cc(CO)c(Br)cc3OC)nc21 ; COC(=O)c1cc(Nc2ncc3c(n2)N(CCCO)C(=O)N(c2c(C)cccc2C)C3)ccc1N1CCN(C)CC1]

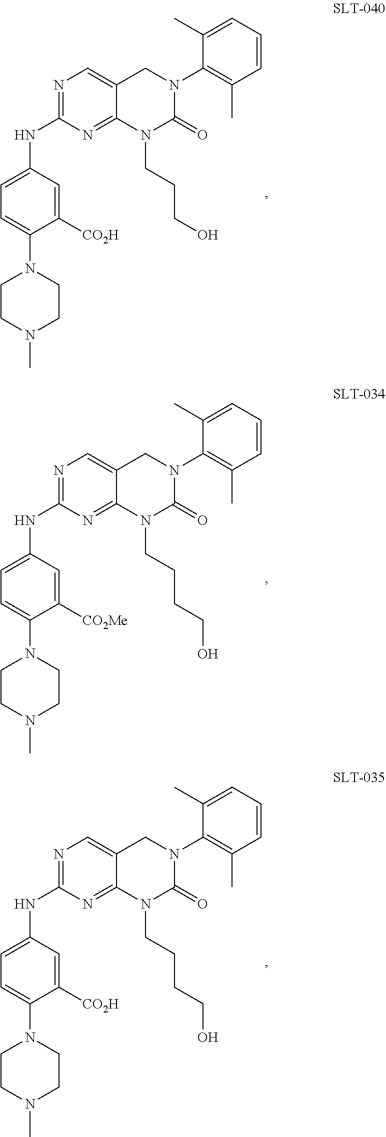 [SMILES: Cc1cccc(C)c1N1Cc2cnc(Nc3ccc(N4CCN(C)CC4)c(C(=O)O)c3)nc2N(CCCO)C1=O ; COC(=O)c1cc(Nc2ncc3c(n2)N(CCCCO)C(=O)N(c2c(C)cccc2C)C3)ccc1N1CCN(C)CC1 ; Cc1cccc(C)c1N1Cc2cnc(Nc3ccc(N4CCN(C)CC4)c(C(=O)O)c3)nc2N(CCCCO)C1=O]

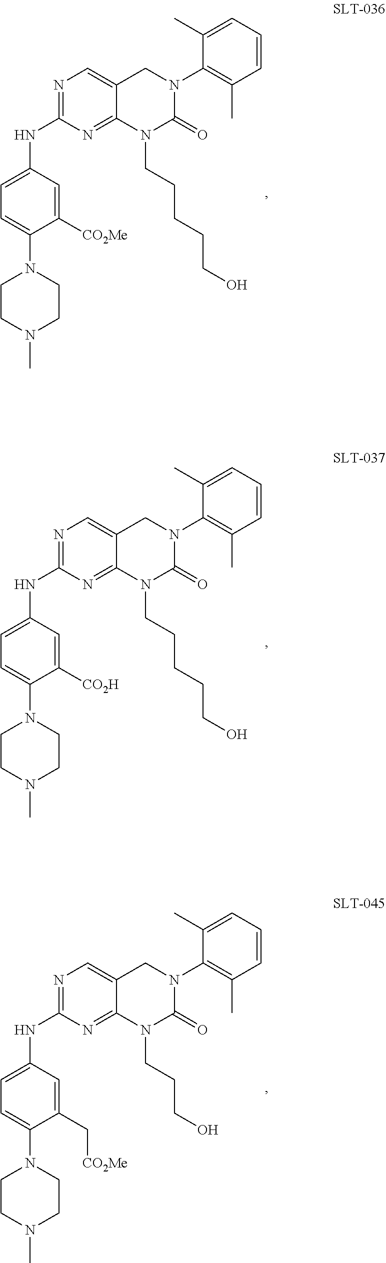 [SMILES: COC(=O)c1cc(Nc2ncc3c(n2)N(CCCCCO)C(=O)N(c2c(C)cccc2C)C3)ccc1N1CCN(C)CC1 ; Cc1cccc(C)c1N1Cc2cnc(Nc3ccc(N4CCN(C)CC4)c(C(=O)O)c3)nc2N(CCCCCO)C1=O ; COC(=O)Cc1cc(Nc2ncc3c(n2)N(CCCO)C(=O)N(c2c(C)cccc2C)C3)ccc1N1CCN(C)CC1]

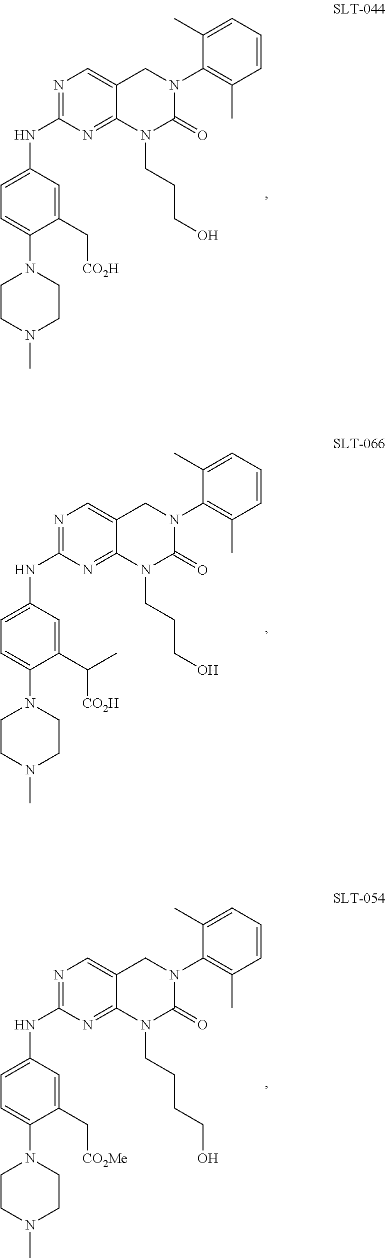 [SMILES: Cc1cccc(C)c1N1Cc2cnc(Nc3ccc(N4CCN(C)CC4)c(CC(=O)O)c3)nc2N(CCCO)C1=O ; Cc1cccc(C)c1N1Cc2cnc(Nc3ccc(N4CCN(C)CC4)c(C(C)C(=O)O)c3)nc2N(CCCO)C1=O ; COC(=O)Cc1cc(Nc2ncc3c(n2)N(CCCCO)C(=O)N(c2c(C)cccc2C)C3)ccc1N1CCN(C)CC1]

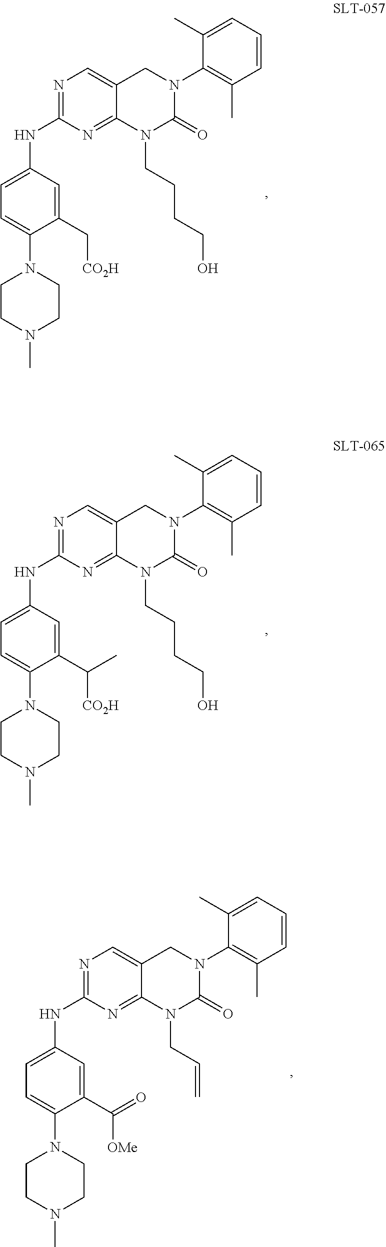 [SMILES: Cc1cccc(C)c1N1Cc2cnc(Nc3ccc(N4CCN(C)CC4)c(CC(=O)O)c3)nc2N(CCCCO)C1=O ; Cc1cccc(C)c1N1Cc2cnc(Nc3ccc(N4CCN(C)CC4)c(C(C)C(=O)O)c3)nc2N(CCCCO)C1=O ; C=CCN1C(=O)N(c2c(C)cccc2C)Cc2cnc(Nc3ccc(N4CCN(C)CC4)c(C(=O)OC)c3)nc21]

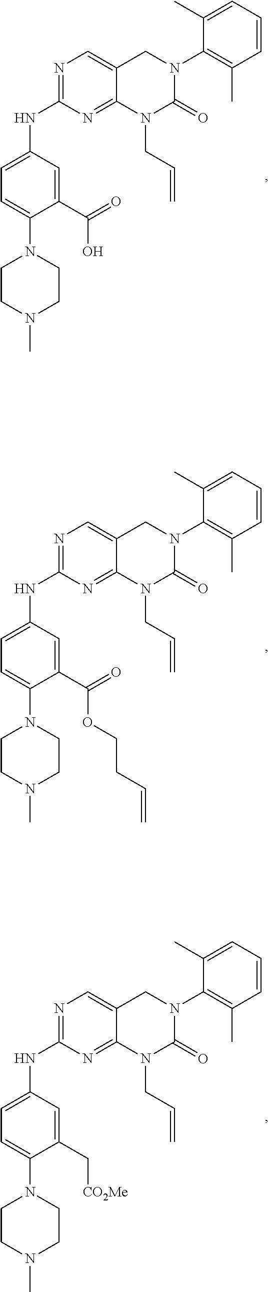 [SMILES: C=CCN1C(=O)N(c2c(C)cccc2C)Cc2cnc(Nc3ccc(N4CCN(C)CC4)c(C(=O)O)c3)nc21 ; C=CCCOC(=O)c1cc(Nc2ncc3c(n2)N(CC=C)C(=O)N(c2c(C)cccc2C)C3)ccc1N1CCN(C)CC1 ; C=CCN1C(=O)N(c2c(C)cccc2C)Cc2cnc(Nc3ccc(N4CCN(C)CC4)c(CC(=O)OC)c3)nc21]

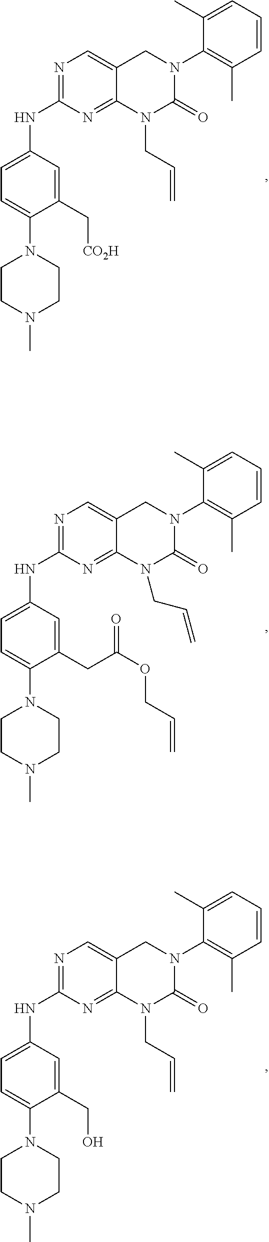 [SMILES: C=CCN1C(=O)N(c2c(C)cccc2C)Cc2cnc(Nc3ccc(N4CCN(C)CC4)c(CC(=O)O)c3)nc21 ; C=CCOC(=O)Cc1cc(Nc2ncc3c(n2)N(CC=C)C(=O)N(c2c(C)cccc2C)C3)ccc1N1CCN(C)CC1 ; C=CCN1C(=O)N(c2c(C)cccc2C)Cc2cnc(Nc3ccc(N4CCN(C)CC4)c(CO)c3)nc21]

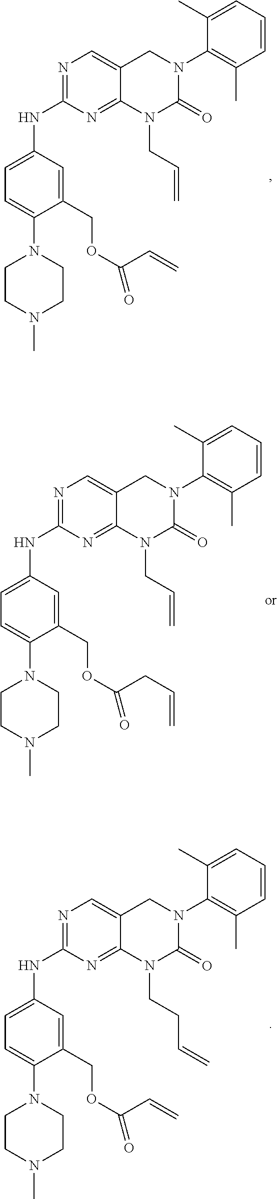 [SMILES: C=CCN1C(=O)N(c2c(C)cccc2C)Cc2cnc(Nc3ccc(N4CCN(C)CC4)c(COC(=O)C=C)c3)nc21 ; C=CCC(=O)OCc1cc(Nc2ncc3c(n2)N(CC=C)C(=O)N(c2c(C)cccc2C)C3)ccc1N1CCN(C)CC1 ; C=CCCN1C(=O)N(c2c(C)cccc2C)Cc2cnc(Nc3ccc(N4CCN(C)CC4)c(COC(=O)C=C)c3)nc21]

31. A method of producing a compound having the structure of formula (I) or (II), 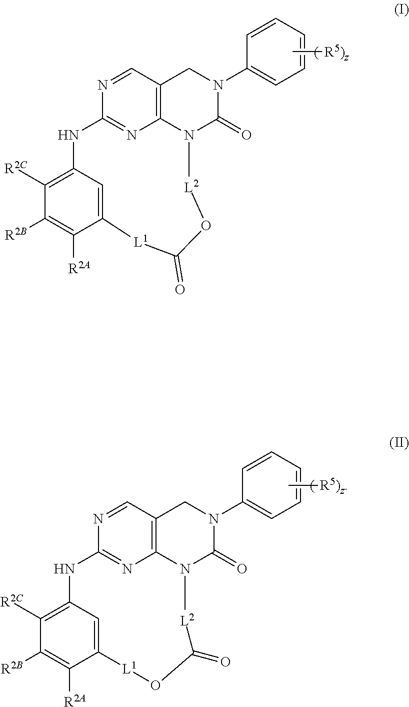
the method comprising
providing a compound having the structure of formula (III) or (IV), 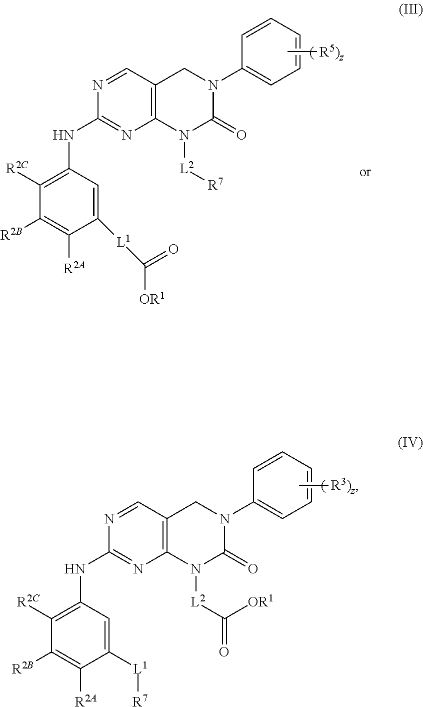
wherein, in Formulae (I) to (IV):
R¹ is independently a hydrogen, substituted or unsubstituted alkyl, or substituted or unsubstituted alkenyl;
each L¹ and L² is independently a bond, substituted or unsubstituted alkylene, or substituted or unsubstituted alkenylene;
each R^{2A}, R^{2B} and R^{2C} is independently hydrogen, halogen, —CX², —CHX²₂, —CH₂X², —OCX²₃, —OCH₂X², —OCHX²₂, —OR^{2F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is independently halogen, —CX⁵₃, —CHX⁵₂, —CH₂X⁵, —OCX⁵₃, —OCH₂X⁵, —OCHX⁵₂, —OR^{5F}, or substituted or unsubstituted C₁-C₄ alkyl;
z is an integer of 0 to 5;
R⁷ is —OR^{7F}, or substituted or unsubstituted alkenyl;
each X² and X⁵ is independently —F, —Br, —Cl, or —I; and
each R^{2F}, R^{5F} and R^{7F} is independently hydrogen, or substituted or unsubstituted alkyl.

32. The method of claim 31, wherein z is 2.

33. The method of any one of claims 31 to 32, wherein each R⁵ is unsubstituted C₁-C₄ alkyl.

34. The method of any one of claims 31 to 33, wherein each R¹ is hydrogen or unsubstituted C₁-C₄ alkyl.

35. The method of any one of claims 31 to 34, R⁷ is independently —OH, or unsubstituted C₁-C₄ alkenyl.

36. The method of any one of claims 31 to 35, wherein R^{2A} is substituted or unsubstituted heterocycloalkyl, or substituted or unsubstituted aryl.

37. The method of any one of claims 31 to 36, wherein R^{2A} is substituted or unsubstituted piperazinyl.

38. The method of any one of claims 31 to 34, wherein R^{2A} is halogen.

39. The method of any one of claims 31 to 38, wherein each R^{2B} and R^{2C} is independently hydrogen, or —OR^{2F}, and R^{2F} is hydrogen or unsubstituted C₁-C₄ alkyl.

40. The method of any one of claim 31 to claim 39, wherein R^{2B} is hydrogen and R^{2C} is —OCH₃.

41. The method of claim 31, wherein the compound of Formula (III) or (IV) is

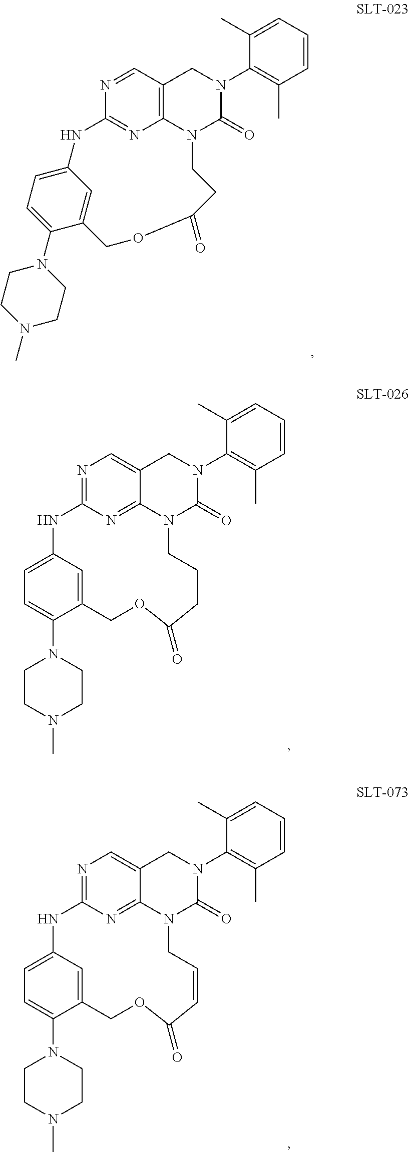 [SMILES: Cc1cccc(C)c1N1Cc2cnc3nc2N(CCC(=O)OCc2cc(ccc2N2CCN(C)CC2)N3)C1=O ; Cc1cccc(C)c1N1Cc2cnc3nc2N(CCCC(=O)OCc2cc(ccc2N2CCN(C)CC2)N3)C1=O ; Cc1cccc(C)c1N1Cc2cnc3nc2N(C/C=C\C(=O)OCc2cc(ccc2N2CCN(C)CC2)N3)C1=O]

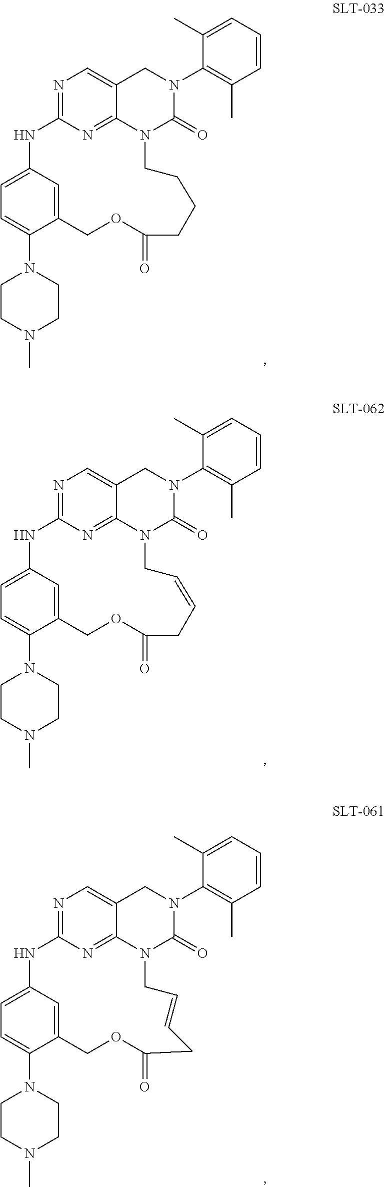 [SMILES: Cc1cccc(C)c1N1Cc2cnc3nc2N(CCCCC(=O)OCc2cc(ccc2N2CCN(C)CC2)N3)C1=O ; Cc1cccc(C)c1N1Cc2cnc3nc2N(C/C=C\CC(=O)OCc2cc(ccc2N2CCN(C)CC2)N3)C1=O ; Cc1cccc(C)c1N1Cc2cnc3nc2N(C/C=C/CC(=O)OCc2cc(ccc2N2CCN(C)CC2)N3)C1=O]

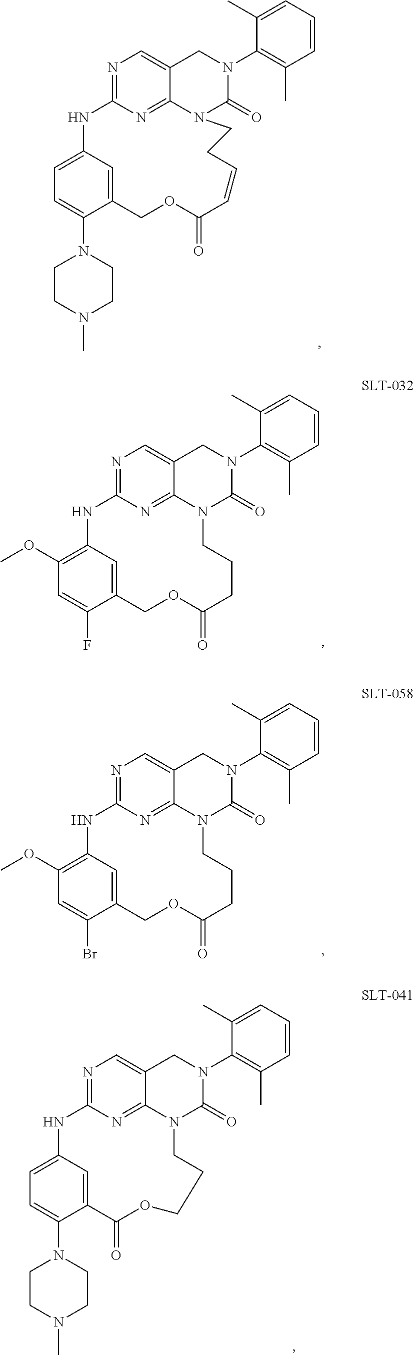 [SMILES: Cc1cccc(C)c1N1Cc2cnc3nc2N(CC/C=C\C(=O)OCc2cc(ccc2N2CCN(C)CC2)N3)C1=O ; COc1cc(F)c2cc1Nc1ncc3c(n1)N(CCCC(=O)OC2)C(=O)N(c1c(C)cccc1C)C3 ; COc1cc(Br)c2cc1Nc1ncc3c(n1)N(CCCC(=O)OC2)C(=O)N(c1c(C)cccc1C)C3 ; Cc1cccc(C)c1N1Cc2cnc3nc2N(CCCOC(=O)c2cc(ccc2N2CCN(C)CC2)N3)C1=O]

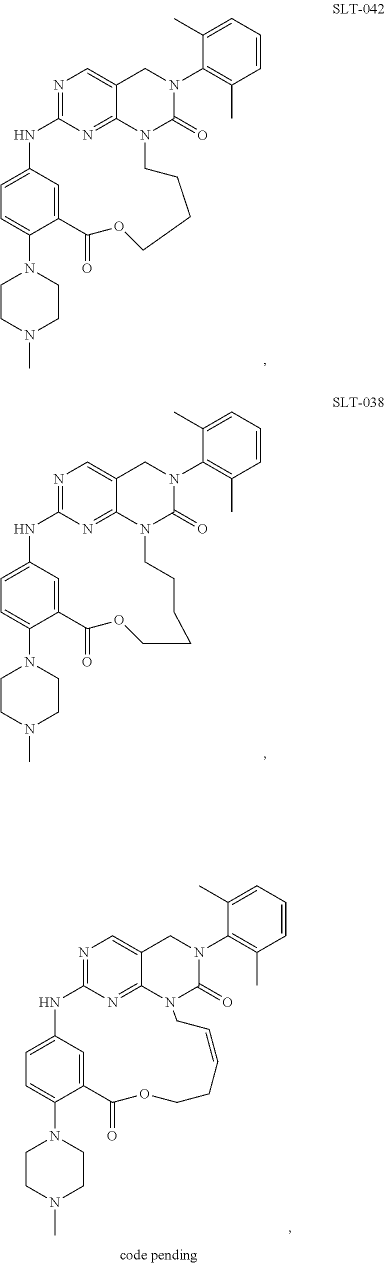 [SMILES: Cc1cccc(C)c1N1Cc2cnc3nc2N(CCCCOC(=O)c2cc(ccc2N2CCN(C)CC2)N3)C1=O ; Cc1cccc(C)c1N1Cc2cnc3nc2N(CCCCCOC(=O)c2cc(ccc2N2CCN(C)CC2)N3)C1=O ; Cc1cccc(C)c1N1Cc2cnc3nc2N(C/C=C\CCOC(=O)c2cc(ccc2N2CCN(C)CC2)N3)C1=O]

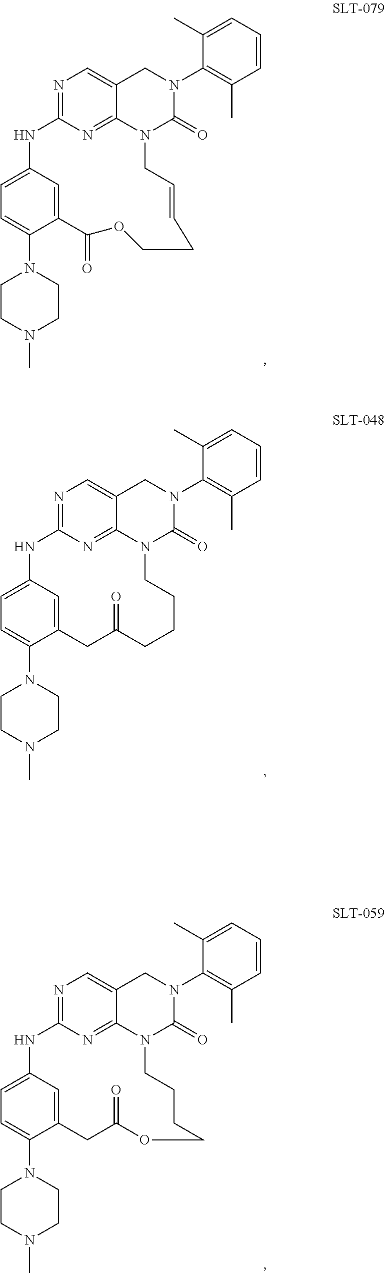 [SMILES: Cc1cccc(C)c1N1Cc2cnc3nc2N(C/C=C/CCOC(=O)c2cc(ccc2N2CCN(C)CC2)N3)C1=O ; Cc1cccc(C)c1N1Cc2cnc3nc2N(CCCCC(=O)Cc2cc(ccc2N2CCN(C)CC2)N3)C1=O ; Cc1cccc(C)c1N1Cc2cnc3nc2N(CCCCOC(=O)Cc2cc(ccc2N2CCN(C)CC2)N3)C1=O]

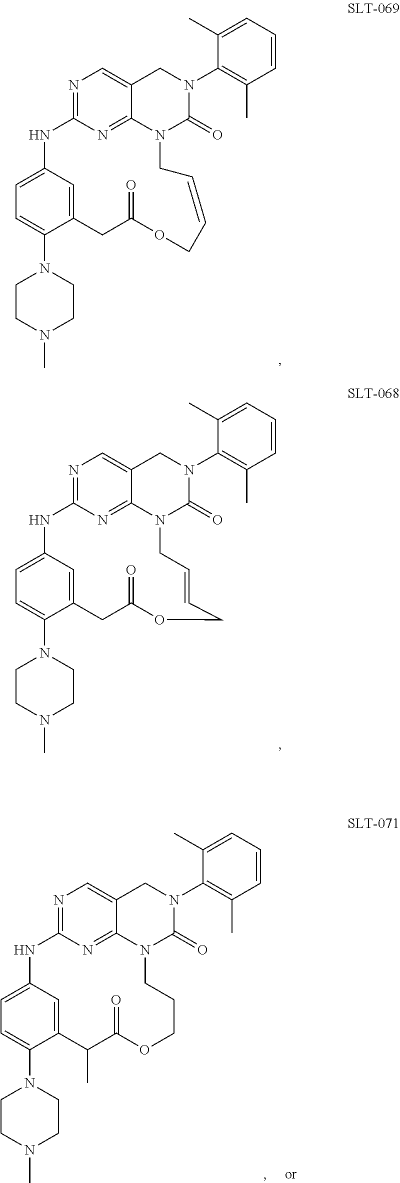 [SMILES: Cc1cccc(C)c1N1Cc2cnc3nc2N(C/C=C\COC(=O)Cc2cc(ccc2N2CCN(C)CC2)N3)C1=O ; Cc1cccc(C)c1N1Cc2cnc3nc2N(C/C=C/COC(=O)Cc2cc(ccc2N2CCN(C)CC2)N3)C1=O ; Cc1cccc(C)c1N1Cc2cnc3nc2N(CCCOC(=O)C(C)c2cc(ccc2N2CCN(C)CC2)N3)C1=O]

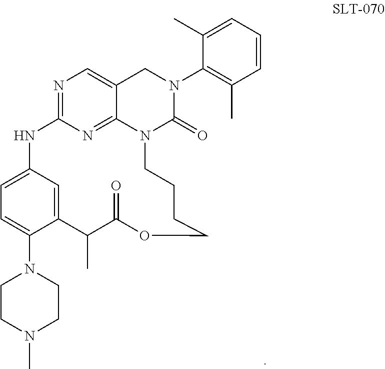 [SMILES: Cc1cccc(C)c1N1Cc2cnc3nc2N(CCCCOC(=O)C(C)c2cc(ccc2N2CCN(C)CC2)N3)C1=O]

42. The method of claim 31, wherein the compound of Formula (III) or (IV) is

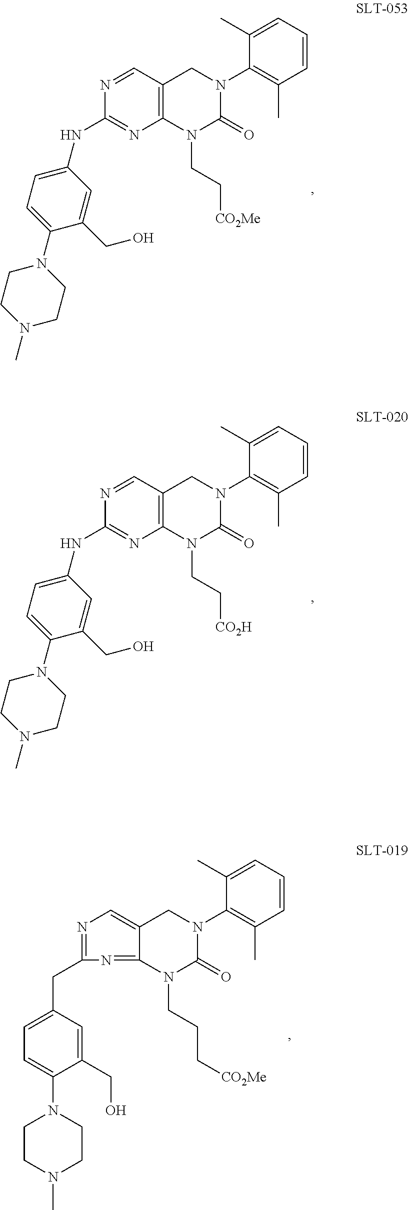 [SMILES: COC(=O)CCN1C(=O)N(c2c(C)cccc2C)Cc2cnc(Nc3ccc(N4CCN(C)CC4)c(CO)c3)nc21 ; Cc1cccc(C)c1N1Cc2cnc(Nc3ccc(N4CCN(C)CC4)c(CO)c3)nc2N(CCC(=O)O)C1=O ; COC(=O)CCCN1C(=O)N(c2c(C)cccc2C)Cc2cnc(Cc3ccc(N4CCN(C)CC4)c(CO)c3)nc21]

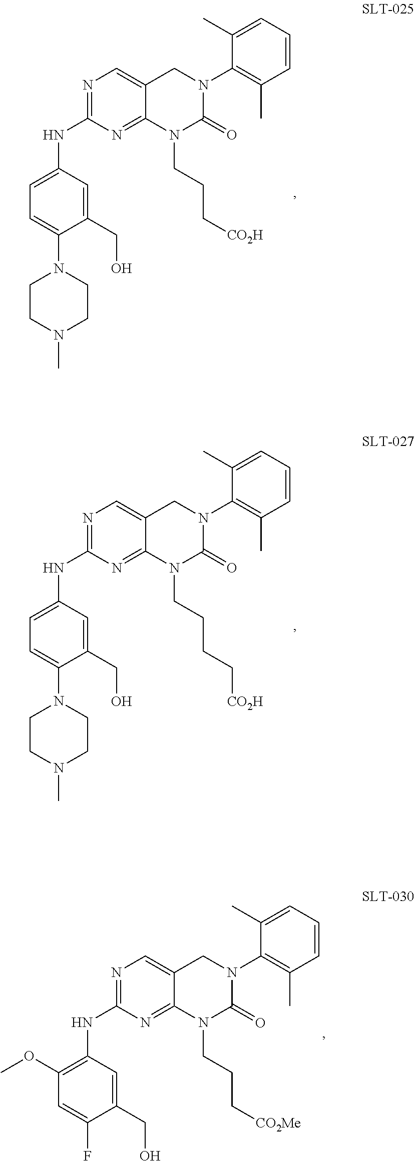 [SMILES: Cc1cccc(C)c1N1Cc2cnc(Nc3ccc(N4CCN(C)CC4)c(CO)c3)nc2N(CCCC(=O)O)C1=O ; Cc1cccc(C)c1N1Cc2cnc(Nc3ccc(N4CCN(C)CC4)c(CO)c3)nc2N(CCCCC(=O)O)C1=O ; COC(=O)CCCN1C(=O)N(c2c(C)cccc2C)Cc2cnc(Nc3cc(CO)c(F)cc3OC)nc21]

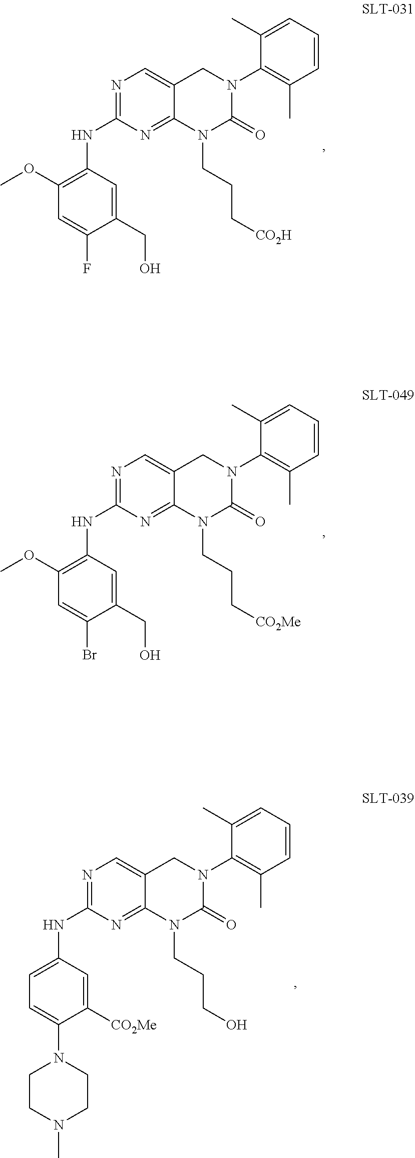 [SMILES: COc1cc(F)c(CO)cc1Nc1ncc2c(n1)N(CCCC(=O)O)C(=O)N(c1c(C)cccc1C)C2 ; COC(=O)CCCN1C(=O)N(c2c(C)cccc2C)Cc2cnc(Nc3cc(CO)c(Br)cc3OC)nc21 ; COC(=O)c1cc(Nc2ncc3c(n2)N(CCCO)C(=O)N(c2c(C)cccc2C)C3)ccc1N1CCN(C)CC1]

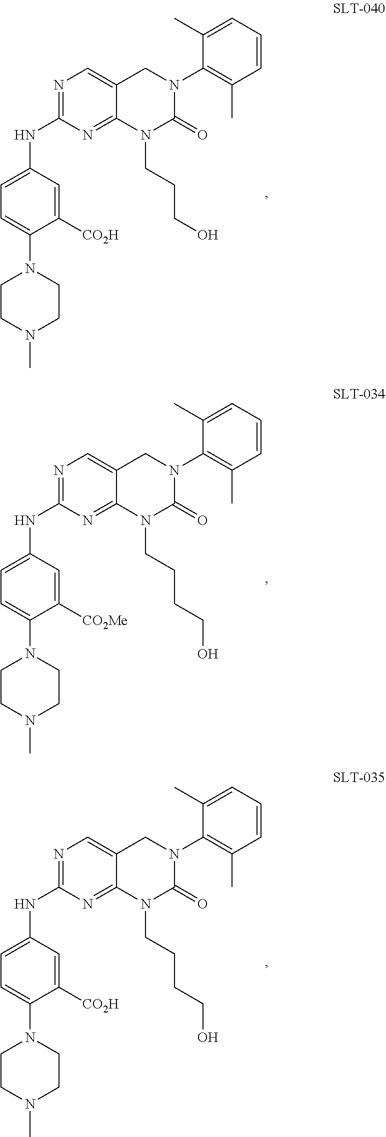 [SMILES: Cc1cccc(C)c1N1Cc2cnc(Nc3ccc(N4CCN(C)CC4)c(C(=O)O)c3)nc2N(CCCO)C1=O ; COC(=O)c1cc(Nc2ncc3c(n2)N(CCCCO)C(=O)N(c2c(C)cccc2C)C3)ccc1N1CCN(C)CC1 ; Cc1cccc(C)c1N1Cc2cnc(Nc3ccc(N4CCN(C)CC4)c(C(=O)O)c3)nc2N(CCCCO)C1=O]

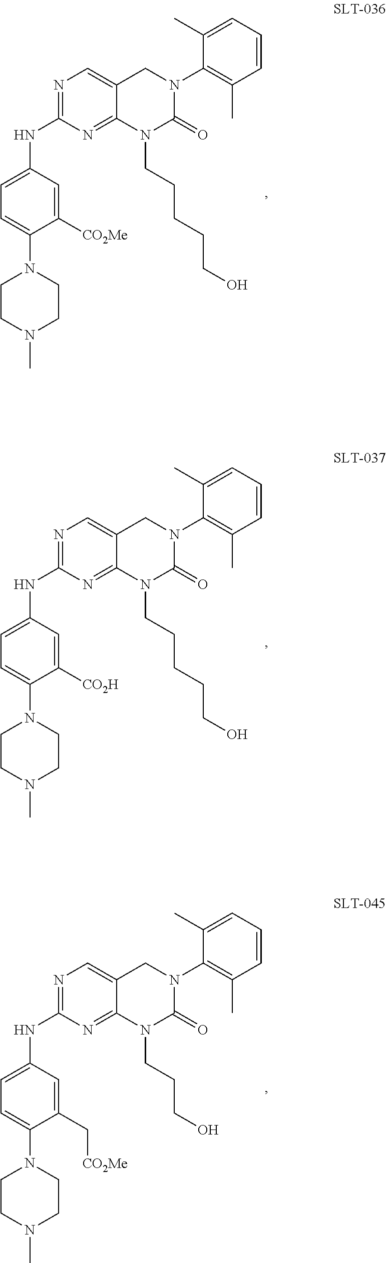 [SMILES: COC(=O)c1cc(Nc2ncc3c(n2)N(CCCCCO)C(=O)N(c2c(C)cccc2C)C3)ccc1N1CCN(C)CC1 ; Cc1cccc(C)c1N1Cc2cnc(Nc3ccc(N4CCN(C)CC4)c(C(=O)O)c3)nc2N(CCCCCO)C1=O ; COC(=O)Cc1cc(Nc2ncc3c(n2)N(CCCO)C(=O)N(c2c(C)cccc2C)C3)ccc1N1CCN(C)CC1]

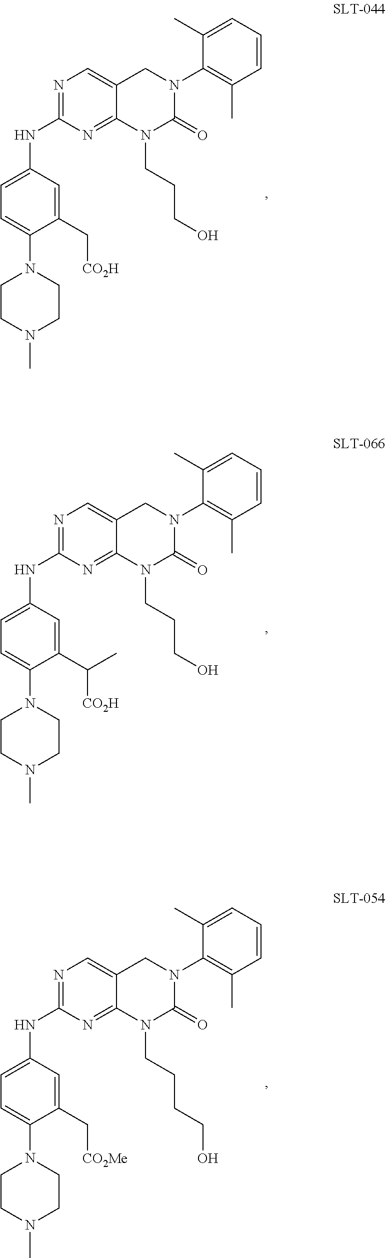 [SMILES: Cc1cccc(C)c1N1Cc2cnc(Nc3ccc(N4CCN(C)CC4)c(CC(=O)O)c3)nc2N(CCCO)C1=O ; Cc1cccc(C)c1N1Cc2cnc(Nc3ccc(N4CCN(C)CC4)c(C(C)C(=O)O)c3)nc2N(CCCO)C1=O ; COC(=O)Cc1cc(Nc2ncc3c(n2)N(CCCCO)C(=O)N(c2c(C)cccc2C)C3)ccc1N1CCN(C)CC1]

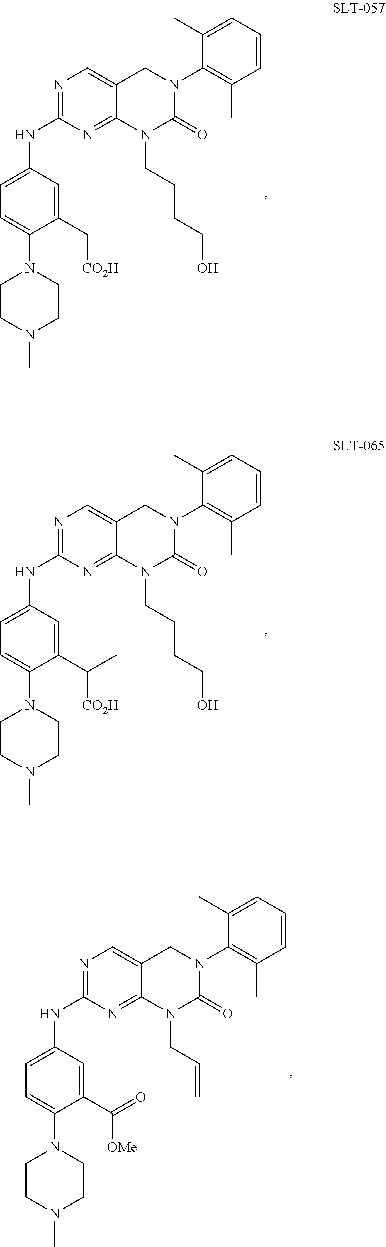 [SMILES: Cc1cccc(C)c1N1Cc2cnc(Nc3ccc(N4CCN(C)CC4)c(CC(=O)O)c3)nc2N(CCCCO)C1=O ; Cc1cccc(C)c1N1Cc2cnc(Nc3ccc(N4CCN(C)CC4)c(C(C)C(=O)O)c3)nc2N(CCCCO)C1=O ; C=CCN1C(=O)N(c2c(C)cccc2C)Cc2cnc(Nc3ccc(N4CCN(C)CC4)c(C(=O)OC)c3)nc21]

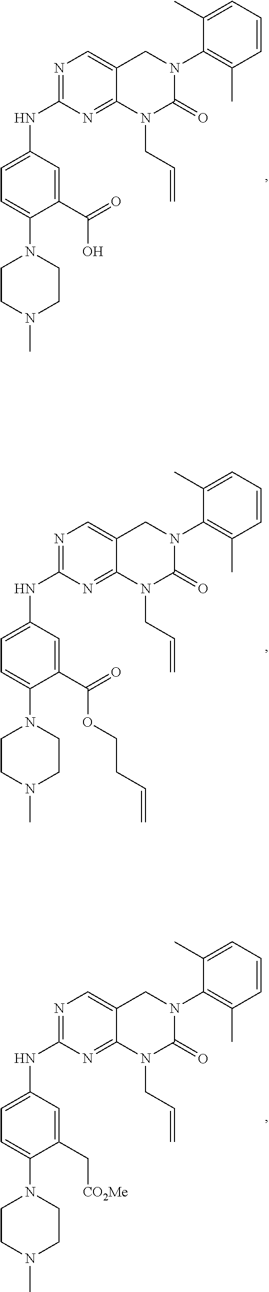 [SMILES: C=CCN1C(=O)N(c2c(C)cccc2C)Cc2cnc(Nc3ccc(N4CCN(C)CC4)c(C(=O)O)c3)nc21 ; C=CCCOC(=O)c1cc(Nc2ncc3c(n2)N(CC=C)C(=O)N(c2c(C)cccc2C)C3)ccc1N1CCN(C)CC1 ; C=CCN1C(=O)N(c2c(C)cccc2C)Cc2cnc(Nc3ccc(N4CCN(C)CC4)c(CC(=O)OC)c3)nc21]

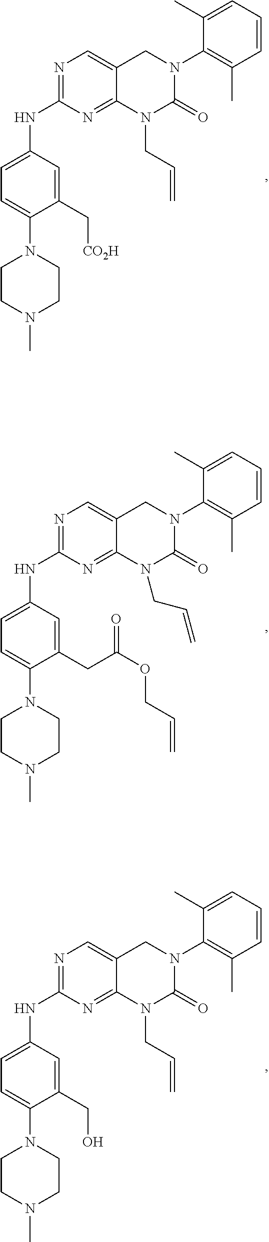 [SMILES: C=CCN1C(=O)N(c2c(C)cccc2C)Cc2cnc(Nc3ccc(N4CCN(C)CC4)c(CC(=O)O)c3)nc21 ; C=CCOC(=O)Cc1cc(Nc2ncc3c(n2)N(CC=C)C(=O)N(c2c(C)cccc2C)C3)ccc1N1CCN(C)CC1 ; C=CCN1C(=O)N(c2c(C)cccc2C)Cc2cnc(Nc3ccc(N4CCN(C)CC4)c(CO)c3)nc21]

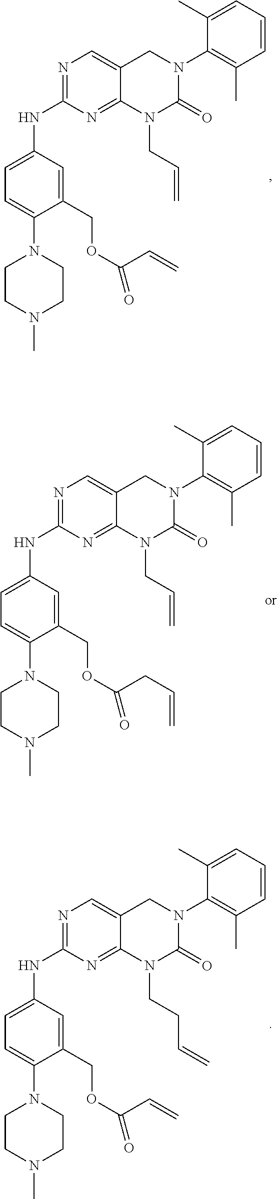 [SMILES: C=CCN1C(=O)N(c2c(C)cccc2C)Cc2cnc(Nc3ccc(N4CCN(C)CC4)c(COC(=O)C=C)c3)nc21 ; C=CCC(=O)OCc1cc(Nc2ncc3c(n2)N(CC=C)C(=O)N(c2c(C)cccc2C)C3)ccc1N1CCN(C)CC1 ; C=CCCN1C(=O)N(c2c(C)cccc2C)Cc2cnc(Nc3ccc(N4CCN(C)CC4)c(COC(=O)C=C)c3)nc21]

43. The method of any one of claims 31 to 42, further comprising treating the compounds of (III) or (IV) with a coupling reagent.

44. The method of any one of claims 31 to 43, further comprising heat-treating.

45. A pharmaceutical composition comprising a compound of any one of claims 1 to 30.

46. A method for treating a subject suffering from or susceptible to a skin-related disorder or disease, comprising
administering to the subject an effective amount of a compound of any one of claims 1 to 30 or composition of claim 45.

47. The method of claim 46, wherein the subject is identified as suffering from a skin-related disorder or disease and the compound or composition in administered to the identified subject.

48. A method for treating a subject suffering from or susceptible to rosacea, comprising, administering to the subject an effective amount of a compound of any one of claims 1 to 30 or composition of claim 45.

49. The method of claim 48, wherein the subject is identified as suffering from rosacea and the compound or composition is administered to the identified subject.

50. The method of any one of claims 48 to 49 wherein the subject is suffering from erythematotelangiectatic rosacea (subtype 1), papulopustular rosacea (subtype 2), phymatous rosacea (subtype 3) and/or ocular rosacea (subtype 4).

51. The method of claim 48, wherein the subject has been identified as suffering from erythematotelangiectatic rosacea (subtype 1), papulopustular rosacea (subtype 2), phymatous rosacea (subtype 3) and/or ocular rosacea (subtype 4) and the compound or composition in administered to the identified subject.

52. A method of increasing pigmentation in a tissue of a subject, said method comprising administering to the subject a compound of any one of claims 1 to 30 or composition of claim 45, in an amount sufficient to increase melanin production, thereby increasing pigmentation in the tissue of the subject.

53. The method of claim 52, wherein the tissue of the subject is skin or hair.

54. A method of increasing cellular DNA stability or repair in the skin tissue of a subject in need thereof, comprising administering to the subject a compound of any one of claims 1 to 30 or composition of claim 45, in an amount sufficient to decrease apoptosis and/or thymine dimer formation in the cellular DNA of the skin tissue, thereby increasing cellular DNA stability or repair in the skin tissue of the subject.
